SEQUENCE LISTING (84 sequences)

SEQ ID NO: 1 - protein, 234 aa, Homo sapiens
MGKWHVGGRRGSPRQWGATARGRDLEAVRRGGCGSVGRRRQRRRRRRRRMRRMRRMWATQGLAVRVALSVLPGSRALRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASAPTDL

SEQ ID NO: 2 - DNA, 771 bp, Homo sapiens
atgaggaggatgaggaggatgtgggccacgcaggggctggcggtgcgcgtggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcaccgaccgatttgtagtctgctcaatctctgttgcacctgagggggaaaaaacagttcaactgcttactcccaaaacagcctttttgtaatttattttttaagtgggctcctgacaatactgtatcagatgtgaagcctggagctttcctgatgatgctggccctacagtacccccatgaggggattcccttccttctgttgctggtgtactctaggacttcaaagtgt

SEQ ID NO: 3 - protein, 187 aa, Homo sapiens
MRRMRRMWATQGLAVALALSVLPGSRALRPGDCEVCISYLGRFYQDLVEGFRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASARTDL

SEQ ID NO: 4 - DNA, 1260 bp, Homo sapiens
  modified_base (1009): a, c, t, g, other or unknown
cggtcgtccttggtggaagtaaccatgaactggcatctccccctcttcctcttggcctctgtgacgctgccttccatctgctcccacttcaatcctctgtctctcgaggaactaggctccaacacggggatccaggttttcaatcagattgtgaagtcgaggcctcatgacaacatcgtgatctctccccatgggattgcgtcggtcctggggatgcttcagctgggggcggacggcaggaccaagaagcagctcgccatggtgatgagatacggcgtaaatggagttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaacgaaaccagggatatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtgggtcgacaagtgcccccaatgatttatggtacaacttcattgaactgccctaccacggggaaagcatcagcatgctgattgcactgccgactgagagctccactccgctgtctgccatcatcccacacatcagcaccaagaccatagacagctggatgagcatcatggtgcccaagagggtgcaggtgatcctgcccaagttcacagctgtagcacaaacagatttgaaggagccgctgaaagttcttggcattactgacatgtttgattcatcaaaggcaaattttgcaaaaataacanggtcagaaaacctccatgtttctcatatcttgcaaaaagcaaaaattgaagtcagtgaagatggaaccaaagcttcagcagcaacaactgcaattctcattgcaagatcatcgcctccctggtttatagtagacagaccttttctgtttttcatccgacataatcctacaggtgctgtgttattcatggggcagataaacaaaccctgaagagtatacaaaagaaaccatgcaaagcaacgactactttgc

SEQ ID NO: 5 - protein, 397 aa, Homo sapiens
  MOD_RES (329): Variable amino acid
MNWHLPLFLLASVTLPSICSHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITXSENLHVSHILQKAKIEVSEDGTKASAATTAILIARSSPPWFIVDRPFLFFIRHNPTGAVLFMGQINKP

SEQ ID NO: 6 - DNA, 1142 bp, Homo sapiens
gccgccagcggctttctcggacgccttgcccagcgggccgcccgaccccctgcaccatggaccccgctcgccccctggggctgtcgattctgctgcttttcctgacggaggctgcactgggcgatgctgctcaggagccaacaggaaataacgcggagatctgtctcctgcccctagactacggaccctgccgggccctacttctccgttactactacgacaggtacacgcagagctgccgccagttcctgtacgggggctgcgagggcaacgccaacaatttctacacctgggaggcttgcgacgatgcttgctggaggatagaaaaagttcccaaagtttgccggctgcaagtgagtgtggacgaccagtgtgaggggtccacagaaaagtatttctttaatctaagttccatgacatgtgaaaaattcttttccggtgggtgtcaccggaaccggattgagaacaggtttccagatgaagctacttgtatgggcttctgcgcaccaaagaaaattccatcattttgctacagtccaaaagatgagggactgtgctctgccaatgtgactcgctattattttaatccaagatacagaacctgtgatgctttcacctatactggctgtggagggaatgacaataactttgttagcagggaggattgcaaacgtgcatgtgcaaaagctttgaaaaagaaaaagaagatgccaaagcttcgctttgccagtagaatccggaaaattcggaagaagcaattttaaacattcttaatatgtcatcttgtttgtctttatggcttatttgcctttatggttgtatctgaagaataatatgacagcatgaggaaacaaatcattggtgatttattcaccagtttttattaatacaagtcactttttcaaaaatttggatttttttatatataactagctgctattcaaatgtgagtctaccatttttaatttatggttcaactgtttgtgagacgaattcttgcaatgcataagatataaaagcaaatatgactcactcatttcttggggtcgtattcctgatttcagaagaggatcataactgaaacaacataagacaatataatcatgtgcttttaacatatttgagaataaaaaggactagcc

SEQ ID NO: 7 - protein, 235 aa, Homo sapiens
MDPARPLGLSILLLFLTEAALGDAAQEPTGNNAEICLLPLDYGPCRALLLRYYYDRYTQSCRQFLYGGCEGNANNFYTWEACDDACWRIEKVPKVCRLQVSVDDQCEGSTEKYFFNLSSMTCEKFFSGGCHRNRIENRFPDEATCMGFCAPKKIPSFCYSPKDEGLCSANVTRYYFNPRYRTCDAFTYTGCGGNDNNFVSREDCKRACAKALKKKKKMPKLRFASRIRKIRKKQF

SEQ ID NO: 8 - DNA, 2366 bp, Homo sapiens
cggtcgcgtgtgctaagcgaggagtccgagtgtgtgagcttgagagccgcgcgctagagcgacccggcgagggatggcggccaccgggaccgcggccgccgcagccacgggcaggctcctgcttctgctgctggtggggctcacggcgcctgccttggcgctggccggctacatcgaggctcttgcagccaatgccggaacaggatttgctgttgctgagcctcaaatcgcaatgttttgtgggaagttaaatatgcatgtgaacattcagactgggaaatgggaacctgatccaacaggcaccaagagctgctttgaaacaaaagaagaagttcttcagtactgtcaggagatgtatccagagctacagatcacaaatgtgatggaggcaaaccagcgggttagtattgacaactggtgccggagggacaaaaagcaatgcaagagtcgctttgttacacctttcaagtgtctcgtgggtgaatttgtaagtgatgtcctgctagttccagaaaagtgccagtttttccacaaagagcggatggaggtgtgtgagaatcaccagcactggcacacggtagtcaaagaggcatgtctgactcagggaatgaccttatatagctacggcatgctgctcccatgtggggtagaccagttccatggcactgaatatgtgtgctgccctcagacaaagattattggatctgtgtcaaaagaagaggaagaggaagatgaagaggaagaggaagaggaagatgaagaggaagactatgatgtttataaaagtgaatttcctactgaagcagatctggaagacttcacagaagcagctgtggatgaggatgatgaggatgaggaagaaggggaggaagtggtggaggaccgagattactactatgacaccttcaaaggagatgactacaatgaggagaatcctactgaacccggcagcgacggcaccatgtcagacaaggaaattactcatgatgtcaaagctgtctgctcccaggaggcgatgacggggccctgccgggccgtgatgcctcgttggtacttcgacctctccaagggaaagtgcgtgcgctttatatatggtggctgcggcggcaacaggaacaattttgagtctgaggattattgtatggctgtgtgtaaagcgatgattcctccaactcctctgccaaccaatgatgttgatgtgtatttcgagacctctgcagatgataatgagcatgctcgcttccagaaggctaaggagcagctggagattcggcaccgcaaccgaatggacagggtaaagaaggaatgggaagaggcagagcttcaagctaagaacctccccaaagcagagaggcagactctgattcagcacttccaagccatggttaaagctttagagaaggaagcagccagtgagaagcagcagctggtggagacccacctggcccgagtggaagctatgctgaatgaccgccgtcggatggctctggagaactacctggctgccttgcagtctgacccgccacggcctcatcgcattctccaggccttacggcgttatgtccgtgctgagaacaaagatcgcttacataccatccgtcattaccagcatgtgttggctgttgacccagaaaaggcggcccagatgaaatcccaggtgatgacacatctccacgtgattgaagaaaggaggaaccaaagcctctctctgctctacaaagtaccttatgtagcccaagaaattcaagaggaaattgatgagctccttcaggagcagcgtgcagatatggaccagttcactgcctcaatctcagagacccctgtggacgtccgggtgagctctgaggagagtgaggagatcccaccgttccaccccttccaccccttcccagccctacctgagaacgaagacactcagccggagttgtaccacccaatgaaaaaaggatctggagtgggagagcaggatgggggactgatcggtgccgaagagaaagtgattaacagtaagaataaagtggatgaaaacatggtcattgacgagactctggatgttaaggaaatgattttcaatgccgagagagttggaggcctcgaggaagagcgggaatccgtgggcccactgcgggaggacttcagtctgagtagcagtgctctcattggcctgctggtcatcgcagtggccattgccacggtcatcgtcatcagcctggtgatgctgaggaagaggcagtatggcaccatcagccacgggatcgtggaggttgatccaatgctcaccccagaagagcgtcacctgaacaagatgcagaaccatggctatgagaaccccacctacaaatacctggagcagatgcagatttagg

SEQ ID NO: 9 - protein, 763 aa, Homo sapiens
MAATGTAAAAATGRLLLLLLVGLTAPALALAGYIEALAANAGTGFAVAEPQIAMFCGKLNMHVNIQTGKWEPDPTGTKSCFETKEEVLQYCQEMYPELQITNVMEANQRVSIDNWCRRDKKQCKSRFVTPFKCLVGEFVSDVLLVPEKCQFFHKERMEVCENHQHWHTVVKEACLTQGMTLYSYGMLLPCGVDQFHGTEYVCCPQTKIIGSVSKEEEEEDEEEEEEEDEEEDYDVYKSEFPTEADLEDFTEAAVDEDDEDEEEGEEVVEDRDYYYDTFKGDDYNEENPTEPGSDGTMSDKEITHDVKAVCSQEAMTGPCRAVMPRWYFDLSKGKCVRFIYGGCGGNRNNFESEDYCMAVCKAMIPPTPLPTNDVDVYFETSADDNEHARFQKAKEQLEIRHRNRMDRVKKEWEEAELQAKNLPKAERQTLIQHFQAMVKALEKEAASEKQQLVETHLARVEAMLNDRRRMALENYLAALQSDPPRPHRILQALRRYVRAENKDRLHTIRHYQHVLAVDPEKAAQMKSQVMTHLHVIEERRNQSLSLLYKVPYVAQEIQEEIDELLQEQRADMDQFTASISETPVDVRVSSEESEEIPPFHPFHPFPALPENEDTQPELYHPMKKGSGVGEQDGGLIGAEEKVINSKNKVDENMVIDETLDVKEMIFNAERVGGLEEERESVGPLREDFSLSSSALIGLLVIAVAIATVIVISLVMLRKRQYGTISHGIVEVDPMLTPEERHLNKMQNHGYENPTYKYLEQMQI

SEQ ID NO: 10 - DNA, 8 bp, Artificial Sequence
  Description of Artificial Sequence Illustrative CDEI motif
gtcacatg

SEQ ID NO: 11 - DNA, 301692 bp, Homo sapiens
gatcatgattggtaaatctgtagtgactaattgcccactgctgcctattccatctgacctaaattcctcaggtcttctaacattaagacctcttcctggccggttgtgctggctcatgcctgtaattccaacactttaggcagctgaggcaggcagatcacttgaggtcaagagttcaagaccagcctggccaacatggtgaagccctgtctgtactaaaaaatacaaaaattagccggacctggtggtgcgtgcctgtaatcccagctactcgggaggctgagtcaggagaatcacttgaacccggggcagcgggggaggctgcagtgagtggagatcaaaccaccgcactccagcccaggtgacagagcaagagtcagtctcaaaaaaaaaaaaacaaaaaaaaaaacctcttcctatagctaactcccacttaccacccccatcatgaacactcttgatgtatttacatggtttctccttcgaacatcctcctttcttctttcttaatggttgttatcaaataccctgataaaaaacaaaaacaaaaaacctcctctgaaggtcccttattcacccttccaacgctacaggtctgtaactctcattttctttttaaaaaatttttatttttttaatttattttattttttttttcagacggagtcttgctctgtcgcccaggctggagtgcagtgacacgatctcggctcactgcaacctccacttcccaggttcaagcaattctcctgcctcagcctcctgagttgctgggattacaggtgcctaccaccacacctgacctcaagtaacccacccacctcgacctcccaaagtgctgagaatacaggtgtgagccatcatgcctggccaaaatttttaaattttaaaaaatatattttattttttgtagagacagggtctcattttgagcccaaactggtcttgaactcctaggctcaagtgatcctcctgccttggcctcccaaaatgctgggattataggcacaagccaccaggcctgatccttacttttcttctgatgaattcacatatatgtgcacaaatactttatactaaattgtatttactgatgtacttttttcactgtgccttttctttttcttgcccagatatttttctcatataaacattagctccttaatgggagcaaatgaaccagtttttttttaattcccacccaaagtgagaatataaaaattttttattgatccaccaatactgaacactttcatttctaatagttatatttaactgaataaattacacacgggacaaaaatgttatttaagggataaagttgggtgtttgctcagggacaacgttgtatattgaatgatttggtgcttttgtgaatttatcattcaaaagaccatcgtgatggctaaataacagaaaggagagctttattggcaatatcaatttgcaaacccggaagacatagtcttcggtgtatgctgaatgtggtctctcttcaaaagagaggaaggacagttgggtttcatgcctcacagggtctgtttcacacagtggagtcatacatattcagcaggtttggaggaaaagatatacatatttatgaggggagctgagtgcatgtgcaatgggtaaatatgtatgtgacatcccatgtacactttggggcagggttttagtgttaaaatgaggtaaaatttggctctttacatcaaaaggtgaactacaggacccaaagacagtttgtgcacagcctctaataaactggctgacactggcttaaggtctgcaattgcttatcagaaaagaatgtttgtaaggctggtcctcattccaattagagttgtagtggtctgggttgtaaatcacaggatggggctgatagttcctattattagggagtttagagccatagaaattgagaaattggtcatgccagccagtccccgaaccctaaccctgtaggtaactttgtttccttaaccttacagtccatcttaggtgataaaggggtgtctgttttggtatctcacatcacaaattgttggttggtttgtgtgtttgtttcatcattcaggatgttgtttctttagggaatgtgaacctgaattctcaaggcttgttagactgtaatgttcccattcattttaggtttagctcatgcttctctagccacagccttcacttggattttaaaagttgaattactcatcaaagtctctaggacacgaaagacaatccttaggtatgatttgaccagtaaaaaagagatccagctgccttgaagcataagatcccctcggctccaatgtctatcactaatattcagtgtggcaaggatcccaggccacagagctgtggcttcctgcagctgctctggggagtgactctcttggagcatgtgatgtggtcttccattgtgcaggaccagcccagtggcatcctttcaacacctctggcaagcagcctttccaagcacgggtgccgtctgaaaacaggaggcatatctttcacatcctaggcacacgccctagggagtggtcagggttttgtccagttctcagcaaactagctacagctccatcccttactcccacactcaagagagatactagaatacaactgagagtagcctgatatgatgctaacctcgagttgcttttatttaaattaaaataaatcaaccagacacagtggctcatgcctataatccctgcattttaggagatcaaggagggtggatcacgagctcaggagtttaagaccaacctggataacatggcaagaccccatctctacaaaaagtacacaaattagctggacatggtagtgcgcacctgtagccccagctactctggaggctgaggtgaaggatcacttgagcccaggaggtagaggttgcagtgagctgagattgtgccactgctaataattaattaaataattaattaaattaataaatcgtgccactttattaaataaataaaacaagagtaaatcactcacaaatttggagcttttattagcaaaacattacttaggaaatctaaataaataacacggggttgacagccattgttctaactggcagcccctggcaagctcaaagccaggattatgctggtcacttaagtgacagctattgcgaattgttgttctctcaagaaaaaagaaccgatttctatggtaaaccaggcactgtgctgggtgcctttacaattcatcaccacaccacctaatgaaaggagcattcttcagaaactgtagtgctcaggctttctcaaggcctgagttcttttccaccagagcatattgttgccctattatccaaagttctctaaggaagagaactgacgtaagacccacatggctccattacatcttctggctacttgattgattttcatactccctacctctggggttggtatgtactatctatttctttctcctctcgttcttcctttttattccataaaatacaggaatattcctgtacattagtccttgcagcaaccttggaattactacattcctcaaacaagttatggaagccagctgccaatattggtccctggttaaacagtgaattctgttgttccatagagttactactgaaatacctaagccattttgtaaaatataatttagttgatctgaaggctgtctctaaagcagttttatgtagtgattacagagaaggactaatttcaagagtattttattgtttaaaaaaatgtaaacattttatggatgcactagtgaagtaaagaccaataaatgaagcagtaactttaataaaagggtaagtaaaatgtcacatcctctgcctatattcaggtctgttaggtatgtgtagttaaatgtaggtaagttagttgataattatttatttaagcatttctttatgtctactcattaaaaagaaaaaaagattaaaagaatgttactatgtgaaaaactgcccatcactggggaaaagaattttattatgcaaagcttcaacgctatttacagtttagacttttgtagctattgaaggctgacattgagataaagaagttaatcatgtccttctgtcttggaggaggtagaaagagatgagaatgaatacaattcaggatctacttctggtctttgatgaggagttagcacacggttctgggaggaaagacaggttaagaggcatgtgaaactctcaaatacgtcactgcgtctgccaacgtacatgatacccagcaagctcacatcttcatggaaagcatggtaattcccaacactaccggaagtctggagtggctaagtaatccatatattcaaccaggaagcagctaaagaaatattctaattacctaggaaggtttctgatttcaaaaggacatgaataaaaagtagaaggaatccactcccaaggacggacatcagagtagcttaaaatgtgagaataattttaggggaattttagaggtttggttatagacttatgttcccccaaaattcatatgttgaagccctaacccccagtaccttagaacatgactgtatttgggtagggcctttgaagagctaattaaattaaggccactggcgtgggccctaatataatctggctggtattcttgtaagaggaggagattaggacacacagaaataccagaggtacctgtgcagaggaaagaacgtgtgaggacttagcaagggtgcagccatctgcaagccaaggagacctctgaggattccaatcctatctgcatcttgatcttagacttttctggaactgtgagaaaataaatttcttggtttaagccacccagtctgtgatattttgttatggcagctctagtaaactaatacagattttaaatgtcattaaatgtcaatgtttaagctttgacaaaattttctaaaggaaagtataaaaggtcattttctttcttttcagagcctgatgattgcgggaggggtaagccagctgcatggggatcatgatgcaatgctgatgcaggacagacagaaagtagatctcttccatttctattttttttttttctgttgagttgaatgatcttcagactgaaaatgaaagaaaggtcactggaaataaaggccaaagatgagtgacaggattatagaataagtcttagctgttctaaagaaggacatattatgtacccccacccccaaattcatatgttgaagtcctaacccgacagtgtctcaaaatgtgaccatatttggagatagggtcaaagatgtaattaaggttaaatgaggtcattagcatggatcctaacccaatatctgctgtccttataacaagaggagattagggcacagtaagacacagagggaagaccatgtgagaatacagggagaaggtggccatctgcaagccaaggagagaggcctcagaagtaaccaactcagccaacacctcgatttcagacttccagcctcctgaaatgtgaggaaatacatttctggtgtttgatccatccagtctatggtaagttatggcacccctgcagggttcatctggctcagacttaacgattgcttttggtgatatttatagggcacagataacagcctaaacacaagacgacagaaacgcggcccagcagactatgcataaaatagaaatggggtatctggaccaattggagtctgcagtgggatgcggttactaaaacagtcaaatgcaacatgaggctccaggcagagtagtgggcaacatctcccatgttgcagcagtcagagcacacttcgagtactgtaaaaagacacagacaagccagaacacatttagagaatggccaaggtgtggaaggaaccagaaaccatgccattatgcaactgttgaaggaagtgcctgttttaccttgtgaagagaagactctagaggaagaagtagcatgaaaaccgctggcaaatttgtaaagatctgaagtgtggaaaagaattattctgcttggtcactggggatacaaggatatctgagtgggagtttaaaggcgggggatgtgagctttaaatgggataagaacattctagtaaccagaaatgcccaaagatagaatgcacagtctggagagccagtgaatatctcacaaatggagacacttgaaactaggatggggatgctgttgtaggaattccagcagacaagtggttgttggttccttccccaactttgtagggttataactagggatgttcctgcgttttctgcttggaggatctgcaagacacctcagggcaggaaatggcattaaatgcagaacagagctagtggctgaaaagcaaaaagccatcaggatctctgagtagtgaaggaaccagagaacatgcaggcaatgtccatcattctgacgcaatcagcagcataatcatcttcccccaggaacatcttgaccagggaatgtgtcagtgtggtgaatttcaacagtggaaagagaaactgctaaatctaagaactttaatttttatagattatgatctcatctctacaattttgaatttcatgctcaataaaagttccttactctctttttttttttttgagacggagtctcgctctgtcgcccaggctggagtgcagtggcgcgatctcggctcacttcaagctcagcctcccgggttcacgccattctcctgcctcagcctccccagtagctgggactacaggcgcccgccacgacgcccggctaattttttgtatttttagtagagacggggtttcaccgtgttagccaggatggtgttgatctcctgacctcgtgatccgcccgcctcagcctcccaaagaaaagtccctcactcttaaagttgcctcctccttcccagggctggcttcatgggcatgcaaccctggagagtctcacaggccctgcggtgggaggagccccatgcttggtttaacgctctgccattgccatcttaaaattcttaatttaattttttttcttttttttgaggtggagtctcgctctgtcgcccaggctggagtgcaatggcacaatcttggctcactgcaacctccgcctcccaggttcaagcgattctcctgcctcagcctctggagtagctgggattacaggcaggagtaaccacgctcggctaatttttgcatttttagtagagatgggggtttcaccatgttggccaggctggtctagaactcctgacctcaggtgatctgcccacctgggcctcctaaagtgctgggattacaggcatgagccaccaggcccggccttaaaattcttaataatgtaacaaagggtctcacgtttgcattttgcagtggactctgcaagatttgtagctttggaccacgtttctctttgcattcagataccttcttttttgccttatttgctcatgcagacccggaacaaatacggaattgcggtgggtaaatgtggtgcagaaagtgaacaactgggtttgtcctgtcactttaggcttttccctgctgtcccagcttcatgtcacttacttgctattagatttgggagttcattagcttcattttcctgatgtataaataggaataatagtaacagcctctttggcttttgtaggaagtaaatgacatgaagcgtataaacaaatactgcatgacaataaatatttgtccttatttgttgaggacatccaaaggacattcaggggcaaaagtaatccaagagtcaagactgaatgcctagtgcgggaaaagacacacaagacaacatttaggggagctggtacagaaatgacttcccaggaaggaagtctgtaccccgctggctgagccatccttcccgggcctaggcacccttgtcagcgcaatgagcaagggagagaaggcaggctgcagtgcagccctcagaagggccagagcactccctggcttcagtccttcgctccaagccctgtgtggagtgggctgtggcttggtaactaaatgctacttcaggtcaagagcaggggatatatctgggcagttctagagcattctaaactatctggacactaactggacagtggacggtttgtgtttaatccaggagaaagtggcatggcagaaggttcatttctataattcaggacagacacaatgaagaacaagggcagcgtttgaggtcagaagtcctcatttacgggggtcgaatacgaatgatctctcctaatttttccttcttccccaactcagatggatgttacatccctgcttaacaacaaaaaaagaccccccgccccgcaaaatccacactgaccaccccctttaacaaaacaaaaccaaaaacaaacaaaaatataagaaagaaacaaaacccaagcccagaaccctgctttcaagaagaagtaaatgggttggccgcttctttgccaggtcctgcgccttgctcctttggttcgttctaaagatagaaattccaggttgctcgtgcctgcttttgacgttgggggttaaaaaatgaggttttgctgtctcaacaagcaaagaaaatcctatttcctttaagcttcactcgttctcattctcttccagaaacgcctgccccacctctccaaaccgagagaaaaaacgaaatgcggataaaaacgcaccctagcagcagtcctttatacgacacccccgggaggcctgcggggtcggatgattcaagctcacggggacgagcaggagcgctctcgacttttctagagcctcagcgtcctaggactcacctttccctgatcctgcaccgtccctctcctggccccagactctccctcccactgttcacgaagcccaggtgggccgtcggccggggagcggagggggcgcgtggggtgcaggcggcgccaagggcgcgtgcacctgtgggcgcggggcgcgagggcccctcccggcgcgagcgggcgcagttccccggcggcgccgctaggggtctctctcgggtgccgagcggggtgggccggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtgggtgccgcgcctcggaaggcggggggaggctgcacggtggggacgcgataccccccaagaccttaacccaagtctttaatgcagagaagccgggggtccgtcaatgggacccctctcctctccgcccccgcttgcggacgtccagcgcatccccgctttcggcccagccctgccccagggagtcgcgctccggcccgctgagagggagcgggcgaggcgctggtctccctggttccgcgccagcccggggcgagaagggtagggggcgaccctgagcccagaccccgacttagtccctgccttggaagcgggggtcgggggaggcgagagacattcagacaggggggaagggggaaggagcacgtggggaaaaccgaaaacgcagcgtccctaaagccagtccttcgctcttgaatcgttgccctttcctttctcttgggccctggggaggaggaggaggagtcgggacagcccaggaagctaggagcccctcgcttttccctgcgttctccgcctcgtttcaccttcctctctcacccccatcccccttccttgctccaacctcttttcgcggctgctggcggcggcagcctgggtgcgaccctagcccgagccggcgtcggggtcgcgcaccgccttccacaggcaaactttgtgcatgtccgcgtctcccttttgtgtaacttgcgagaaatgggaggggtcggagacccatagccaccccgcgccttccccaagtctggacgtcccgggagttcgctcgcaccctaagctctgtctgagaggcagaaggtccgcgggaacaaaagccgcgacaccccgccctcgccgtgggccgagccgcgttagtctcagtgcgccaccgaggctcccgcccggctcccttcccggggctgcgcgtccgcctgggagggggcagagacgctgccggggcttctggcggagcctcggggcccctctgcttcccaccctcggatcacctccgaggcttaattctgtgctgggctgtaaagtccttggcggggaggaaataaggcggagagtgggatgtgcgtctggaaagggcagtggcggatggtggggaggcacgggctctgtcctgtgcatccaaggaggcgtttgtctgattaaggcgcggcctcttccctggcagctctggggactctggtttagttcccctgggggcacaggatgctggggagggtccgaagggtcttttttttagggtgcagataaaaggatcgaattgagtgaagattaagacggagaagatggcgcctctgcagtgcagcaaagaaaagctgtgtggaggctgcagcctagtgaaatccacccaccactaggtgtataacaagtcttccccattcatccaactctcgaacattcagctaacgcctgtgcacaatgactgtttttctttacatctgaagatgcttagaagttgtttgcatgacagttgggccagaggaggctgtatttcacttcggacttcggttgggttcctgtttggcatcttaacatttgcaaccttccttacccgtttttcccctcttttcccccagcctcctagtaattttccaaaagaaaaaaagcaatttccgggcagatgttttcctgtaattccactgaggctgtacagactatcacaccattagttttttagtttctaatttgcaaggtcattgaacttttttctcttctgatttttacccacaagtggctcaggaatcaccttagggctagttaagacaggttgttttaagagttaaagaatttcgttttccatgttttaaaaaaatgcacttaactaagagaattctgatttctctcatgacttacttacatttatttgtagaattattccagttttcccgaatggtcttactacagattaaaaaccatacatttaaggaattaaaagcatatgataaccgtatttttgaagcttttgtcaaaacttaaatatgtaactttgttgagcaatatttctttctgtataaaaagaggaagcatgtaggcagattcgaatttagtttttggtctgtttaattgcattaatatgctctttctgccatcttttgggaccttttcagtagctattgttaactcctttaaagggtggaggtgagattcacattatgccattttgcgaagtgttggtagaagggtaggtaattcagtctactgaggtttgagggtggtttatctggttctctctgtgtccactgaaaagatgtaattacgagttgactgtcttttatagagcttagaaaataggtctgctgcaggtggctgtagaaaagaaatgaataagggcaatgataatggggccttggctatcgggggaaacatggttattatctgggttgatgattttggctggtatgttgatagaggtgtgagtttaacaaaccctaatctatatgatgtttcactaccctaatatttatttatttatttatttttaaatctagcttcctcttaagttttatttccctctgttgagggtttaacctagtcacaatcatcttacctttgtggtagtgctaatttatagtttatggaaacctgctgctactgactagtacctcgctaaaggtagaagccgacacacagaatagtaaagctcaggtttgatggtgatctgtaaggagtcacggttcttgctaatcgatgagaaaccatgataactggcagactgcagaagcttttctcttgtcaccggaaccagcagaggctcctgaacgacatttgcaaggctattagtactgcggagaagttactagatggaataatactggtcctgactgaggaaatgaaaaggaacatgcaagttcacgaggttaaatgaggaatggctgattcaaaaaaattaaaaagaaataacgtgatcaacaatattggtttgaaggaatgggagaggttgtctttatgttaagtcttgttgctagaggatctctattttatgttactgttttttgcatactagagtcttctacagatatttgtatgaatgttgtcagtcattctggaaataagcatagctattacttgcatttcacgtggccactaaactatcagtgagaagtgcattaaattcagtagaaatgtacattgtttctgcaaattgtattaccttgagaatcttacaaagcatctttcattaatacatttcttttaaaatctctatcaattacaaatgtaaagataaacctgtacagatgcattttaaatttaaagcttccagaatgtaaggtttagttcgactgcatacctcattacctccccttccctcctccctccccttactcccaggaagtttgcagggtaaataaaacatgcacatgataataaaacaaagtggaatgtactaaaataccaaaaggaaaatacagattaggtatactgagtaaatatgttacagagaaaacttcctgtatttaaaaagtaaggaacaatgggatgagatcatagataatcctggagcatttcctggtagtaaatttccagccaggtttatatcccaggtctggggattggaggttaagtttctgcatatacaatatttgtttacattcctctctgcctctaccagacagacagacagacacacacacacacacacacacacaccctctgtctctttctctctctttctctctctctctctctctctctctctctctctcacttgagtgacagatgctggtgcagggtattctcaacaggagtagggaatttgaaataaggagttgagaggtcaaaggtaaggggctgaatgcaggaccagagagcttcagtagcatcttgatgggcagagtatgagatgaacagtggcaacgaggaggatagggaaaaatcacattttctgtttgtgttttgtttttgtctggtggttttcaacctctctctgtttcacttgactatgcttgattatgaaatcttttttttttctttttttaatttaaagatctcaactggctttgttgtgattctacattcaggcaacactattctgtaaaatagtataaatgttccgatgagttgaacagaggagattggctttgtagacagaaaagggctgaagaaaacagaaataaaaaaatcttttccttttcatagtgagagctcagatgcaaatgtatggagtggggatggatgggaaatgccgtcttttgcaaacccttggaaacgctgtcaccctgtgggtctcacagccagtgtacagataacaccctcatctgtacacagaagacagaaggtggcaggggaaggaatgagcacaccccagaatgagtagcaagtttaatcaataaaatatgtatttctttttttattgttgtataagaagaaaagcagacagaactcaaaccctgaagctgattttgcaagtgaaaagcttagtaaagggggaaatgttatgtaactattttttcagtttagtgtggatggcttaagaaataatcattactgtgaggacactagtatttttcttggaaaaaaaagtttggcaaattttcctctttagattaatcttggttatagattattattatgcttgagtggtacctcttcagaagtagaaatctggcaaaaactggagtacaatgattgctctaggaataaattcacatatttaaatttaaaagcctggatatgtgcatcaattaatagctttatgaaacagggatgcttagtagattagctagctgagtattcagagagggccacagaagtctcaggttcacatgtattaaatctgctgaaatagaaacctaagagatcattttatttaggtttattttgtgtggctacatgttgaagaaacatgcaatagagatttagcttgaaccttgcgagactcacagtatcttacgaaagaagatagttaaaatggggaagatgagaggtttttaaaaatgtcacctctgtagaacgaaccagagttgttctcaatttataatgttgaatttgatgccaggcgtgatgttaggccaataacactgagtcattgagtgtttctcaggtatgtagagaaggcagcattgcacactaatcaggagagtttagaccagggcaggaatgcatgggcataaattctgtctttgtttcttttgagctagtgaccatgggcaagtttctgtgcctcagttttttcctttgtaaaatgggatgagtacttctagttattgaggatcgaattaatatctttgttaaatgcttgaaacagtgtgtgagttaatatatatgtaacctgatggcatagcataagcactgtgtgttagttgctgctatgtatggtgatgatgttgatgtacaatgggtatatgtaagtttcctgcatagttgaaagttaaaaatctagatgatgtttcacttattgacaaattacttattggccaattaagactattgcaggaaattgacagattgttgtctatgagtttgatctaaatttaaaaagcacatggtatctcagatacaatttttagaatacatgttataaaatttcatttattaaatatgtattgtgtatttcaattggttttttgaaaaatgaaaataattcatgctcattgaaaaatttaaaacagtacagaaatgtgcaaaaagcataagcattttttacacaaatgggaaatacctagtttttttttttctttttttccacataacatggatgtagaccttttttttttttttttgagacagaatctccctctgtcacccaggctggagtgcagtggcgcgatctcggctcactgcaacctccgcctcctgggttcaagcgattctcctgcctcagcctcctgagtagctgggactatgggcactcgccaccatgcccagctaatttttgtattttttaagtagagatggggtttcaccatgttggccaggctggtctcaaactcctgacctcaagtgatctgcccacctcggcctcccagagacctattttttaatgttgtacacactggcagcttgccacagatgtctaatattgagcaagtttatacactggcttgattattaactattagttggctatcagctatgtgcaaggaactgtgaacagacttggtttagttttcagtagaatctttgctagcaaaacgaataaacaggctttgccaatctgggtagaaatggaagatttcaaatgccagcaaaagaattttggtcctaatatggaagcttgaagttatgttcacccaaggacagggaaaccaaaaagcagattgcttgagaatgcggcatttctctaggaaatcagtttcaaaataaaatgaattttttgttttgttttgtttttgagacggagtctcgccctgtcgcccaagctggaatgcagtggtgcgatctcagctcactgcaatctccgcctctcaggttcaagcgattctcctgcctcagcctcccgagtagctgggattacaggcgtgtgccactatgcccagctaatttttatatttttagtagagacggggtttcactatgttggtcaggctggtctcaaactcttgacctcgtgatccgcctgcctcggcctcccaaagtgctgcgattacaggcgtgagccaccgcgcccagctgcagtaaagtgaatttgtgtaaccagccatgttatggctgttacatctttaaaactttatagtgatctgctgtttaattgaaattatatattttttttaaaaatcagcatatgttgctcaaaaataaaaggtttttagcttcaaaagcaggccaaggatatttgaaagccttgagtgtatgaaactggtgatactgtggttaaatgatgatgaacgcagggaaaaatcaagaagcagagtcccaaagaattcacatgggtttttgttaaccagctcatgtaattataattgtcctgtccaactaaagacaagaagacaagaagtgggttcacatacgaggatgtgtaatgacagaagccatatttgtgataaacatcctgattacagtagatacagtgaggagtgattaagatggttccagaagcaggagtggagggttcatgagcagggatgaggaataaataggacaaacattttggaaaagggaaaagatgaataagcctgttttgtaaattaatggatcctgatgaatgaagtgcagtgattccttaaggtcttattaatgatggaccccaaacagtgtttgaatctcttaatttccagatggttcctttttttcttcacaaagtcagaaggttttcttatgagaaatttgattcaacaagaataattgtcacttcttttgttttttggtgatttttttttttttttttttttttttttttgagactgagttttactgtgtcacccaggctggagtgcagtggcgtgtcttggcttactgcaacctctgcctcccgggttcaagcaattctcatgcctcagcctcctgaatagctgggtttacaggcatgtgccaccacgcctggctaatttttgtttttgttttttttttttttagtagagacagggtttcactacattggccagggtggtctcgaactcctgacttcaagtgatccacccacctaggcctcccaaagtgctgggataacaggtgtgagccactgcacctggccaattttttggtgattttgataggaatttttttttttgcctgatgctttttttgagagaatctaataatttgtctcttatttaataatttggctttcattagcttccagaaagccacatactgtcttcattgaaaaatgtaaggcatcctagatgatacagactgataaattacttttcagccataaaagatacttatgagaataagagatctgttgcataaagaaaagtcatttgtgttaagtgaagaaatgcattattactcttgctgctttgctaaatttcaaacattgaataactaatatattttgaccccctggagatattttacaggaagaaaaatgagaggtttaatcctactgttgagatagacttaacgtgttttagacctgcccctcatcccaagttactacaaactcatttgtttagtttttccttgtctgtgtgctcgttatttcacagtaaaattagtgaggcaacataaattttatttttaagagaaagtaaagaaaatggctttatccattaaaggtttattcatcttaaaaaaggacttgttatatctactcctactattggatgaattttcaaaagcatattgatactgtgtttggttttgtttgttttcgggacaagtcctcactctgtcacccaggctgaagtgcagtggtgtgatctgtagccgtggcctcctgggctgaagccatcttcctgccccagcctccagagtagctgggactacaggcatgtgccaccacatctgactaatttcttaaatgttttgtagagatggggtccccactatgttgcccaggctgatcttgaactcctgggctcaagcgatcctcctgcctcgaccttccaaagtgctagattacaggtgtgagctattgtgcctggccaatgttgtttttataaatatcattactcagttattttgtttataattgaattgataggctcatagaataaagatggataagcactgtttcttaactgtatttattgatttttttatctttcaaaaaaaaaatcctcatctacttcaaagattaagcctttaaaaccgatgcaaagtttttgaagtcttagcttaagtcttcagagactaacattaaaacccatcatatgaaaagtgatatgtagtagcaaatattctccatcctgatgctctcaattgcttattatggtttaacacaaatataattttgtcattacagtaaattagtttgaggatataactgaagagtacctaagaaactcccatagaaatgtgaaaatatgcagactaattattctaagaataattaatcatagatacttacatattaaattgattagcaatttaaaattaaagccaaatttctgtggaaattagcttattttcatacacaaagaaacctagattcttaggggtacaaccaagaaagggccactccttcatttcacaaatattttcgaggtccttgcccccaagagggtagacagaataaaaaggaggaaggaagtgacttggaaaaaattaagttgggtaaggaatataagagaatgctacgtagaaggatgtgtgttgtgaatgcaatagtttctgtagtattgtcaaagttctctgatcatttcagtatgtgaaggtaaacttagataactgggggaaggaactatatatttactacattgtaaaagaataattgtagacagaagatctactttgggtgattaaatgttgaaaacaagtggtgggagagagaagttagtgttgattgtgatcacacacttttctatacagtaatttgtttgatcactcatgataatgccttacattttgtatgtactaaataaatgagttctctagaaattctatgttgtggtgataaagaattacgttcagattgaagattcgaacagctgtgcaaccttctcaacatttaagaaactgcgaacaagcatagaaagaagattaaagccttttaaataagattcttttttttcagcctctttttggaacattttattgtgaaaaggtttcataaatactgtcaatacatcacgttcagtcatttgcaattgtatttcaaaaattcagttggcctgcaaaaagaaaaaatgcctcatttttgaaatatttcatccatgccctgattgattgggacactttcaatagagggtgcctagacataattttgtgtgaagaagttagtagagtttggaaagaaaatgtgtattggaattaaatttagtggcatgaaaatgaaaatttctctgtcattgtctttttttttggctccttttttggccattttacagctagtgtttagtgtgacaaggatcaaaattggccctttaaaaaaatatattgaattctattacctctgttatctaaaagaattgtatttttcaatttgaagagcttgaagccaatactactatataccattaacttgacaggaattgaaaatctaattgaggtcattatgattagccttgtatcttgctagtattttctcaatcttattttgaatgttttgtgtcaagtgttaaatcctcttataccacagaatccagaaaatttaaatgcaacagaagtaaaacgaatataaaaattctatggttgttgtggaggggcgttcttggctaacccatagggtgtttgaatgtaaaggcagcttacagaactgctgctagttgaattgtggagggtttcacttttaagagtacatttgagacaaaattgcaattttggaatcacttattcctgcttgttcagtgaaataggagaggggggaattcctgagggaagctgggagatttccaggtgatgatagagtagccatgtgagtatttggctgaaacagagtagaagccacctctgtctgttggacagaggtggccttaacactgagtatcaatcagcatggccattagagctgtccaggacaaacataatgaatgttgtggtggcagagagggtatacttaaatgaacaaaattggaggctgactttccttgtttcttggtagttcctttcttcgtatttgctatttggatatttcacttcacttttgtgagctagactcaaaggttaaatttatatcattgtgaattttctgggtcatattgtagggacacagatgggaagcatcccttcagtttattgagagtatttcttatgcgcatgtgtttttaattgtatttattgatttaataaatagccgatgagatgagaaaaaaatattagtgtcttgttgttgcagagaaaaagacattggatgcattcttagaatttattatcacatgcgaagttatttagatttacaaagcaatctgacatgttgacttcattgctcgtttgctcatttctatcagaggttgaacacaaagccaagcctaagcccgtgattcttctgtgtgcagatgagggggaaggtttaatgcagaatgtttagctgaatatttggattgtgcattccccttttatgtctctactgcaagttttctcagggcatttgtgattgacttctgtgatgttgacatttatttggagttagatgtttaaaaaattactaatttatggcctggtgcggtggcttacacctgtaatcccagcactttgggaggccaaggcgggtggatcacgaggtcaggagatccagtccatcctggctaacatggtgaaaccctgtctccactaaaaatacaacaaattaaccgggcgtggtgacaggcacctgtagtcccagctactcgggaggctgaggcaggagaatggtgtgaacctgggaaggcagagcttgcagtgagccatcgtgccactgcactccagcctaggcaacagagcgagactccgtctcaaaaaaaaaaaaaatttactatcttattgtttctctcattttgaatttgtgaaattattattcattttatggacatactgcatggattctgtgtttcagttatgacaggagatctagaatctgggtggggctgtgagtaaatagaaaggtaactcagtctgtgccttgaggaattccgcagtccagtggaggaagaaagagacggagaaataattaggtacagtgtgccaaaggctataatagaggaggtacaaactcttcccagagaaggcaggcagaatttgaactaggtttcaaggatgtgtgaaggtgaactggggtggggagggcttcccagacagaaaaaacagcgagggtgtagagtttcaaagggggaagcccaggggtataataagtgaaggagacagtgtgaaattataaattttggatgcccttaatgagtttgaacttcattttcttagaaatggagtacttttaaatgttcctaaaatggggacgaggcattgatattcatggttcagttaggtttaggagcttttgtagaagtccttttcaaggtctgcttaatctcatgggccagatagtgacacctcgatgcaagggagcatgtaaatgtgctctattagctgggagcattgctatcagaaattacatttccggctctcttcttaagggtaaaggagaggtaggatattgagtggcaactggcattctgggatgtagaagaaatacagtaccataaagggatagaggaaaatgggtagacttaacgttctgaaccaaagctaagattctccgtgagcgattagagtaggcttttgggatttgcttaccttctcttatctgctagccagccacacaggaagcacatctttgtggagtgacagaggaggaggtaggaaatgctttctcatttttatggtagaaacatccaagagctgagccttcattctggctgagagaactctagatggttcagacttgcattcctgggaaggccttccagctagagtgatggaacaaagcaatgatttctgtggccctgtgtggacagcagtggcagtcagtttactctccctgttggttaactgtctttcgtttgatctagagacttcctcaaaacaattttgttgtgcttgttctttgaaagcaacagcagacaaaacaagctttgaggtttttagaaaagagcttgacactgagcacctcttttgagctagatctgtctgtcatgctgaacctgttttctgtggctgcgtagcttttcaaatctgtttatgcttgctggtgttattgaaacagaatgggaagatctatgcaaaaaggaacattgatcttaataaatttatgtcagtaagaatgggactgccattttaaaatttattttaattccttacctattttcttgttcaattattctaaaagtgattaaatgaagatacgtcacagaaagtatgctttgaaatcaagatgtggggaaaacctgtagttctgagtaggcctgatagaatcatcattgttagagtatttggaggttttgtagtagggattttgtcttcacttaaatatttagttgaaatatgttccaatagtttgacacattacagaatctgtctgaaagaaatgtctaaaatatgattcctcagaagctgctcttggttttactttctgctgttggttttactttctaacatttcattttgatcccgcagttttaagagtaatcaaaaaggccgggcacggtggctcacacctgtaatcccagcacttcgggagactgaggtgggtggatcacttgaggtcaggagttcaaggccagcctggccaacatggtgaaaccccgtctctacaaaaaatacaaaaatcagccgagcgtggtggtgcatgcctgtagtctcagttactcgggaggctgagacaggagaatcccttgaacccaggaggcggaggttgcagtgagccgagatcataccactgcattccagcctgggcaacagagccagactctgtctcaaaaaaaaagagtaatcaaaaagctacattatttaaatcttggcatgtctttaaaaaatgttaaactgtcaggaaaattatcacttctggataccaccttcagaaaaacacatgaaaatttgtgcatgtctcaaatagttgttaatttatgaaaaattagaaagagtgtttgaatgtatatttctgtcatattatattgctcatatttttaaattatactttttttacccgtgaagcccatatagttaagtttgaaaattttaattatattagaatttgaaaattaaataaatctcctttaattcatgcataattttctaatagtcatatagtttgtattatataatttgagaaacttggttctaaaaaaattgtctgccaaagaatgtatatttctgaattctgttctgctttcatacatctgaaaatatttgagctatcataactggctatgcttcaaacccagtctgaaatgggtgacctttgctggctttggattgcagagctgggggctgttgtttgctttatctgatttaaaatagtcagtagtttattttgcaggcagttagatgtatacctttgcataaaatcattgaactgaaaaggttggcactccaagaatacgttgtgagaaaagagaacttgagtactcactctcgactcacctgaatcttattaatattttattgattaatatctgttgtatttttaagtaaataagctctctagaaactatctctgcttctttgttcatttaacatatattaaacaaattgatacagattgcaaaaattagttgcaacgttcctagttagatatcaatgaaataaaaatggtctattcatacgagttcattttagttattcctactaatatcaaatgtgttttaaaaaatgggagattataatctttttatgaagtaagataaattattactttgtatatttaattcaaagaaaacaaaaaggccacaagtgcctgcataaaggcacatccacttaggattattgttaaggcctacagttgataattaggattcaggcatagcataaatgaagtggtatctttttttttttttttttttttgagacgaagtctcgctctgttgtccaggctggagtgcagtggcacggtctcggctcactgcaagctctgcctccctcctgggttcacgccattctcctccctcagcctcccgagtagctgggactacacgttccgccaccactcccagctacgtttttttgtatttttggtagagacagggtttcaccatgttagccaggatggtcttgatctcctgacctcatgatccacccgtctcggcctcccaaagtgctgggattacaggcatgagccaccgcgcccagccagatatcaattatttttagacccaatgatatttgctgttgatttattgttggtgcacaaaataagaaaacaaaagcatggttttaaaacagccctttacacatttaatatggaaatggtaacctacaggtctgtattccatacagattcctgggctcattttatctgtgaattcctctgggcgtcatgcatgcccaggactgactttaaagcagaattatccagtagaggtataataggaccgttagatctaatttaaagttttcaagtagccacataaaaaaataagcaggcgaaattaattttagtctgtcactaaattcaaaatgttatttcaacagggggtcaatataaaagctatttatgaaatacattattttttcatattatgtcttcaaaatgttcacatttcacatatttattttttattttattattactatttttttgagacggagtctcactcagtcggttgcccaggctagagtgcaatctagcttgctcattgcaatcttggctcattgcaacctccacctcctgggttcaagcagttttcctgcctcagcctcctgagtagctgggattacaggcgtgtgccatcacacccagctaatttttttatttttggtagggatgagatttcaccatgttggccaggctggtcttgaactcctgacctcaagtgacttgccgctttggcctcccaaagtgccgggattacagttgtgagccaccgcacctgattacatatttcacatattttaattcagaccagctgcatgtcaagtacttagtagccacagatggctagtggttactgtattggacagtgcagctctaggagagtctcttcaacctgcagctgtctaggaagagaatcatagtttctggaacaggtagaatctataagacctcctttttaagcttctccactttacagatgaagaaagtaatatctggagatgttgagttgttcaaaatcacccacattttaaggatcaagagcccagtccctgttctctaacttctaatctctttcttcatcactttttctgcttcatgttgctgcatcccctttagttactaaggtactaacgttaggttgttgctatcagttaaatattaagccaggagctttaagacaatctggcccacttgaaggcatggcctgatttggacaaaacttgtggggtaggatgaatagagttaccaggcatatcttcaacctctctgtggactagtattatacatatatgagatgatgtagggtccagtgatcagttcatctttttctattgtgagcactgttggaataggaatggggggaccttttgggacccaggatatgtgattctgaaccctgaaatgtcagatgcccactaaaaggaaggggagagatggacctgtcattaccttacacattttattgtctgtatgggtatgtaaacatgtacagattataaggaattgcacttcatttctgggcttctggaaacacaggtattaagatttcatttacctccccttgttttggaaggcttattgtgtgtgtgcccatagttcttacccagttacacttaaagtatgatactttttgacagcttaagcctattgcattcctggcatgtaaaatagttattttcagaatgtaagcaaatgtggaaagcttactgcctgacatggagggttggagaagtgggattggtgttcttttgttgtaaactcatttttgtgaaatatttgctttacaacgtgaagcatgcatgatggatgaacttttagaattgaaagttatttttcatttcattcctgaaatatttaccagcatggatgttgactgagtgctttgaagagtttgcttagtcatataacttaggttttatgattttagatttaattaaacttaaagttttcctgtttgatcctcaggacttctcatctgtggcaaggctttactcacaaaggaaatatcaaccttgaagttaagcatgccctgtgtttctttatattctgatgaaaacgattgaagtttgccagtgctgtgtctgggacaatgcttagaggattcatggcttgtaggaatgagttaaaagtacaaggatattggctggcatggtgactgtcgcctgtagtcccaaaactttgagagtctgaggtcagaggattgcttgagctcaggagtttgagacaagcctgggcagcataaggagatcccatctctatgttttttaaaaaagtgaaaaacaacaacaacaacaaggtacaggggtatctgcgtttccttaggcagctgtcttatatacctttaaccttaaaaaaatccaccatttttaaaacagtgttttctgttgtttttcttagtaaatataaacaataggaactgaagtttagggcttaaaatttttgagagaataatttttatttcagtagcttttggggtacaagtggtttcttgttacttagttgaattacgtagtggtgaattctgagattttagtgcacttaagtagggtaccttgtacctaatgtctagtttttcttttaatccctagtcccctattaccctcctcctcgtgagtctctaaagtccaatatgtccctctgtgtgcctttgcttattcctaacttagttcccacttataagagagaacatacggtttttggttttccaattcctgaattactttacttagaataatggcctccagctctatccaagttgttgcaaaagacattattttgttccttgtaatggctgagtagtagtccctgatatatatatgccacatcttctttatcctctcattagtcagtggccacttaggttggttctgcatctttgcatttgtgaattgtggtcctgtaaacatatgtgtacaagtgtctttttcatacaatgacttcttttcctccgggtagatacccagtattaagattgctggatcaaatggtagatctacttttaactctttaggaaatctccataatgttttccatagaggtggtactagtttacattctgagagaataatttttgtaaaattatgtttaagctagatttggaattgggaaagaattacattctttataggaagcttttgaaaaatatatttaaaaatatataaaaatatgcataatcctaccacccacaaatgtctgtattattatgtcatttctaaaccatttttgagattatgctatagattacgttttagatcctacattttatttcttaacattaaatctcatactattttaagttctttgtacatgtaatgtttactgaatgggtaaatctgtttagggaaactggagctatgtagttgtggacttgtgcttttgttttgttttgttttgtttgtttgtacatttgctttttagaaatgtctattcagatccttggcccatttttttttttttttttttttttgagatggagtcttgctctgtcgcccagactggagtgcagtggcgcgatctcggctcactgcaagctccgcctcccgggttcacgccattctcccgcctcagcctcccgagtagctgggactacgggcgcccgccaccacgcccggctaattttgtttttgttttgtatttttagtagagatagggtttcaccatgttggccaggatggtcttgatctcctgacctcgtgatccccccgcctcggcctcccaaagtgctgggattacaggcgtgagccaccgtgcccagacagacatgtgcttttttatttggcatttctagcctgaacataggctggaagtagtgacttagagagggattgtttgccaaatatcaggtaatattttaattatttattatttattgtaatgctgtgaatcaggagtccctaacccctgggccatggactggtaccggtctgtggcgtgttaggagctttgctgcacagcagtaggtgagcaatgggtgagtgagtgttgctgcctgagccctgcctcttgtcacatcagcagggcattagatgctcataggagcgcaaaccctattgtgaaccgcatgtaagaaagtgagagctgcgttgcacactccttattagaatctaatgcctgatgatctgaggtggagcagtttcatcccgaaaccaaaccacaaaattgtctttcatcaaaccattccctggtgccagaaaggctggggaccactgctataaattgctgctgtcaagtagtgttacgcatggtggtctttaacataaaaaaaaaaggtaatgaacgtacaccacacagattagcaaaaataaggaaaggctgatctagtagttcaaacaagcaatcctcttatttaatggttctgtgattgctcgtgtaggcagaagcttaactgtaagttgttgcatttgacaggcaaatgtagctggagctggaatgtagatattagtgaaattgtgatttcagtttactggagccattcagtgggagcatgaagtgggttacatgattacacggatataaataaagcgtcctccttgccacttcactgctgttaaagtctctcctgatgttatgtggtgaaaaacaagtcaaaaacattaattctgtcaagtttccaaagatataagaatctttgaccagaaaagagacggtatttatcttcaaatttaacaaattgtgatgaaatgctgctgttacagaacctagtgtaaggtcgtggagtccattcagccagacacatgtaattttgattattaactgccgtttctgtggtcagtcggccagttttatatatttcacattataggtatttggagcaaagacagtgttgtttattgtttactttaaaaaaatacaatactgtaactacataatgagtaaatgttgatactgttgaaaactaataaaaacaaattcttacactatcaacatcacagaaattctaattgtaagaaaatattgatataatatggaaatccgttcatttatttggtgcattaattgaacaccaacactatgccaagcattgggatacaaccatgaaagaggccagcttcctgatctcacagagcttgtactttgttgggtggggcagagagcaaatcagtcaatctgtctgtctgtctgtctatctatctgtctatctatctgtctatctagatgtcaatcagtatactctgtgccacttggtggtcagtgctatgaagaaaaattaagccagtgaggagctggatagagacacagtctggaagggtatgaaggggatcagaatatgctacccccaaatatgccatttcagcaaagtactaatttgagctgaagacaatcgagaaatagcagacccaggacaagctttaccccctttttataaaaattttgctgtgtgaaggtatcctccttcccctctccatccctggagagaccactagaggtggcaccatcgcctgcagaaacaaaccttattgaaataacccttatcttctattagtttcccccatatatttaccttaccacacttcccctggaagccctaatcccctctcctttgtcttgtgacttctccacagtttattgctctttgttaaaatggtgtgtatgctctcaggcctaactgcttcttttgggtttttgcttttctgtggagctccctcctctcccctccctcatgtaaaaataataacattaaataaaatttgtgttttttcccctgcgaatctgtctcgtcagtttaattggcaaggcctcatgtgctgaacctaacagggtagagaaaaaaggtttttttccccttttctacaagtattaaacctggagagaacaatacagtgtaaaggctgtgaggtgggagcatgctggggtgttggaagaagcacatgagcttacaaacattccatcaatttgcattcattccaaaaattgtatgcaatttttcagtgacgattcccattgatcaatcatttataaaaactaatggtgagggtcttccatactcagtttactcccttcttggaaaagaatgtacattttaatcaagatgatttcatgaaagacattctgtactccaaacatttaaaagagcgtttagagtatacagcaattcatcttttcttctgtattttaaaatttaagacagggagaattattgtattatacttagttcagatttggtggtggttttgttttttggttccagtgaaaagaaattgcatcagtaacctggcggtcaaccgtgccatattgtttgtgatttattagtctggaaaaaataactgtatttctggcatatgtaaaaatagtttgctgagaggcctccttggttgtggcaatagtaatactaattaccaggtactgtatgtagctcattctatgaaccaggccttgttgaaaagtgctttcacttaattttttcttttcagcagcctgtgaggtacctactgttatccccagctgggagggagctggccagtggactgggtttaaactatgttttcattgtctttactcttaaccacttatcttctctgcctccagcaaaataagtcaaagagttcagccagtgtttcttcttggactaagcagtcagcctcctaagtaagcaagccttggaatgcaattgattggtaagatactaaagtaatcagtaaggccctcagttcctggctgccagcttctttcaccctctcctgcaggaagttctgaatgctgatatctctacttggtgtcttctctccccctcccaattccattatttccctttttctcaatcccaagtgaaggaagttctgagtgtggatgttggagtcattcagaccttggatctgagtcacaactctttcaatttactagaaatatgactgcaagaaaatgatgtaagcttccctaaaccaatttccacatctacaaagtatctaaatagattgcttaggagaaaaattgaattaaattgaataaagtgcaaagcacagaacatggccaggagtaggccctggatgaatgagggaaaaatggttctgatttttatttttgagcctctttgtaacatttgttggccccttcagaataatccttagagttcatatcagccacccttcatccctcctgggatgggccaggggagaggcccagttgcctgtgacactgcttcgtttcacatttcactgctttagcatctcaacatttcattgataacatttttgatgcctgaaaacagaataacttatttgtaattctacatacattttgtttgtgatcaagttgttagagtttgaaatgagcctcatttccttgggttttgtccctccccatctgaccttcctcttctcagtgaaccctgaagaaaaaacactagcaaaggaatctcaagaatgttggttcgggacacagctctctccatctactctcctgccctagtcctgcccctccttgtgttttcagcggtgaccaggcactggtgacagccttggctgcttggaccagctggaggtagctgcaaaatgcctgcctgctagctttgaaaaatctctgccaacctcattgaaataattgtttaccacatatcagaggggactgggaagtgggtaattgagagatctttttgaaggggtttgtggcttctccacttgcagaggcttcctgaaatggtgggtaatgctcatgcctcagggagaatcaatatccttctcctaaaacctgctcacacgtagctctaagtattagacatgcatttttgttaacattttatcagtgcatttttctacctgtgtcatttggaccttagattcgggagaaaatcactgtgtatctttggtggtttcaggtggctcttaaatttcattaattcacaaaatctgaattgaatggaatagtctgttttgggaggtttttttgggggggttggggagggattggatttttcagtccttccaagagacttcaggttttttttttgatgttctgattagcccggtgtgtgtaaggccctatcaggtgttcgcctgtgtgatcctatcacacccacatcctgtagctgcatccaagtgctttgaggccgacttaatggggattaacttaacagttgctctgtgggaacagatgctctttgccgcttatttctaataaattgattgagccatttaaaagattttttttttcttttaagttacatcttcttaagaaactgtagacaataaagcaaaactaaaatacttgggtctaaaattagtgtattatatttaaaacactgggcttcagactgattatatcatgctcttttactgactttctagtcagatgcttatttataaaaataaagcttatataaagggcagcattcaaatgttacaccagtcggttttagcagtttgaccttccacagtagctaaatattttttacactgagagaagaattctttaacattatatgctatcagcatgtcaagtgaattctgagcttgcatgcaacagatgagaccctaggtggaaacataaaaacataccagtgtgaaagagaaaagcaggctctctgtctttgggaaggagttctgcagggaggggtgtttataagtgtagtagatacttaggaaaattaatacaattattataaaagtcatctgggaaaaaccattaggtaagaatagccaataaaaaaccagaagcaaaatgtaaagatatagaaattaaaactgcacatcccatgcatagataggtcaatggggcaaactggaagtctagaaataaagttgtgcatatattggaaattagtgcaagataaaagtgtctttttacatcagaaaggggaaagatggattatttagttaatggtgttgggttatctgactgtacatagaaggaaggtgggtaattatttacccagtaccaatactggattacctagtattatgtacccagtacagtttgtcagcaacaaaaggcaaacttttttattccatgaagtgttcttacaaatcagtaagaaatactaaatatgaacatcttcagagaaaaatgaacatttttgaaaacttattatgaaaagaattaaacatacacaagagagaatagtgcgctgaatgctcagcaatgatcagctctcacccagtcttgttttatctgtattcccctttctcaacctgctcaattctcttttccctcactgggttattttgaaataatttcaattctataattttatgtataaatatttcagtgtggatgtctaacagaatctctattagacgtggacaaaaaaggcaaagaattttatagtacacaggaaaaaatacacagccaataaacctatgaagatatgctcaatctcatagtcgtcagggaaatgcaaagtaaaacagtgatataacattgttttttaaaaccctgatgggcaaagactaaaaaggcttgccagattggcattttcatctactgtcaattgataacagatatttctttgcttggagtacagtggcacagtctggactcactgcaacctctgcctcccaggttcaagtgattctcctgcctcagcctcccgagtacctgggattacaggtgtgagccaccacgcctggctcattttttgtatttttagtagaagcagggtttcaccatgttggccaggctggtctcgaattcctgacctcaagtgatctgcccactgcaggcatgagccgctgcatgcagctggtacagaaattttttagatcagtcatttagaattttatgtacatatcccttgatctaggaattctagaaactaattagttcatagtatattgtataaatgcgcacagatgaggatattatgagagcattatttgtatcaactaagcaagcctttaaaaaactcttatacagtagaatattatgctgatacattaaaagtgggcataaccatactgatgtatagaaatatctaaaatcataccgatataagtgaaaacattcagaatgcaggcataatcacagattaaacagtaatttatataaaaaacagaaaatttatatgtgtattatgtcagtagcagattcaagtgaatctgacaaaatctgactctgggcaatgagaacattggaatggaggagaaggtagtacatatttactttgtatattatcaccttttgtactatttgagtctttttaactgtacgcagatactttagagcaggggtccccaacacccagccatggacaggtactggtccatggcctgctaggaaccgggccacacagcaggaggtgagtggcaggcaggtgagccagtgagcattactgcctgagtgctgcttcctggcagtagattctcacaggagcacaaaacctattgtgaactaaacatgggagggatccaggttgcctggtccagtccttgtgagaatctaactaatgtctgatgatctgaggtggaagagtttcatcccaaaaccatcccatcccatccagtctcatgccatcccacccccaacaccccatggataaatggtcttccacaaaaccagtccctggtgccaaaaaggttagagatcactgccttagaggctgctccctcttggcacatcttgaatgtaggactccttggttctgccattcctggtaggtagtcagccagcttttgatagttctccccagagacttcatcccacaggcaagccatcccattccacgtaaaacatctctaatgttaggaaggcacgtattttgatgtgatatctagaatcctcccagctgttcttcagaggaacattacttttataaagttgaaaagaacgttttattgtattataaaactaggtcatttcagtgaatttcatctattttcatgggtaactgattcttatatcaaaacttgcttttgaaagtactggaagcataaattattacttcaggcccatttgctttactttgttcagcacattgaacatcaaggagcaactccctcgatctaaaatttctagcattggtgcttgtaatatcatttttcaagttgacattaatgctatctttggaattctcatggtaagtcagaagaaagcagtttagtggaactgttctttctcccactgaattgtatactgagatctgttatattttctgcattttaacccagaaagttgttcaagagaattgaccatgataagacatctgtagcttattttgtatttttttaatggaaaattgtaactgacttttaaatttaagattgagtgtagcctttcccaaaccaatagctaatgtttttataactatttagtgtaggagattataataaaatccacattgcttaagatataaaatgtttgtgcagtaataggctgtataaaaacagtaaattgagattgtatcaggaataatgtattataaaccatatatgaaaccaggtactaacttggtattctgcatagctttatcagaaaggcagagcaaatactatctcaaaatgaagtataaacaaaatgaggttcagtgagtgatgtggtttggctccatccccacctggatctcatcttgaattgtagtccccataatccccatgtgtagtgggagggaccaggtggagataactgaatcatagaggcggtttcccccatcctgttctggtaatagtgagttagttctcacgagaactgatcactttataagggacttttccccccttttgcttggcacttcttcttcctgccaccatgtgaagaaggacgtgtttgcctccccgtctaccctaattgtaaggttcctgaggcctccccagccatgctgaactgtgagtcagttcaacctgtttcttttataaattactgagtcttggatatgtcttcattagcagcgtgagatcagattaatacagggaggctacataatttgacccaagtagcaataaacaataggggtgaggttgaaatccaggcccctggtacttatggccttaagctaagtgaaataagccagacacagaaaaacaaatactgcatgatttcatttatatgtggaacctaaaacactcaaactcatggaaacaagagtagaagggtggtgttaccagggtttggatgtgggggaagtgaggcgatgttggtcaaagggtgcaaacttgcagttatgagattaatacctaatgtacagcctagtgactagttaataatagtgaattgtacacttgaaaacgtctgcgagagtaaatgttaaatattcttaccacacagaacaaaaaaagtaactctgaggtgatggatatgttaattagcttgattgtgataatcatttcacagtgtatacatatatcaaaacatcacgttgtatacattgaatatacataatttttgtcaattatacttcagtaaagctagggaagaataaaagaagtgacctaaccaggacacctatctgtagctctatggtaaagccaaaagggataataacttttgattatcaaaacttcatcatcattattattttttaagactgggtcttgctatgttacttaggctggtctcaaactcctaggctaagtgatcctcctgccttggcctcccgacatattaggattataggcatgagccatcacacccagccagttttgaaggtatctttattacatttttttgtttaaatatttaatagtaagtgtttaacttgaagcatttttttctgccttaaaattgattttcccactcattttatggatcaaaactattatcagattttaattcccactccaccacattgtttataaaagttgacaattatggaaagtaaagtaatactgaatttttctaatgcctatctttgacatggaataaagattataaaatttcttataacttgaatgagtttttaaaaattggacatctttttataaaaatctagcattgaccatttcaacataagttttacttatctctgaaatacaggtagtacctatttttattaattttcttaagtgatgctgagtgacactatttttgttttttttcactttttctttacgttttttttgagacaggatctcagtctgctgccaaggctagagtgcagtggtgtggtcttagctcactgcagcctcggcttcctgggctcaagcgatgctgccacctcagcctcctgagtagctgggactacaggtttgcaccaccaggcccagcagttttaaagattttttgtagagatggggtcttgctgtgttgcccagactggtcttgaactcctggcctcaggcgagcctccgacctctatctcccaaagtgctggaattatagacatcaaccaccgtgcctggccccagtgacactgtcttaattttgaatagggaagtatctaatggtattaaaatggtctggcagagaaggggatatgaggcttggtgaaaataagcacaggatgcaccatctttgagtaagtgttagtttctgcagtgagccagcagcaacattcttagcagagcctagaaagatcgagtgagggctgtgtggtggtgcagcctcttaagtctctctggttccacaacatgctcagattggtggctttgatattttctccatccaggacgcattgtgctaagcttagggaaggtccagtggatatttaccaagcctcttatactgccccgcatgttgccatgagacagcagcgttatcaccttaaatggtgcaaatactccttgtgcataccctacccctgagatctaacatctggattctggatactgtttcctattcttccatcttgctgcatggttcccaggttatgtatatagtgtggctaggcaccacacatggccctggctccttgcagccatacagaggtgacctaactggatgtaattgggtatgtaccgggtgttggctacctgtgcaggttgagggccttccacaggtctagcaaagcaattcattgcctttctgaatttttgaatgcttcatttgtgaaacccttgatgtaataactgttgatattatttctttttgaaaactccccttactcttccctgtttcttcatttgtaaactggaaccgatagaaaagtaccaaacttagggttgttgtgaggcgtaaaagaaataatacagcccagagaacatagtatatactcagcaagtatttattaatatgactactaagccagcattggtgattgattggctcatagtgtcttctatttgccagatgttttcagaggctctgttcatgttcatacccatgtttgtaaatgacagaatgtaaatttcttaatcctctgtcttctcctttagcatcagctcttgtccccaaagtaaaagaactcagtcatccagaacccagttttgtagatctatttttcaaaattgggcagttaatagaaaattaaaagcagtctagggaaaagcctttcctttgggtttgttgctttcttttctgtcataataagactgtgcccaaactgtagggaaaaatcaagaaactgtaaaatacatcatcagggtgtttatagtaaatgtaaaacgttaagtaacatttgagttaatctgtgcttgtacaaacctcagaagcttaaataatgaaccaatgacatataatttagaaagttttcattagactctttgcgttggtatttgcaatatcagatttgcatgtcatcattattagccttcctctattttgagtgttgactaggtgccaggcgtcatgttgagaaatttatgtgcttatttaattctcacactgactccatgagatagatactccattttacagatgaggaccccacatcataaccttttggccagaattacagttagtgttagaatttaaacctatgcagtctcattccaaatcctctgtctttactcctccagaatgctccattaaatgaattgtttcttataatccggttgttttaacgttggaaaagaacttgagaacatttagcccaaccaccctatgaagtttggcagtacttcttgccattctttcaatagataatttatttgagtttaagggagagtctatgaattaacactagtaccaaaactcccatgagaaaataatgtagacagcttttgttaaaaaagcagaatttttaaaggtgttcattcagttatcttcttgcctaacaagttcctgtgttgtgatagactttgaatttgttaaatatgaattggcttcctgattctagctgttttgccagaggcctgatttagagaagggattaaaccactggtcaaccttggatggaatttgggccagcagttccttctccacagcccctgacacttggctttgtgccaagaaaatcctacaaaaggaaattgatttactctacccaagacagagttccggaggctttggttgtccattgttagcttagaagttggcgcagtgtgcgtgtgatccacgcctaaatagcacagccttgctgtgcgtggtagaagttgggttagtgttgacatgctgttgactcaccctcccgaggatggaagctctggcctgggtcaagttgtggtcactgcagttaacagtttgttgatctcagggagtattccacagttgctgatgtaattgacaatgattggagccagctcttccccagattcaaatggaccaattagaggacttgttggttctgtttatcaactatgtaagtgagtactgtgaagttggcattgtttttagtctgtatttgggtaagatgaaactctgttctcagacagtaatatctttcaaaagtacaatgttacttattgtgaccaaagtagggatgaagtttgcaaattatttggtgtaatgatttaatttggccatccatgcagtgcagttctctaccagtgtgattgttctctttgtaaaatactcttactttgccagaaaattgtttgtttgtttgtttgtttgtttgtttgtttgtttgttttgagacggagtctcgctctgttgccaggctggagtgcagtggcccgatattggctcactgcatccttcaagcaattctcctccctcagcctcctgagtagctgggactacaggtgtgtgccaccacgtccagctaatttctgtatttttaatagagacgaggtttcatcatgttggccaggatggtcttgatctcttgacctcgtgatctgcctgcctcggcctcctaaagtgctgggatacaggcgtgagccactgagccctgttgaaaattcttattaataagatgtttctcatagaatgtttcaaaaaattgtgactggtttcaaaatcagatgatttgcaagaattagccatagtataaacacatagttctcaaaccgttttctgggagtgggcccagagatctatttaacaaaaccttgagtagagtctggtgcaagttaaaattggagaaccactaatgtaaccttaccttatctgtggaccacattttgagctttggaagaaataaccctttctatccttcacaaggtaacagatgatttttttgcacaattatttgataatgagcttatatttgagattaaggtatgcacacgataatgtacattctcactctaggtctggggaccttgattctttgactcttctgtccttttctctccagttacttggttttgtgcattaacaaacttttcaatttacttatttttttttacttatttacttatttctcatcccttacgtaatgttgcatcattgttttttgtagcctctgcagttcaagatactaatcacccctttctaccacattaaaatacagactctacctaaaatcatgtatgggagtaacttgatatatacgaaagtgtaaccgtgtcacaattccccgccccaccccgccagtctacataaaactgcaaacactttattgcttaccaagaaagtagacaataatatcgaagaaagccctccttttcctcttctttgaccaacaagggagcagttcctgacaaatgtcagtttgctttttgagttccttaggtttctgtgttgtgtttgcaatggataaatattgagtaacaggtactgactgtattgtatacattgaaaactatccaccaacagaatatttgcaggaggattttgttttgttttgttttgttttgttttgttttgttttctgagacaggctatctctctgtcacccaggatggagtgccatggagtcatcatggcttactgtagccttaacctccttgtctcaagcgatcctcccgcctcagcctcctaagtagctaggactacaggagaggaacaccatgcctggctaatttttttttttttttttgtagagacagagtttcgctgtgttggccaggctagtgttgaactcctgggctcaagaaatctcccaaagtgctgggattataggcatcagccactgtgcctggcttgagaatgacattctataagtagtttcagtcccttagtttaatcccatattttgatcctataacatgtacaatatttgtgtatagtatatgatctgccaactctctaagaagattttaaaacttattatgttaaatttcagacatatacatcatccagcttcaaaaattgaccacattttgctgttcttgtttcagcacctccccctgccccattttcttttctgaagcattttaaaataattctcaggtatcatatcattttacttctgttcataggtttctgaaaggatttgatgcagaatgttattattaaaccagtatacagagagaaaggccactggatattagtacatgttttggtttcagacggtccttttaagagtatattaaatcacataaggattttctgagtacccattagaaccacaaaaaatacctatgatacccttcttactgcagtagaagatgagtttcctagggtagccctttgttgatggaacaaacatgtattttgtttcatcaggaaaatcattggaacagccatgactcagatccactaaatgaaggaacaaaaataggacctgcctcacctcctgatttttttttaaatctctaggatgagtaaccagaatatttttatgttgtgctaagtaggtttgtttgtttgttttttactctgacttatattatttgaattgtagaaatatattgtttttacttttttctttaaaattctatctggagtttctaaatgagagattgtagctcctaaaacctttcttagttttggtgtcttgctttctgttcttagcgtctgtggtcatttctaactcccagtgaggaaagagctctctggtcttgtttctttgttaaatgaaggagcaaacctccgtctcgttgatccactcccaacccacttgttttgtaacaatgaaagttatctcatacttaaaagacaacttgaaattataacacacctgtaacaaagtaaagggtatgggtttttacttaggtttaaaaaaagggcagtaacccttttcataaaaaagcaaaatttagatgggatagaacccccgtttataggaaaagattttaaaatctcatttgctctcattgaaaccttaaccttctctcaaatactggcgcacctctgaggaacttcaaatacggtttgaaaactaatggtttaaggagggccaggcgcagtggctcatgcctgtaaccccagcactttgggaggccgaggtgggcggatcacttgatgttagcctgggcaacatggccaaatgctgtctctactgaaaataggaaaaattagctgggtgtggtggcgcatgcctgtagtcccagctactcaggaggcggaggtggaggcgggaggatctcttgaacccaggaggcataggtgcagtgagcagagatggatctcttgaacccaggaggcataggtgcagtgagcagagatcgcgccactgcactccagcctgggtgacattgagactgtctcaaaaaggaagacgaatggtctaaggaaactagtttgtggtttctcagcggagaatgacattctagtttctacagatctggcaacagaagacaggcacataaactggcctgctttggagtcagggccaagacttaaatccagatttaaaaccagctttttttttttcttcttctaaaacaaccaacctgtgttttaggtccagtgtctgccttgtaagttgctcaaagctcagatacaaggaagtaatcagcacacatttttgtgataacattcctgaaaagaaaaagatgaggtggactaaggtaaatacactgctccttaaaaatccatatttttagattatttataaatcaaatgggcatggttaccaaagatgaattctagaggtgtaaaactccagtcttatacctcaaatttcagttttccttagggagcaaaacacttccatgatcttactgaggacctaaaaaagcctgtacatattttaaaaatagcactccctagataaagcctgatggatccagggtacagtagaatcatgaaattgcctgtttctttttaaaagatgtatatgattgctcggtggctgtatattgtccatcgtgtgcgtgcctgtgtggtatgttggttctttcagagaggcagtggcgtgcagagtgagaggatcacagatgtgtatgtgacaaaactatttcttttctttcttttcttttctttctttctttctttctttctttctttcttttctttcttttcttttcttttcttttcttttttcttttttgagacagagtctcactctgttgcccaggctggagtgcaatggtgtgatcttggcttactgcaacctctgcctcctgggttcaagcgattctcctgcctcagcctcctgagtagctggattacaggtgtgcaccacgatgcctggctaatttttgtatttttagtagaggcagggtttcactgtgttgttcaggctggtctcgaacttctgacctcgtgatccgcctgcctcggccacccagcgtgctgggattacaggtgtgagccaccgtgcctggctgacaaaactatttctaaaacacaagtccagatattcctaccaagggctctctggggcttcgttttgatttatatgcaaaacactcctaatgtggtgacattggggagataaagatctgggatgatgagagagaaaggcaggtaggacacatgcccttccagtaactttcccagctagggcttgcatttaacctctgatggaggcctcaccatccaggcagatgtacttgcttaggaatgagtttgagataataaggctaaatgtgctgccttctgtatataatttgcatttgtcctaatgtacgactaaggaaatgaaaaatcaaagtttgagtgactaaaataaaagaggtctgtggagtaattggattgaatttgcaccaagtcaaggagaaaacttctgtattttgtatgttttgtcatggttctatgattacttgttagaaagtgatataaaattcaataacttagtttgatatgaacttggctatctttaagttatagaagtcgtgcatataatgtggctgttttcaacccccataccctttctgagtggttggagatgtcttgaatttcgaagtgagaaggtctggtgctgttacatagataatcatgttagccagctttaccttctgataagctctcaatttaagaaagtacttctaatcctggctttatctttggcttgctatcttattatgcacagagcataattgtaagaagggaataatatctcttgctgtctattcactaagaatagtgtgaggacagggctcttaaaaacatgaagagatgggcactattgtatagatatagagatttgatgaaatgttaaaatgttggatgattttaaatgacaagtggagtggtgagaagtttatttttaaccttatgtggataccttggatatttagaaacatcgaagttttaaggaagaacaaatatcttgactccaagaaaatggtcatgtgatactgtggcatctatttggtcttcctgatgtatatgtttcctgacatttagctcctaaaatccttggaatctccagtgctaagaaggtcttatatgctaatgagatgactggtggctgggtaacctaccatgtgattagagggttaggactttcagtcaccctccccaacctccaggagctgaagtttgagttgataaactaatgatcaatgatacctacataatgaagctttcttaacaaccctgaaggaccgcgttctgggaacttccagatagctgaacacatggaggttcctggagggttggatgtcaggaaagggcatggaatctcgttttctcttcagccgcacctcaccccgtgcatctcttccatctggctgttcatctgtgtgtcctttgtaatatcctttaaaataaatgggtgaatgtaagcaagtatttctcttagttctctgaaggtgctccagcaatgaatcaaaacgaagtaaggggtctggggaatggtagccagttggtcagaagcacaggctacaacctggggcttgtgattgacatctgaagttgggggagttttgtggtactgagccctcagcctgtgggatgtgtaatctccaagtacagtgtcagaatccagttgaattagaggacactcagctggcatccattattggcatgcagtattccttgtcatgagcctgggcaacatagggagacctcatctctacaaatatagctgggagtggtggtgtgcacctgtggttccagctactcaggaggctgagtgggaagatcgcttgagtccaggaggcaaacattgcttctcctccaaatgctgtactttaacatgtttggaaactcctgcaaaggaatttcaacataagcgaagtcaatgggttataaaggactgaatgccattttgtaaatgtcagaaatcaaagtcacaaccaagttaagttctagattagggccaggaagagaaatgtattatctggtcaaccatatcaaatctgtattttggtggaagagcattttcttatattggttctgattggtttgatacggacagggaactgttttctttattccagttatttgttttgttttgtttctgagacagagtctcgctctgtcgcccaggctggagtgcagtggtgcgatctcggctcactgcaacctctgcccctcaggttcaagcgattctcctgcctcagcctcccgagtagctgggattacaagcgtgctgtagtttttgtatttttagtagagatgaggtttcaccatcttggccaggctggtcttcaactcctgacctcatgatcccacccgcctcggcctcccaaagtgctgggattacaggcatgagccaccgcgcccggcctattctagttgttaatcagttaacatagccaaaagatatagaataagattgtagactgaatccatagtctaatgatttgtcttcctagtaattgtgaataattcagacattcaaaattagtctggcatcatacaaacattagactagtcctaaaatgaggatattggcaagtgtgttaataaaaattgcatatgcagtattaataggttccttacgtaggctgccgattactcaggaactgtcagatgttttgttttgttttgtctttgagagatggggcagggttttactctgctggagtgcagtggcacaatcttggctgactgcagccttgacctgctgggctcaagcgatcctccctccacagcctcctaagtagctgggactacaggcatgcaccaccatgcccagctaatttttgtatctttttttagagacggggttttgccatgttgcccaggctggtctccaactcctgagctcagggatctacccacccatgcctccctaagtgctgtgattacaggcatgagccactgtgcccagcccccagatttttaatcttctctttgacattgagtttcagaaatgaagtataaatggtgaatttttagtgtagaatatgatgcactaggaagatcacttacccgtttattcactttcttagaccatgtcaacccgattctgaatttagggttctgttgtgatggtgttctttagaagtgattttaacactcaaaatactatagtctgtaacataatttactgattttttaatttttgcaaactaagttgaagtaatcaagtatttatgagttctttgggtgagaccatctgtcaaagaattctgggcttaatggagatacatatatgttgtgaatactaaacagatatccaagtaataaagcagaaatcatatgctgtgtaacagaaatgtaaagttctgaattagttcacgagtaggagagaaacttttttttaaaggtagaggagaactaaaagtaatttcttaaaagagtgcctattttacatcttgaatattaaaagtctaattcagtgccttgggatcttgttctttttgtagattttgatttgaatgaacttcagtttgctgaaatgtctaatgggtgcaaagtacagatttaattataaggttgagctaaaatcagaatttaagcatatggcatatataataccaatttaatctttcccccttaaatttagttatttttagtgagtcccaaacaccatatttgagacatcacttttttcttttttaaagagatagggtctccctatgtgaggctggagtgcagtagctattcacaggctcgatcatagcaaactacagccttgaactcctggccccagtgagtgtaatgtctcaccatgccaaagtacttttatcttaaattgcttatttttttgtttatttttttaactgactctgtttacaaaattaaccttttatctagtgacagctagattgtatcacatttgtcatctatggacaactgatttttagttgtttaatatggtaagtttattattgtttttccttatttaagaaaacaggatctgagtgaaaaaaaaaaaaatcttaattcaactatcacgattccagtaggaagtagacagacattcatctgtatacattaggtgtcaaagtagagttttgtggtttatcattctgaagatttgagattcaagacatgactatctttcagtcatattgtgcaatagataacatataatgtttctcactgatgagaacagttggttagatctgccacactgagcgtgtagctggggttgacgacacacgtgttgcagtgtcgttaccagtttgtctcttttagagacaacttggcaggcagacgtcttgcagataaacaaggaaagattgtctattttaaatggcataagacattctttaaaccagtgtgtcatatgtggttatatagtcagcattttctttagtgcaggaaatcctctctcagggttcctgaataaatagcaggtatctttgtagggatagctcgcaggagatttcaaatgctgtgtattcatcagttcaggctccccagatgtcttacgaacgtgctcattttagttttcttttcttttccctcttcctgctagatacaagttgagacaatatgattagctcttaaagtgcttactctccattttatgtatattactgcattttaatcctcataatatcctgtgaggtgggatgttactatgtccatttgagagataaaggaaatcaagaggtaaagttaactggctttctccaaacagctggtaattactaaatacagaattgacttttgccttttgattccaaagatcatattggtactttcatgtgtgggtcccttcagccagttcctacagtgaccctgacccagctgtaagtcccctgaggtctctagagatatagacagtgatggattagaccctgttcatagttgcagatacaagacatagagaggtttctagcaataatttttttagttatatattttcatagggcttttgatttatgtaacctattagtatatatttgttattccagtagagttaatacattattcaaagagattccagtttttaagccttttctccgtttgtctaagttgttcattatgaaatgattggctagaatttgaaagtctttacctacagagagattctgactggcttgcttggtcttggaaccctagttatgagtgcattgtcttgcttcctggtgaactttgcccagaaatagttaccacctcattttttttttttaaatgaaagctggataattaaccctcaagatttcttaattaaaagattactattataaattgacccacagggagttaatacaataaaattatttttgatcattttatatgaaataattatggggcaatcttctgtaatcttgagttgaaggggttatcttgcttaatgttaataacttttgatacaccaactaccttttaaaaattaacgttttttttttttgcttctgtggaagggaggctgtattttgtaggatttacagtttacatagttattgttttatatgatgtcagttggattaattatgttttgagtggaaactctgtttggttcaaccaatatttgaatgctaattttgttctgtgaacaaatagaaatacagagaacagtaagacatgactctttccccctaagtaaaggcttaggttggagttttcaacctgggaattactgacattttgagccaaataattgtttatgttgggtgggggagggcctacagcggaggctgcctgtacattgtaggatgtttagtaacatccctagcttctacctactagatagatgccagtagcaccacccaagtcgtgacaatcatctttgccaagaactttgaacctctacctagaaacagttcatagggctcaaaaagcaaagtggcctaggacccatgtagttaacaagcttaccctgttggagaaccactgcatagctgaacaacatgggtgagactagtgtcagggtaaaggagctcttgactggagattggccgtacacagaaaacaaagtgagtgttatggatatttggggatttaccttcattagggcacagttggttagcgttaggtgtacaccaacattattccctctttagaaacagccatgcacgtaacattttagcacatctgaagaggcacttctattctaattacctaaaattcttattatctagaattacatgctatgaacaacagcagtaaagtcagaagccagaatctatgtgatgttatctgttttgcgatcacagttgtgttcttctcttagtttatttgaacaagtttgtgaaaaaatttttgtgcttatgtaaatactctttgcttagattttttggtagaatgtaaatctttcccacacagtgcaatacccctcctgcccaagggcgagatatagattgtacagtttgttatttctgtgttttagggcttattcctctgtagacatgtcaaaaagacatccgtcgggggtaaggaatgcattagcaattcaagctgataatctgcatggtgagagtccagataattttttccccaggtcacataggcagctctgcacaagtctgttaaacatatgctagttttgaaaattaaaataagttcccatcactcaaggcaatgaaacgtagatgaattgctctgccagattgactcttagaactcctgatgtgatgtggggctagacagaatgggatgtttgctaataaatcagggaggtagtgattgttttccttctttttctcccccacccccgtcaggacaaactcacattctgtagcctggtgcactctaataatgcctgttgaccttggccatgaaagcactctagactcaccccctaaacaccgcaccgaatgtgctcatccttgaaatttacaagccttagaatcttccctacctccactcctgtttactgcctcctgttactcctggtgcttccagtcagaccgtagtcttggctgtgcagtacctttaggagccctcaaaaatgtttaatttcttttaaaatcagaaggaaaaataaagaacaagataataatcatgaatatacaataatgaatccagcctgacttagagttgtctttataccaatcaacagagccctaaaatatagcttttaatgtttttttaagagagaaagaggcccacgaaagttgtcctggggcccccgcatcctcctcatacactggtgggatgctgctcttgcttgctttggaatgtggcagcagctcccagtgacttcacacgtttattacaaattcaaggtcatttggacgctagccttgtgctacctttcatgcttcctgtccgacccatactcagcctgggcatgttttctgctctgatctttggtctgccttccttctccttctccaagtcaaaccagtcactctttcctgagagctcttcggtgtacccttgccagacctctgcattaacactccagcacctttggttagccaatttggccttatttgtgtctccaccattggattatctgtccatcaaggtcaggaatgttttcgggttaccccactgtccccaactttgaatatgttcactgcctggaaaatgtttatctgaatataaggcatcaagccagaacttgcccaaaacttaacctaggactcaaacagctttgaaatcttatctgtgtagagtgggaacggcatgtgtttgataaccatcagcatttgctgttttgcttagccatcaacaatccgtttctatctcttcctcatagtattttcagctgtatttttgtacagcagtcattcacgttaggtgttaggttacactgaaatctctgacaaccagttctgtgctactgaaggcttttagcagcaggattaaaaaaatagttgattgcacattcgaagtctcactgctcacactgtttatgtaattcttggagtccagttagatcatctgatttttaaaatgttgcctgaattacaaaggagttcaggaatgtgttatcaaagaattaggaaattaacattgaaatttattgagcaacatagctaataggttgtcttgctttttaagccttcttgttttctaggaggagcctgaagtccgtggacaagttgaactgtagctggtatttaggaattggtaaagacagaggtgtggatgctatgttttcttttctctgttaggaaattactctcctggtgccacaataaaaaatttcacaggtatctaaagtggaatttattttgtattgttccacacctttgtcaataattacattttctaccttctagtttattactgttattatttttcatgccaggtgtttcttaattgttggcataagtatttattgaatgcttatatgtgggagtgagagagtcaggccagtgctaaagagtagggaataacccctagacagtgaagtaacttttgtcttgtttgcacattaaaaaaaaaaaaagattaggaatccttgctctatatactagaaatgtttgatgttctcttttcttctcttcaaacagagaaaacttagatgaatacttataggctctactgtcaactaactgcaccctcctaatattggcgtctgcatatttttaccattgtcaatatcagattgcctctgatgttgtgtcactgaaactgatagtaacattacatccaagagtaagactgcagtgtgatttttggctcttggtcttctgttctttcggcaacaattttttataaatatagtcaagaaaatagaaataagagtcttcactgctacctccccttcagaattattttctcctagtattgtggttcctgatagcctttattcctatagaaatggcaattcgaaaaattccttctgtgaatctgagggctctacaaagggagagcttctttgatagtgctttggtgtaagttatttaagaaggacaatctacccaggcttatcaagcaaataacctggaatttaaacctggagtattctttagtgttccaagtctgcaggaaaagaagtaatcttggcttaaactaaataagttaacagaatacaatttgaacaatgaacaagaatgctgtttgaaaattatcttgggtttttatgtcaagttaatattctcactgaatttctattatgtttgtaaagcagctttctcctagaccaaaaatcctagtaataaaaccacaacagtatgagagttataagcacacagtaaaggctgatgagtaaatgttatgacactttgaaaggaaataaaccagacgatataatgtaaatatcctttgtgcttatcaaaaataagtgacttcatgttctgttcctgcaatatttctgtaaatctgctaaaaatagaaaggtaaaggtctaagatcataaatctctgatgccctttccttaaaagtaaggaattcttgtcttggattcttatataatatacttatttataatctcacttgttctgattgcaggtggcctcttactatctttacatccatgtgaatgtgtgtgctttgatgtgtacatatgagagacaagaacaaaagtaaatgtcaaatatccgaattcctgtttcagtgagctttcacggagtaaattctttttgtctacttgcctgtgatctcttctaatatgacttgcgttaatgacctttaagaaatcttttaataaaataaaaaattttattaaaatcttttaataaaaaaattttattaaaatcttctaataaaaaattttattaaaatcttcttcatacgtgttacttgaatttactcatggcaaggaccagttagtaccattttatgaaatcacaagctcatgggcttgtgataaaacatataagtgctttaaatttggcattggtaacggcaggccgactcacttgaaaaatgctctgaactgctctgcctcatgctggtgggaagtcagatggaaatgactttctagagagtttagctggcctccaagcttagtgggtactcactctccttatcccccgatagctgctctgagattttaatgcacattgaagacaatatgataacttaacctggaaaattagcattttggtaagggtctttaacttcattagtattttcataccactttggtattagaaattgtaaagatattaaagggtatggacaatgtggaatgtatgtaccagactcagcagtgtatactgactccctcactatagcccagaactggaaatcccgctaactacctctccccatcctagcctgaattgccctctccacctatgttggttttacaatggcctgacgcctgaaagctggtagaaactgtataggaaaatagggttgggataaatttctgggctgacaaaggatagaggaatatgcttctttttccccttgtccaagtatatcagcaactctaagatgcagatttatcagtatggctctactgggcatgttctagttaaatggtagacccactacctcttctggaatcttgttttccattatattcccacctgcagcttttcaaagacttattctcttctctaatggtctcttggttcttttttttgcccatgctctacgttccgagtgggtcaacattgcagtgggtcaatgagaagaaatagaggggaaaaataaaagaaccagaaagaaacctgttaagtttgcacttacttgtgcattaagcatattagcgtaagccttaaactctagaaaactcactgtagtgctgtaaagtaccactgataaagtaaaagttcatttttatgctgtgtcgttatttagtgatgaatatgatattgcataaaagatgaggatctgtaaggccaggcgcggtggctcacgaggtcaggagttcgagatcagcctgaccaacatgatgaaaccccatctctactaaaaatacaaaaattagccgggtgtggtgacgcgcgcctgtaatcccagctactcaggaggctgaggcaggagaatcgcttgaaccagggaagtggaggttgcagtgagctgagatcacgctactgcactccagcctgggcgacagagtgagactccgtctccaaaaaaaaaagatgaggatctgtgtataactgccaggtgttgtgtgaggtaccatgaataacctggcaatatcctaactgtgcaggtatgaggagtgacccccaggaatataattgcaattaaacttgcaaattatgtggggatactctatttcagggcaataaaataaattatgtctagagctcataactcttacacatatttggctaggtctatgcagcaaaacccaggtgataactgaggctttaactttgggatttgtttggtgctgactctgccacatggtgaaatttggtaggatctcagtgcgagtgtcacatgatcagtctgcttgacccaacatgccagaattccatttctcagcccactctcttctctgcatacatacctacctcccggagagggctgggcgggagcctacgtgtggcaagagccctcactgcttttgttacacagatttcaacacatatggcttgatggagtgtgattgcagtagcatgaatgtttggtgatgaaatttgcatgtcctttgctgggagaagtataattataagatttttgtattcacatagccacatgtgaggtatgagcaggaacggggtctatttaatagcattagattgctttcagatttgaaatatttccttttagaaatatattgtgtaaacaaaatatgtttaattttttttaaaaactatgcctttgtacaaattgtttatcatttgcagaggtctaaaaaatgtaggagaaaagaagctcacaaaatcaatcagatctaaagattgcaaagattgattaccatctccttaaaagcttcagtacatgtgtttacagtatcattctatttgcctctgctcagattctttccaattctaggacaacccaaagtgtatagatcataagccataactgatcctaccttacataatacattaaatataaaatatttatggccaggcatggtgaccagttctgtaatcccagcactttaggagaccaaggcaggaggaacacttgaggccaggaagttgagaccagcctcggcaacatactgagaccttgtctctacagaaaaattagccaggtatggtggtatgggcctgtactcccagctgcctaaaaaaaggcaacttgagaaagtttctaccttctgaaacaatctatgccttgattctatcacttagtattttagttattcctatcaaatacatgactgtgttaatttcttttgccacaaaaatgctgcaaagccacaaactcactccaaaatttagtggcttagaacaataaccatttattatagcccacaagctcaagggtcagctgatcccggcttagcttggctacatttgatataaaacagtagaaatttagaaattgaatatatgtagaatatctcttgtaccaaaaatgaagaggaataatagattttgatttttctttgttgtattctgtgctgggtgctaatctgtcctagctgtcagaacaagtagaagccatactggaaaatcatttatttgttactatcaaatttatttcagtgaatgccattcaaaccactctctctgaggagcccagttaccaagtcctccaccttgttagcaacatggacttaaatgcactaattctagaatgagatcttagcaaagtaccagcatttgagatttgtgcaaattgagctcttaaactgtgtgtatatgtgtgtgtgtgtgtgtgtgttcattctagcactcccactacataccatacaagtagctgtatagcaggtatatatacatgtgtatatgtacataggtaggtatatgtcaactgttgaaaaactagaaaatacacattaaataaagtaagggttgataaacattagtagttggaacattgggagatttggacttctactttgcagattcagttggctgcattagatttaaggaaagaactaactttggagaaatttgtgtttgcatttcaggagagatttcttaaagaggaaactaccagaggttagtagatattaacatctagtgatttaaatttgagaaatgcaaattagacatttaagctgtgggtttgcttcccgttttacttgctctaaaagtaatttttttaaaagccagtaacaaagacatgcaaaatagtactgccatgggggaaaaattacttgaaaataatattcaagattgaagaattaaaaaagcttgacatctagtaccaggaatgccagctggcttgttctgtcaaaaagcaagcatgtgctttggaataacactcttgagtagactctaggcgggttttgagcccttctgattatctaaatagatgtggaagtgacttatcttggtttttctgagtcaaatgagataatgagcaaaggccttagtaggtctttgatttttgcctctaaccctttttctatctgcatgttcacaaggtcaaggctgatgagatatggcaatattgagagagtcagaataggataaagatctcaaatactatatatatatttttttttcatttttttttccctttggtgactgtatgagacatgtcagtaaaactttaaagacaggccaggcgttgtggctcatgcgtataatcccagcactttgggaggccgaggcgggcagatcacttgaggtcaggagtggcaacatggcaaaaacccgtctctactaaaaaatacaaaaattagccaggcgtagtggtgcacgcctgtagtcccagctactcaggaggttaaagcacaagaatcgcttgaacccaggaggcagaggttgcggtgagccgagatcaccccgctgcactccagcctgggcgactgagcaagactctgtctcagaaaacgaaacaaaacaaaagacacacagaaaaactgtaaagacagaatgaaagagatgaactttttacatctcgctcatttcatcacatactatatagtatgtgtatggtttcatatatgttagtgtgtgtgtatgtatatatatgaagttaaaagaaaacattcaccttttaagactaaatggacattgactagattgtaaactgtttttgaggctaagagtttgtttgccatgcataaggctctattttttgtaattccacttttatattcatttatatcagtcctagcagtagatgccactcttactgggtcctgcctaaatgcatgcttcaaatgttggttattaatttcacgagtagtgaataacaaagactcctattgagggacctcttgatttcctactgggcaccagcagcaagcatgacttactggccttcttatgagtgggtaggagttttgggtcaacttgagaacatctatcctttgagtagtaaaaaagcttggtattaggttggcacaaaagtaattgcgatttttgcaccaatctaatatcaacagaggccgtgaaaaacatcaagggaggatagcttgtaattagaagccagatcaacagtgacccaaaaagaatttcaggtttccaggagtgaaatatgtagattactcctctgaaaggaattgtttttctcctattcgctttcatggcaaatcattttttaagccgaaactcaagctctgaaataattgtgtagcaaatagtggctgtgaatagatgtggcaactctccacaagggtaaatagaaaccacttgcagtttcagtcatcacattttatttttcttttttaatagcttctctatttttgctcaccacaaatacatgcacacacacacacaggggcaaagccttggaaaattgcaattttccattttaatttgagagacagcccggtcacattaacagaacccttgacggaataggttgaattcccttctctgtccctataaccccatggccttggacatatccttacctggtgccctttcctccctcttatggtccacttttgtgattctgattttaatttcagttctctgttagtgatgaaatgagcaactgcagatgttccgcttacactggaaatacttttttggcatttaaaagaaaaccctacagatgaattattcccctgaaaggggttcactcagattttctagattatgattctagaaaatagttctttacatttgacaatggagaatttaaatgttttaatatgaaaatgtaaagcactcttgtggggtatagttgaattatgcccaggttaagggaatccagtatttatgtcaggaaaccatggatgtgatagaaatagtatcgctacaaagctgggcacagtggctcatgcctgtaatcccaacattttgggaggccgaggcaggaggatcacttgaggccaggagttcgagaccagcctggccaacatggtgaaaccctgtctctactaaaaatacaaaaattatctgggcgtggtggcacaggcctgtagtcccagctacttgggaggctgaggcaggagaatagcttgaacctaggaggcggagaggttgcagtgagccgagatcatgccactgcactctagtctgggtggtggagtgtgacagtgtatccaaaaaaaaatactgagattatgcaagaatctgactcgaagaagtttgaactgattaaaaagtaccaaatacttcattatatgaggccagctatttagactagtcagcgggtggttcaaatgcagaaaacagatggctttcattagccagaaaccttaaaattacataaatccacggatgcaactaagctatggatctgtgccctctccttgccaaacaggacctctcagctagcaaaacagagagctccttgttcacttttccctcctagttgagaccctgttggggaggaatgctgccaagcctgctctataaggaattttgatctcctagatgataacagagagaaactttttggagaaagggaacaccctgtttgaaaggacaacaagagaacttgttcaagaaggtggctttgcatttaattgaaaattgtagcaactgtaggcgtagggtttccagattcacatctgcagactggtccagattaggtatatatgtttatattcgtttatgttatccccttaagcctcttttaaagccatttctaaaaattgctttggaaagtccatttcacttcagtattctttatattgtggctcaaaaatattttttttctggtaacaaaaatattttctttctgtttttaagacttgtagcatttttaagtttgatatgtacgtctctgtagttcagagcacttggtaaacagcctcttgcttttgaggcaatttatgaatgcttcattgtttttagctggttattttgttcattgtcaggaacactgttttcccatgggaaagcatctccagttttatgatgtcagtggcaagtcattcagtttacaagaattaccttcatattcagtcttagaaaagcatagaccacaaacggccagttatatcacagttctcatcttttaaatgttgtcttagatttttgcaaagtaatcttaactcaccttagaatagtgttaagacataaatagaactaaaatgtagtagttaagtggttatatttcccatctactaaggtatttatatttcttttcttttagaaaataaaaatgtttaaaaaattaggaataattatctttaggatgactttgtaagactgtgcttttttactggcagcaaatacctgccagatttcaggtatttggcaggtaaattgaatcttaaaagatttcaattttaagagtgtccatattaacagatcctgaataggaccctgtgtatcatttattaagtcatttcttggaggcttcactcaggaaaagggagttaacccttggatggcaatgtttaatgtggccacatttccttctggctgaatgccagggagtggctgcgcttagcaaccaggctttgtttcataaatgggttatttgatgcaggtgaggatgccgtgagttgtttgtcttgttttaattcagggagacaggcatatgtagtgaaagcatggaattgtttgcagatgggcttcagtaagaataggaaggtttctggttcaatttgtatctccaagctaattgctctgatttccatatacacatctggtgttggttcggggggaaaaaattttacttgagaaacatggcttttgtgagttgatatttatttgtgaagggcctcttgtaatgtcaaatgtaagaagatctatacacacaaccaattttacaatttctaattcaacagcttaccctattattgaggattcgcagtttaaattattaaatgatctccacttgaagaaaatagagcatgttaatcatcacgattctccaaacatttatttttaaagttaatattaaatctcatctggtggaaaacctgacaggaactgaagcatatattttctagtctaaactctcattgttaaaatgaagacaacatgagacagttatagcgccgtaatcaagttatttgaaaaaaatgttttaaaaattaccactttttggtagtcaacatacacacatatgccaacagatggcattaaaaaatggcaagaacatgagacaattatagggtcataataaatttatttgaaaaaaatgtttaaaaaattaccactttttgatagtcagcatacacacatatgccaacagatgacattaaaatagctgtaaagtcaagaagcagggaggatgacaaagtgaaaaatagaagaaatcaaaggaagttaagtcaaaagaggaaaaaaaaatgaagcatttacttggtttgtcaggtagggtgatccagtgtattttgccaaaaactttcctggttttagcaccaaatgtctcagaacatggcagctgatgacctccctcacagtgagtgatcccagagagcaatgaagaattgtgatgcccttcatgaccttgttttggatgttacacatcatcatgcccttcagtgacctttctggtagcaaagggtttgtttttaaaagagttgcagcatctttaagtctaatatatacatgctcggaagttacaccatcatcacttctgctgtactccatttgttgcacagacagaccctgatacattgtggagtttcccagggtccatgaatagcaggaggcagggactattgggggccttcttggaggctggctaccctggggagagaaattgtcaatttcctctccttgcggggaggaaatggaatcaagtgcatttgtattgacaagaaggtagaacgctttccttagacattgtaatgaacagattgtggataaacaaaagtacagagagagtttgaggcagaggaggagactgtttcaacagaatatttaccagctatggtaaaggctcattagtgtctccgtacagttaacaacatgctgccaagactggaaatctttgggacaggaatgaattcggaaggtgaacagaataaagtactgtgggaacaatggcgattccattagtggattgtacattccttacgggcagggccctccatagcatctgatgtagggccttatccacaggtatctgattattatttgactttctttaatgtttttgcagtgttattatctatactacttatttcagtaagattgacaacatttgcatttcgatttctaagatcaagacaatgaatttcctcttaatccctcaggaaagctgtgaaatttaaatttatagagcattctacttagcctgctgctttgcatctgtcagagaatgagaccccccagtttcatggaagtcacaggaagtggcaaggggctcagatggagttgaggggttctagctatttctgcagatgatctggatattttggcatcggctgctttgacttaaccatctggcaatgtaaaggcagaatttcgttaaatattggaaaagttacttgaaaacatggctctagagattttgccagcagcttttgcactcatgacttggttcaatctaagacctgttttgtttttttgttgtaaggacctaagtcctctgttgggttttgacgaagtatgtgtaaaaatgactaaaatggccagaggtatcttattttacatttatgtgggaagaaatgtttggaaactgtttcatttctttaactgaaaatttctcagtgcaattttagttggtgtttttcctcctgtggacagtttttaggaatgatacaagttatcgtggaatcacccagttcttgagtgtgtgaacgaatggcttctgctggaagttaattcttcttggcatgggtgtctcacatttccccagcagctggatttgacaagggagggcctatcatgattaatatgccttctaattgattggtttaataacacctttactcctgtggatatatttttcatttcttgctgaatttttttttctttttgagataccagaacctgttaagtgacacggtatagatatttcaggacttccattttctcttttaaaaatatttttgtaaaggtaatgcctgtacacagaagagtttgaaaatacagaaggcaccctttttcatgaagttattttgtgaagtggtctattgcctgcaatggtaatagctaggaccccttacttactacccagcaggcactatgtagagctccaaatgagaaaatgttggctccttgcagatggttgggagaggagacaccagcactcttaacagacttgttaagagttgagactgataaccagagaatattctgggggaaaccaggagggatcgccaacatactcagttatatttgtggatggtgaattctacagtgaatttttctgttcaaaatcataatacagtcacttgctaactaccaacgccatacttgctctcagtgctgagaggtggcacaaatattattttcagtcttggtcatcaggcagggcctggcaggcatcatgctctgtgtgagtcagtggagtttattataagaatgcggtagcctccacagtgattatatttattataggaaaacgtgtaattttgttgtcagaacctttcagcaggtaccacaaaatctcatatgggaaaacgtgtaaatttttcatgaataaattcctttcggtattggtaatttccttctctgttttagtataaaagaaagcattgccttataggtggctaagaacatctgtagcatacataacacttaatctaaaggagtgttgaagaccgggctgattcctaattgagaatgagcaagaatagaactctttttgatagttatccctgttcttcctccaagcctctgccttggagctatggatactataactaactgaagcttcttctttcaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtaagtcctgtccggtggctagcaattcacgttggatcacatgcatttgttttcaaaaaatttaacttcttgtattttgcatcagtattttaaccctacagtaaaaatcttggttcctaatgattcaccataccattaatatatttatttgcattaccctatgatatacatataaatgtttttaaaattatgatgtcgtattatgaccatcactaaacagtagtttaagatgtcacagcacttttttttttcctcactctgtcacccaggttggagtgcagtggcaagattatggcttactgtagccttgacctactgggctcaagcaatcctcccacctcagcctcccaagtagctggactataggcacatgccaccaggcctgactgattttttttaatttttagtagagatggggtctccttgtgttggccaggctgtctgaaactcctgggctcaagcgatcctctggctggacctcccaaagtgctgagattacgggtatgagccaccatgctcagccctccacactcctttcatcttcagtatatatctcaccacggctctacagtcaaggtcttttggttaaaagtgctagcattaatttttttctgttaccagcttgatacatattttagttcatttgtatctgtttgtattctattatagtttggttttttcctcccttattaattttactttttgactatcatgttagttctatgaaatatactcagaaagctcacttttcactcattctctattgataaacattttcattgttcctgatttatttttttggcaaaagaaaatacatatttttccttcttgtacaaaaaaagtacaaagttcactgttctgcactttgcttttttttttttttttttttttaacttaactttgtatcctggaaatcatcctttgtcatttcgtggagatcttcatttttgtttaattctttgtagtctttttgctgttactatacagggtgctcctttcttattcaatttaaaatagctggagttccaggcattttatctcacacttcttagatagttatctctgcattgtgactgcatcttaagaacattagaagtaattagtcttatgcttgttggcaaagcagaattctcgacactcattataaacagcagatagcctggcaacattttcttttgttgtaatcctttttgtactagaaatttttctttagcctacttggataatgaatgtttgttgtttaattaatattactattctattgagatccttttattttaaattgtaaatactaaaattattattattaaaaggtttaatttgttttgatagcgctctgtttaagaaaggacaagctctaattactaattggtgttttgagaagcaaaaagcatctacattatcacaatatattacatattaagtaatttttatcttgaaaggagtatgaaccatgatgtgaaatgatgaatagtggaaatcactaatacttaagttcattatatgtattttttgttttgttttgtttttttttgagatgacatcttgctctgtcgcccaggatggagtgcagtggtgcaatctcagcttactgcaacctccacctcccgggtataaatgattctcctgcctcagcctcctgagtagatgggactataggcacccgccaccacgcccagctaaatttttttgtatttttagtagagacagggtttcaccatattggccaggctggtcttgaactactgacattgtgatccacccatgtcagcctcccaaagtgctgggattatagccgtgagctaccacgtatggtccattatatgtatttttacaagaacattctgtgtgctgatttactaagtatggattgataataaatcagcttttgcctgggtgtgacttttataaagtgtcaatgttagaaataatatttacattctgctgaagagttcttgtagtacaattgtgtagataagttataaattttataaatcagaactctgtgcatacttatatgtttttgataccatttttttgatatatgcatacagactatgtattctgagtgtcaaataaactttttttaaaggctagaattaaaatttagtttatgacattgaccaaattgtccttttctggtgaatcgactattttgatacatgtgtcatagtttgcatctaagtttttcttcccttagtttcacgtatgaataaccagatgttttcacatttgtatggtggccataccaaaaattactcttcttgaccccaggaacacccctcttttcccgtttcacttagagatcctactaatgcctgggtagctttactttctgtcttagcctgttgtttgtcccagggtgtattagtctgttctcacattgctgtaataaactacctgagactgggtaatttataaagaaaagaggtttaagtggttcacggttccaaaggctgtatgggaagcatggctggggaggcctcaggaaacttagaatcatggcaggaggtgaaggggaagaagacacatgttcacatggccagagcaagaaagagaaagagtgtgaagggagaggtgctacacactttcaaacacccggatctcatgagaacactgtcacgggacagcactcgagggatggtgcttaaacattgaaaccacccccatgatccaatcccttcccaccagaccccacttccaacactgggaattacaattcgacatgagatttgggtagggacacagagccaaactgtatcaaagggttgagcacagagtggttcttcccagttctggactttaccagattgttggtggccaccatgagtggggagaattagccctttgctgtcaccttcctcttgcaattctggtattatctgtcctctgcctaccacccacccacggaagtcttgttggatgatacacaccagccagattcttcaacattaggcgccataagtaagaaagggccaaaacatcataccttgtgagccattttaatggatttgggctttattttgtagcaacaggaagtcactagactttattttttatctatttatttttttctttttttttcttttgtttttgagacagagtcttgctcttgtggcccaggctgcagtgcagtggcacaatctcagctcactgcaacctctgcctcctgggttcaaatgattctcctgcctcagcctcccgagtagctgggattataggcgcctgccaccacgccgggattttttttatttttagtagagacagggtttcaccatgttggccaggctggtctcgaactcctgaccttgcgatccaccctcttcggcctcccagagtgctgggattacaggcgtgagccacccgcacccggcctagactttattttttcagagaagaggagtgattgtagatttgcatttaaacaaatcaccttcttgctttatcactatgtcccctaccatatatcttcgttctcccagagactgccatgctgcagcctctagccgtgtgctgtccagtactgtagccactggccctatgtggttgtttggcacttgaaatgtggctagtggaaactgatgtgtgctataagtgtaagatagataccgtatctcaaagatataatgtgagaaaacctgtaaaatgatcacattcatttttatataggtttaacattgaaatagttatgttttgaatatgttgttaaatacatcataaatgttactgagttgtaactatgtgattaaaattgatttcacttgtttcttttgacgtttcacgtggcagtgaggaaattgaggttccataggcagttgcattctgtttccgtggaacagtgctgttctctgtgctatcccgagccaccagtgactctgagctgtaagggtcttagatcccatgatctggcaagtgggagagggaggtagaaatgagtccctccataccttcaaccagaacttcactgcatgttgcaagtcagggaacaaggcttgtttggggtccttttaaatacatctttcattaaatacatttggatgtaagatgattatatgcctttctagaaccctgtggtgctctcttcctcctcagacataatgtctttggatactaagggtgcattgatgaacagaacacagggcctgttcttcaaagggggagaaaagcttgtaaaatcataattttagcaatctaaaaactacatggtgacagtgacactaataccatcagcattgagcattcacaaagggtcacaaagggtcagctactatgctaagtacttgcactcattttacccactcagtgacacctgtggagaagaaccgaagaaagcaccttggggaattgcagcattgaaggcaaacaaagagagacgattccccatgagggagatgagcagtggccagagagataggataggaagtaggaggcgttacatgctggaaggcaagccatgtaaatttctaaatgaggggaaagataagagtgcaagcatgatgcaaacggaaagcagttgaagtgtagtttttgacatggtaaaatcatgtctgtcatgcagactgaacacatgtcaaatattttattcaattcattaatgagatgaactgtaagatgtgaaaaatggttcattttgacttggagagatttaaaattccttccccagcacacttgagttgcatggctatgaacaggaatcatctggaattatctggaatatcgtcaaattatttccatggtacaggaacacttgcattgctatggggtgtctgcagtatccatctttacaaagctctaagggtgtggaatagcttgtaaggatgtaagtaataagggtgtaagaattttaagggtgtaagtcgtaaaatagcttatttagtagcaagtcaactggaggaacatgctgtagaatcagataatcctttccccgccaccctcagggtctgcaagacaacacctgcatttcaatgctaggacacccagcaatctcttttgctcatatcttgtttttggacatttgtatgtgtgtgtgacaaaacattgttaggatctggcaaatagaaggtcattgttgaccctgtggaatagcatggggagaagttacgttgtagtccattgcaggaaaaacaacgtgggtcaatggtgagtcagctgtggactcctggaggaaagacctagaggcaaaaagctttgaggtcattgaagacaaaaggaaaggctttgtgattttgatttttaaatggaaggaatttgggcctaaggaagcttaaactaaactgtgggaaggactgatacccatcctaagcactagttgcaggtatctcttaagaactaggttttggccgagctccgtggctcagacctgtagtcgcagcgctttgagtgagaggatcgcttgggcccaagagtacaaggttacagtaagctatgactatggtattcagttctgtaagaactaggcagaaaggagatccagtggatatgtgattgcctgggtcttggaaaggggagggaacatctccttttctgaggaagaatggggagatagggtgggttaattaggcagtttcttttgccaggtgataaaaaccccctaaacaatttgtattttatatatggagaatattttctcccactgatgggaaggatcgtagtgtgggggatgcttacccaatttcttaaaaatgtacacagaaatgagatccctaggtgctggaaccgagggtcagtactgcctagaatttccccattagggtctcttttgtttctgcttaccttcctgctgtgtgtttactccttttctcctgctaagatgggtgtcctcaaggagaccagggatgttggtagccacgacctctggtagtgttagtcctggtttggcatcccaactttgttctgcccatgggcattatcagggagtgcttctggttggcactgctttgagttacacgtctgcccttggagccaccaccgtatctgcagggactcagtatcaggactagcccatcttggccttgtgggtgtggccgtgtgatgtggggagaggagctgttgccagaagagggatgaggaatggcatgggcaaaatagaagaatgaccccagtgctgcaacacagtgggtatggattagaaaaggcttgtaggctggacagggcgaggtgagggagtgcatcatgcattaggattttaagtggctttttggggagtattgagcaggtggcaggggagacaagttcactcagaagttttagacagtgttgacattttctttcaaattgtgcatcttgaattctctagaacattctttatagatatttcaggatcttcctgccactttccacattgtagctattctggtgtttttcttttataattagaaataatgaatagttctcatttgtaggcttggattttcatgcgtgtgttggaaagtgttcgttttgccttttttttttaacttcatgtttttattttttaaaccgtattgatggattcattgctaggaacggcaaggaaaagaatactctcacaatgttaccacgtttccttctcacaggttttataagttgtattatttttatattttcaaagttataatcttaccatctcctctgttgccatacttctcattcatagggtctgtctttaactggactcattgtttacagctagtcttttcatcagatcatctcaaaccctgtattacattaatattttgattccgttttgttggatgactttcatcttaatcattcttattgttgaacaatgtttttcttattattgaacaattgcttcattaggtatagaattcctggttgatgggtgtttgattcagcaccatgaaaattcacggtattctggtatctgtcattgggcaaattcttcttctttttttttttttttttttttgaaaaaaatgcgtggaaaccattagttaccattgacagagctactcatttaatgattttttttttctcccaatagtggtggatgaattttttactttgcgttctcactttgtgtctctcatgaccaaaatattttagagataactagctggctaagtctgaggtggctgttaacagtctggtcctaggatagaacaaaaccaacagaactcatcaccgtcacgtcgttttgactttttgctcacaagtttaactcattgtttaatttgtttttccaaaggattgctttgcagtatacatgttggtgcatagcttaacatgatgataaatacataactttgactgtgaattatctactctgctcctttcatagtctcatgcttctctgaatttgtattttgtgttactgggttgttttgttgttatcattcatagtgttttttttgcccttgattctttgaaccaggttttggtcaagggcaaggttcaagagaaaaagtaaaaacacagtcttttatttttgctcttggttataaacccttgtaaagtttgattgagaagataacttggatcttatggtttaaattgagtctttgtttaatcccaagtcttccaaattctttgtgcttatgtattctttcctctttcccaatcgtaatagttttgcttctgcctagtcattggtcaaatacctgcctttcaatgcatttaaaaaatattttgtgttagcaccatttgagatttttccccccttttttttcaagtatgtattgaggactgttatgggatgaatgtttgtgttcccccaaaatttagatattgaaaccctaaccccaggctattcggagatggtattcggagatgatacctttgggaggtgataactgttagatgaggtcatgagtatgtgcccccgtgatggaactagtgcccttataagaaaagataccaaagagcttgcctttttctccctgctcaaacgaagaagtcacataaggacacagcaaggaggtggctgtctgcaagcaaggaagagcggcctcaccagaactcggtcatgctggcaccttggttttggacttccagcttccagagctgtgacaaaagacatttttgttgtttaagccattcacagtaaggcagcattttgagctaagacaaagacctactgtggcttctgtaggcaaaagccctacagacccaaagtgttgtgagaattaacatttctagggatcatgccttacagtggattttatccaacacttagctctgacctagagcacgctagtcgtcgttaggtaaatgcttgtggatggtagtttctggcacagagattggagcggtaatgggcaggagttctgtatccagcttatgctgagagagcttcccataaaatggagcctatactttccttttgcagagaccatttctatccattgatagaattcttttttttctttttcttttttttgacgggatctcactccatcgcccaggctggagtgcagtggcatgatgtcaacccactgcaacctctgcctcctgggttcaagtgattctcatgcctcagcctcctgagtagctgggattacaaggtgcacgccaccacatgtggctaatttttgtattttggtagagatggggtttcaccatgttggctaggctggtcttgaactctagtgatttacctgcctcggcctcccaaagtgctgggattacaggcatgagccactgcacccagccttccatcgacagaattctaaagacatgtttaggcaagagttggtggaatgttggcgtgtagtccagatttgccttcagtttgtttttatccgcttcaaatgcaaaggccatgataattcaatagtaaagtcataaatccatagtatgacttaaaatatttggttgtaatattacaaagtatgcttccaaacacattaagcatgcaaaatccttatattgttctttttgtttagtgtttagggtagtttgtgtgaatcatagatgaccacagtagactgttgagttgttattttcttttgacattctttgatagaaaacagtgcttttcctctgttatttatactagaagcaaaaagtatctctcatagcattatggaaacaactcgaatacaactacgtcaattattcatcttttttttttttttttttttttttttgagacagagtcccactccatcaaccaggctggagtgcagtggtgcagtcttggctcactgcattctttgcatcctgggctcaagtgattctcgtgcctcagcctcccaagtagctgagattacaggtgcccacaccattctcagataatttttgtatttttactagagatggggtttcaccatgttggccaggctggtctcgaactcctgacctcaagtgatccacctgtcttggcctcccaaagtgctgggattacaggtgcgaaccactgtgcctggcagttattcatcatttaacagcataaagtggccacttgctttaataaactattatagtaccaaaattttggcctgaggaatttataaatataaatagaacaaaacaaaaataatattccccttgaaacatattctaaaaccaggggctaaaagttagaattgttagattttattgtcctgtttctttgggatgaaatgaaattttttaaaaaccatagttttggccgggcgcagtggctcacgcctgtaatcccagcactttgggaggccgaggcgggcagatcacgaggtcaggagatcgagaccatcccggctaacacggtgaaaccctgtctctactaaaaatacaaaaatatttgccgggcgtggtggtgggcacctgtagtcccagctacttgggaggctgaggcaggagaatggcgtgaacccgggaggtggagcttgcggtgagccgagatcgcgccgctgcactccagcctgggcgacaagagcgagactccggctccaaaaaaaaaaaaaaaaaaaaaaccacatagttttacttaaaccaaaggctttgggattccagaagaataaaggaagtgtacttcaattctacatctcaagaatctatttcttcccaaatttaatacaggcaaatttagtctatatttattgggcactgttaggtttttgtgtcaggtgttgctgagtggcgggataccattcttggcccataaggaccttataaatctattaatacaattaaattgtgaagaaaatttagaaaattgcaaaggtagaatgaggttgcaaattggagggtcttattctggagataggtggaagctatggcagatttttagcatgaatgctttctggtcaagacacttttagaaaaattagcttgtttccaagagtagattagattggggagagaaggaattcaaattacaaaataatagtgtgaaaagcattgtgggagatcatcctgtccatcccctcattcatgatatcctgaggtgctgtattaagggcatcattgctctttgtcacccgagcagtccaggaaggctgcctgttcagtcttagactgtatcctcacagtcttaaaccatatcctcactttatgatagaaacacacaggcaagtgttgtaagagttacatattgtcagttaaacaagaaattggatgagtccccatactattcttcctcttttaaagagagaacttgggccgggtgccatggctcacacctgtaatcccagcacattgggaggccgagtcgggctggtgacgagatcaagagatcaagaccatcctagccaacatggtgaaaccccgtctctactaaaaatacaaaaattagatgggcatggtggcgcacgcctgtagtcccagctactcgggagactgaggcaggagaattgcttgaactcggaggccgaggttgcagtgggccgagatcacgccactgcactccagcctggcgacagagtgagactccatcccaaaaaaaaaaaaaaaaaaaaaaaggaaaaaaaaagagataacttgaaacaggctcttccagcctctctattcttttaataacatgcagtgcacttcttggagctaggggctagggtctaggggcttcttgaaggtgttgatgtctctggtaacttacactgcagggcaggtctctggagggaactttgaacaggttaagcctcaaatgcaggcagagatttccaattgggaaagttaggtgaacgtggctttatcatctcttttattggtttatgggtattttctctttttcctgttttctttcccaccatttaacattatctgttctctgcctttttaaccttctggctttaaatgaacagttgcttaatctatagattaacaggatcagcaaacaaagcacagcagtgcctgcacaaagcagaaactgcctgttagatgagttgagcaaaaccatttcctttgcccaactttctcctctaaaatacataactgaacatgggattcccagacacgaattgtttctcaacctttctttttccattctacagttgtatgaaattaaaattaaataaaaagcaaacaacagaggtagtctttttaatcttaccgagttggaaggtatactatatacattatcctatggtagaaggtctggtacattcttgaccattattactttgctatgggcacccttcacccttgtaggctgaaaattggcaaatcataggcacgtatacattgtggttttggaatttgaaagatctcaaattcatattgagtgagacagtttttgaatggagggatcaggctgtctatatgtggatatattttatgcatttctggtctgtataaatgtaaggattgctttactgaccgtagatttcttctagggttaaaaagttgaaggcagtttggatattttaaaataagaaaattagccctacattgaaagagagaataggaaaatctaggctatgtgtacacatctgcttgtgttttcacaattgtgtcctgtcctatttatttttatttctctcccatctcttccctcccacccacaatttgtttttcattcaacactttgaaattagaatagcttatctatcaaacaaggttctttgacaggattttccaactctgagtatgaaagtgtggatataacaatatatttatatagaatgtcagaagcagagtttggccagattattgggaaatattctaacgccctgcagcattttaccgcctttgcttttacataaggccatcatttggaaacatagcatgctgatacctcccattattgcagtagcgtttcatcagctgcacgctgcctctcagcgcctcattaagttgcaagcagagaacgttttcgttttccagttctctttctcaattaagttagagattgtattttcagtattcctcattattaaaagaatttttgttgagaatagtttaagatgtgatatttctttgtattccaggaaaatttaaagaatgtcattgaaatccagaaacagaagtatatattgtctaggacatgctttgtcttggtttatatgcctaagtccgagtagtttaaaatttatgattaatatactctttgaatgcttagaaatggcatcgattttagttgttctagtttcatcttaaataaattggagtaattactaactcaggattatgatgtgctacacataaaacatttgcagtgatgtctggtacttaggaaaccctcagttatgacatttttcttactttcttcttggatatgttctttgaaaaagggactggtatggaaaagctttgagacgatgaaaatatacaagataggctatctctgaaacaatttggtatagagcatttgattctctgtttcatgtatatattcatatatgctagctgcagtcatgcagacacaatttggatctgggtttttttttttttcctttttcacatgcagttataaatttagaaacctggctgtttcctctggactaaatgtatttatttaaattttcagtgctttaaatgcaaaattacaagcatatgtacaagtagaaagaatagtaaaatgaaccctcccatctatctgtcactgaattgcagtaatgatcaaatctagtcagtcttgttcctgagtcagtctggaatgcaggttttttcccttaactattcaggttctccagcaatcctggcaaaacacatgcgggtttcagctcatctctttcagtgacctgtaaattaagatgtgctaaagcatcacatgttactcattgacgttttgttaatatatttaaaacattttattagtaccacataatttttagcgttttcaaaaagaaataagaatttggctaaaggtgaattttaaaaattgaatgactaagtagaaaagtaggcaaactgttttcagaattagtagcaatttccaattccatttactctgttcattaactttgttttcagccttttgttagccatgtttgattttgtgtttttgaaatacctttggtcattttaggacttttggcattaatcacctgtcttaggtggcctgtttacaaaagggacttttcggttctccatttgcccttaatgttgggaatagagacaaagtttctgtaggactctgacaacatatggctttacaaagtttttttttaatatatacatttctcttcttggctttcttaaatatcatcaatgtcttttcttgctttacgtatttagcaacttaaatgtcatcttgtctgagatcaatatatttccaacttggagagatagtgagaagagagacttcagatttttaaaatgtggaacaaaacattctgatggtgaccaggttttaaggtatttcttcagtagacaaaactacttaggctaccgatgataagcttttaatattaataataattattatatataaaaatattaatataatatgatctaagattgtttctattccccctctttcctggttgttaggatttggaatttcttatgcactctttttttttttttttttttttttctgagacagagtctggctctgcctcgcaggctggagtgcagtggcgcgatctcggctcactgcaagctccgcctcccaggttcacgccattctcctgcctcagcctcccaagtagctgagactacaggcgcctgccaccatgcccggctaatttttgtattttttagtagagacggggtttcaccatgttagccaggatggtctcgatctactgaccttgtgatccacccgtcttggcctcccaaagtgctgggattacaggcctgagccaccgcgcccggcctatgcactcttaaaattatttcatgtactgatacattcactttcaatgataaaacatggctgtatgaccaggtaggtaggctgcttttcagactcctgtactaactgaatgggccccattagcccaagtgagagcttaacttgtcactcttcctaaatgccttcctcagctgagtgcttatttacagtaatctactgcttatagcagatgctatgatttgtataagctgtccatatctacttggaagaacaatattgaaaggcattgagatacttactttccctcaaaagcccataatccaacaactatttagaatatcttatttaagagataggtttataatacccaaagggaacttaatgtatactgaaagtccctgattgaatggattctgaccttaaccagaaatattaattacatgcttatcagacacttactgtgctatcccagggtttgccactgttgagggaacaccagatgaatcatattgtccctgccttcaaggattatatattctaataggacagacagcccatcagcagaataggaggtagcaagtggtcagggatcagataaagtgatcaaggcaagtggaggatgcagtggtaggacagaatggtctgcgcacagagattgagggcatcatgtggtgggaattagtgaactagcatgcaagccgggcctttttcttggcttttgttcaattggaaggagcataaagctactaaaggagcttatgtttagaaatagggcttattgaaggggacaaggcatctcctagaatccaaatgcacatcccaagacttaaaagagttagagggcacaggcaggccctgtcagatccggtctctgccagatccagtcttgtctgtttctcatcttcccttgtctctctgagtattccctgttttcctttcctggactaactgcttcttccagatgcctagtttgagtccacccatggttttttgggggcttttctttgtttttttgttacgttctgtttttgttttttgtttttttgaggcggggtctcactctgttgcccagactggagtgcagtgacgtgatcccagttcactgcagcctcgacctcctcgcttaagctgtcctcccacctcagcctcccgagtagctgggactataggcgcaagccaccatgcacccatgggtgttgacatgccacctgtggctcagacttatgctcacctccagtcaagtttcttccttatctaacgacagcctttgtgtgttcgtgaaagcccttgagggaaagaatgctacgtacagtttggttttcttgggctctaataagctataggggatggtgctgcagagtttgaaatacagtcactaatgagtcctccttcagagagctgagggtgggagcaccacataaaaggagttgggggtcagcagcctcctgatagtctgaaagccaaagatgatggatgtggggctccacggggaggctataacatgccacgttatgttttctgcccttgaggaaagcttcttttcaccaggcaggcagtacagatacaaacttaatagcgatgtgaggcacactgataaataccagaaaacattctgtaagaggggttagcattatttcactgtgtgcagaggaggtttgtccatgtggggggaccctaatgggcgtaggcatttgaagtgtagtctaagggatcttaggtaagagtgtgtttggaacacagtgtacgtatttatacagcaaatgctctgtgtggctataatttaggaaaatttgtaggacttgaggacagcttatttttctactagctgacagaggcttgggtttgcttctgctttaagtataagtatctcgagtccttttttaaaaaggaaaatcaaggaaatagttggaattgggtttcacataaaaaacattttagaaaagaaggggagtatgagagttaggatttgctaatgtttgctgtattagcaaattatttgcaatgcttgtcatattagcaatacatgtatgttttggaaaacacatgcaaattaagcagcttaaggaacttttatctttatttcctatgtaagatttgagatgctgcaaagaaaatattaaaacattgaaccgatacttacagtagtcctcctatatctgcagaggataacttccaagacccccagtgaatgcccgaaaccttagatagtaccaaacccaatatatactatgtttttttcctataggtacatacctgtgataaagttgaatttacaaattaggcacagtaagtaattcacaacaactaataatagaatagagcaattatgacactacgccaccatcactactcttgcactttggggccgttattaagtcaaataagagttacttgaacacaagcgctgtgatgaagtgatcaatggcaggtagcatgtacagcatggagacgctggacaaagggatgattcactttctggactgtgtgggtttgagatttcatcacactgctcagaatggtgcgcaatttaaaacttattgttggctgggcatggtggctcatgcctgtaatcctggtactttgggaggccaggacaggaggatctcttgaggccaggagtttgagaccagcctgggcaacataggatgaccctatctctttcttaaaattaaatataggccgggcacggtggctcacgcctgtaatcccaacactttgggaggccaaggcgggtggatcacaaggtcaggagattgagagcatcctggccaacatggtgaaaccctgtctttactgaaaatacaaaaattagccgggtgtggtagtgggtgcctgtagtcccagctactcaggaggctgaggcaggagaatcgcttgaacctgggagatggaggttacagtgagctgagattgcaccactgcactgcaacctgggcgacagagtgagagtccatctcaaaaaaaaaataaattaattaaataaatagaaaaataaaactcatttatttatagagctttctatttaatatttttggactgaaactatggaaagcaaaaccacagataagggaggactactatagttatgataaaaatcaggaacttaatctagccacgagaacatttgtatttctgatgaaatttttattcttgtctaaaatgttgaactatatttatatttatatctttaaatataaatgaactatatttatatctttataaatgagctatatatttatattttatataaatataaatatttttatatctatgtaaacataaaaaggctttttttcctctctggatgtttactggggttatttaataaaaacctagggatgatattgggaatttttgttggaattttaaattttaacgtgtttgtttctttttttgttttgtttcgttgtttgctttctgagctgggagttggctgtgcatgtggcagttgctttatcaaaagggtcttgctctgcttcagaagaagttttgttaccttgattgctaatgttctcccccttaaataaacctcctgatgttggaacagcaaaattgcattatgtttggaaaatctaaattaataatcccttatctctgaataaaagtaactgaatcaggttgaatttactgcttccattttaataggacagtaggaaaaaactatgcatgggctttatgtggttcaaatttttttttttttttttttgagaccgagtctcgctctgtcaccaggctggagtgcagtggtgtgatctcagctcactgcaacatccgcttcctgggttcctgcgattctcctgcctcagcctccggagtagctgggacttaacaggcgtgtgccatgacgcccagctaatttttgtatttttagtagagacagggtttcaccatgttggccaggatggtcttgatctcttgacctcgtgagccgcccgcctcggcctcccaaagtgctgggattacaggtgtgtgccactgtctccggcctggttcaaattttgtaatctccacatccatctgcatattaacaattgtcattgttggtgttatagagagagaagctgaagcacagaaaataacacgcatgttctagatttcctggtgggaactggaagtaaattctgtgtttggctgatcactgtataaagttctagtactagtctttagggaaaagccccgtcttcatggattttaatttgtcaatttgcaattctacctacttggaaatagtcaacctaatcagttactgccctgttgcaccaactattaccataggaatctgaagatggtatgcaaccaacatgaaaaagagatccttttctcctggtaatcagtttgtaccctacaaacaattcgttggttttaagttgtatagagggaaccatgatgtgaatatgcctgccactgggtttatagaaaaccgatgactcctgaggaaattctcagcagggttgtctttcagtgaaaagaaccctttagaaagatgcagtaactcacactttactgcacataaagtcatgtccctcagggtggactctcaggtgtcatacagtttatgactcatgagtgctgcagctttgtgatctaataaacaagtaaagacggtgggagaattatattcctgtacaaaaagccacttcacctttcattatataggaaaaaagccatgggaatattaaatttttagtggcatttttagagcattttctgtgatctaaagtgcctgcattgagatattaagatacttaagaacaaagtaaaataaagagaagtattaatagcaggatccctttggagctaggttactgtggcagtggtttcaagaataactctctcagcacccaccccaggattctgatttagagatttctttgtgtgttctcttagcatatacctctaccactgagtttatcccctgctgctacactgattgataccttatttttaaatttttgtttttaaaatagacggttttttagagcagttttagattcacagaaaagttgagaggaaggtacagaggtttcccatatacttgctacctgcacacatgcttagcctctcctattataaacatcccccacagaatggtacatttgttacaatcgatgaaccaacattgatacatcattagcacccaaagtccatagtttccctgagggttcactcttggcggtatacattttttgggtttggacaaatgtataatgacatatgtccatcattgtagtaccatacagagtagtttcactgccctaaaaatcttctgtgctctatctgttcatcctccccctgcccccaaccacccaaacccgtggcaaccactgatctttttagtctccatagttctgccttttccagaatgtcatatagtgggaagaatgtcatatagtatggagcctattcagatgggcttctttcacttagcaatattagttaagttcctccatgtcttttcatgggttcatagcccatttccttttagtgttgaatgacattccattttctaggtgtgtaccccacagtgttatttattcattcacagtaccttattttttaactgatttttttggggtctgggactaaggtgaggtaaacaaggcagtcaggaattgagattaagaatttttaatgtaatatttttaaaaatcaaaattactgcaaaaaaactccatgatgaacaaaatatcaactttttagacagagattgagtcacgctcaattgacatcaagccatgcatagttctcctactgccttattaaaatggaagcagtaaattcagcctgattcagttacttttattcaaagataagggattattaatttagatttgtcaagccctctgaatgtcatggacaattgtgagtgaaattccgtgtatcgtctaactccccatggatgttatcttgctcagaaaccttgttctccctccaaggaaggcactacttctcctgtagccttctcaggtcgaagttcttttctcaaaatcctcacagcgttgcccctagaggtgggaatgtcctcttttcctcttcatttcctcttttcctcttcatttttgtttccaaaccactcttcctttctcttccctaaaagcccccctacctcagcttgaagtgcatgctttcagacaaatcactttgctcctccacctgttggtcatctccagacctctttctacctcatcgatgttcaccattcagactttgaagattttagctcctgccttgctgtcatcaggtgtagctgcttcatttgctgattcttaagtgatcctaatattgatctcaatgatccttccagtattccaatttgtcagttcatgattctcatccctccagtgaccttgttttcatcattctccatcacccattgcttacaatggtcatattatactccttgcaatgaccaatcactgcaactcctccaaaattttaatttcaattatttgactctccaagtactacttattaccacctcactttctccagtcctgtgcgccatacattctttagcccctggagtgtgatcagtggactcagattccactggtccctaccaccttttacctgtgtctaattcccctcatgttctcacttttgccacttgagattgcatggcccagcattctaatcattcccttgcttataccgtcacttctctattcgttgttatttatctggcaaaacaaccaccctgttgcaatgcaaggtttatgcaagggaatgcacattttcgaaatagatttttatgtttatgtttttctttcaaatctctactcattaataaatagaagtggtttgatatttcacaaatgtgatttaattgatcaagtttcacatttcattctaagtgctcagttaaacaccatgatgtattcacaacagtaatcaaagcaaggggctgtagttagttataggagcaaagagaattcatgagtttatttttatctaaatgacacccttgataacttttcgataagttattttcatgtaaattacttggaataagagcaggatttttatatagaaagctttctatgtgtcatatcctgtaaaatgtttgtccgattagtgagagatagggggaggttgaattttactaaacataatatccccagccatatctactgtaacgtttgtttaggacattacagactattatcctacaccaatttatttcacatcaaaaatcaccccatgcttgtaagaatcctcctttccccctcttgtttctattttagctcccctggaaaaataatattttaagtctaaatgagtacaataaattaaaaagttgcattgcttaatattttgaatggaatagctagatgcttgttcatttgctatctttttctttgtactgaatttctttagcttggtcagaggtagagcaggcttgactcttagtggatagtggtataggttgagtatcccaaatttaaaaatccagagtccgaaatgctcaaaaacccaaaactatttgcatgtcaacatgatgctcaaaggaaatgctcattggagtattttggattttggactttccggattccagatgctcaaccagtaagtatataatgcagatgttctaaaatcggaaaaagtcagagaccttgaaagcacttctggtcccaagcattttggataagggacactccacctgtaccttacagtggaggcttgttagatgcttgtaaatgccagcccctgcctcaagtaacaattgattctttttgtgtgctctcccaggtctaccctgaactgcagatcaccaatgtggtagaagccaaccaaccagtgaccatccagaactggtgcaagcggggccgcaagcagtgcaagacccatccccactttgtgattccctaccgctgcttaggtgagccggccggccgtggggctggtgttgattgggggcctggtcttgagggaagaaaaagaggatgctcctgttaggtcacatacacagacttgttcttcagcacattgccactctgtgttgtactgtgttttggactcttgcagttacattctgtgcactgaccctataggagcagtatttttgagttccctgcctcagaatgaatttacccagggtgtatattgaaattacaaattcctgggccagttccaggactcctgaatgaaaaatgcctatagtagcggatccgggaattcttattttaccgtatcgcatagatgattctcatgaacaggggccttgtgtgtttcttcacatagactttctagaagaaagaatctaatgtgaagctgcagcattttgttaatttctaaaaaaaaaaaaaaaaaaaaaaaaaaggctgttctacaataactactcctcttatttggtgatagtagaggaattggaattgggaaggctttttttctgcttacatcaaactggaggaaacagtctgattactaatgtttaatgtttgttttgtcacaaacctcaaatttacctcaacattttatttaaaaaaattgagtagttttatgtggtatcgtgctcttttcctctggaaactgtcatactccatatttaatctaacttggttattagtctgttcctagttgttgtttttatgtgaccagtcattgcagaagtcaagttctttgctctggtatcttacatctagatgttttccacaagtgtattcatcagtaggcaggttgtaaataaggatatagagattgtctgtggagattgtggacaaatattgatgtatattctgatgacttcttataaaggttaaaaggaatatttcaaagaaaatccaacagcaaacttacaaagtaaggatatttactgaagattccggagatctatgtaatatatggtcaaaaatgaatgtcctaagagcatactgattgctgttcattcatgtgcataatttgagacataaaatgaaaacactgggcattagaaagaagatttcacctggtgaatttacttacaatgtaattgatgaagagagtgtagaaaactagatttcttaaacatctgataattcaagagtttcctttttcctccttattccttgttcctatatgctttataaggcctcaactcttttctggttcatcaaaaagtgcagtttaattagatgaaaataagcagcacttaaattctagatacagaactcctactcaaaaaaatatagaagtcatctgtccccaggaactgtgtattttatactcacttagttattacaccaagaaataacacacagagattgaacaattcagtacaagttaggaagtagatcatgagtctgttaatttgatgggccaggctttgttaatgatttgtttcttttggttcttacttacctgttctatgaagtattccaaatatattttcattagaatgtaagctcactgagaacaggtagggctttggatgggcattattgctcctggtatgacactcaaaactgcagtcatgtttcagaagtgaacttatcattttacatagatttttctgcttcatattctattcagaatgatttgtattaaagttgatggaaagatgaaatccacattaagattgttatttactacctagtgccagttgttgtagcaaatatcctttgaaccccaggtctattttatcctatctgttgatcacttcctttgcccctgctcttcaagcctcctcatctctgaaaagctctccttccactgggtgtggtggctcaagcctataatcccagcactgtaagaggccgaggtgggaagattgcttgagcccagcagttagagaccagcctgggcaacataatgagaccccgtctcttaaaaaaacaaaaacaaacaaaaaaacctccaagtgaacgaacagtacaacaagtaataaaggatataaaagatgttctcagatctcaatatcaagtcagaagattttataataatattataattacacggagactgtccaaatatttgtatttgttgtgctacactgcattatgcaccatttagggaaacaacagcctaagtaatgtggttttaaataattaatctcatctaccaaatacctgtttgatagtaaagttaatgacctgtcctcaccttatgtttttaaccattatggaaagagaaccacttgaaaatatatcattccactgacagataattacccagctgtgaagcacaggctggagactggcaaggagaaatggagagaagcctataaaataattctttctcagggaaggagtttattcctgccaaggctttgccatcaaccattatcagatttctctttcatcctccgaacagattctgggctcctttgatgtggaggtacataattcaaaaagttgggtcacttgtgacatgttaagcaaatttctcagttctggttgtgtctgatgtgaaatgagcacgttgacactaactgttcttgactactatgggactagataatcatttgctatttcctgtaggtacttggcgtgttgaatcattttaccatattttctcattttatgaatcactggtccttttccattggacctgaaattagaccctaatatgacaagtagagcatatgttgtctcttactttgttattaaccttagattagaattaaggaatataggtgctgcttttttcttaggtagtggcaaattttccaaaggtctcaagcccagagtataatacagagttgataaaataacctggtagagtatgtagagcatgccttgtctttcaggaaaaggaactgaaaaacataggttttccaataggtgtaacataggtaacatagggacataggtgtaactgatgtacattttacttgcacttaatagacaggcagtgttagttcttcgtctctcttcacaggctatacaactaccaagatccagagaggttgaatttcttgctataactcacagcaaataaatgggagagtcagcactacagtccaagctctcttgattattggtccagtgttttttacagtatacacaaaaatttgttttaaaacaaatattcactatgaattagagacaatagcactaaaaaagaaactcacagctatcgttgtgttgacagaggagacaaccatgacctagtataggataaatctccatgtgatatgttttctactttatagtagtagtaggtaacattcttacatattattcctgcttaacaaagaattactgttcagcattaacataagtatgctctaaaatctcttgactgtatccaaatatatgttttagacaagagttttttttttttactgacctagcaaacaaaataaaattatcttaaacattaagcgtgttaaaattcaaaacaaagcttgaagagagaaaaatgcttgttatgtgtggcatacaaccttcctaacctctttatcagatgaaggatccagtgtggcactggattatttttctgtaatattttgcctcggacatatgtgctatacatttcttgtctgcatggggaaagtatgtcaggttgctgttgccactttcttcccatggagtggataatttcccttgagaataagttggctttctcatctcaccctttgcttttggtagtacctctggaaccagagggtaagattcatcttaatcatgtgaacattttattaattatctcatgtgtgattcaagcacatcattactgttactcaaggagaatattttctttgcaaagctttcaaccctttccatttgaatgactgctcttcatggtgtaatagatccactgttgaaattggcacttttctgtacttgtctctgaccagcaaaggcaaggaacattgcgtaaaataaattactgttgaattattggggcaaagtcaggttaatagcttaatccaaaaaaggaattacaagtgaaagaattttaccttaaatgtcattctgtttcattttgtaattaagaccttccaaatgtgtaagtagtaatgaccagaaatcttatcagatatggtcaaatgagaaaggtattcaattcatattaattttgaattttattgttttaatacaattttaggaaaagcatgccttgttttctgggtttcttggctgcctttgtatcaattatctacagctatcttctttagcatggaaaggatgtatttgaatactgtgacattttataccagggtaattctttgaatttatgtaaacaacaactctcttgagacacatcagtacaacacaatagtcagaaatgtcacatgagagttcaccaaactttaaagatggcgatgaggatggaaaaacatttttattgtagactttaaggatcttgtaaatccccgtatgtaatccagtgtgctagagaattggtgcccatggcgcctcctctccccaccgacctgcactgtggaaatcctactaattttgaagtcatgggagatgcatgtgtgggacctatacatgcactgtgatgagaatgatgacttcatcattctcattatttaatgagcccagctgctgcatacaagaaggatgcaaattcttgaatgttcttgattgtttacatcatggtctcataaggaatgaccagaattgctgggcatttcttcatcaggaaaggctaatcagccatccttggagagacatcagactcctttatatgtggccttcccatgggacctttgaatggtcaacgttaatcatgcttcagaaccagcatactaagtaaaagaaaacatgtccggaaaagaaatcttatcagtgtttatctcccaacagtgggattacaggtgattttgattttatttaagcaattttctatgactttgtataataaatgagtagcttttataatcagcaaaagatagactatgaaatcagtacatataattttgtatttgtatgtagtaccattgaaggataatagaaaaaagtcaaaagacttgaaagtcgacacagaatgatcattaatttgttttatctcttggtagttgtactttatgagctttatacatacagtatgggaaaatacactttagttttgaaaacaccatgatgccaaaataaaagaaaataacgttcattcatacgatcttcaacaaaatttaagcagcattaatcatcactgattcttcaggaagattcttgatcttccttaattttttttccctccaaaattgcatatggcagccacatttgtttcaggccagaaggaagaatgggaagagcttacatttatctcacagattccttctgggtgaggaagacttagaaagtgaagccctcttccatttgattaagcaagaacacctataaaaatggtgtcttcggccgggcgcggtgctcacgcctgtaatcccagaacttcgggaggccgagatgggcggatcgcaaggtcaggagattgagactgtcttggctaacaacggtgaaaccccgtctctactaaaaatacaaaaaaattagccaggcgtggtgcgggcgcctgtaatcccagctactcgggaggctgaggcaggagaatggcgtgaacccgggaggcggagcttgccgtgagccgagatcgcgccactgcgctccagcctgggggacagagcgagactcagtctcaaaaaaaaaaaaaaaaaaaaaaaaaaaaatggagcccttctaggtaggtcatcctccatggctcactgcagcacttccttggccctgtcccaagttccataattgcctcaattgcattggctcctgcttgatttgctttctaaaactcaggacagaggatgagttgaggagactgacttttttaacaatcgcattttgaaaagaaggaagcgtagtggtgtttcttggctccgtttccagttttgtttcttttcattttaattattgtacctggtattgtgtcctctttctgggacttttaacaactaatgttttgattgataagagctctaatagcatgggtgatttatacttcattacatcttcagttttgtcttctttactggagttgtattcaggtgtcattaagtaacgaggcattaaaaagaaacgataacttttgaaaaactgctgttatggcttatgagtgggctttgctgatattttcttgaagcacagaatttatctatctctgtaggtcctagatgataggtgagaaaaagaaacccagcttatttggcatgatattgtcccaaggcttgttagtaattgctttgaaaaatgaatagcaaggctaatggacatctgatttgtagtacatgctaagaattgcaaatgtgtgctcaaaagaaatttctctgattacaaaagtaacatatattcaacgtacaaagttgagaaaatacaaccactagctaaaaataatcaaaaccatttggcttccttgcagtctacatttttttcatgtatgttttgcttttgtttttagtgttatttaatcgtttcctgttttgtgacttgctttttacacctaacatttttagaatactctacctgtgaaattaaatattcttggagagcataatttttaatgtagcatcacaccagacactgtaatgtgtttactcttttttatggtcatttaggtggttttctttattttgtggtggtgtaatagttgttataaatgatgctgtgataaacatcctggtagataaatttgtgtttagttgatttttatataacagttattggcggaatgccttgcaaatcttatagttggtgtaataaaatatatgcgccacataattgtttatgaaaatatggaggaaaattttaatactgatttatgatcctgagtttaacataagcatggatacccctacatttagctatgatttttttattgatagagtaaaagcgcctgtaatttctacttaaggccatttaaaataattactatttaaatctatttaaacaattttaaatattgcttttccaaatttccaccttaggaagtagttaattgaatattctcagagaaataagtcctacaaaattgaccaataaaaggtcagttacgtattcattgtgtacgttttattgatcacccacagtgtgtgagcccctggaggtgggtgaaatgtgggcctccctcttacagagttgatagtgggcctgaaggggatgggatttcagctcaggaatttaaggaagacttgacaggaagtagcattgtgagccaagatttcaaggataagatttcaaggcaagaaaggagatgtattttaggtattcagacagagggtcaacaaagtctttaaggtaggaactagttcacttttcaaaaagaagaggtaattggtcatgataagagtagtagtttttaaaaatcattactgttttcattcatttataatgaatactattattttataattttaaagtttatatccaaataattgttttcaagatggtaatgaatgtatattgcttttatgtatacacatagtactttttgctttttggtaactaagtttataatttttgagggcaggaaatatttttgtttttcacttacaaagaaactatattttaataagagttttttgacagcagtgctttatgccaaaggaaaataaagcaacatttaacatatcaaagaaagccaatgcaaaacaaggattatttatttatttatttattttattcatttttgagatggagtttcactcttgttgccccggctggagtgcaatggcgtgatctcggctcactgcaacctccacttcctgggatcaagcgattctcctgcctcggcctcctgagtagctgggattactggcacccgccaccacgcccagctaattttttgtctttttagtagagatggggtttcatcatgttggccaggttggtctcaaactcctgacctcaggtgatccacccatcttggcctcccaaagtcctgggattacagacatgagccatcatgcccagccaaaacaaggattttatattcaatcaaactgacattcagatatgatcacatgaaataaatatcaacgtacaataactcagggcatattttcacattattttcctgtagaatctgctagagaacaagtttcagatacccaaaatacctagagaaaaactgatataaggatgggtaatgagcattaaggagaaacataaaaactaagactcaacgatggtaaaaagtatgataatatagtattccacagctacatgctgtgacaataaagagattctgtaactgtcaggcctctgagcccaagctaaaccatcccctgtgacctgcacgtattcgcccagatggcccgaagcaagtgaagaatcacaaaagaagtgaaaatggcctgttcctgccttaactgatgacattccaccacaaaagaagtgaaaatggccggtccttgccttaagtgatgacattatcttgtgaaattccttcttctggctcatcctggctcaaaaactcccccactgagcaccctgtgacccccacccctgccccccagagaacaaccccctttgactgcaattttcctttacctacccgaatcttacaaaatggccctaccccatctccctttcctgactctttttcggactcagcctgcctgcacccaggtgaaataaacagccttgttgtgacagtaactttaatggggaagaatgagaagaagatgtgggtaaagaaacaactgtttttctattaatggtggtagtattagtgtgtaattctgagcctggtgcataagtaatatggtgtaacataagtgaatagttgtgtgatattttaattcaatcatcccatgtatttgagaatcagaattttgatgtggtaaaagaagattcacatgtaacatataatctattaaaaagataccctgtagtcatgagtttgacttttaggtatcatttctcagtataacatattgttgtagatttcagataacattttttccccataaatactgtccgctgcaggcctaaaaataatgaaaaggcctaaaagaccaactcagtagcggtaaacaccagtactgcacctagattatggtctttaggaaccattttccagtaaaagataccagtaatctttggaaagcaggatttttttttctttttttttttttttgagatagagtctagcttcgtcgcccaggctggagtgcagtggtgcgatcttggctcactgcaacctctgcctcctgagttcaagccattctctgcctcagcctaccgagtagctgggattacaggtgcctgccaccactactggctaatttttttttgtatttttagtagagacggaatttgaccaccttggccagtctggtcttgaactcctgacctcgtgatccacccgcctcagcctcccaaagtactgagattacaggcatgagccaccacaacccggcggaaagcaggaaattttttaaaagctattttaattcagtcatgtaaaaaaaatgagcagaaagaaacaaaaaacttaaaaaaaaacttaaaaaatgagcagaatcttaggaatcacctgcgactttggttattcatgcttagccatttctataggaaattccagcactatttgagttagaccactaattatatgtgtgttgtagctttctccaagagataatggaattcacaaatcaaaactttgagattattaagtgttacaagtgaattataaccccagtcatttgctttcaaatgaacagctaaacatattttgtgttaatttcttatggatgctgctttgtttgtttgtttgtttattgagacggaatttcactcctgtcacctaggctgaagtgcaatggtgagatctcggctcactgcaacctctgcctcccgggttcaagtgattctcctgcctcagcctcccgagtagctgggattacaggcgtgcaccaccacacctagcagatttttgtatttttaatagagacagggtttcaccatgttggccaggctggtttcgaacttctgacctcaggtgatccacccaccttggtctcccaaaatgctgggattacaggcgtgagccaccattcccggcctagataggcttttagttggtttccatatacagtgtatatagggagaaacagactaagacagacactggcatccacagaagtaaaaataaaaagtaaggtataatttaaatttcacacggtcatggtcacatttttcagctttttgcacctatgttgtataaaaaagacaaaaatgcctttagaggctcaagttttcaggacttcctgaagctgtgtcatagtttaaaaatgccataaaaatgccgtcagatttaataacgtatcagctgaacactggatttctgttatgttgagtcttcaaaaatttctccctgtatgaggaattaatactgttatgtctgatctggtggtctgaaatgtgaacttttctgggccaaccagtaaacagctgctgttgctttatagctgcgtaattcagtcttggaaattattagataattcccacaccatttcagcttgctgttttaccataaatttaatgtgaacagtatttctagctaacctcaagctgtgtttattgtcgtggaattttctcttttattctgaggtaaatgattacaaatggttatgatgaggaatcaaagaagaaagtggactttcttcaaagttctttttctactctagccgcttctgagcatctttctcaagcactgtgtttccatcagtccctaccccaagcagagaaaggaaacagttttcccctttgtcttcagaaccctcttctcatcgtgtcattttccaaagcaaataaaaatcttgagatattgtcttgagtttgattttttttttttttttttgagatggagtttcactctttgttgcccaggctggagtgcaatggcacaatctcagctcaccccaacctccgcctcctgggttcaagtgattctcctgcctaagcctgccaagtagctgggattacaggcatgtgccaccatgcccagctatttttctgtttttagtagagacggggtttctccgtgttggtcaggctggtctcgagctcccgacctcaggtgatccgcgcctcggcctcccaaagtgctgggattacaggcgtgagccaccacaccctgccgagtttgatgttttttttaaccattcaagccagtaaataactttccagacagctgtagtagaaaacagaatttgtgtgtgacaaaacttaagattttgggattgagagctctatggtttatgccatgtggataattgtagccagatgaagaatatgaaatttaaggaagtgtttagagtggtacagacagctattattacgattcttggggtagaagagtagcttacatacaacataaaataatggcattaactgaattaacttcagcctttgccatgaaacccaagctttcattatgttttaaataatgaactacattaaaaatagtatataaatatgtaaaatatattagacatagacttctcataataaattatgggaacttttgatgtatttcttccctaggaaaagaactaaacaagtgcttctttgtggacttaaaatttttcagtttactattgtgaattaggtacagcatatctttataattccagataatacccataaaagcagaactgtttatttacttattatgaagggaatactgaaatgaaattaaggacaatttttttctcacgtattatatttttaaaaagttatttgatgtttgtgattaaataaagaatgaatttttaaagccagttctcaattagcactgaggaaaagaatatgctttttttcaggtacacaaataagcatcatggatataagaaacgacataaagatgaatctacagtctggattaagacgtattttaatttcagtgtcaaaggctgttaggaaatattcagattttagtacttgacaaagaaggttctcaatatttgtttcagtgttcagtgaaaggataaattctgtcgtctttctcttttatcatattagtcactgagtggtttcattaaacattccctcttccatatcccctgataaatttcaacttgttggtgatgcatcccaggagtgtctgtctttttttttttttttttttccttctcctgtcagtgagtcttaggctttggctttgaaaccctatagtgaagggaaatctcaggcaacagctgtgcaaccgtctggagagcagctagtccagattggaggagacgaacgaggctggtcgcaggagagaggcctccaggaaaaaatggcattgatgcattacctgcgttaaagattttcctgtacttagcatgcttcttagggttctactggagcgtttgggaaagatacagtattgatggatgcatagaaaatgaggcaaattttttaaatggggcaaatattaactgtagacaaagaaaagccaaaagtacaaaataacagcgttgtaagatacgaagataaaaatacgcctaagtatgagcgttctggcagactcacaatacagatgccatatatggggtaaaaccataccagtaggatggtgggaagggaaaggggtgggctcatgttccatagtgggaagacctacagcggaaaaatcaggagaatagttatttaagcacagagtttagggagtaagttgcagaagagacaactaaatgagatggccgtgaatgatttttatgacctcctcttgttatggagaaatcgatcaaggaggcctatatttgatggcaggaagaagttgagtttaaggtttccagacatttgttttaatctggaaaatttctttttttctttctttcttttttttttttttttgagacagagtctcactctgtcgcccaggctggagcgcagtggcgcagtcttggctcatcgcagcctccgcctcctgggttcacgccattctcctgtctcagcctcttgagtagctgggactacaggcgcccacaaccacgcccggctaattttttgtatttttagtggagactgggtttcatcgtgttagccaggatggtctccatctcctgatctcgtgatccgcccacctcggcctcccaaagtgccgggattacaggcgtgagccactgcgcccagcctaaaatttcttttaagcttagaagttgcaagtcaagttactgttaaaggcgtcctaactttgaattagataaagctaaactgtggtttaattgagctgagtttgttgtatgctatatctcaacttcacaagatgtttgctttttcttggctataacctaagattatacaaatataaaattacctttatgcccatttgatagactttagggctgaatttccctgtgtaagatgttgtagaggtttgtacttgtgattgtttttactctagaagcatgtttatcttcactcctttagtgacacgtgtttgttcaggaacttcaaactcaatgtgatcaaaactagactcatgttccctgaatagtctctcctgactacaaatctgttggaaacttccaatcctaattaataattgtaaactttttaatgtaagtcatatgaggggttgttgtgagcataggaggtagggagatggtagtgtgagctggagcatatggtaaaagttgtttgaaagaaagtagacccatgatgtggatcctgagggatacgtaaagaaaatgaagaggtgtagatttaaaaatgtttatggggcctgggcgtggtagctcacacctgtaatcccagcactttgggaggccgaggtgggcagatcacctgaggttaggagttggagaccagcctggccaacatagtgaaaccccgtctctactaaaagtacaaaaattagccaggcatggtgatgggcgcctgtaatcccagctactcaggaggctgaggcaggagaatcacttgaaaccgggaagcggagattgcagtgagccgagatcgcgccactgcacgccagcctggatgacaagagcgaaactccgtctcaaaaaaaattaaacaataaataaataaatatattcatggggatgttccagggaaagtagtttgtgttggtagccttgagaaataataatagaaaattagaatagaactaagttggttcattaaagttggaacacttgtagagatttttgatttggacagacattaattcatttgattatttctcttattcgatgtccagaaaaatatctgtacattcaatgtggttttatttgtactgcacatgtcattaataggctaaagttacctctaagatacctgctttcttagtccttgttacgctgtaaattctgggcatcaggatatagctttttattatgttagctcttttaatagatgatacaattggattggggaaaaaactgattatactctggagtaatactttcatagcctgtagtatcctgcaaatagctcctggtcatcttcgaattttactagcaaacttgtagggtaataggttgggacaaagtattataatcagtggctttgagggcctgataagaggaaacaataattgtgcagctatattgtgaataagtagtgcatgatgctgatggttaaccctttcctgcggtgctcaaacattgggaaataattgctttttgttcaaaatgtaagctcttcgataattgagacttttgtcaattcactgaaatattcccagagcttgaacatgtctggtacatagtacatatttaataaatatatagatttaatgaatgaattgaggatgtagccaaatattggaaaaggaaagggaaaagaaagaatagcccaaacattagaaagacagaagatgtggagcagcaatttcacatgcctaaaagaatttctgagattatggcaaaatcatttactttacttactccttattgatcaacaaaggaagagcaggttttatgtaacaaccaagaagtaacatagggatctcagaagagaagaaaccaagtgtctttggatcttaataaaaagaacattcaactccagaaatagggttaaacctatcctacacatgtctagtaaaaagtcagtgtatcagaggcatctgtgtttagagataaaacataccattagattcatttataatagtttcattgtccttagtttttaaggctaatgataaagatttttttttttttgtgagacacagtctctctgtcacccaggctggagtgcagtgacgcaatctctgctcactgcaacctccgcctcccgggttcaagcgattctcttgcctcagccacccaagtagctaggactacaggcacacgccatcatgcctggctaatttttgtatttttagtagagagggggtttcaccatgttgcccacgatggtctcgatctcctgacctcaacagatctgcccgcctcggcctagtgctggaattacaggcgtgagccaccacgcctggccttaagaattaattttcacagaaatctgcttctaaatattattcagtgataatagattttagaaatttcagattcttatgttgtaccttaaaggttaggatactatttataaatgctaaacatgaactcctgtcatcctattttaatgtctttattaagtgattacatgaatgtggcgtttcagtaggtgtttggtgtattaatagatgtaggtactcccctcttaaacctttaagtattttcatgccatgaaaaatttggggggcattctctttatttattgattaggtacctgtattttcaagaatattactcagcttcagttcccccaaatcttttttagtgttaactaccttccgagaacgtggtataacattttaaaatatattttttgaaggtttggatgtcactgaagatccccctatttattggatggtatctttatcagctgggaagataattagtttcccaatgttgctatatttttatgaataggtcctgtagctcttgctttcttgaaagcttagaattgattgtaagccatggactaatgatattctccaagcatatttaacatgaataggctcatgttcagtatgtcaagaagttaccctgttgtactatttaaataatttgagcttagttttctttttccacctttgttctaaattcttaggtgagtctgaattcctgggcatctgtgcacacagcttcttcagtaggtcctttactctcattgtatctgatctttgacttcataaagacccttggcctactgaccctgtatttaatttcacatctcaatagtatttagccttctcatatttttcctcttctttagagctccctgatccctcactccagtcattcaactctgtgtattcctttaacttccaccctcgctctgtctcttcacagatatgcttctttatatggtagtcagaagttcctgtgtgatcttcgtaacctccagttccctccacagtcacctgttatcaggcatctgttccttgagctccagggaatgagctctttgctatcattctggttgtcggactcactgggcactgttgtgtcctccacagtctctcagtggcatcggttgatgggtgcattctccccacactgatgtcattctgctgcttctgattttctttctgctgtcctccattttcctttcaggctcccttccccgtcctccccgcgccaccttttgcaatatttaggattcagaaattccaaatattgcaaaatatttagaatcagaattcagaaattctaaatatcagtgtttaataggagtaccctggggttgtccattttctcacacaacacatacttgctaggagatctcataagtgcagtggtttttcagaggcctgcattgatgattcctgagtcattatgtccaggccaggcctcatacatactatttactggttctctttctgcgggtgctccaccaagaattcagactcaatggaatatttcttctaagatgtacaccagttccttatcaaaaaatggcaaattcagacttcttaggttaaacagagaactcgtgaacatggcatatgtctgttattccatttttctctgttggcttattgctgagtaccatttgggataaaaatctgtttcaggcaaaggataggattgttttcagcttttattaaattctcctagacccttgattttatgcagagaactaattaaatttgaatgaatttaatgtagaaaccctgctattgatgcagaaatcaacctgattctggactagcaccagaatcaactatttttgttgtttttgctttctttttttttgcttttgctttctctgccggtaaataacaactagtatgtaagaatcaaatgaatcaagcataaatccctaggaaatatttatgcattcatttttaacaatttaaagatgtgaagatatataattaagcataatcctctcaaagtttgacgttgaaaagtatttcagatacttcaacttaaatatattttttgcaaaatatcttttttaaaaaaacagatgtggtctcagcaatgtaattattacagtgtgaggagagcttacagttgtaccaaaatttataactaggaagaagagaatgtgaaataatccggaaacctacaaaactgtcctatacaggaataccaattaaacagctactaaaaatgagtgtaaaatgacaatatggctttatcaagtgcttagggtctggaaaaatgcacatattgcctgttatttattgttatacttaatgaatgttcttttgcacgtgagaactgttataaaagatgtcttttgtacatacttggtacaccagttggctttttaacccattagctgttttcccccaaaggtggcctactctagatgacgttaaaaatacacattttccagggacttatataaatagtcaactgccctattatggaaacttaaaagcgaaggccctgaaatatccagacagtttgaacataaaccaaaaatgaagtgaatctctagaattgctgtgtaatagcgtgagtggggagggggtccctcaagagagggaactgacctttttcttcctcgtgggtgaccttgagtacacactttatttagtcattaaaagggtcacctgaagcttagagaatgaagatcctacagcctgttaattttatcattgttagaaactctctgcctttggcctgaaaatggtaaatcacctttcttagaagattctcatatccagatattcttaccaaatcagaatcttttccagcattttcttcaaaaaaccacagttctgggttaaaacacagcagatcctaactgtaattttcctttcatgtacattttagtcatctttataattttaaatgaaagacaggaactacattatttgaaataagggttaaccccctctaattataagcattaatcacacacagttttctcatttctaaatgttaaatataaaatgggagctatctcttcagacacttttctgttgccttctgtgtgcggcaggaccttacttttaagcttagtttgatttaaaggctgagtttctgcttgcttggaatgcttgctcttcaaaatatttgctccctggagagggaaactgaagatggagctttcacgtgactttggattgctgacctggaagacgtttggggtaataggatgggctagggcatggttccactgctttctgtttgagtggggtcaggaaaggaaggcggggctcgctgtgtgcaaacaggaaaacatagtctgaagatgtttgaagcctgagtgcactttcatttgatgccttgctttattgcttaagagaattttaaactgccgaatccgcaaatgtgctgcagcctataatgacacatatttaaatgatttttgcatctaaacaaatgcattcactctttgcctggtgtgtacaatttgaatgacactcgtgctctgcctggaaggaacagacgtttttcattataaaattcagtcaagcagaaaagtagcaaaggaagcagaaatttccatttacatctatgattgaagttgtagtgataaaagaggatgactaagttgaagagtagccttgttctcattctgctatctgacatgggatcttgtcactttggtaatagtttttaaacttacattattgaggaaggctctgatcagaagtctcaaattatacatgttcttcaaccttagatttttctgggtgttttgtttcaaaagtctcaaattatatatgttcttcaaccttagatttttctgggtgttttgtttggttttttggttatctatagatagatttgagtatctctttttttcttaaaatccgttacttactagtaataaagacttaagtggtagctttacgctccattatcacagagattcacagtactttttagtttttacttgtgagcctcagcaggtgtagacagataccttagttgccactgataacatccagttgccactagatcaagaaatggttcaaaattctccaggggcattttctgctaaagaagagttggtgatcttccttttggaagccttaatgtacttaaaccttcttccttagaagtccagaaattcaattatttcagctctctgtggagtgataagtcctgccaaattgatttacaataatgaaactaagccttgtttacaaatctaagtggtgatttctaagtccacagaggcgtggtaacagagagagagattctgatttatctgggggtctatccgtgtttgaacagattgtgtgatttttgaaacatgattcaaaagggatgttgataaattctaaaatacatttttattgaagagcttgatgaatcatgtatacttactttatttgttattctattagatattgtcttcctcatccctaggtggtgttgctagtacagtgacagaattcaaatccttctttttcctctgctctaacagagctttgtgaactcatagcaatattgtaatgcctggtaaaaggtggtgccagtttcaagtactatatcctaattacttcatggctaactcttgttattgttaacaatattgttaactcttttgttaaatgcagtgacatcttttttctttctcattctctgtgtactttttgccctaaatagttctgcatcttttaagatacaagtttgctttttcttggaggtttttatttggaaaatgcctgaaatatatagaccaacatttgttcctccaatggttaaattacattgtctcccaaagtgaggcattttaagtctggactatacccaggagaatctaaaagaggcagttaataagctcttgaagagcacagtggggtggttgtgtatttattgagtctcacacttaggagagaaataggctggaccaaatgagcctgggcatcaaaagcatgtgggtggtcgttggtgatatgtgcagtgatgagtgtgtgtgctgagggtcctagaccaggctggcagggaaatgaactgaaggcagtgaagcctcccctgataactgcagagagtgctgcagcatccaggacctcaggatcatgatgaacaagctgtagtggatggaagggagaggaactatgggaaggatgagtttctggccagaaagtggtttactggagtgctgtgtctgagtattacatgatctgaggaccatgaggtccagctttgggagtatggagagtttgagtggagtagagttatagtcccatggtcttctgattcacagacattcatgagcgataagtatctattgagaaagtggaagcatgatttttatgacctcctcttgttgtggggaaatcgatcaacggggcctatattggttgggttctacaaccaaatagaatgtgttcagcaggagtcatgttttgggctcctcacaatgttcagtgttagttctgttcctttctcttgcttccacataggaaagactttgacccaatgaggaattagtcattgtcctttgggtacacttccggaatttgataatcagttgggcacttgattctagtacatttgctaaaagtaattaataccctgcaattggaagcatgcagcaagcgactgggaatgctaagtgaaaggtcagaattgaaagagtggaggaggctgttgacccttaggcatggatgatatgtctgagtgagaaagcaagaagagtagaagacagagccttactttataatcagaattatgaaaagaggagccaatgagagggaaagaatgattggcttgagatagattggtggaaactggagaagattgaatttaaagaagaaaagtgggccaagttcagtggcccatgcctagaatcccagcaaattgggaggctgaagctaggagagttgcttgaggccaggagtttgagatcagcttgggcaacgtggcgagacccctgtctctacaaaaaataattaaaaaaaaaattagccaggcatgttggcatgtgccggtagtcccggctacttggaaggctgagacaggaaggttgtctgagcccaggaggctgcagtgagctatgatcacagcactgcactccagcctggttgacagagggtatccccgtctctttaaaaaaaaaaaaaaaaagagaatgtggcagagtagtcagagaactggagggctgagaagagactgatggcatttgttgttcttgaccttgaaagaagagttgcagattgttggagcaaggccagatggtaataggttggaaagaacaagtgaggggcgtgagagtgacagtctacaaccccgttaagaagttatctgtgaaaatgcctcttcctgtcttgattatagcctccctcgcacatggctttctgagtatggtatgtgaaaacagcatgctgtatatttttaccttcccatatgagttaaggttttggtaatgacttgtggttcttttgcctcacggaacaccaacctggttggtattttgtcggcaattatgtggtttttaaaaagccccgtatataaccttaattaattgcgtctctaaaatctatcaggctttgagttttttgttttttttttttaaataaggcagaaagaaaggaaagtgttaggagaaagcaaccagtgagaatacctatattttgaagaaacaaagtagggaatttagaattatctctactatatctgctttttcagctcatctctcgggatacagaggactgaactaagctaaagtaggttcaagtctaaaggaaaccttaaattaatttctgtattagaattaatcacacacctataaatacaatttatgatatgagaactgcaacaagagaaatggggggagatgagcagtttgctgatcaatagggttaatgatggtaacatagaatcctagtgatatgcctcccattgactaaaatgtatgcgttaagatagcatagcgcataaaatggtgtactgcagtctcagtgtattcaagcttattatggtttctctgcctaggtttgtgtctttggctttagattttctcccataaccccaacctgtccacattcaagtcactactccttgaaatatattattgatctctaaaatctgatgctaagtctgtcttgcccaacggtggccactagccacccatggccattgaatatttgaaacctggctagtccaaactatgctgtgagtataaaaaacccaccagatattaaaaagttagtctgggagaaaaaaacaaatgtagaatgtcttaatgataattatttacattgattgcatgttgaaatgatgaaatttaggatgtactgggttaaaatatattaataaaattaattttacctgtttctttttacctttttatgttaatgtggctacaggaaattttaaattagatatgtggcctctattatatttctgtgagacaaagctgctccaagctgttttggtccagaactatatctcggttcttttgtatcctacatggacctgcatagaatggtatttagtaatactcagcagtagcagagatgatctcttatcaaattatgttaagttttaatttttattcttgtctctcaccctatgtgcacctctctggccaggaggcatttatcatatcaccttttaatatttggaaatcacttgatctgcttgagtcattgtgttgttttcaaatctagaactctttctaccttattttgtaacttctataattaaacaaaaggcgaaccaggagcagcttggctcttagggatgtgtgaaaaggcgctcagtccttaagttggtgccaaaagtgacaaatctataggatgagatgcaggtttatggaagagaagcatgaaaatttttattttttaaaaggacacagcaagctagtcaccaaaagacagatacttgattctacttatatgcggtacctttttgaatttgaagagtcaaattcaaaaagatggaaaacagaatggtggtttccaggttctggaggtgggggaatggggagtggaggtgggggaatggggagtggaggtgggggaatggggagttgtttaatggtgtgcagggttttagtttagcaagatgaaaacagttctggagatggaaggtagtgatggttgtacaacgatatgaatctacttaacactaaactgtataattaaaagtgggtaagcaaagagtgtcttacaaagcaataacttgtaacttaaaatttttaaaaaccttaatgagattagttgtaatgggttatcttaagtagacccttactgccggaaggaacagctgaatcacatcacttgggagcttgttagaaaagcagactcagtcccaccctagacttgtccaatcaaaacctgcattttaacaaggcccccaggtgattcataggcactgtaaagcttgagaagccctgctccaaatatcatttctcaaagtgtgatccatgagccacctgaagcagagccaccctgggaaggtctgttttcaggcgcgtcccagaacacgtaaatcagatctaggctgtggagctagggagaagatgaatctgcagactgatcaggatcccaggtgattttgaagcatggtttgagtttgaaaacccctaatctggaaagctttaacaactattactcttacaattccatcttttaacgaatgttggaattattaaatacttgtaacaagaagtcctgttattgataaaatcaagatatgactaatagtgcagtcaaaaagaggcaacaaaaaagcacctataacttatcttgggttggttaatgaggaagctgtcattcaaaattagtaagactactgtaacatttcttggggtcctgaacattggaatgagagtcggttttcagagttgagcccacctctgtcattagctttgtgcaaacctcatcaggctcaagattcctcatttgtaaaggggaatgaattctctgaccagttagtttacattcctaagacaaggttaaatttggaaagcaaagaattttcggtatatatgtgataattttgaaattcttgaatttttaaaaaatttaaatgccacattacttatatttttttctaaaaaaaagtcaaaccaagcgctgtctagttattaatagataccacagatgagatgcctgagttggtaagctctttttaattctagttcttttcaactacaataattttatagacttttttttgaaggctagaatgttgatgtggaatcaaataggtatgcctctatgtatggtagatgcacctgacagggatcacttgacttgaacatgccctgacaatgatcctgtgtggcaggtgcacctgattctgagttctgagttaagaaatccgggagtgggccgggcggggtggccggtgcctgtaatcccagcactttgggaggccgaggcaggtggatcatttgaggtcggaagtccaagaccagcctggccaatgtggcaaaaccccgtctctactaaaaatacaaaaaccagctgggcgtggtggcgcaggcctgtagtcccagctactttggaggctgaggcaggagaattgcttgaacccgggaggtggaggttgcagtgagccaagatcacgccagtgcactccagcctgggcgacagaatgagactctgtttcaaacaacagcaacaacaaaacccaaaacaaaaaaacaacaacaaagaaatccaggagtggctaacccagagagccattccttatctgtaaggaacatctgagccctcatctccttctgtggaacatgggcctcgccggggattgagaccctttgttatgagttaagtggaagttgccaggtggagattgttagggggagggtgttaaatgagaatgctgtagagaccacatgccttttgcaagcggctgcggttcctctgtccaacccaccaccactggactctctaccctgtatgcaagcccccagtaaaaccctatgagacatacggttcactagctctgggcctcttctggctcttgaacctggtgccatccacattgcagtcagtaggagtttggcatgacaccttattatgtgaacttttatatcaagccattaaatttttttaaaatagactttatattttacaacagttttaaatttacaggaaaattgagaggatagtccagagagttcccataggctttgcatccagttttccctattgttaacatcttatattaatatggtacatttgttgcagtcagtaaaccaattttgatacatggttattaactaaagtctatactttattcagatgtccttagtttttgtctaatgtcctttctgtgttccaggatgccatccaggaaggcgcattatatttaattgtcaggtctccatagcctcctttcctcttggctatgacagtttctttatacttctctagttttttaataaccttaaaggttttgtattggtcagataatttgtaggattcccctctgtgggaattttctcatgctttgcctggggttattggttttaagaaggaagaccacagaggtaaaatgcccatttcatcacatcatatcaagggtacgtgtttatcaacatgatacagccctgttggcattgaccttgatcacctggctcaggtggtggttgttgagtttctccacttgaaaattactcgtcttcccactttatgtgctctactctttggaaggaagtcagtttgcacagcccacaattaaggagcagagaattatgcttcattttcttgagggcagagtatctacataaattatttggaattcttctgcgcaggagatttgtctcttttttcctatttatgtattcattcattcagtcatttagactcatggatatttacattgtactttgatttctaatccagcactacttcataatttttattgctcaaattattccggctttagctattagaagccctttcacttggttcctatgctcatttgacatagcctcttttttttttttcttacaagatgctgcagtctcatcttgcatattttcagccctaatcctagaatcagctgttcctccaaggagcccttgttccttttattggagagtaatgttattggagaatagtattaaaatctggatgttaaggatgctcgttgccactggagtttctttgcttttaagacttctcagttgatagagcaaggcaatgtatttgtccatgttcacccttatgttatgcagatctataaatatttctatagctaactagttgtatttatattaagctataatatctagtctatattaagctaaatacaagttcatccggatgtttcctactctaacgtattaccacatggacactatatctctcctccttggttatgtataaattctcactccatccaccatctatgtacttaattgttcagtttcagtatatgtgcgtagccgtatcagaattgttaacccaggctgggtacagtggctcatgcctgtaatcccaatactttgggaggctgaggcaggtggattgcttgagcccaggaatttgagaccagcctgggcaacatggcataaccccatgtctactaaaaatacaaaaaaattgctggtcatggtagcatgtgcctgtagtcccagctacccgagaagctgaggtgggaggatcacctgagcccaggaagtcaaggctgcagtgagctgtgatcgtgccattgcactccagcctgggagacagagtaagactctttcttaaaaaaataaaaaataaaaaatataaaaaaatttgttaacccacacctatatggatctttttaaaagctgcaagactgctaacaaatatttatttatttatttatttatttatttatttgagacagagtctccctctgtcacccaggctggagtgcaatgacacaatctcagcccactgcaacctctgcttccccggctcaggcgattctcctgccccagcctcccgagtagctaagattacaggcacccgccaccatgcccagctaattttgtatttttagtagagacggggtttcaccatgttagccaggctggtctcgaactcctgacctcaggtgatccacttgcctcggcctcccaaattgctgggattataggtgtgagccaccatgcctggccataaatacttattttgttcccgtggttttttttttcttttctcttttttaaagtaaatcctttgcactatttttattattttaagaccagtgttattcagagcctgggttaaacactattctgtgtgtgtgtgtgtgtgtgtgtgtgtgtttgtttgtttgtttgttttttgagacagagtctcgctctgtcacccatgctggagtgcagtggtgtgatcttggctcactgcaacctccgcctccctggttcgggagattcttcggcctcagcctcccgagtagctgggactataggtgcgcgccaacatgctcagctaatttttgtatttttagtagagacggagtgtcaccatattggccaggctggtttcaaactcctgaccttgtgatccacctcggcctcccaaagtgctgggattacaggcatgagccaccactcccggccagcactattctgtcttatactcactctgccatctgtgtgtgtatatggtacgtatgtgaggctggatctgtgcacttggaaaagttggagagatgatttaggtaaaacttttagttttcacttcttttgcctttttgcttatcttgatccatttagttcctaccatctggcaaatagttctcatctgaagaatcccatctgtcagttctcactattatccggtaatagttacagtgaaatgctggtgacggcaacagccactttttaaaaggatgtgtaattcaccctcatgtcctctttgagaacccagtaactgcccacactctgaagctaaaaccataacagttttatcacatggaaagtttgttgagaaatgtttggctgtttctgtgaagtaatcgtcccgtctcttgagtacagatgcctcccaggaaaattttcccagcctatttgttaatgtgaaagttacaaaagatagcatataatgagcacattcaagacaaaaaaatttgtggagtaagagtctgaaatggtaaaataaaaaatcacttaagaaaacatttatctgtagctgtcattttcatatacacatatctagtgtatacaagttagtaagtacataattctcaaaagttctacctgtctacacatctacacagaaaaattaggaaagtcttgtccagacttttccatcatcttcagtataaagcatcttctagctgttggaatagaaacaagctagattcttgactcctttctatttgattatttagatctgaccttcagacgacatccagaatctgagaacacgcaccacctctactgctctcatcctggtccaagtcattgtcatctcccacctggattaatgccagagcccccttcctggtcttctcacttctagtcttgaccccctagggcaagaataattagcttagaaaaccaccccagagccttccagcattactctgagtactggccagagttggacaatgtttaaggccccgtgcagtcagctgtccctgtcctgtcctggctgtcactcactcgcttgcccactgatctcttccacatcattcctccacgacagcactgatctccttgttcttctgtgagttcaccaggcatgcatctgcctcaagactttgtgtaatacttgttgcttcctctggctagaatattcttccctttgattctgaatactttgttccctcacttcctcaagtcttcactgaaatgtcatattttcaagtaggcattccctgaccaccctatttaaaatttcacccctcagaaaaaacaaacaaaccacccctttctctgctttatctatgtaacatttattaccatccaacagacactgtatttttatctgtattttttatcgcttccccgctaggatcatatatcgttttatctgttttgttcactgctgtattcttagcactgagacataattgtaagtatgcagtgaagagttagtggataaaggacagtggctgcaagaggggatttaggaaacaccccctcccagccaggaccagcactcggtgcaagagttgaaagagttgatcactcagattctctgcttctccagtcgtagggactccaccccgctgtgttgattgctgtcatgtattcaccccatcataagcagtactcacatgtaaaacagtaccatgtcatctctgtggggctacaagtaggtggtgatggtgcaatgaacatataaaaccccagtgtcttcagtaggtgcgaaataggtgattagtgaatgatgaagagtagctgttgctgttttgattactatgattattattatgagaaactactgtggttgatgagatagatactggggagaaggatactattaatataatttattgagcaactttgttctgggcactagttgagtactttagggatattaattcatttaattctcccagttctgccatttactctctatgtgacctggactgttacatagtctctcagtctcaccctctctttttttttttttttttttttttttttttttttgagacggagtctcgctctttcgctcagtctggactgcagtggtgctatcttggctcactgcaacctccacctcccaggttcaagcgattctcctgcctcagcctactgagtagctgggattacaggcatgcaccaccacgcctggctaatttttttgtatttttagtagagacggggtttcaccatattagccagactggtctcgaactcctgaccttgtgatccacccgtctcggcctcccaaagtgctgggattacagacttgagccactgtgcccggcccttgccttctcttataaagtgaaatcattgtagctgctttggtgtggttctgaggacggtgagagaagacgcacgtgaagtattgagggtggtggcaggcacacagttcagagttactaacaatactggtgttttatcttcacaaggcagatctgggcccatagcacaaggaaaccgaggctttggcattttacagtaattacattgccagaacatttcccccactttcttgaatcatctttacctcttgagagatgaggaagagagggcacacctcgggcagaagtccaaagacctcaggcatttttggcctttcattcccatgattctggccaacatcattttagtccccagcagttcatagaaatattgtcttttaatctgatattcaaataatctgtgccttgaagaacctgtgtatggctctagaatcttttacattaatctactcaaccccactctgatgctcaaaacttcagcagtctgttttttttttattttaatgtctttattattatttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgctaccacgcccggctaattttttgtatttttagtagagacggggtttcaccgtgttagccaggatggtctcgatctcctgaccttgtgatccgcccgcctcggcctcccaaagtgctgggattacaggcgtgagccaccgcgcccggcctaatgtctttattttaaacatttatttttagaggtagggtctctctaagttatgcaggctggcctcaaattcctgggctcaagtgatcctcccaccacagcctcccaagtagctgggatgacacccccaggtgccgccatcctggcagcagagtcagttttgatacatctttcccttctcattcttcccaatgtcatggattgggaagttactgtggttttagttgcagtactttttactctgaattctgtttgcttcctggaattctgtctgtcttttgaaaagacaacataaagaaatcgaaaaatatactttaaattccatagtgtgggagcctctgctactcgcctgtttgatattcattcccattttttcctttctaaaagaattccagattttttttgttcagggtgacaatgtgctctgctgaaaatactttcacagattcccttgcaggtgcatgtgaccagatcacagtaatggcacatatggcctcagtggaatttgctgaggagtatgtccctcccaagtaacagcaaagctttattagaggaaaaaacaacaacagaaaacctttcacctttgtcttgtttctcctgccttctgtctggaacttggacacagagtcttgaatttctgtgaccataaaacacgaggataaaaccaacgtgctcaggatggcagtacagaaagataggaatacgctggtccctgatggcatcatggaatatctgcagtggccctagcttcagtgtcacttcttaggtgagatacataatcctaccgtgtgtttaagccacggtttgacatgtggtctattctctgccgctgactgcattcccagctgttctatatggcttgctagctaatttaagtctagttttattgtcaaattgaccacattgtagattataatattaaagtacttcacagtctaaaagtgaactacttttgtaaaagccaaaggaaatgggccaaatgaaccattttgaacagagtttaaatgagtgagttgagttaccactcttggatggagaactggagacctggattttcagtctagttcttctgccagttggctctgtgggcaagtcacttgacctgagctcttttcctcccatctgcaaaatgaggtaatttgagctagacagtcctcccaaagaccaccaaagagctagcagattgaggcctgcagactcctattgaatttgccatccttgttcaccttgccaagacttcatagccaagaagccagacccagattttgttcattagctttgaaaacaccctttcagttgggtgtgtgtgtgtgtgtgtgtgtgtgtggtgtgtgacatgcgttttgaatataaattgtgaattgttttagcaagaaaccaggttgtatgtttctcaataaacatgcatactgtatattgatgaatttatccacttgaaataaatggtgccaaattgcctaaagtttgctgtaaacaggaacatgatttctcattatgatgtttaatttgttttagtctttgatctcttacatcagaattctgttaagaactctttttacctgaaagatcaaagcaaataagcagaagcatttataaaaataaggtatacgtggctgtggagagaaactccaggtatatacgatcataggctttttaaaattaataaactttgtttttaaaaacaaagcagtggaagttcacagcaagtttgagtagaaagtacagtttccatgtaactgctgtcctagcaaagatttttaagtacaatattttttaattaaaaagagaaaatataaaccgtgctaagccagttggtgaaaaaggcttcattatacttttagcactcttaacgcttattaaaagtttttaaaaacagatttttgtggttgaatcgcttacaatttctgatgtgtaaccttctaacatgtttcatcaaattttaaaagtaatgtaagcagtgctgtccactgctaaattttattttaatgttagaatacaatgaggtttctattttgccattagtcctgagagcttattaagtaaccagttttaatcctttaaaaaaccattctcattttgagggacacatatttcttagctttgtgcaatataagaagctaagaccatcttggcagtatcaaatgaaatgttttgcaagttagaaagttcagtaaccacttataaacataatgaacaattgttatagtcagtctaatgcagaaatacaggtgttgggggcaggggggtggtgggtgggcagacttggtccacaagtagtagtttgccaacctctgtcctaaacacaagttcaagaatatggttctcttggatagattttctgccagatgaaagtaagagtaacatccttagctaatgaactcctaatatccaaggcaacttttaattaattcaggcttttaagaaaagcttgtttaccaactcctcattaaaaatgttcagactggctggtcgtggtggctcatgtctgtaatcccggcactttgggaggctgaggcaggcagattgcttgagcctaggagttcgagaccagcctgggcaacatatctagaccctgtctctgcaaaaagtgaaataattagctgggcatgatggtgcacagctgtagtcccagctactcaggaggctgaggtgggaggattgcatgacccccggaggtcgaagctgcagtgagccattgatccaaccactacattccagccagggtgacagagtgagaccttgtctgaaaacaaacaaacaaaaagattaatgcagtacatgtccttttggtgaataagtaaatatgttttggtaactttagctttgcattataatctagtcgtcagcagattgactaagggcagataaaacaagatgatttggttttgtggtttggcacttactgtctgatttatgcccccaagaactcttgcaggccaaggtcgagtgtatctgatacctgctctggctcttctcacgaactgatttagtagctttgggccagcattagattttttaaactctttgacctatttgtacatgacaggcatttaatctttccagtagttacttgtcaaatagaaaagcatcacttcttagtttggacatttgcaaatatgactgagcagagctgacagatgaggtgggcatcttcaggccatagggtacttctggcctcctgtaacctagaaaattgggatgcctcagatatcaggtattcttccaagcagctagggttagtccctgacctcagaaaccctgagagccctcagaaaccctgagaaatccacctgggaatggtgtatttgttttggatgagattacctttactttccattctccaggtcttcttgtgttttttgtttttgttttgttttgttttgttttatatcattctatgtaaaagagaaatgtggtatcatttgcattatcatgttatttctttccttataatttatatcttaatgcttatctctggggctaggttataatctcctgagttaggtagccttaggtttcttggcggagattcctcccctgggaagtgaggtttataaaagaacccgtgatgtggccctgtggagctgcatggggtgtgtgctcagtactcactggtttcttccctgcaaagtgacacctgcagcctttggggtaccatgtctcataatttttgcattccctgcaacacacactatactgctatgggcatcattgatcattttaaagatttaaggaatgacaagatacagtgcttattctcccctatgatttgcttagatttttatctaaatagtacttattgaaataagtacttcagtatcatgctacatcttacatgtttgagctagaaacagactaaagtttaaacccttttatgagaatgaaaagttacttccttccccttcttagggcttttaatccctctttatgttgcatttcagaaaatgccctatagcgtactatgaaagaaagacattcattcctgtctttgctgctgtgtggacgatctgtattccttgctccgtgggagttgtgggtttatgtctattcttgcatgttttgcctgaaggaccacagtgacctacttggtggtctccctgctttcatggtttcactatgctgatctatccttcacttaattatagagtctatcttaaaaaaaaaaaaaaaaaaaaaaaaacttttggcctatgttgcttcttccaaaatggcatccaaaacctaagcctctttaaaattggagtccagcatgcttccccagtgctacttccttctggactccctttgccacttgctttctgcttccacccatatccagatgggctgcaaatatgccttgctggttctttactctgtctttgcacaggaggctccctcattttgaagggtcttttgctacccatccatccatccatttggattctataatagagttcttgatgtttgacagaactaggccaggaattgtgggggtataatggcaagcaagacagagtctatccttacgttatggaagttcaattcatttcccatgtggcatactacttctcaccattcagattcatctcgaatattgtttcttctttgaggtttttcccactgcacccaagatactggcctgacactttccttatctctattacagaatttaacttgttcattcattaacagatatttattgcctactatgtgcaagaatctgtgctaaaagctgggaatattgcagtaaacaaaaattaagtttacagcttacatataatgtggtaattatccatcttttaccttcattagactgagttcatgtaggactttatgtaatatctttatatcttcaataccagaaatgcctagaaataatttgcataatatacattttctttaaagatttaacaaaagtctgagggacagttttcagaataaactgaattaaactctctctctcttatgtgtgttatgtaaacatatatataaaatacacttttgggttttttgcttaggaagtagaaaaatgctaaaatttctgttacttacaagcagattaaaatgtccaagaaagaaaagtagggatttatattgattctgctcaaatgcttacctagacaatgtcaatatttatattttactcccctgttgtagtagaatggtaattggttctctagttaagcagcttttttaggattaattttgaagcaagaaattcagattaattaagggacaatttcacttgcagcatttaggcattattttaaccatcaaacttgaagtggcaagaaacggagttgcttaaggaggccagacgtaacattctaaaggtagtagggtcttgattgggttgcttaggcattaaaaggctgtttaacttgtcttgaagtctatctttccttgatgtcttctgcggtaagaacactgtgatacagatggaatgacgggaagtggttttcctttctttcagttggtgagtttgtaagtgatgcccttctcgttcctgacaagtgcaaattcttacaccaggagaggatggatgtttgcgaaactcatcttcactggcacaccgtcgccaaagaggtaccagcccataaattctttcttattgcaaagtgaagatttcctggggacgtgcttaatggcattttaggggtattttgaggcaacagctatgttgataagttatttaagaaaaaaacccagtgctactatccgtaacccagattttagtttttagagtgattttaggttcacagcaaaatcgaacagaaaatagagttcccgtgtaccccctgtccccacacatgcgcagcctccccatccatgaatatttttaaaaagtcaaaaatagggccagttgaactagattcattatgtgcctgtgccgtgtttcgcaacttaaaaatataagttgtctgtgccatgccttataacttaaaaatataatctctaatagttataactttaaagtttacccttcagattatttttatgcacctgttgttttcttttgtcatatacaattttttaataatttagccttttcccctgttatctattagtgaatacagtggtagaacagtgaagtagcttttttataccagcctgaaagctccagttagaattcagtttctcagttataccatccagttttatattcagcaggccatgcctaccatcttgatttttccacagggtgagtctgaccgagcacttggttaacctctgcttttggctaagccaataaaagatgtaagatacttagcatgaaaaggtcactgtgtggtaagaccaccttcacagctgctgtgtccccttgataggtttcctaataaatttatataggtttatttgtggcaagatcagtcttaggctaactagaattaggctcctttatctagctcatagtgcttcacttgagtcaattttgatcccctgtccctcaccggaacatgtctggagacattttttgttattacagcttgagggagtggtgttactggcatctgatagtgcccagaggccagagattctgctaaacatcctacagtcaacaggataaccacccgccacccccaagaattaggcagtccaaaacatcaatagtgctggggttgagaaatctgatctagcttagcttaaccattgtatcatagctttatagctgttagacagaaaataccactgaagatttagtattattttatgcctcttataaaatagtatatttggatccactgtagaataaaatgttttttatgtaaaaatgtgttttggaggtttaagagcttgctgagtttaagaaaacaaaccaaataatttgaagtccagctacctatttgataacctcattttttattccttctgtgttttcaaaagaaaaatcctttgtctctaagaaacggaaatgactcccataaggtcaaaatacagtctgtaatattttcattgtctacctttatgtcttgaattattcatccttcatttatcactagtggatgctctcagaactttatgctaatctagatttgtgcaaatttagcaaatcccagaaaagcaaaaagacattcaaggagaaatctctaatgactgtaagttctgattcttagtggctgcaggggataatttcttctcttcccaggattgttactctgaaagtgaaccaagagagtcagaatttctaggataaggcaggtccatgtgagataaacttccatctgtaagggaggtaggagtgagctggggatctacctggaatgcgcacaatttctggatacacgaattaagccagaaggcattaattctctggtatctgttcaccagtggagactgctacatcttatttcccaattagtttccattccagttgttcgtataaacctctattatataacatcgtggtcttattaaataaataataaacaagaataaaaagagtaccaaagtgtaacccatgctaaaaaaaatcctgttgtaaattcactcaaaaagcaaataatcttttcctttgtgaaattggttcctaatatattgggtctgcatgttgattattttatgtggagttttcttacaatgaaacacatctactctaccactcactgttttctccttacactttgtagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggaggtaaggtggcctttgtgttcagcctcagagatgctgaaacatcttgtatggagtatttgtatcctgtaaattaatctttctgtttatcactgaaaaggtctctgcccactcccatcagagtctgctgttatgcaaaaatctgaactatgaatttttatggcatcctgttgaattaataatatcagtcacccatcacagagttaattttaactatttaatattaaacttgggatcaaaatcccactgataactatcataggttactggtagttctaacagggagttgaaataataatggcgttctgttttggtcattaatttaaaaatatttttaaatgctctctgggattgtcagtcattagtttaaggtgaattttcgttaaaggtttggtttgagattttgggcgttctctgtgtggatgtgtaagggtttttttttggccttttatcatttcttttccttcatagtggaagtcaaaggcattaataaatgcttgttaattttttttaactacctttgatttgttgattgtaaagaaacctattttcgcctctttttcttagggagcttagtcccgttacttcttacctgattctcattcttaactgaaggcaaacatctatattcaaatcataaaattataaaatgagaatctttgggttggaaatgccatctggttcaggcattgattttatgctgtctcatcccccgaagggtgatgattgtatcacttatcttattccttcagtattctcttagatgttccctagagctacagatttccaaacagcacattcaataggagttgtaagtttataatcggcaaactacaggatgcttgtttttttttcttttaatttctggtgtaatctgctactagggcccccatagagtttattgtctatatcagctagaacactgggaccacagtcataaactgaaggatccaggcaaactgggtcatatggttacccccaccaattacagtcttcagtgatgatgtcactatccaggttgatattcagttgaaatgctagccagctccttcctagttaccttcatttacatcccatttcagtacctgaacacagtcatgctttagactctgaaattccttagaaatagtaccataaggtttatagctttgatattcctctctgatcatgatcttccctcataatcacctccccagttcaggcttttagtccattatcctctttttccctgcctgcctttctaatcaggatggaatcaatggataaccattcttacctattccattgaacatccttgacttggctgaaacccttcacattttctaaacatatttttgtcaattctccatagctaggaaaaattaatttctttctttagttgcttctgggatttctaaagtatcaagtcatgtaaccttaccacttaattccatttcttattcatgctgtaacatcagctgggatcttaatgttactagatgcacctaattatctctgtcttgatgacctatcatcttccccatagcagttgatccagcccaacttcattctccttggttcccagcgtaactctctctcatgctctacagatgacccctccaccgtcatccattgtgaatttctgcaacatggtacctgacatttggtaggtgcccagtacgtgtttgttgaataaaccattgaattttctcctgtacttcagcaactcttgtaccttcttctatccctttgtttcctgtcttcccttttatgattatgttctgctggactctcaatcatctcctctccctttcttgcattttcagtctccctctcccccacacccccaggcttcatccaccctcctcccagataattctttcccatgtgttactatctttctactatgtgtttttaatcctgttgacagttggtaggaaatatttgatggtttattctacctgtagaaaagtccaaattctcttgcagggcattcagaagtctcttacatggcttcagcctgcattttcagccttatacatgtatgtgactctcctggcatcctaaaaccattgaccttttctttctctaggctttcattcccaccgttctgccccatctgaaatatttttcctttttttgtcaaatggggttaatgtatgataaagatatttgtcaactctaaagtgccacttcttatgacccgtcaaaatctatatatttataaatgttcacctgaaaagatgcagcctttacaagtctttacaggtgccttgctctgctatactacccttgctttacctttttcttataagaccataacttatcttccaattaattgtagtcatatctctttttatctgttagaatccaaactatagtttatgtgtctggtatgtcttgcacataggtcctcaataaatattagacaaagtaaattgtatattttaaattttttttcatagcttttggtaaggacttcatggtccacgattggatattgtagaaagcttatccagcccactgcccaggacgactttgaatgcggaccaacacaaattcataaactttcttaaaacattatgaaatttttttgcctttttttttctataggccctgttatagagcctcccatcttttagcaaagaactgtctaatacaaatgtaatgtgagccacatactttttcgttttctttttttaagatggagtctcgctctgtcacccaggctggagtgcagtggcgtgatctcggctcactgcaacctccacctcccgggttcaagcgattctcctgcctcagcctcctaagtagctgggattacagacatgtgccagcatgcccagctaatttttttttttttgtacttttagtagaggtggggtttcaccatgttggccgggctgatctcgaactcctgacctcgtcatccaccaacctcagcctcccaaagtgctgggattacaggcttgagccaccgtgcctggccttactttttcattttctaatagacagtttaaaaatagtaaaaagaaacaggtgaatttaattttaacaacattttatttaatccagtatatctgaaatatcatgtgaccaatatgaaaaatttaataagatagttcacattctttttttatgtttctttctttcgtaagaagtcttaacacttcttttaacatttaacagcacgtctcaattcatactagccacacactgtgtttgatagttacatgtggctggtgactactatatttggacagtgtagttcctggatgacttgaaatttcttatcactctttatttttccattaagacagacggtttgtataacctagtaatgttttctaaggctgagccactgaatttttattattgtttttaactattcttgtttttaaaatttaaaaaaattggtttggattcagtataattagtatatcaggtcacttgcaccattttattttttaacttcttttcctgtagccctgcaggcgggcagggcttctttcatgtggacaaaaccagaaaggtgttgcatcactgatccacacacaaggtgggctcctgccaactggcagcctccttgttccaggaaccagccctccttttgacacttgaaatgtaaaatcagaatagaagtttaacgtacctgacttaaaacatcttcaggggatgctacgtgacaattcttgaagaaggagaaacgagctaccattttcctggaaacaatatttgattcacagtattttgttgtttggtagttaatgaggcttgttttgatagaagatgagtgtttttctatcgctaatggggcatttagagaaacattccttgtaggatgagctctgttttatgacctcattgaaaaagaggtatgaaaaagaggtaaatctttaacataggcaaaacaggtagtgtttcagaaatttatgtgtaaattaccgagtatatttttctccagtgttgtaatggtcatattctcagatcacctcaaaatacctatttatgcagtctgtaatgtagcataatccagactgttactaacattatgtagcataatgttaataataataattacgtacataatgttaataatacacagactccagccatcgtgggtaagatttttttccatgggaaaattcatttggaaagctaacttgtaagactagccaggtaggcagcctagccctggaggcagagctgctcctcacctggaactgcagaatctgattgaacccagtggtccatgggggttgctgaattcgtaaagcaaatgaaatgttagaagaccctctaattgttgtagagtcacacttttcctcttgcagaggaggtactcacattttttttaagaaacaatctttaattatcgatgtattcttttaggtttaggtttgcagacactggtgagcgtgtgctgagaggtaacttgatgtatctttctggataattagatgattttttttttctatttctttgaacattttttttttaaagagctgcttaagatctggaggagtgattccttttgaccgtgtcttattttcattaagactgtataacttggaagagtttttttttttctttttctttttcttttttttttaagatgaagtttttgctcttgttgcccaggctggagtacagtggtgtgatcttggctcactgcaacctctgcctcctgggttcaaacgattctcctgactcagcttcccgagtaattgggattacaggtgttcaccaccacacccagctaatttttgtatttttagtagagacagggtttcaccatgttggccaggttagttaatgttactctctggtttggggggcagtgatatgttttggcttttttcccacccagatctcttcttgaattcccatgtgttgtggaagggactcggtgggaggcaattgaatcatgggggcaagtctttcccatgctgttcctgtgaagtgaataagtctcacgagatctgatggttttaaaaagaggggttcccctgcacaagttctctctctttgcctgctgccatacatgtaagacatgacttgctcctccttgccttccaccgtgattgtgaggcttccccagccatgtggaactgtaagtccagttaaacctctttcttctgtaaattacccagtctcaggtatgcctttatcggcaacatgcaaatgaactaatacagctcggggtggcttgtgtgggtgcgtatggccatgtatgcaggtgtgcgtgcattggaatatacaattccgtattgaacccagtttaaacttcatcagttctggtgattgctggagtgtcacatatggcaagacgtcaccttgccctctaactacaccagaaaaccaggccagcaccattgactatctagatacagtgggccgtttatattattattgtttcatatttttagattatttggtaaacaatttacagaactaagaataaaggaatagtgagaaataactattagcaattttaataagagttaacatagtaaatattttaattttacgaagaatttaaatatggtaaacccacatgtttattctgtattttcccagttttgaaggtatggatttctgaaatgagcattatcctttggaatgcagctagctgctaatcacctgccatgcacgaacctgaaatatatccgtcttacaaatactggttctacatttcaaatacatctctattaggttcatcatatttcttggtcttttttttttatacttgcaatttcatttaattattgtaaaaatttaagatatttaaaacattgacagctcataattttattgaagagagaatatagccatcttccctcaggaataaaatgttttcaagtttggatttgtaaacagctattaaaatgatcaagccagtctttttttccccctttttttctgcattatctcttctaatcaccttagagagattcctgccttgggcatttgtccacttcctaatggtatcaaataaatgtgttgcacagacataaaatgtgttgcataagcatcataaaatggcaaggaatactgtcacatcattttagcaatgatggttaatgtttttgtcacataatattgagtgatgatatccttcctgttgaatgactaagtggatgagaataccatatttcctttttattcttagaaatacattgctcagccgggcgcagtagctcacgcctgtaatcccagcactttgggaggccgaggtgggccgatcgcctgaggttgggagttcgagaccagcctgaccaacatggagaaagcccatccctactaaaaatacaaaattagctaggcgtggtggcgcatgcctgtaatcccagctactcgggaggctgaggcaggagaatcaattgaaccagggaggcagaggttgcagtgagccaagattgcaccatcacactccagcctgggcaataagagcgaaactccacctcaaaaaagaaagaaagaaagaaagaaatacattgctcacttgtcaattcttgagcttgagaaaattcatctaggatatcatgtgaaggctcatagcctaaggttttacttacgtgatcaatttcattcttcttctggtttatctaattggtaaaatgcctccttctgtcagttgttcctatttgccaatatgtggaagatgatttttgactatattcagtgagtctccagacttctttttatcatgctttttaaagagatgggacctcactctgttgcccagacttgagcgcagtggctattcactggtgtgagtatggtgcattacagctgcaaactcctgggctcaagtgatcctcatgcctcaacatcccaagtagctgggactatgcagactttttttaaaccatcttgaaagatgatgcagtactttgggaacacagcaataaaaatgtgataatttccctattgcatcacctttctaggcaattatatcattctttcatgctcatattattttgtcttctttagcacagtttttaaatagttgatggctaatgatgaaataattcttcggatgataaatattttgtaacataggaacacaatataggaaaaagatcgaggttcataccatatctcagactcatggttaagtacaagctagaatgagttttagtcgattaaatttctttttctctatgaatggataaagtgagaatatcagcccacaagtagttagaagtgctcagaactcaaaggtaaaattgtgtccaagacctgatggtattctaaaggttaacagcctatcctacttaccaagaatttacatttattatgaggcctgtacaacctgatgcaggaaaatacttggcataattttctataagttaggtagttaggtgctcaacaagtatttgagttgaatcacattggcattcacatcaaacacatacacctacgtcatcatgttaacttaaggttaatagtgtcaccaaaactaactggtctttaaaaaagaaaaactaggccaggcatggtggctcatgcctgtaatcccagcactttgggaggccgaggctggcagatctcttgatgtcaggagttcaagaccagcctggccaacatggaaaaaccccctctctactaaaaatacaaaatagccaggcatggtggcatgtgcctgtaatcccagctactggggagactgccagagaatcgcttgaacccaggaggtggaggttgcagtgagctgagatcgcaccactgcacttcagcctgggcaacagagcaagactctgtctcaaaaaaaaaagaaaagaaaagaaaagaaaaactaaggaatatcattctagacatgcagtaattaagtaaaattgcatcaggagtctaataacatagcatgatgggtcccatgtttcattatttccatgggcagaagagggatggctacattataaataaaggaaaattgtaagaataagaaaagatgaagtatgaaatatttccaactcaaaaacaaactgtatctgagcatgcagaggtacaaaatgtcacctttttcctccagtgtaatgaagggcactaaacctcttgagcatttgatatgttccagatactatattaaagtgatttttttttttaggagacaggttcttgctgtcacacatgctggagtgcagtattgcaaccatagatcagtgtaaccttgaactcctgggctcaagtgatcctcctgcctcagcctcccaaatggctaagactacaaacacattgccaccatgcctgcctaatttattttgatttatttattttttttgtagagacaggggtctcactatgttgcccagactgttttcaaactcctggcctcaagtgatcctcctgcctgggcctcccaaagtgttgagattacaggcatgagccaccacgccagaccaaggggtgaaatcttgataacaataatcaatgcagtcatcataaaacgcttgcacaggctcaatttgtaggtggcacagtgctacatctagtcaaaagcaaatttaattatggtcattgtgctctgagaacttttctcagaaaataataaccagatggtattatagatggtgcatttcacaaatacatgatttgtagttagtgaaaacttgtcattcaatcatgtaataaattatgcacatgccggtcttccacttagctagtgatgttgaaacattcatagccttacctgatgagctcaggctgtcatgagaatttatgtctattcagtactttgtctttaaaattaggtgagagagaaagagaggctgtatttgactatatatctttttatttgtatctctggagttacaagtttttgaatatataatctttcagctgcctttctgcattgcattttgagaaatgatattaaatgctcaaggaaaaaaaacctaacaactttagagggccgcaaagtgaatcatacaaaagataaattaaagctttaaatctttgccagtggatgagtcaagtaagagtggatttaatgaaatggctcatggctttgagaagcagctgtcataatgctgggttggcagcttccaggactgtgtgaggctttctgactgtctatactgaaataggagaactcatttgaaaatgacgaataggagaatagcagaaacttgcattttcttttgagggaactggctgccagttttgaggtccttatttagaaatgctcgattcctgtattgaataaacttggactacatgtatgtcaaaaagaaaagcaaaacacttttatagccttaagcagattacagatgtgtggccaaaccagtggggtctgtttttgtttccaagcaggtgatggctggaagaggagatgcctctgtagtgcagggcttgagttgctgctgtctgagctacatctggcaatgagactgtgctgcttactgagacctaatggcacaggttgccccagtaacaggatattgctgaatgagaaatactgtaggattcatttgtaagagttatttggagggttggattattcagtgctaaggatcctaagctactgatgcatggactcttttcttccctccagtatcttttaattaaactgtacaaattccctggcttaaaaatagtaggaaaagaggaagaaaatacagattcttatgattttaactggtctctggcaatagtacaccgaaacattaatttcttgttgaaaccatctggatatttgtatcttcagtgtaaaacccctaatttagataggactctccattgtgacatggaaaaataaccctttaattagttaattaattttactgtggcctcaaaatctacatattagcatatcaagccaaaaactgttggctttacttgggagccatgtggcttaaactagtaccagtaacttaccacaaggaactcagcaaaattataagtccatgtttgttttggaaggaaggcaattattacgaaaataatttagagatgttcaaactatttcctttgagtagaggtagagagtgaacaaaatctgtcccaagtgggagctaactagcttccttttttgccctccctgtttacaaagtaacacttttagtttactaggtaacatttgcagtatccccagtgtttcaatgtaaaattgtaggtggtagactaatgccacttagccaaagccatgatcacctgaatttttaatatatatttttgtatggagtgagatactgggagaacagtcttcattagcttaaactctagaatgtactgtttgtttgtttgtttgtttgtttgttttgagatagagtctcgctctgtcacccaggctgcagtgcaatggtgcaatctcagctcactgcaacctcccctcccaagttcaagtggtactcctgcctcagtctcccgaggattacaggtgcctgcttccacactcagctttttttttttttttttaatggagacggagtctcactctgttgcccaggctggagtgcagtggtgtgatttgggctcactgcaacctctgcctcccaggttcaagcgattctcctgccttagcctcccgagtcgctgggattacaggtgccaaccaccatgcctggctaatttttgtatttttagtagagacgaggtttcaccatgttggtcaggctggtctcgaattcctgacctcaagtgatccactggcctcggcctcccaaagtgctgggattacaggcataagctaccatgcccagccaagaatgtacaatatttttaaagaaatgcggtttattaccataaaatacaggttgagcatccctaatccaaaatctgaaatgccctaaaatccaaaatgttttgagcactgacatgacagcaaacgtagaaaattatgtgactttatgtgttcaatgtatacaaacttcctttctgaaacaaaaattatttaaaatgttgtataaaattgccttcaggctgtgtgtataaggtatttttgaaacataaatgaattttatgtttatacttgggtcccatctccaacatatcttatatgtatggaaatatcccaaaatttgaaaaaaatctgaaatctgaaacacttctggtcccaggcagtttggataaggattactcaatctgtaaatacagtagctcaaattgattttgtaaggcagtcaagttattatttatagtattttaatttgtattattcttttaatatagcaattcattttatatatttatataaataaattttttgcaattgagcaaattaaaaaaattaagtgcttccttcagatctacattttattttctatttgttatcaatttaataagtaaaggacacacagatcatctgattgtgttgtcagacatttgaaggtgcaactgatgaatgttcactgttgtactttgttttgtcatgtaaattgttttaaagagaagtgaatgttgagcatatagtaggccacacgtgcctgtggattggctcattgttttaggaatgtgtaattctggaaaagatttgccaagggtattcaaaagtatagtttgatattagaaattatttgaaaataatctttatagcatagcaccattttatttagatcaatagtaattcaagcagtatacataatttgctattgtaagcgtagcatataacctaagttttcttttacatttgagaccactgtttgctgttggtcgaatttggtcttccaagattttttgcttagtgacctaattgccagcctttgtgaagagctgttacttattgtcaaaccaggcagtaagggtttgcttttatctttttatcagttctttcaatgctagtagtcatgagattaatgtagttggatactaaaatgttaatacgtgttagataacaaactgggaaagagatgaatgacatcatgaaggaaagggcagtattgcacttacagtgatttcatgatgatagaactaacctgcctaaaaagacattgggtggaaacagaaaccaggtgtaatagacgcatccaagtagtcaaggaaagaaaagacttgtaagtaaataagataatctttgcttcaaaaatatcttcaaaggtttaaaatgtgctttaccctcatatataatctttttgagcctatttctttacctcaaacatggggaaaattgcaataatctgtatcacggtatttgcttccaaaacgactaattggtgtaaatgcatatcaagccggtaaagtttacagtggacttgtttaaaatgcgtagattggaattgttgggggagcaggccagcagcatgggatgttttttttgtttgtttgtttgtttaagactctgtcgcccagactggagtgcagtggcacaatctcggctcactgcaacctccgtctcctgagttcaagcaattctcctacctcagcctcccaagtagctgggattacgggtgcctgccaccacgcccagctagtttttacatttttagtagagacagggtatcaccacgttggccagactggtctcgaactcctgacctcaggtgatccgcctgcctcgggctcccaaagtgctgggattacaggcgtgagacaccgcgccagctggggtgatttttttcttagtcaaatcacttctctacccatcatctcacactgtcgaatacaaagactctccttggggctcaatgactcatctcccacctctggtttgataatcatggctattataacatgttccctttttaattagttattttaaaattattgtaggtcttgtaatcataatcaataataatacgtaatggctccttaagcacttctatcctagataaggttgagaacattattaaaattagttttggtagtaatcaagtaatgataattcttatttatgaatttaaatatgtaggagtttttttattttagggaagcaaaaaggactagaaagtatgatgtctcatgattagaaaactgctataaaaatgaaggtagaattgatttgggctgttttggacattttaaatagaaatttaaataggtattcaaatagcaaagtcattctactttaaaacttctatacttttttaggatcttcaattttatatttaaattatatttttgaaatggttttagaagaaatattgtatcttaaactctaactactaataagtaagagaagatatatcaaaggtttgatcttttatctcctagaatatactattcattcgtttcccattgtacgtcatttgaatgttaatggttagcctcaaatggttttcaacataattgtatagctactattattggaaaattggagccaaggactcatacacatatcagaagtggtaatgaagcactttggttttatgaaaatatgtttacaaggtagtcacaggatagcaatattgcatttactgtaatacaatgatggtggaactaagtgcagtggaacaggatgctgagaccacagttaccgcatctctaagtgcctcgtctagttatggtccaacctcagatataactcattggtgttgaaacacacctagcaacattttaagcatagaccgttcattaaaaggtacctttttaaaaatttttatttttaaaaattgacatgtaataattgtacacatttacagggtacacagtgatcagatcagactgatcagcatattcatctcaggcatgtatcatttctttgtgttgggaaaattcaatatcctgcttctagctgtttgagattatgtcatatattactgttaactgtagtcatcctacagtgctgtagagcactagaacttattcctcttatctagctgtaatttggtatcctttaacaaatctttctctatcctcagtttctacctacccttcccagcctccatcatcctctgttctactctttacgtctatgaaatcaacattttctagcttctgcatatgagagcatgtggtgtttaactttctgtttttggcttatttcacttaacatatgtcctccagttccatccatgttgcttcaaatgacaggattttattcttttgtatggcttaatagtatttcattatacgtgcatgcacgcacggacacacacacatacacacacacacacacatcacattttcttcatccattagtctctttggacatctaggttattccatatcttggctattgtgagtagtgctgcagtaaatatgggagtgcagatacctcttttaatatactgatttctcttcctttagataaatgcccagtagtgggattgctggatcgtatggtagctctatttttagttttttgaggaatctccatactatcctcaaatggttgtactagaacagagcttttttaccttccttccatttgggaatcttcttacccaacatgtatattagtaacttggctatgtatgttccattctctgtctcaaatactgtgtctaggacatctgcaatctttggctatgaccacgactgtaaagtgcatgacgcttttgctctgtcatcagtattagttgacaggcaagtaagatagtacttctcttcataacccctttacctgcccagcagaataccagtacagacagacccagaatatctctgggctcaagaatccagacatttataggatgggacttttatctataaatcaccttaagattagtaagttcttagaattggttacctttgccatttctcagaagccctttgtctagtaacctgtgtgtcactgtcatttccagcctgtctttcctacacattgtttatgcaggctttaggagtgcccaccccccagttctaccccccacccccaccctagcctttgtggttttaattatcctgtcttggctgattttgttttgttttgacatttcatccattaaattcctgacactggctatctctttacaattattatactatttttctttctggctcgctcttcctgattgctgtaatccatgcccttgactttaaataccatttgtaagtagactaggccaaggtttatttctctaggccagactctacctagttatttcataggcatatgaatcctaacatgtccaaatgagagttcttaattcccctgctacccagtcctcaatgtggttctctcaaagctattctatctcagtaatggtcctatgccctctcctagggattattgctgagaacctagaactcatcatcgtctcttctgcacatcctatgaattctggatttatccacttatctccatcttttccagcactaatctaaactcccattgtttcttgctggagttatttccgtatcctgctaatctctgctctcgttctctctccctgacacatgcgctgctttcttctcccacaccaagccattctctgtacagcagccaggatgtagagtgtttatttacataaagcagatcctggaacccccaaatttaagttgtgttcgtggcttaccaatacgcttaaaaaccaagtgcacagagagaaaacaatgaagccttatttggatatgctgaggataattgataacgggtaacaactaacaaacacgtagttggtgtcaggtttgacctcctacattccttacaggtattagtgtatttgacccagctcctccctacctccgcagtatcaccccatgcctctctttcccccgactctcccctgctcagtgtcacacaagtcaatatccccagcttgctaacccccgggctgacagatctcattgtctataatggatgttccaaggtcccaccttcagcctcagttttgttgccatctttccagaatgaattcccttgatcattctgttttttggagacaaggtcctgctctgtcacctaggctggagtgcagtggcatgatcatggctcactgtagccttgacctccctggctctggagatcctcctgagtagctgggactacaggctgcacctggctaatttttttttaatttttagtggagacaagatctcactttgttgcccaggcttgtctcaaactcctgaactcaagcagtcctcccacctccgtctcccagagtgctagaattacaggcatgagccaccatgcctggccctgatcattcttttctgaagaaaacaccccttcttcattattctcaatcctagcagcctatttacttccattctgaaatgaatcacattttagtattttattggttcgttgtcgttctctccccccatagaatgtaatctctctgagtacagggagcttatctttttcccttttggagcctcggatccagcagaatgccagacacttagcagccactagcatttgatgagtgaatggcttttcatacacaattttagacattgttcctgattggtctctctagtcaataatagatttatcaaatctaatcgataatagatttctttttcttttctttttttctttttctttttctttttttttttgagaagtcttgctctgtcgcccaggctggagtgcagtggcatgatcttggctcactgcaggctctgcctcccaggttcacgccattctcctgcctcagcctcccaagtaactgggactataggcgcccgccaccatgcccagctattttttgtgttttagtagagatggggtttcactgtgttagccaggatggtctcgatctcctgtcctcaagatccgcccgcctcagcctcccagagtgctgggattacaggcctgagccaccgtgcctggccgatagtagatttcttttattgattagagagactaatcagcaacaatgttttaaaattctcacatagatctctgaatgggaaagcagtcttgaattatgcttgcagggagagagaggggaaggaaggaggaggaatggtataattagacttcataaggctagattttgtttctacttaaataatggtatcacagtgggacatattatccatgcagaaagtgagtgggcatctggtctattatctgtggtctttcaggaatggcagctctttagtttaaaaaacaacaaagtgcagcactgtttatttaatggaaatagaaaagcagtgcctgtgtattagtggctcagatttttttgttctttgttttcagctgtctgctgtatgcctctccagcggggtcttacagaatctcaagtctaccatgatcaaagctgaacccattagctccatttcttaattcctcctgtatcctctgttaccattagtaactattctccaactcaaagcatcagattcctcctcttactcactcccttcatccccagtcaccagttctgtgctgttattttatccaagttctcagtaggcttcacgagcaccgccttcacatcccaactgtcgctcctccacctatctgtcctccatgcttctgctagctcatgtttgttgcaatacaactaaccttgccactcttctgcttcaggcttttctactaggctctgcatctagatgatggtccagactcctcagcctgtgaaacaaagcccttcataaaagagcagagctacagctagtcagctcccttcctgcagtccaccccctcctccaccatactgagaattggcaattcctgtagcactctttgtatgagtccattttcacactgctataaagatataaccaagactgggtagtttataaaggaaagaggtttaattgactcacagttctgcatggctgaggaggcctcaggaaacttacaatcatggcagaaggggaagcaaacacatccttcttcataaggcggcaggaaagagaagagcgaggagcagagtgggaacagccccttataaaactgtcagatctcctgagaactccctcactgtcacgagaacagcatggggaaaccactcccatgatccaattccgtcccaccaggtctttctctagacacatggggattatgaggattacaattcaagatgagatttgagtgaggacacagccaaaccatatcactcttcatctgctacgggtgccattccctgtttctacagtgtcctgtttgctaccttctttgtagatttctattcattcatcatgtcccagtttacctgttacctcttcaacagattattcttcagtctgtccctcccaaactccaccaatgaacattctttgttcagcccattattgttaatatgcacttcactgaaaacactgttttatgcttgtttacatcccatctctgcagggctctgagcctatagtcagcagttgggaacttactaggcatagcgcccagtaaaggatagctctttatatatatatatatattttttttttttctaaaataataagccccaagaaaaacattacctgtgaaaaacaaatgatatttttataccattttcaaaacctaatagcaataaaaagaaaagaaatgtcataagtatacatttaagctgtagttaaaaacagtaaataggcacagtatttaactcttatcatgtgctgttccaagtgtgttatttacatcattggctgttcacaccatcccctgaggtggttaatatttccatcccatgttaaagatgaggaaaccaagtcacagcagcctaagtgacatgttcatagtcacattggagctggcaggtggcagcactgagaatgaaacccacgtggctgagctctggaaagtcaccattaatcaccacactagaccttctctctaggatctacataactagaacatacaattaactggagtatatgtaagtgtagtcgttccttgagaaaggaaaagaattcacaaatttcatagcctcatcccttgtgcatttgtggcattgtgctaggggtgtctatttgatatttgaccctacagatttctcagatgccaaccaaccacagatactttaaaaaaagatacttaatacttaccaatgcaagaaagtgggcttgttggaagaggaaaaatgttagctcactaatggaaaatcaactaaaagtcttacctctccagaacatgcttttcctaaatgttgtggctaacccaagaagctacatcatctttacctttatgatacaatttaaagtacttttctacttacttcaagccctcagagcacagaggcaagggaggccttgggctataaaccctgtagcctgagccacatggcttcttctcctggacagatacctggcatattctgccactgaggatcagtggtgcagatgtatctgttttctcctaggaatggggctccacttacgaatctggggcaggacaaggtattgctgcattttggggagcgtgctggaaggaggagcagagtttgttgtttttgcttcctgatatggactggctgtgtctccattcaaatatcaacttcaattatatctcccagaattcccacatgttgtggagggacccaggggaggtaattgattcatgggggctgatcttctctgtactattcttgcgataatgaataagtctcacaagatctcatgggtttatcaggggtttctgcttttgcttcttcctcatttttctcttgccttttcctcatttttctcttgccgccaccatgtaagaaatgcctttcacctcccgccatgattctgaggcctccccagccatgtggaactgtaagtcccattaaacctctttttctccccaatcttgggtatgtctttatcagcagcatgaaaacagactgatacacttccttaacagaaccaattattttttcccaaaaaagagggggagaagcccagaagcatgcagcttctctcttctttctaaggaataggtcttcacaaagtgcccccaaaagaaaaggaccaggcttttctcttactttgtgactttcctaggttgctttgtagccattttggacacagaccacctatagctgtgtagtagttctcatgtgtatggacacagtggaaggagatgccaggcacatagttcttgctgcattgacaccttttcagggcatcttcttttaccatcttctgtagaaactgcaacagcccggccaactaccagccccagcccacccctagctttgctgctttcattttctctgtaacacttgtcaccttgtaatgtgctagtgtaacatcgttgttttagttttcttatatctttccttccctgtagaatgtaaataccaggaaggcagggaattttgtctgctttattctctgcagcatccctagactccagagcagaccctggaactcaaatatttgttagacagccaaaggtcatatacttccaactctccacccaaaatatatatggatcacccatccttagtagcagctaaacctttggacatgaggaatttgctcgttcacagttttctggcctagcatctgattaaccaaactttttcacatatcaagaatttaaaatgactccaaaactttttatgaccttaggtgcttctccttaattctagttggttcataaagctgtcagaacaataatctgcttttgataatatatgacttttcttgcctccaagatagcctttaaaaaagcaatttagccttttactgtaattatattccaaatttaagtcttgccagaatatcattttccccctttaaacatactttttatgatgccaagatattttaaagaaaagttcagataggaagcatatttaagatgtcagtatatgtctctaaaatctaagaacatttttctacagagtccagatgataccatctcacccacaaaattaacataaatttttagtaccattggataccatgtccatattcagattctccctgagttccagaaaggtccttcacaggttatgcatttcacatcctatccggcaccagggccagcatcttacatccttaattcttaggattcagaatagcactttttatatcctgacactgacttgctgaagaagctgtaccagttttcctataaactgtccactccttggatttgtcttatatcatgtcccaggggtattgtttagattgttgctctattgctaggatattcatgttaccctaagtgtgtactgagatagcagagtaaagagagattgtcttttaaaggttctgcccaggaactgctggtttacacaaaagctgggctttagtgcttctcggatctctttgcttgtttcagtaatttttttttagaaattcactaattcactaacgtaagtggggcagggtttacaggagcatgtgttgagagaagttggtttgactttaaatggtcatttttacaatccgtatttaggccttacaggtcgcatacaatctctgtcacatattgctgtcatttttaccccctttcttttctttttataatcctttaaaaaatgcaaagcccacccttagctcacaggatgtacaagaacaagttgcgggttgcagtttgccaagacctgctctgtgccactgctgacttgccattcttggagcccactttctgttcttgtcagccaattccctttcaggtgctcaggtttccctccctcctgtcaccgggcctttgcacatgctctacccaccctgcctagtggagagctatctttggcccctggacttcattcacttcggaccttctttgggtcacatttcagtcatgcctcactagacacctggaatctctgcctcactagacacactagcactcactcttttgggtcttctggctgtatgtaattgttatcagggcagtaattttgcatttatacaggtgctgaggtcagtgtctggcctttggctacttgccttgtgagccacctttgctttccagtgtacccctagctcctggcccagtgtccagcttcccagcatccattcatgttttccctggagaccattctgtgcccagcagccagagacatcattgcaacttaaatctcattgagtcgttccagttgctgtaagaaatgtaaccgtactgggtaacactatgcctgtctgtctccaccaccagatcttatacttcctccctcatctgctactccctaagctcactggccctttgttgtttttgctgaacgtgctaatctcttcctcacacctgggcctttgcacttgctcttacccctgcctcagccatccatgcactctcttggttttgccgtgcccgacttcagcatttttagttcacatggctcttccgtggggcccttccctgaccaccctgtttgaaatgctacccatccctggcacacatctatcatagtcattcattagcaattagccccatctcctttccctatagtacttttccataattttttttcttgctcatattacacctccccaactaatagatagattccaagaaattgagtgctttatcttcactacctggaatagtactggcacttggtaaagcacagcgaatattgttgattgaattactaggggttggcttagagaggtacttaatggaatgattcaatggatgaatgaatgaatgcattgccacttttttgctcattatcatcaatttgtacatatgcctttcaactcaaatatttatccattggctaaagaaaaaaaatacctgaatccccgtgtctctgcagtttacccttcctcactgttaaaactcagtgcctgtgtaaaatttcattatattttatttacttcttattccacttgtgcattatgtggatattcctgcctcatccccacagccacctatcacttccggccaactcagatgcctctggtcgcaacaatcacttctctgttgaatttctgaagcgtttaatctgcagtatgtctgttactcatacagcagttagtgtgaattgtctcactcagaataagtattcattaaatgtgtaagaatgatgatatagcctaatttcagatcaagattgtgatttatagctcattcttaatcagctgtgtcatgactttatttatttattccttttttttttttttttgagacagagtctcactctgtcacccaggctggagtgcaatgcagcttctgcctcctgagttcaagtgattctcatgcctcagcctcccaagtagctgggattacaggcgtgcacccccacgtccagctaattttttgtatttttagtagagacggaatttcactatgttggccaggctggtctcgaactcctggccccaagtgatctgcctgcctcggccccgcaacgtgctgggattacaggcatgagccacctcctcaggcctgagtcatgaatttttggtagtccagtcatgtggttatcttcctaaagctggaggccttaaacctagggccccaggtgggcttatgcctttagctgcaagtaacaagaaacccttttcaacctttttactgatttatttagagagaagtacacataagaggtaatgcaagtaagcaaagttttttaagaccacacaactagatagccagcatccggaccagaacacagagacttgctggcccctagaatcctctctcctggcccctgccagttactggcctttcccctgagttctatttgcctgcagtttgcttgttttcatatttaattcaaatagaattaggtgatgtgtccccttgtgtttcgcttctaacagtgcctttatgcgggagaagtactggctatttcttactttgccctccctccccggaaacattttcctaatttagtagcagtaatttcaaaaactgctttcctttcggacaaactgacttatccagccattgtaccttccagtttagactctggcagtttggagagttgagacagagagaaggagaaccgtctgtttatcttcttgaaaaaaactactgtattttctctctcttctgactctgggagcctctagactcttttttcccattttcatatccacagaggaagagtctgaacctcttcctgctgagctttcccctggttggttgcatttcaggaactgacattggttgtcttccctcttccctcatcagtgcaagtaggttggatctcatgatagtccatattcacataaccatgagaatcagagctttctctctgtcatttcctccctgtttatctgtagcaagaagaaaaaagtccgtcttaacagagactgtgaaagaataaatatagactttattgttttagagcagttttaggttcacagcaaaattaggcagaaggtactgagattctcccatatactccctgttcatacacttgcacagcctctcccattatcagcgtcccccttagagcgatttgttacaatcagtgaacctacactgacaccattattatcacccaaagtccatagtttacattagcattctctcagcattgtttattctgtgagtttggaaaactgtctaatgacatgtacccaccattgtagtgtcatactgagtacttttactatcccccaaatcctctgtgttcctcctgttcgaccctctcttgcctttaacctttccaagccactgatctgttactgtctataattttgccatttctagaatgtcatatggctttcttagaaaatacaaatggtgacttttttggcatgactttaataaacagaaatgaaaattggtgtgtgaatcaaaatctttcactctgtaaagggacttatcaggcggcacctgcatgtggaattatatcaaggtcagctttagttagaaacagaaggagagtcacctttgtgatgtcatttctccttcatccaagtaaaatctcgcatgctgacacataaaattttatcatcagtgatttatttcctcgtaggaatgtccctttgaccttattaaattcagatactctggggatggtcagcattttcagatggttcccttgttctgtaaaaatgtttgttattggtgtttaaaagctgtaaaagccacccactataaattgatagccactaaagtctgcgaatgtggtatgaatgttccactatgttgccaagcgtttattttattgtgagttgttatttgattgtttgtaaggaaattgttttatgatgctcagctttatatttccataaactgttcctcccttgcaactccttaaactgtgatgagcttttcaaaacaagtccacaggacaacagtctgtccagcatttgaagaagtggtggaaaagtcatttttttcctttcagtgtttctagctgttcatgaaaactgcagttaaaagttgtaatgttgttgtgtttggcattctttgggaagatgcagcattattcaaacaaaaccgtgtgctcttgttttaaatagtgtccaaatgaataattgaattcatggacatctaaatttaattccagaaatcaaagctatggtaataagaacttggacctgagttctcttaggccaagtaatttaattccttagccggttgaactatttattacgtgtctccgttcctggttattatgccagtaaattgctcaggcagatgcagggatggatgatgggcatataattcactctttttcacggctagacacataccagaatgcagatcacactaaacagctgtttttacaaacatggtttctattcagagctttaagggccacatccaagtaataacaaagaagcctggagaaggaacctggggatacccagggtcatgttttcaccatcccacctggaaacacggtgctgtccaaaccctggatggtacacttggacatcgtcattccagtcatggttcaagatcacgtgtatacatcatgatttaaagagaatactggtttgatttttcttcctgactttccctgaattttccacttaaattggaaaacagcgctagttagtgcccatgatttgattgaataagtaagagatctcaaacacggtaaattaactgatgacatgcatcaggtcatctttctttgattcaccagtaatacaacagtgagatcctacagaaaaatgcaaaggcagaagctcagggcagctgagcaactgttaaggattcttttgaaatgtagccccacgtagtaaactttcgtgcaataatttgggcagtcaaaaaaaaaagtcagttatttcacctcatttatttctacgaggagataaaggcttccaacatctgattttcagtgccagtgttttctgtttctcttttcacctatttcgtcctagtcttgtggaaatgttacaataactgattaggattttctctgtattaacacttcatttattaacatatgagtaagttgctattaataaattattctgcttaatcagtttctttttattctttagccacatgctaaacttcttcggatgtcttcacttaatatccttacatcatatttaatcagagttagaaaggcgttccacttgggggtcatacacatttttttgaaacttttataaacgccctttatactattcttgtatcctacgtaatcagagtcaggtgttccatggggatcatacacatctttatacaacttgtgtaacacatgctttaaatcctgaggtaatcagagaattcctgccattgatggattaatctctttacatatctttccctttcactctcagcaaaactacttatggcggtcctgttagactttgaaagcctttcaggtttatggaggagtcttcctttctattgtcgtagatcatggaatcatagtgttcgcttgtctcacgtgtttggaatctgatttcctacataatgctgtgctctcactacgagtccaaggcttcctgtggctacaaatcccgggttcctatcacttttcccaatgatgagcttctccttgttggccagaatcagctctgtggtaataattcccttctgctttctccctctcgcttttttttttccttctgggtacattataatgtcagcatagcagcttttaattctattactatggtacttctcgaatagacctgtggtagttgccccagaaaggttgaaaccccctgccacatctaggtctttctcctgtattggttttgtgatctgatcaaggacatctattcctctagtcctgctaggtggtatacgataggcttttactattagatattcaaatgctgtcaaccatgcctgccttgacaggtacatgatttccacaaggatttcctaataggcctttttaattttgttctagacatgcccttcttttgccatccaaatttgcttattttttacaaactatgatgcatcagcatgtaaatgtttaataccataaatatttctgatcctcttcccaatttcactgagttactggttatctcttgccattattctcagcatattgcagtggatctcagaatattttcccctgaccaccagcatcagtattacctgggaatttgaaatgcacattctcaggccccaccaaggcctcatgattcagaaactctggagctgcattagaaactgccacaggcacctggtgatctgtgttggtttttgtttgtttttaaggagacagtgtctttgtctccctggctggaatgcagtgatgcagtcatagctcacagcagcctcgaactcctgggctcaagcaatcctcccgccccagccttctgagtagctggaactacaggtgcacaccaccatgcctggctggtttttgtttgtttttaaggaggcagtgtctttgtctccctagctggaatgcagtgatgcagtcatagctcacagcagcctcgaactcctgggctcaagcaatcctcccgccccagccttctgagtagctggaactacaggtgcacaccaccatgcctggcttttttttttttttttttttaatttttttggagacagggtctcaccgtgtggcccaggctggtctcaaactcctggcctctaggttttttcccacctcagccttccacagtgctgggattacaggcatgagccaccacatttagcaggtaggtgatctgtgtttcaataagccttccaggtgattctgatgctcactgaaatttgagaaccactgaaatatttggttttctacaaaacataagcagttagtaaccatagggtacgcaactaaatagttccagcaggcatccctgaccacagggcattgcagatttacttaagtcagtttcatcttccacctgtgtgagttccagcaggcatccctgaccacagggcattgcaggtttacttaagtcagtttcatcttccacctgtgtgagttccagcaggcatccctgaccacagggcattgcaggtttacttaagtcagtttcatcttccacctgtgtgagttccagcaggcatccctgaccacagggcattgcaggtttacttaagtcagcttcatcttccacctgtgtgtccagtttaagctctcagttgcagtttttaaaccaatttccaccagcatatactgagtatccgctaccgtgcaatgcgttgcggctcttgtatgtgcactatgagaagtaattcccataatacggcaaactcagttttgatatccaccaccccactttttataatggactacatttttcagagcagttttaggtttacaaagaattcaagcagaaactacaaaaagttccctctacctctcacttccccactccccaacagtttcccctggattaacattttgccttagtatggtacgtttgaaccaatatcgatacactattattaaccagtattcctgttttaacatgagaggcgactttttgtgtagtatagtctcttggttttgacaaatgcttaatgctgtgtatctattattacttatgacagtatcgtgcagagtagtttctaaaaatcctctctactccacctattcatcccttccccactccccctgaaccactggcaaccgctgatctttttattgtctccgtaattttcccttttccagaatgtcctgtggttagactcatggcatccccttttagcaactgcagaaactaagaactgtccccaaggataaaaatttttgcagacccactgagctgggatttatacttagacctgctgaattgaagttttgggtgtgtttcttctcaaattgccaaaattccatatggacgacttttcttttccttccctgaaatgtgtttaattgactttttctgttatttgtgtttgccttcacagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgaggtaatccaccatttgcttggattccccccacccccaaggaaaagaaagcgtaataccagagttggaaatatccaccctagcaccactgccttccccaatcaaaaacatgtttttttttccaaaaggcttcttatgcttgtgaaatttttttggtttaacaagcaaacaatttcaaataatgtgaaatctttattatacagtttgttttgtaccttgtatatgctgcttggcaaatcccaagttaattctacaagactccgcccaaccaggtagtcatctaatttgacaaacactgagttaggtgactccttggtattcctttggcagtgagtgtttgttatataatattgtctgttaccacctagacgatgagctccttgagggtaaggacttgattttattcagtattgcacagagtgcctggcttgtagtttcgataaatcttagttgagtgagtagttgtgattagatttcattaaagaaggagatttgggccaggcacagtgcctcacacctgtaatcccagcactttgggaggccgaggcaggcggatcacctgaggccaggagttcaagaccagtctggccaacatggtgaaaacccatctctactaaaaatacaaaaatgagccaggcgtggtggcacgcacctgtaatcccagctatttgggaggctgagacaggagaattgcttgaacccaggaggcggaggttgcagtgagctgagatcacaccattgcactccagcctgggtgacagagcaagattgtctcaaaaaaaaaaaaaaaaaaaaagatttggtgagtatcgcattattttatgctatgaattttaacagaggtgatttatgttgagtttacattgttttattgggaagtacggcagtgctttcttcaagaagtaaatatagtattaatgtcttagactttctctattgaattaatgtttgtgttgcaaatattgtgaactaaaagaactaaaggtcatacttgtctccatgaaataaaaacaaacattcagttgatattaaatgaccaaatgttacagaaacgagtaaactgttggattgagttccttaaagagtatgcatttgttccttaaagagttctgcatttcatatctgccatctgcttggcagatagtaaaagatgcttctagagctagattgaatgaatatgtacttgactgaattttaatgtactgcaaatacttataaaggtcaaccacaaatgttacagcatttacaaaaagtggctttctctcactttttgctaaagtttaatgatttcagatttattgttctattagagcttaacatgggctattttgtctccctcaactatattcatagttgtcactatagtattttcttccctttgttgcattttttaccttattaaggtgtaatttacatataagtttcatttgcttttaagtatataattcagcggctttaagaaatttgccaagctgtgcaactatcaccataatctaatcttagaacattttcgtcactccagtaagatcacacatactcactcccagttaagccctctactcacctctagccccaagcaacaactaatctactttctgtctctatagatttgcccttttttggactgttattataagtgggatcatataatacatggtcttttgtgtccgacttctttcacttaacacagtatttttgaggttctacatttctttaacttgagcttttaaaattgagattattctgaattaaacactcagcgttagcttaaaatttattgaaagttttacatgttctttgaaaaaaaagaacagagtgcaagatggattaagtttatcattccctacttaatgacagtttcttactcactttagcacataagtaaatgtgctagtaaatgcgcattttgtttaaaccaccatttttcaaatactgagacatgctttctcagggtgtttttcatacaactgttaggaccaactatggcagatctggtcaaatgaacttgttagcctgatgcctgtagcagggaaggacagcttttcagggcttctcttctcgaaacctaaatgaagtgtgaatacaaattacagcagctagcagcctgcagtgggactgaaagtccttggtgaatctccatcaagctgggctgtttaatagaaccagtttagtgtcccattttacttgaaacattagattttgagcacctgcttggtacaagccattttgctaggccatgtggaatggatgatgagcacacacagaccagctaaccctagtggatgttaaaatctcttaggaaatagaagtcaaagaagtacaaagccatctgtgttgtaagaaatgtgaacacagaacctgcctccgagagtaagagggaacttctggattgatcagagaaggttccatggagagcatgacatttgaactagagccaaaagtgtgggtgggtggtgtgtctaccagtggattttgctgtagagtcatcaggactagcaaaacaaatgtggtgccatggaaaggcatatttaggagacaggattggtccaatttaagctagagcagatgtcttttaggttgggaccaaaatatagagaccttcaatgccaggctggcaaagatgggcattaactgcaggtgattaattaacaaatattaattcagaagctactgtgcgctgcagatttttccaggagacaataaagagattttgttttgatgctagtactgttgaggtaacaggtgaacaaggagtgacatcagttatgcagtacaatgagaggatgccacataaacttaggcccatattctagaacaagacaatttgcttgagataaattggtctcattatcgtctgcattagtccagttctttttgcttgagttttgcaggtagtattatttgatattaagacggaacacagagatgttgtattgttttatttcctgtagaaagacgaaggcagttaaaagaatttcataaacaaaacactccaggctaaaggcaaaaaagaaatgaaccttaaaaaataacaagttaataattttgttggcaagaagagcaacaaaagcaaccttctgaaagcaagatacctggtcattgagagatagcaactgttaatgccccagaaatagaggaatctttaatgcaggtccattcattccacctcaaactcctgaacaaaagctctaattgaggcaagtcttcctgggaaagggggcctaacagaaacatgttatggtcagttgtgcccatgcgagataatttttttgtgcttgtagctaattccagtaaagaagtagatgttttagagggtaacgttaaaggggtcagctgcaaagagtccttttcctcctcctcactgttacaaaaggtgacttgtggttcttccctaattgagagcaatggcaacgccataggtgattactcattttgtaaaactctaattacaagtaggttgcttctgtgttcaacataaaaattagaagtgacttgcaaagatgagatactgcagggaggcttacaatcttgttttcagtgttagtgtttcagtacctggaacaaaatgtgatttacagccatcctttctaagaaaattgtcatcattaccaacatattgctactgataccagtccttaatgtgcttcataaaaaactcaataatttcataacccgttaaaaccggaaagaaatggagggaagaaaaataagacacttttagcgtagtcataatcttctctgccatattgtgttagaatcgatgatacccaaatgagaagatgtctgttctctgtcactgattccagtagaataaaattattgtttaaaagtaagcacattcgctgcactccagcctgggcaacagagtgggaccctgtctcaaaaaaataaaaataaaagtgagcacacgattgcaagatctatctggttttgggggcttattgtcatctttggttgttaaaaaaatacttggatattattggttattggtctagtggaataagccattaccgatttgagtacaacagtggtgtatttagatacttgctcctaattttctgttcttcaagaagggattgtacttcagaaaccagagatatcagggtagttttgattgagccataaattctaaattctgattgaaatgggtattgtaataaatggcataatcttttatttaatctagccaattgtgccctgtccagaggaactggacatttcttccttatgttccatttattcagcaactattcgttgtacccctgctgatggctaggctctttgctactgtggaaggcatcgttggggcagagcagacagcattttacctcctgggagactggcagatttttgaagcctttgttgcagatatagattttcacggtcaccactcttgccctttaatgtaatttaaatttttttttttttttttttgacacgggattttgctatgtttcctaggctggattcaaactcctgagttcaaacctcagcctcccaagtagccgggaaaacaggcctcttgtgctttaaagcagtgattctctgtctttaaaccttgtaactatgctgtgtggtggaaattttacttccccgtttaaagccaagatctctaaggccaagaattttttttttttttttttttgagatacagtctcgctctgtcacccaggctggagtgtagtggtgcgatcttggctcactgcaagctcggcctcccgggttcacaccattctcccgcttcagcctcccgagtagctgggactacaggtgcctgccacgatgcccggctaatttttttgtatttttagtagagtcagggtttcaccatgttagccaggatggtctcgatttcctgacctcatgatctgcccaccttggcggcaccccccgccccccgccccccgccccccacaaagtgctgggaatacaggccaccatgcgtggccgaaaaaaatttttttaattggcctgggctgggcgcaatggctcatgcctataatcccagcaccttgggaggccgaggcaggcggataacgaggtcaggagattaaggccatcctggctaacacggtgaaaccccatctgtactaaagatacaaaaattagccaggtgtggtggcatgcgcctgtaatcctagctacacaggaggctgaggcaggagaatcgcttgaacctaggaggcggaggttgcagtgagctgagattgtgccactgcactccagcctgggcaacagagcgaaacttcgtctcaaaaaaagaataaaataaattgtcctgggtcacataactggttaagtgatgaaatcagaatttggacctagataggccaggatatttctgctacactcatgtcaaaattacactttttattttctttataaatacacacaaattcatttcaacctgtgttcccttgtaaatgcatatacctttgatataggcatatattcatctattattttacactgtgcatttctgaatttttagaatttttacttgaagtgatcaggctggcgctgtggctcacgcctgtaatcccagcactttgggaggctgaggtgagcagatcacctgaggtcaggagttagagaccagcctggccaacatggtgaaaccccgtcttcactaaaaatacaaaaaaattagctaggcatggtggcgcgtgcccataatcccagctactcaggtggccaaggcaggggaattgcttggacctgggatgcggaggttgcagtgagctgagattgcgccactgcactccagcctggcgacaagagcgagactccgtctcaaaaaaaaaaaatagaaaaatgatcgggggtccacaaataagagtaatgatcaagggtcagcaaattggggccaaaaatagcctgttttcctgttttctacacaaaagttttattaggcacagcccatcccatttgtttaaaaattgtctatggatgttttcatgttttggaagtagagttgaggatcgcaaaagagattgttttgcctgcaaaactgaaaacatttactatctggccttttcagtaaaccatttgctaacccctgattctcactataagacggtgactgtgaagttctttctatttcacatactgccatatcaagttgatttcttttgaggtagggtttgtttttttttggttagagatagactagtatagtgtttgagagaatttttatgaccttgggccagttaagtaacctgttttttatgtgtgtagtgtagatggtaatgttatcttgcatgtttcttgtaagaatgactttaggtaacttatgttaaacgcttaccatcatgcccagtaagtgttcaatttacaaactcaagctgtccagattcaaatctttgccttacaagctactaactgtgtaaatttgtataaatcaaataaccttgaatcctcattgcttctttttgtaaaatagggattaataagacctatccaatatttaactattttgtgaggattaaattaattaatttataggaaacactttgcataattcctggtatacagtaactgctcaattaaccttagttgctctcttagttatggttatatcatcattccgatcctccattgcatttgaaacattagcaaactaatgtttactactaatgaggaatgttaaaggtaaaccaataagttgacttttaattataaaaggcataaatgtaacctatccatatctattggtataataaagctgatatttagagagaacattatgttatgattaaaagaaacatttcttgcttaaattttttttgtgtgaatatacactgtatattgaaatatctgcacttaaattttaaatccagctgaaaattggataagatagtattagatgccactgcatttcaagatagagttttatttcttgcaggtattttaagccttgtgggagttcttctgttaagtgaacagatagagacactgatacagatggctctttcaccgtctcatgtgatgataaaagctaaaaggtttgatccgaatattgcttatattgggctaagtgattgaccttggcagaattaaattatttttcttcttctggtgacaattaatctgggagaagtaccatttgatttattgctatacatttcaccaatatccacaaggcacttgtaggtaccagacatggcattggttgctgggggcacagagataggaacaaagtggcttgctcaggttgcttctagccatatctgcattgtccttgagagggatgagtctctcaggcctgcctaggaaggaattttggcagcatggagaagagaaagtgctcagcccggaacaaaagttttcacctactatgttgcagagaagttggcgtatcagtaattataaaggaagaatagggatttactgggtgatagagcaccaaatagaatgaatgcaatacatacttgaagtaaatcagagtagcttgtattgatctcaaggggagaagaaacaaagcttagcttaattctcatgcctctgttgttacttgtttgtaatggacatgacacttacaaggtatcctataattcactaacgggatttttacttatgattaatatttaactctccacaaagcttggggtaaactgtgaacttaacttgctgtgaacccgaacatgaactcacatacttcagttggaggcctcagtagtagcatgctgtacaaaaatgaaactaaagctccagggagtatttgggaacagggcaaaagggtaatggctcctccttatagagcaagcctcaggcctttacctttgtgttttgaagatggagaggatgcacttctactctcgcccgggttgtagcatgcttgctaatgatgtcaagagaactagtgtcagcactgaccatagatgtgacaggtaacagcctgttattttgtgccccatgtcatggcagccattgattccgaggtgtttgatacacactttgatagaggcagagtgaagcaggacatggacaagcatctttccgtgtagtttaatggtctcaatctagaaagcccactcagataattaggttgcttaaaatgtgatgagactagggaatagagccatatagaagtgtgtgtgagagagaattcagtggtttagtatattcacaaagatacacatttgacaccagcctgggcaacatagtgagaacctggctttaccaattaaaaaaaaatagaatagcagaagtaatcagtgaaaggcattttaatacaattatatctagatttattaaaaataaaattttagtccaatttaattaaaggccagaaactagatttttaaagaatattagtctttctcatattttccccaaatcagttgctttaaaaaaaaaaaagtgtgcagatgcctcaggcacaataactgtactcaactcaagaatttgcaattaagaaaaaaaaaaatcaaacctcaaagaagaaatccagcaactcagaggttcaggttcatttaaacatgcatacatttacatgggcacactttttttgttttgttttgtttttaagatggagtttttgtttgtcgcccaggctggagtgcaatggcgcgatctcggctcactgcaacctctgcctcccgggttcaagtgagtctcctctctcagcctcctgagtagctgggattacaggcgcacacaaccgcgcctggctaactgttgtatttttagtagagacaggttttcaccatgttggccaggctggtcttgaactcctgacctcaggtgatccacctgccttggcctcctaaagtgctagcattacaggtgtgagccactgcaccagctgggtgcacctttttgtttgtttgttttgttttgttttgttttgttttgttttgttttgttgacatggagtctcagtcacccaggctgggattcagtggcacgatcttggctcactgcaacctccgtctcccaggttcaagcaattctcaattctcctgcctcagcttcccaagtagctgggactacaggtgtgtgcaaccacacctggctaatttttgtatttttggtagagatgggtttcaccacgttggccagactggtctcgaacttctgacctcaggtgattcacccaccttggcctcccaaagtgctgggttactggtttgagccaccacacccaaccgggcacacgtttttagccaagccataggtctgattaagtacagtcagattccttgagcaggttacaaagctgtggatttgaatggagatctttagttcccttgcagaacaatgagtttccaaaagcagagtggcttacagcctaaccagctgcttctctcagcctttaaccattttgccctcctgaaaattcatctttggtttccaaccacctcttccaatatgcctgactttttgtgattctgttagtagaagagtaaaaagtcaaggggaggcaagaatgaatgaatctcatataaaaatgatgctcaagagaatcccttaattttcaagacagcagcccaaattgtaaacattgagacttttacagtgttaaaaaaaaaaaagtgtttacaggccaggcatggtatctcacacctgttatcccagcactttgggaagctgaggcaggatgatcgcctgaccccaggagtttgcaagcagcctgggccccatagggagaccaaaaaaaaaaattgttgttttggctgtagtgtcacaggcttatagtcccagctactcaggaggctgaggtgggagggtcgctggagcccaggtgaggtcaagcctgcagtgagccgtgatcacaccaccacactccagccttggtgacagtgagaccctgtccaaaaaaaaaaaagattaaggcagaggaaatgttaaaggttacgctatatttagttgataataatggtaagactgttcaggtgttttaactatgagagtagggttgcctctttcttaaatttatatatgttcttattgggtataatttctagacagaatatgcaagtatcacagaatatggatggcaattttatgttgcttgcacgtgactttaggttcttatgtttcatgataggagcaaagaaaagtcttgcttgccaattcttatgattgcatctgttctggaaatttactagcatttgttacccaggatttctgaagtgctggtgactgctgcccagtagtcttggtgggcaaagttactaaaatgcatcacgaatccttctggtgtttgcttgggctatggcagggtttctggaccttagtactattgatgttttgggccagataattgttgcggatgattgtcctgtgcattgtaggatgtttagctgcatcactagcctctactcgatgccagcagcacccccaagtgatgacaatcaaaaatgtcccctgggaggagtaggggatagactctctctggttgcaaaacactgggctatggtgttgagatctgcagtgatgagcagctttagatatgtgaacagaagcccaggcgtggtggctcacacttgtaaccccagcactttgggaggctgaggtgggcggattacttgagatcaggagtttgaggccaacctggccaacatggtgaaaccccgtctctactaaaaatataaaaattagccgggcatgatgatgggtgcctataatcccagctacttgggaggctgaggcaaagaaatcacttgaacctgggaggttgaggttgcagtgagctgagatcgtgccattgcactccagtctcggcagacagactgagatgccgtctcaaaaataaataaataaaaataaaaaagtgaacagagaaatacccgtgcaatactgtgtctcatcccccaaagacggagtcttgctgtcgcccaggctggagtgcagtgacacgatctcagttcactgcaatctctgcctccatggttcaagcgattcacctgcctctgtctccgagtacctgggattacaggtgtgcctcaccaagaccagctaagttttgtgtgttttttttgttttgttgttttttgcttttttttttttttttttttttgagacgaagtctcactctgtcacccaggctggaaggcagtggcacagtcttggctcactgcaacctctgctgcccaggttcaagcgattctcttgcctcagcctcccaagtagctgggattacaggcacctgccactgcgcccggctaatatattttttttttttgtagttttagtagagacagggtttcaccatgttagccaggctgatcttgaactgctgacctcaagtgatccatccatctcggcctcccaaagtgctgggtttacaggcatgagccaccaagcccggctggtctcattttaatgtaagctctaacttctgtatcactgctttgcacattgtagataaccagtgaacattttttattgtataagaaaaagtaaaataaacaaaaatatgccaaagcaaatatgataaatacacatttagtaaggtaaaagcaagtatttgataaaatgtgtaatgtagtctagtagctttatttagaaacagaaaacaacataaaatgttgacttgaaacattgcgtatgcatatttgccatgtagacagtgagatatggaggtaaactgctttcctagttttgttttgtttttttttttttttgttcaaaaggaattcactctccccttgtccactgttataaaatagcaaatggtgattagcatctgtgctgtaaccagagacactgctgttcccagctggaatctgtaggttgtttgcctgctagagattgtgacaagaagccagttaacatggagaggtgtacatgtcagtcttccccagaaaaggacatgaaacagtaggaccatccttccaacccatgacctacatctagaggcattctttttttctttttttttttttaaaccggaacagaaatatcatctaattctttctttacagagaagtggtataaggaagtacattctggccaggcacgatggctcatgctgtaatcccagcactttgggaggccgaggtgggtgcatcacctgaggtcaggagtttgagaccagcctggccaacatggtgaaacccctcgctactaaaaatacaaaaattagccgggcgtggtggcacacacctgtggtcccagctactcgggaggctgaagcaggagaatcgcttgaacccgggagacggaggttgcagtaagccgagttcactccattgtactctagcctgggtgacagagcgagattcgtctcaaaaaaaaaaaaagaaaagaaaagaaaaaggaagtatattctaagcccttgaattgatcttggctttcttcattatttttacaagtaaaaataaaaactttgatatttctgatgttctgggatattataggaatgataccacttgaattcagctttctaagaagtctggtatttcaatagtcttaatatgagtaagaaaattatgatcttggtactggatctaaggcagaggtatcgaagctgttttaatgttgcttgagataggacagaacttcagagtaaaataaatgtgtatagacaatctgttcaatctgttcaaattaagttgttgagctgtatatttgaatcttaagactactaattttgttgtatctgtcttttcccaaatatatgtgtacttcttttaataagccaaaacctatgcaacttttactaattcttccctaactacttcttttggaaaatgattaaatcttatgtctcacgttttgacatatctattgcatttaaaagataaggttgatcatgctgcttctcaaactgagatatccattcgagttatccaaggcccttggaaaacaatcgttgttgagtctgtatggggggcctgagtcccaggtggtgttgactctgctggtccatgatcctattttgagtagtagactctagtagcgtctcctgccttccatatggtgcagatttgcagcattgagacaggaagtaagaaactgaaaaggaaaaccattcttacctttggtgttattgttcttggtatattacagataccaacaagtatgaggcaatcctgagacatctacctactgagcttttctttccttaaattcacacctcaagaccaaggaaaccgaaaaactcttacagtctccctacatgtgttcacttgtgcccctatttcctgtcattaacatggtagtgtagccaactcctgctagcctgtaaaagctaattattacagtatagaggaattttgtaagccatagccattagtagcttgaaataccaatggtgggagtatttataccatggcagttggcatacactgtaactcaagagtgttctatttgtttgtttttctagacagtgccagtttcccagcatactcctgtctctcattcagtaagacaaagcacttccccttattttccaatacaggacatgatatcccgagttttgagcttcttgttaatagcctgttgcacgctaaccacgcagctcagtgaagagcaagtttattcccataacaaacaggaaacaaaggttgaggaaaaaccacagtggtacccttttgtgagtgttccctaacaccgtcacgggagtcctgattagtctaaatactttacaagtaaatctccttcaggtggcattttataaacaatctttcatcatggagagatgagaattgggtcaggtataattagaatgacagatgtattggctagcctgagagaattaacgcaattcagtaaaaaggctagccaactgcggatatcttctagttacttccgtgacaaattagcaccatcctcttgtactgaagcagtgctttcattagcatgcacagctgtttgcatcatctttgtttgtatttcaatccatatttggagcttaagtgagatttgtcataagacttcagttcaagcttgcttgcctaattttacatgcttctatttagtaaaatatgattactaggtcataaatatttgataatttattgccttattagagtaattttctaaacatgttttttgacgactgcttgccactacgtacaagcacttgaaaaatgttcttttctattaaaagtacatggttccagaattgggaaaatcacgttcttaaaaaataaccagttttctattgtgttggcttttaaaattttttcatcagtagaatcaaatgcaaatattgtctgcttatttttcaacagaatacaagtttgattttaataacttaggctcatgaaatgtcaccacgtagtaatacttagaattacagcaataacttaggctaaaaaaataaagttgttatgaactacattgagctcagacagatatatgtattatgtaaacatttatataaaatgtatataaattgtatattgtattatataattatatattgtaatatatagtatataatatataatttataaatataatacataaattatatttataaattataaagtttttatataatatattataatctaatatattataattatatttataaattataacgtttatttgaaatattgaagttttgtatacttagtggattttaatatttatgtcgttttcattttattccactgttacattaaggaagtaacttattacaaaaataaattatatttgtcttacatttgccctttttactctacttaaaattggagaaaaagagttcaagatagggaatttctgtcagaaaggagaaatagttgtattatgtgaagtttaagtagatattcttgtagagtatggagaaaataatgctataaaatgttgccttttaaaaaaaagttcagaaaatatagttcctagctctctctaaatgactagtttgattgtgactgctttcccaaaatttgtaaatttaaaatttatacatcacatggaatatagttttatttcacatacattccagtaggctgagaatacttgaaattcaatgattaaaaatgaaataataataaccagctttgctttattctaaaaccagctttattctttatttaccactttatttacagcgaattatttgctaatctgttaactttgcttcataagaaggcattataattatcgtacatttgaaagtggagattttctccccattctggttgccagtttcacaactgtgttcattctaaatttattaaaatatgtaaaatcagctttctaaccttggttgtccacaaagaatttcactccaatttgaaatatctattcagaactaattttttgtggttgagcattattttttgaacaatgaattgcataatcattttgtataatccagggagctctggcttagattctgtattattttgtgttatactgatttttagaaaatggaatgtagcgttgacacgagcacaaatctcagcaagggcctaccttacaaagataaaatgcgtggagttttattagaagcacatgattttgctttgtgtttcgtatacaccagcatttcagtgtagtaaatggcattttgtcttgaagagtgagcagagttgttacaaaagaaggcataatgcttccttgccagcagaattcagataagtgaatatcagaaaagctatttgtgatcttgaaagtgtaagaaattcaacatcaataaaggattgtggttccttagatagccatgtaagtctttgcctatgtttctaacatttctctttaggaaatattcttgcttttatttttctgaacaaattttctccattattatctctgatatttactttgaacagaaaggtggattggaaatcaaaaaataagtttgatttcaaaaatgatttcaaaatgttagttttctcagtattttacattttgagaagagaacaaagaaggcagatgaagagttttttgttttttcttaaatttgttttcttttcaactttatttttaattgacataataattacatatagtatacattttgaagatacggttttaattttcacaggaatcttatttgggtttagttatataaggtctaattttgcatcctctacatttagtggctgatttataaatttccactgagtgataatagtaattaatgtgaaacaggttttttgctgatgtttttagaagtttcagtagattaggaaggaagaaattatgctattgagaaaaatagaaaatgtaaacataagcatttgaactctctcttgagatatttttatcttccatgaaactagatgcggcttattgaagatgtgtttcaggtctcagtgctctaaaactcttctacttggtgagcatcttgattggttagatctcttactaatatcccattgctagagagggatgtaaaggaaattttctcaaactgagatttccaaatcttccaatctctgagtcaagtaaagagacaaggttagaactcttaggcaaactgaaaaatcaatattcctatattatcattatttttttctttttttgacaccaagtctcactctgtcgcccaggctggagtgcagtggcactattttggctcactgcaatgtacgcttcccaggttcaagtgattccagtgcttcggcctcgcgagtaggtgagattatatgtgcacgccaccatgcccagctaatttttgtatttttagtagagacagggtttcgccatgttgactacgttggtcttgaactcctgacctcaggtgatctgcccacctcggcctcccaaagtgctgggattacaggcttgagccaccatgcctggcttcctatattcatttttttatgtgggctttttaaaatcttttcaatagcaaagacttgacaccaacccaaatgtccatcaatgatagactggattaagaaaatgtggcacatatacaccatgaaatactatgcagccataaaaaaggatgagttcacgtcctttgtagggacatggatgaagctggaaaccatcattctcagcagactatcacaaggacagaaaaccaaactctgcatgttctcactcataggtggaaattgaacaatgagaacacttggacacagggcggggaacatcacacaccagggcctgttgggggggtggggggttgggggagggaaagcatcaggagaaatacctaatgtaaatgacgagtttatgggtgcagcaaacaacatggcacatgtatacctatgtaacaaacctgcacgttgtgcacatgtaccctagaacttaaagtataataaaaaagaaaaaaaattaaaaatcttttctttgagacaattgtagatttacatgcagttgaaagatttaatagaggccgggcgcggtgcctcacgcctgtaatcccagcactttgggaggccaaggcaggcggatcatgaggtcaggagatcgagaccatcctggctaacacagagaaaccccgtgtctactaaaaaatcgaaaaaaattagccggtgtgtggcgggtgcttgtaatcccagctactcaggaggctgaggcaggagaatggcgtgaacctgggaggcggagcttggcgaagcctgcagtgagtggagatagtgccactgcattccagcctgggcaacagagcgagactctgtctcaaaaaaaaaaaaaaaaaaaaaaaaagaattaatacaaagagaccctgcatagtcttcactcagtacagtatcacaactattaaattgacattaatcccttaacactgctttaacccaagtctatgctgctgcttctccgtgttcacttctgagaggcactggccatttcttgtgtgcttattcatactttgtgtaaaagcattagcatcatacatcgctggtgctgaccctaccctcagtcccagcttctcccagagtaggctaagattctcttttctactacatggaaaattgttctggaataataaagggttagcaaaaaagtcagagaaaacaaacagttgcattccctttccctgtagtagagtaggaatataattatgagtgatagaggatacgttccattcctttcccagtcctctccgaagtcagcttcactgctgtaggaagttgctgttctcattatttccctgagggaaagtgttgggaagtgagagttgaggtgatggggaatcagggttgctgacaattgggggaattataacgagaagctctttttcaccaggagaggggttgcatgttaagagaaggcctatctgctcatcctccatgtcagcagatgagaaccagttggacaaggtagctgtctcttagagattgagaagtaacaaaagcacactgtcagtcctgttcttccagcctcacgtacatcttctccatacattatgtatttgacattaaattaacatacactgttacacccattcctgaccgcattatctcaggtagccttttgtatttgagtattgtgggatctgttgctattgcccatgatgcccacttggactccaccaaggcagtgaaactcttgggggccgttcaaagtcaagagtgccaactgcccctaacaggattgggagaaccaaccaacactccccgaatagggactaactcactcttttcttggcctttatgaaaggaaggagttaaggctcttacagctggagactccagcaagagttgtaggctcaacgtgtagcttatactatttacttaattaacatgtaaatggattttgagttctgaatgcttgttctagttgattagggctcatttagcctggcttctacgaattagctataacaaatggtttcatagctgttatagaatgaggcagaagaaaataaaatgaatgtagcattatgtttaattagtggtataggaaaatgttaatataatgccatttagattctggactggggagattgtgcctttatcataagtaagaaattgttgctaggtttcacataagggcacatagttatcatgaatcatctccttcgatcatcattcttttatcccattacttgacttgcacccatccaaaattctaccacttttatttaatatatagactcataaagacaaggtttgtgcatctgcagagctgtgatgttcactgcgaggcccgtggcacacatctataagaagacacatatttggtactctaagatgatacttttttttaatgccaaaatgtgagggtcagcataaattgccaaggcatggtaaattttgttgccaatgctcccgtgactatggggtgaagggattctgtgcctcccttggtagcaatgaccacggtgcttgcaatcccagtgccaattcccggcacagtgcaggctctggaggcactgctgccttgcagtcttattggaattaacactgcctgagtaagagattaataatgggtattgttgtgtcttagaaacgttaaagagcatgtgacatgaatatgtacacattggtgaaggagatacagagaaggaaagcaaagtgaagctgtagatgtttgttacaactgaaggtaagctctgggagcagagaccactgctggctatagttgatgggatccacaatgtcaatacaaatggggactaactattgttttacttccctatacacgttgctctttcagtagatgcaataaagtactggtaaaaccagaggtggctaccatcacgatgatgtcaacaggagggacagtcagcactaagcccagaaggtgtcaaacactccgcaggagaaatgcgccatgcaacggacatgaagatgatctgacactcttcacgtggttttcagatggaaacgtggctacgaaagcatcaacctcattatcatccatcattaaggccatctcactcagtactgctgctttcaaagtccacgctcccaaagcaaattggatttctgtacacaatactcttacaggatgaaacccaaccaacttgttgacgtaagtatcccatctacttcgcaattttatttatcttccaaaattaaaggactggcaccctgatttattaaaagtgaattgggtctagggaccatatcccctctgagttactgacagagcagcttctggcctgtgaagctcaaagccatgcctagatgtgatggatccttgtctttgattggctaattagaatcaatctgttgaaggaccagtggcagtggctcacgcctgtaatcccagcacttagagaggctgaggcaggtgtatcacttgaggccaggagttcaagaccagcctggcctacctggtgaaaccccatctctactaaaaataccaaaaaattagccaggcgtgctggtacaagggtaatcccagctacttgggaggctgaggcgcaagaatcacttgaactcaggaggtagaggttgcagtgagccaagattgtgccactgggcggcagtgcaagaccctgtctcataaaaagaaaaaaagaatctgttgaatattcctaacttggaagtaatgggtgatgcgtggctttcatcttttagaaaggtgttttttcctctgtcagtaaaactaagaagacatctgtgtgtgctaaggcgtcagtctagcaagggcagggcacaaacccagagagagaattgggctgttgtttcacttccaataaaaatgggatatacatgggaggtagagttataattctactcaggattgactttatacatgcaattggatgggtcactgattgactttgaggcaatcagggatgagtgaaactgatcttggggagggtggcaagacatgttgacaggtaagatgtgttttaatgtataacagtgcgtggtgtgtattaatcagttggagttgattgactgcgttttattgtttggaacattcctgcccaagggtcaggtaaatatacaaaatatacaaataatacagatttttaatacataaatatcaattaagtagcataattaaataatacattgttatagttgtacttaatgtgtatgtaaatatatacaaataatacagatttttaatatataaatatcaattaaatagcagaattaataatacgttgttatagatgtacttcatgcatatgtaattaaattattaatgtataatatataaatacatattttaatatacaaataaaatggtcttaaatcagagttaatttaagaccgtttatattagtacatatcttaatatacaaatgaatggtcttaagttaatgtcattgagtataagtcacatttacagatatgacatgtgtttccagataaagtcaatggacagacagcttccaacactggaaaacattcaaaatatatgcaattaattttatatttgagttaaggcaatcatttattaaggaaagcaatcagatgtttgtgagggcttaaactttcgcttcataaccaaagcagtttccttacatgaaagttgacttcttaaaaagagaagtttattttttatgatggaggattgttgtgatgtgcagataccttccgtcatttcatactcatctaaaagcagccaaatgatcttttcacctttggccggaatccttttattgctaccgagtcctgcactcattaaaccagatgacagtgcatgctctgagaggtgtaaatctctagtcctggtggccagttaaattcctcagtaaatgtttggtagatgctgcctaataaaccagtccaggttgccactgggaggattaaaagaagtaaacgtgtatacatgaacagagagacagtgccttttcatgctaaatgtggttccccacatctcctctgattagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccagtaagtggacccttcttcgagcctggccacctttcgtctctctcgccactgactctgctttttgtaacagattgattttcctggttcttgggaatgggcctgttgctaccactaaccacatttctgtccacttctctaattgctcagagtctccgcagtatgttcaatcatgagcacacctctccgtctttccctgataaagcatggccatggatgtgttctcttcctagctgtagcacatatgtcttgcaatccagagggacttttgagtgcttctcttttaaacaaagctggagtggctgttttgtcttctgcagccagccgtttctgcaaatgcatacttatgtgtttgtccgtgtacatatgtgttcatgtttatgcatgtgcactacctgtgcagatactgtctaccactggaagcctggctgatgcagcttgagttgcattgataaggtgtatggctgtgatgaatacctttaagttggttcataacatgtcttttattgtgctgatgagggaatgtgattgaattgagctcatgcatctcaaatgccggagggtagaagtgcccgtcttgccccctccgagctcagcacacagacatttgccaggtcaaacttctgcttcttctcttgaaagtagaagaaaacttttgacttaacgtgagaaactttcaatgacataaggctcgaggaacataattttattttttttgtttttttaaagacagggtctcattctgtctcacaggctggagtgcaatggcgtgatcttggctcactgcaacctccgcctcccgggttcaagtgattctcctgcctcagcctccccagcagctaggatcacaggcacatgccactgcgcccggctaattttttgtatttttggtagagacaaggcttcaccatgttgaccaggctggtctcgaactcctgacctcaagtgatccacctgccttggccatccaaagtgctgggattacaggcgtgagccactgcaccccgccagaacataattttcaaagtctttctcttgaacattaagcttttcataattccgggggataaaaatatgaagtcatgatgacttaagtgcatgatgaaattttccccaaagaggtaattctcaggaggtccagtaccagatgtgagacttccatgtcgttaacagacttccaagtgaaattcccaattgtgcttatttatttatttatttatttatttgttgttgttgttcatgagtaggaagtatgaactactcaagaagtaattattcagctcagcttgataactaaaaatgaaactcaataactgtgtattacttttttgagagactcctgcctaattctccgctgtgttgcaagggtggcagccacatttcctgcctgagattcctagggattatataaattgttttgcagcaagatcttaggattaagcaaaacaacagaagggcctgcaccattaatacccccactgtaaaggagcccaggatctaagaggaatgtaatggaaaagctcctgttggtctcctgccatcgccagtgaatgagatgactgcttcagtcccatgtgtgaacaggtgtaggaactgataacctcaagggctttttttttttttttttctggctctgattggatgattgcagcttctggatcaaaggactcccttcttcagtttcaaacaaagcagtcaattaggtcaggtattgcgaacacaatggagaattctgttgcctgcagcctttggaacaaaggccttttgttacagtgattctcactggtttagccggtgggagaggtttcctggcagatttgcaacttttttctccctttccctgggtggattcattttctgaaatatcatgtatagtttttggtaataagagatttatttggttttaattattagcagtgatgaatttgcttcaagagattaaaattctctccacctcaccccagtactgccatcccttcacctaacactctctccctttgatcagtttgggctgggagtgaccagtgggcacctgtccaggtgtcctgggtggcacacagtttgctgagggttttgcaaaaggtcgatgctcccaacagagatgtattagatgaggctagctatgcctgattttagaggggaaaggtgacagaataatgtatgaagaaggcaaaggctgaagatcagttagatgggtggacaagaataagtagagagtataagcctcagcaaacagccagagccgagagccacatgctgtcctatgaatgagtgaaataaatggagtttttgtgaaaaaatgcatcgaagggaattggggatatttggcacaaaaggagcaagaaatcctcttctgttaagaaacctgttgttactcggatatactatcagagacaaaatatccagcattctcaaatgttaacttcttacgaaaatagatcttatgtttatatgttcattttggttttgttggagggaccaaacctaagtgagtgattttgtttgttaggttgtttttttgtcagtggactcgtgcatttcagccatcattcccatgtttctctttttgtttttagttatgttctcttattttttccatagtgtcccaaagtttactcaagactacccaggaacctcttgcccgagatcctgttaaacgtacgttgtcattcacctgagggaagggaagaggggaggaggatgctgcttggttcacataactccagcatcatcaccttctttgcatggttttgtgtttcttgaacacctgtcttagtaaaatgtttcttcccattaccttgcttgtaattacatctgattttgccagacagcttgagatgttgggctaagagcatcattgactaagtttcttctatttctgaccaatttcctttttatttagtctggttttattgaatattatatggacaacatcattgtattgtatttgccattactattttatttcctaaaagctatcagtgtaactgagagcaggcttagcctctcactgcttttgcagaactgaagaacaagggctaggtgcagtggaaggaaagtgactttacttagcaaagctagcaatggggaaatggtccaggctcctgcctcaaagcaaccatctcaaattttggattaaaaaacaaaggcttaaaaaggggagcttggaatgcagggcatgaaggaggggtgaggaggtgctgctatacaggacttgttccaaagacttgagttattatccagttcggtaagtgggctggcgcatcccgcacaatcgggttgtaaattaactgcagccttgaggtgatctcctgatgagggagaattctgaaggtgcctaagtcagtcagtggaacttctaagcaagcattaataattttaataataattataagcgtaattagataatggaagcgctgtgcagggagtgcctggtgggaagaaagagagcaaaggttaccatttcattactaaaactggaaggaaaagaaaggaagaaagatctttaaaatagagtactcggttacatcaagatgccatgatttaacaagagactataatacataagtaacttgtttcctcagaaaaagtacttaatattcatggtattgtagtacatggactaattcgtacacgcagatttcaatttatgtgtgttataagacccggtttcaactttagacatgaagggtcacttttagacattaggaacccctctttctcctctcccagaaggcataatggtaggttagaggtgctcaccttttgactaggaagtagagaaagagcctcctataaagaaatgcattgtccagccactgcaggccttacttacagtgtcttatgttttgtttttatttcagttgtaatatcagacgagtttagttttaaaatgtggttttagtactcatatttgaatttggattccatccagaaatagatttatccagtgttaggttagggatgatatatgccatttatgcttatgattgctggaagaagaggcacaatttcagttgtaacttagatattttataattagcatttgtgtacattatcctgaaaaaaaaaaaccctcatctcaaaactgatgaagccagttatcttttttaaagatgataaattatagatgaaggattaggtattttaaaaaagactgcatcctaaactataaagttttttaatggcctccataaaatattcagaataattcacattatgtataacttttgaaggatgtggtttgtactgctgcaacataaacgtttcttttgctgttgtgaaaaaagcatttttctctaaaaacactgtaagtacagcaagtgggttgacagactaggaaaaagctctagctattccactatagtgttgacataggtacgcatgttgagttgcattgtaccgagcattttcacagatcatttaatttttcttcagaacactttgtgggcattttcctcattccttttgtacatacgagaaaaaatgaggcttcaagagcttagtgacttgtcccaagccatcgaacatggcaggaccaggctttgaaacccaaaatgacgttttggccgcactccacaccaaatggctaagcattggttctgacattgttttgccggttagtgaaatcacatttgcgattcagaagcacattttaagaatacagaaaacagtgaaaagaggatgaagaatgttaacattttttcctaaatgccatgaatagagcacgcatcagtaaattaaatgtctttcccattcttttataaaacagtgattatttttctcttgtttcattttagcatttataagaattctatttttccatttttaataactgatggaaacaagcatcattgtctgataaatttattttcatgctgtacacgtaactaccactttcatcatccactgttccaaacagatactgaaagtctcttttgtaattttctccttcccagtaattttctccatctacttgcagtgtatttgggatcctgacacctttccattctaggaggatgaagtgctttgggcagctatattttcttgtctgcctacagagactattcaatcacagcaagtgaagggttttgcagtgctagtacaaacagacaaggaaaaaaacatataatttctagttatttatagcctgtgcagctatgtggtcttatttatagcaaactgcacatttgatttgaaaaagtcatggtcatctggaggtcaaattcagatggcagactcaattcagagaaaaggagctgaactagaaggtaagcaagcctgttagactgggcaacttggacttgactttgctccttcaacaatctagagccagttggatgatatttctgcacaaaggtggttggttttacaggttgctcagtctaagctgcttctagtcaaataacaaatgtgaaaggagatagaaaaagctcttcagttgaactgataaaagattacacttagggagaaaatgtatgatggtttgcattataaaaacaataatttgccataaatcaggcatttctaggtttcaaacttttgtcataaaaaccaattttgagcattgcaattttagggacatattttattgttgttattttaaataaaaatctaaaccacaaatataaggctgagagctttcattttattcgtgaacataggaattcaaatcattaaaaaaaaaccctttctcatttgggataaagaatcttacgagagctgagttaatttctgacattagttttgcacctgcccatttgggtttaggacagtgcaaaaggtttcacatttaatcttcaaaatatagtatttgtagacaataaaattatataaatatttgtggattcatttatattttaaatatgagagtgtgaccattttttcctggtgctgtctccatcagactgcatcccaaaaggcaaacagaccaactccttaatggcatttaatctctcaaagagcatcatgccaatttttagtcagcagattgcaccatgtgatctggccaaagtgtgtcccatctcaggctccttcatttgcgctgacatgtggtacaaacaactaaaccaaggtttctcaacctcagcactattaacattttgattcaaataattaatttttaggagtctgtcctaggcatatgggtgtttagcagtacccctgacccctacccactaaatgccattaacacaactccagctgtgataactaaaaatgtctctagacattgctaaatgtgcccagggggtcacattctggttgagaatcctgttctagagtagtagtttccccacatctgactcagagatagaactctcctggtgtaaacacgtgtactttccacaaatttgggggcatgttgaaatgttagaatgtcagcatcatccaattctcgacagatgtttgaaattgaatttgttagactattgtccaagctctcaatatttgcaaggtaaagtggaagttgctgttgctgtcctgactatagtattgaaggtctcgtagttttcctgctctaatgtaagctactgtggcagcagaagagaaaccagcaaatcccttagtttgaagtcattggttcacctatgcaccacccctttctcctctctgtttcatcttagtttcctagaatcccaggatatgctggaggacaaccatctgtagagatgtcctatagtcttgctacacaggttatttgtactttaggattatgtttgatggccttatagtttgtgcagaggtgtaggcctgaccccatgcaggattgagggagggaaaaagattgatttctaggcacatcagaatggtccagatgacatgaaacagactggtacacttacagagaatatgaaaagaattggaacttaacaagtcaggccccaagttccatccccaggttccaagtttgttgccgcagaagctcataattttcaaatcgtagaaaactcacaccacgcagcctttcaatggaatatgccaccttaaccccagggtcgttgtaaagacaggagccataagacagaagtctgaatgcctaggccatttaatctttggccaaatttgaactattgttcttaatcacactattcttgttcttttcactgtgcagtagcaaatgttagttcccattagcacattctgcaataccattggaactaaaactgccaaattagattaggcaacctgggattacactttgctccccaaccaacctagggagggagaacaattttaaagcattaaaagatgtgatggtgaattaaactacctgtcacgtcagtgccagtatattctatcagattaacaagtgtgaaaagctaaatctttggtatctggaaatgcttagataggaagaatggctttttcttcaaagccataataattagagatgaccctaaaattgaaaggaactggattgcttagcaactcagcattttttaaaaagaagggccatctactttgaatacaggttaaatatctcttctctaaaatgggaccagaagtgcttcagattttggatttcggaatattgatgtattcatgatgaaataacatgaaattcatttatgtttcatgtacaccttatacaaaagtctgaagataattttatacaatttttttttttttttgagatggagtctcgctctgttgcccaggctggagtgcagtggcgtgatctcggctcactgcaacctctgcctcctgggttcaagagatccccctgtctcagcctcccaagtagctggaatgaatgacaggcgcctgccaccacacacagctaatttttgtgttttttagtagagatagggtttcaccatgttggccaggctggtcttgaactcctgacctgaagtgatccaccagcttcggcctcccgaagtacttcgattacacgtgtgagccaccacgcttggcctatacagtatttttgataattttgttcatgaaacaaagtttctgtacgttgcactgtgagagaacaaaggtgttgctatcccagccaccatgatgtcatgtcagtgctcaaaaattatcagattttggagcatttcagattctcagattagagatgttctacctgtggtaaaagaatgttcattatgtctctgctctgttgtttagctatttgccaaaagagggtaagtggatcgtttaataaagtggtcagcaaatatttttcatacaaggccagattatagatattttaggctttgtagatcatgcagtttgtcataactacttaactatgcagtggtagcatgaaagcaacaatagacaataagtaaacaaatagacaaggctgtgttccgataatactacttttataaaagtagccagcagctgggtgcagtggctcacgcctgtaatcccagcactttgggaggccgaggcgggcagatcacctgaggtcaggaattcgagaccagcctggccaacatggcgaaaccccatctctgctaaaaacacaaaaactagctgggcgtggtgatgggcgcctgtaatcccacctactcaggaggctgaggcaggagaatcacttgaacccgggaggcagaggttgcagtgagctgagatcacaccatcgcactccagcctgggggacaagagcgagacttcatctcaagggggaaaaaaaaggtagccagcaagctgatccttggtttaatacagcaggtttttttatttttatttttattttttttccaatagctttggggatacaagtggttttagttacggggatgaagtgtatagtggtgatgtctgagagtttagtgtccccatttcactgagtagtgtacattgtacccaatacgtagttttttatccctctcctccttcccactcttcctcattctgagtctccaatgtccattatgccactctatgcctttgcataatcatagcttagctcccaatttataagggaatacatactgtatttccattcctgattacttcacttagaataatggcctccacctccatccaagttgctgcaaaagacttttattttgtggtttttctggcctactagtattccgtggtatatatacaccacattttctttatccctcattggttgatgggcacttaggttggttccgtatctttgcagttgtgaactgtgcagcagttatttttgaccccaggatgtgcgaggccaggtattttgagagacaccagcaaaggaatctgagtactggctatcaatacctgataattttattaatgagttgagacccagatatgccgtagaggtaaataaccatataaaggaaatatagaagtgtaggagaagattgctctggaaggctggggtaaaatgaaatcagctttatccttaagacctccatagaggttaactgtaccatctccttttactgctacagtttatatgaaactggacaatattgaagattttgacttttcgaactcatctattgagtgttgagcagctatgacaggttatttctttatgtgcctccattaacgaagtcaagaatagcttctgacaaattgacaaaaaacaataacctaaggttaacccattctggacgccagttattgctattgcttcacatatattttttagattcaaacttgaggttgcttttaagttttagtgtgatgtgctgaatagagaatagatgttaataaaaaaataatgccagcattcgttccttcacattcagttcatatatttgaaaaaattcccatgtcagtatgtgtccatggacataaaaaagaaatgtagcttgactagagtaaacacatcatagattatatttttattaccttatgtccatgtgatgcattgatagtgttctccaccaactatgattgcactgactataaaattgtaatacaagggcatattagggggagaatgcctttgttttttcttttgttgttattttgtttttaagagagtctggtattacactggaggatcacaaactgtattttcttgttataaatggctcctctatcttggacttaatggttaggataaatatttgaatgtagaatgcgttaggagttttaactatttttgaaattaggaaaatggagagacactttttttttttttagagacactttttttttaagttataataagcatggcactcggaaagctctgccatgtttcaagattaaagtttcacatgaacgctaacaagtttatatttcactcatatataaatgatcctccttgtctcctttcctaaattatagccatctcaaatgacaacattgctgggtcggtatcaaatagcattttcaatgataaggcaatgatttatataggtcttaatttgttttaatcttctgttaccaaggctgtagagataaattgcttttctgctgatgaaaggagactaacccaattttcttgttagaatctaatattaatatatcgactgaatgccattcataagccttggtttagggaaacattcagccttctggcctgttggagatggccacggtcattgctgtttgaaggtgggtcatttcttcttttgtgtcctgagtccattgtgactgtaaaattatgggtcaccccagaagcctgaggaaacaaatgcatatcaaaatgataggggcaatctttcacagtgcctttaattttgaccccacattcttatatgaatgtgaagtaccacattagtatgaatatgaaatggagtgctccgcaatgattacaaaatcagaactaaaatgagggaaatcagtgcatgtacagttcaacttaatggattatcctctaaggagtcttcagacaggctaggagagtgacgtctgcctcatcctgaagatattagcaggttttcccctgttttaaagtaacttcccttcaaatgctaacagttttctcagaggaaacacaggtatggaaaatgtttcgttggtgtcttggccccagtgatggaggagcattttgtgataggaacgttgggtgcagcatcgccatttatgaataaactgaaagctaacctgtgttacttcaccccagaaaacagagctctggcaggtattgagaatagatcacagtgtgaagtggcagtattgtgacaagcatgcgtgtatcagtattaaaattgaaatgagccctcacgcacatgtgaaagcagtttctcccgttttcatattcagttccctgaggtaatacagcattttggtgtgagagaaagaagggaatctgacccttctcgtgtgggaagggggctgagggcaacaacctcctgtgacagccaccctggtccaagccaccttcatctttgtctcctttttttttttttttttttttgagacagagtttcgctgttatcacccaggctggggtgcaatggcacaatcttggctcaccacaacttccgcctcccgggttcaagcaattctcctgcctcagcctcccgagtagctgggattacaggcgcctgccaccacgcccagctaattttagtatttttagtagagacagagtttctccatgttggtcaggctggtctcaaactcctgacctcaggtgatctgcccgcctcagactcccaaagtgctgggattacaggcgtgagccactgcacccagcctgtcttttcctcttaaaagatcttccttctacttttgtcccatagcagccagagtgatccttcaacaaaacgccccatcacatatactccccctcaaacagcccacgctggtccctcagaatccttactgtggctttaaggctgtacatgatgtcccccatttcttccctgcctctcccactacttcctctcttgcttctttagctctggccatgcgggtctcgtggaatttgccagccagcttcaactcttagggcttccgtgctagttttgtcatctgcctgccatgtccttccagcagccagctgggaagatctgaatgtcacctcctcacttaagactctgcctggtcgctcacttactgcggtctacctttcccctctcccctcagctctcccgatccttctgaactctgctctgtttttctttttcctctttttctccataatacaaatcacatattaacaatatgacttactggcttttaaaaaattcttgtggtaattgtttctttccctagaatataattccaccaggatcagggccttcatgtttttctcattggtttctcccatgtgcctcacatataataagggctgcaatgttggtgggctggaggaataaaagaatgaacaggcgcactaaaggcaggatagtttgtccatgtcagagctgctgtctcagctgctttgatacctctgacttcccagggtggcaaagaataaggatgtggcttatgtagaaggcgttagtcatccaaaatgccactgtatatttggaagcaaatattcaccttgaagcagtgatgtactaggaattagaactgcttttctgttacattttttcatttgttatgagtgtttagatacacatgatcactattaaaaggaagggaggtaatagcctatatatcctaccatgcccctcaaggctctgttaggagactaaaatacaaaggtcatttaaaaagttaaaagcttatgcatgtgatgttggtattgctgctaatgactaggcatctagaggtcaaaacctagagctaaggatattgaaccctgtgttttgtctcgttttaagtctaggtttttgtggaggggggaacatacctagccagcttgcttgcagaaggcatgagtgagcaggctgaaggagggacccaaggccagaacgtcctccatattctagctccatgggctctttcacttgatccctgctttatgacccaacttctgttgaaggatttgccagatagtgtttggggcaggaacagctgtttcatagaaaacaagccaacggacatcataacaaagcatatggttgttttgccaagttgtgctgctcattaacaaaacaaaacaacaaaaaccctttacagtgccccagcatgtggatggattggccctttactactctaaaaattagctttacagcctttttatggcttacccaatcttaacagactttatttgtgtgtgtttagggatgcaaatgggtatgttaaatgatagtgttttgcctctacgcaataaaatagggtaattttttcttcccatgtattaaacgagaacacagaacatgcctagcatggtgggctctgatgaaaagaggctgttatcatttggccaacatggaaatcctagcctttatttatttatttatttgagacggagtgccaacatggaaatcctagcctttatttatttgtttgtttgtttgtttgagacggagtttcgctcttgtagcccagactggggtgcactggcatgatctcggctcaccgcaacctccgcctcccaggttcaagcgattctcctgcctcagcctcccaagtagctgggattacaggcatgcgccaccacaccctgctaatttttttattttattttattttagtagagacggggtttctccatgttgatcaggctggtctcaaacttccgtccacctcggcctcccaaagtgctgggattacaggtgtgagccaccccatccggccgaaatcctagcctttatcagaggttccgtggaatctcagaggtctgtgagcaggataaactgctttatggtagtttaaaatgcaaaaccagaaataaaccctcctaaagccagtttgtttccactttctaagtggttacagtgacctgtgttttaagtaccttaaatatatcagtgacctgctgatatacagcttcctgtttcctctgtgtcctgtgacatatcctgacattccttgagccccactcccttcccaaaaggaagtaccttgatgggtgaggacatagggcacacgtgtctctggccttctgtggacataatcacaaaatggtagtcagtcctatgaatttaatctctttaggatataaggatattcacgaaatagaagttgagctctctggacatactccaccttaaaattgctctctccttccgggatcctcaccagctgcagagcagtagtaatgaactattaaattcatttaacaatatcagcatatgctatccttctctgcttgactagacagtaatctggcttgctggttggtatgaatataggataatttcactcacactaattgagatggggagtttgtatgaatggctgaaaaatttgagttaaagataaaaagagacacagcttcctcaactatgggaaataactgggactgttaacaaagatttacttgaagagaaattgtattgctcagggttctccagagaaacaggaccaataggatatatatgtataggaagagattgattctgcagaattgactcacacaacaattatggaggctaagtcccacaatctgttgtctgcaagttggagacccaggagagctggtggtatgatttagtccaaatctaaaggcctgagaaccagggaagacaatggtgtaaatttcagtccagggagaagagaagaccaatatcctgactcaggcagccaggaaataaaaagggtgaactgctccatcctctgcccttttgttttcagaccctcagcagatcgggtgattcctgcccatgttgaggagggtgatttactttactgagtgcaccaattccgatgctaatctcatccagaagcaccctcacaaagacactcagaaacaatgtttaatctgggcaccttgcggcccaatcaagttggcacagaaaattaattaccacagaaatatttctggattcaatggattaagttggtaatatagtattcatattcatttccacgtgcctaaaaagaaggtttttatcctttttaatatcctcagtcctattaaacatacctcacattcatgttccatggttattctcttattatgttgctaagtgcatttctacttaggccaatctgattatagtgattcagattatagtgtttatcttctttcctttatacagtagacagtgttcgcaaaccataggagctgaaaaaatttccttcttatatagtaagttttatgattccagttttccaaaggggtgcttatgttatggttataacagttacagccagatgactatcttcatcagcaggtataaccccaaatatttatacattttcatttgtttcaaacataaaatatggtttgcctaaacatgtcattctgccagattggactatgcaaaagaatgttactgttttattttctgcttttactttaacttgttctcatttgagctctaccagttaagatgagaagatgactaaagctagggaagcacattttaaatgctaaataccatgttcatttcaagttgggaacttgaaatgcaagtatttttaacttaatcattaagttaaagtcagtaagaacgtggtatttagcatttattcattttaacttaatgattaagttaaaaatacttgcatattggtttgcttgaagctatggagaaaaacatttaccttttttccaaaaaggtcttaatattcaaaattattgtttcaatcttacaaattaacaatttacttatttttgatactttcggccttttttatgtgggctacacctaacattttttgaaaaatacatactgcttttcctgtaggttgcactttgtgatcctgtgtgtaattcattttcagtggttctattttatttattttttgttagtttgtattttcttttgtttctttttctcaatttttaaaattttaaaacttgtccttttcttttttttctttattattgcactcttctaccaaaacattttcagtggctttgaataaaagtatcagaactttcccgtggttagcattagctctgagatttgaacaggatgtattaagcagaggaggagggaaggccactaacatgtatgagctccactgtgtgtcaccctctctgccggatgacttacatgtggtatggtatctcattttcccagctctcctatcatgtcctgcggcatgttatcaccacttgataaaaaaattgaaacatggagtgcttaagtttcctgccccaggtcacagctgatctgaaaaagaaatgggattcaaatccaaatgtgcttaattgcaaatccccacatgactctttccttccttccttccttcccccgttgctgccaatgtaagaaggaacaccactccctcatttgctacagagaagacaggggcagctgtgtaaaggcaccaggaatatgccctgcctccttccttccacttgaaagcaacatctggatagacactcaactgcttttattttttgaaaaatgattattaatcaggattgaagtggagggaaaaaatcaaaacgaatgtttctttttaaaatgaaagatactgtttaattatttaacagccttccggttttagttaatatctttgaagactttggattaagattccagacattttcttgaaggatgtagaaaacgtatttaacttcaagtttagtttcaaaatgacttagctatcaattcttctcattgaagtcctctttgagatattatatcccaaatgtaatacacaggtaataattcaataggaatacttggaacattggtgatatttgaataaccttttgaatgaaacaattgatttgatctcaaaagatagtgaaaacgtctcattttgttttttatgttaaatatgcttcctcttaagtcaaaattcttttttctttttgcttttaagctcataaatactctgaattctcactaatcttttggaaaattgcttgtataggtattataattcacttggctcatgagttacataatatagcagtatatgttgcaattcaaatattgcttagcagtattagttgaattcaaaatagaaatcactttctttttgacaactagcataatgaattcattgactattgtcaaatttatgaagacagaaaagtccttttgtgattcaaaaatgtgtttattgatgtttatatatcttttgtcgtgtttccatactgtctcatgaaggttgtcttcgtaattcagttatctaaatacatcatacatttaccttttccctggggtgccgttattctacaacaggctaattacagatatctgtggtttctgcaggagtcttagtgtatgatattgatgatatcatcatttactttcaaaattgcaaaatagtaatttaggaaagcatggggatttggaaaaacatgtcttcagcaccaactgtttttgctctttgcatgcttgtttcgtaaagaactctaatgctataattgcaaaatggaccattttaaagatttttccttcattctgtacttgggagtggtgaaagacatccttactgtgctgcacagtgtctcatggtgttctcttaaacagcattaacgtcttgtatgcgctgctttactaaattctctgttctgagaaataactgaaaatacggctttctattaaacgagtggattattctgttgttgttggcttttttttctcaaacctccttctcttctactttatagttcctacaacagcagccagtacccctgatgccgttgacaagtatctcgagacacctggggatgagaatgaacatgcccatttccagaaagccaaagagaggcttgaggccaagcaccgagagagaatgtcccaggtaagtctggctcttccatcattcagccctacgatattgggaaacctgagcttgcctctgcctcagtctccccacaggcctgctggctttatcaagatctttaaagatgtaaagttctaattttaaatgtttactgtgtgggcacagtttgtgggttttttgcatcctccgtgaacatgctgcctaggaaggatccaggtatagttggattttgtaagcacaaattaaatgatgtctgtaacattctctctggcgaagaaaggtacttggcattattattctgtgtatatgtagtacctgtaatcaacatgttgtaaattaaaatccatcaaaatgctctttagtaaagatgaggttttgtattaaatcccagactgttataattgtgaacctacgattgtttacagtctgtaaaatcatagagacttaaaactgagctgaaccatagtgattatttcatctagtctcctaagtgatacatagtggaatagaggttcgagagagtggcttcccattttagcacaaagcaaaatcagtgagccagagacaacatttagaatccaggcctttcttgctcctagtacagtactcctttcattatactttgctgaaaaaaaaaaaaaaaaaaaaaacctgttttggtttggtttgtttttttttagcaggtccttaattaagcaaattaaaaaataacttatgtctaaaacatggctaatccatatatactatcaactacaattgaagctatgtctgctaccaggcactggatgttgtattggagtttgataatatggaggattttagcccttgctcagattcctcattcattcattcattcaatcaacaaatatggacttattattttccagtcacttttctgatttgtagtctacaaacctaggagtgaatgtggggggaaaagaagtaggacataaaagtgaatgaataaagatctcaggttaaagtaagccattcatttttagtgttaatattctgtggcaaatatcagaagataggagaaatttttgtcttagcacagatgcttaatctcttgagagacattaaatgcaatttatgaattagactgctgcgtattttctaagatcacagtcagtctttagtagacaattaatctttcctgtaatttctgatctgggtccctgctgaccccaggaagattcctccctcccagggccaaatgaatggcccctgagcatgtctttcccatgcaaaccagtcaatcaaaagctccttactcccaaccaccttcttatactctgggccactattcacctgctctaatcaccccaggaccaggtaccagaccactagggacaacctcatgccccatcaccattatttgaactaatcagtcctaaacccgcctaccctgcttcgctcattctttccatgaaaaccacaataaaaggctcttgctcatgttttcccctcctgaccaaccctggtgcttcccatatgaccctgcatgtagtggcatgtaccggcttcttgggagctgagtaacaaactgtcttttccatgataatcatctcctgacctcttggcctcacctgaataataagaaaatctacccttgaaaacataaaactaatagtttatctagaaaggtacaacacaggtactgttttttgtttgtttgttttgttttttgtttgtttgttttgccttaagatgaataacaatacttggatctactttggtataataacataaagaggaaaaaagccttataatcacagtacttttggctctggaatataatgtgcagttctagtggctgcccacgatgaaaacctaacagctaattgaactagaaagctagccacagggaattattaagttttcacctgatgccaagaaaatcagatctttccttaggaaagacacaggcactgttcggaaagcattggaggactatgaatcatacttattgccactgaaatgtcctcatttgaatttacatcctatctcccttctttatgaagctttcagaggcttcctgtgccctcacaaatccacttctgggcacatgaagtcgtaaaaaaagaacaatgacaaaaataaacaccgaatgctcagcttgtcattttcagcgcctaccactgtaccctgactctggtcttcctaccacctcttccttctcaactgctacccttgctttctgtacagcgttaagctgctgttctgatcaggccactctgctcatcgtctgttcatctcatcttgggcttccctttgcccttggcattgagttcttctggaaatgccctcccactcttcttcacgctgacacatctcacccattccttacaccccacttcatactcagaatgagacaggcagtcattgctcttacctttcatttaattctaaatatttgctccccggttttgttagtgatttctggacacacctgcattatctttccaagctggatagaaaccttgagacctcaagtcatgaagcctgctacctgcattcatcgtggacccactgtagcattttttttaatgtgtgtttttttaatcctggattttttctcctagtatatcttgttcatttttgaccttattaagaaaacttatttcaaattctctgttagttttagatgtgacaggtcttctttaaaataataatttaacttttagcacatctgtgtaacttgtgacttagttcatgattttgtagattttgttcatcttttagtcatcagatatatctgatataacctaagagatgttgtcccagagcagagaatgttgtctactagattccagtggtcataggaatgtgagacacctgaaatcaatatgtatcttcatagtctgtggtgctgaaatctagggacttttcaactttaaacttgcctgcctcttaaatctctacttagtagtagtcattttctctgcatgaaacagccaagtctttgaagattgataaatccaaatgaaattgatacctttgtgcctgcagtggaaaattgctttataagaatccatagagccactgcaagggccaggtcctgagaattttaaattttaaaaattgtgcggttttttaaattaagttttattgtagtttttgttatatgttttcttacaaatacagaggattttggaattttttcatattttggtttgttgttgccattgtggagtataactctaaacctgtagatttttgttgctaaacctgtagatttttgttgctatatgtcgtgactagcatctagtcaaatagaaatctacagctgaaccatgtgtgagcacttttattacttataaacttactacttttagtatatgcttaaaatgaagtccttcaaattgaaagctgacccaactcataggtttttatttcttatcttttttggagatggagtcttgctctgtcacccagctggagtgcagtggcacaatctagactcactgcaacctccaactccctggttcaagcgattctcctgcctcagcctccccagtaactgggattacaggcacgtgccaccacgccgagctaatttttgtatttttagtagagacagggtttcaccatgttggccaggatggtctcgatctcctgaccttgtgatccacccgcctgggcctcccaaagtgctgggattataggcatgagccactgtgcctggcctaggtttttatttttaagataagcagcaacattttaagagcgcccctttctcctgtcactaaatataaataaacttagctcacctaatgaattttttattttataataaaaaattgggtaccacagggtacaaaaacatgatctttagagccacagagatcttggtttatattcttccctctcccttttttacctgcatagcctcgagctggtaccttaacctggttcgtaaaatgggatggccctggctaatctcacaagctgttgtgaggaatcaattaaattagatagattttgtaaagctcttaatacagggtcagcagtaatgctacttgttatcagtcccacagtgaaattcactgttcacagggagatggtcagcctacatatagtgtgtattcatttgcagtagagtcttagtgggatggtgcaggtgatttgtttatttgtgggggtataggatattgttaaattgttctgaaaatcaaaatgtgagttaatttctatagtaatgcttatttgcaactcattagctaaataaatctctttaattatttcatggattcccccagcttaaagcttaaagtccagtttcttaaagtgtatgaggtaaaatggaaaaaatgtaaatgtaatatgggaaatcagttctcacattgcagtagaacattgaacttctatctcaaactaattttgagattccttgcttgaagaattgttggtagagtaccaccactgtgaacattcccaaagcaggaaattgaaatatttatattgcaaggcttatctcatctataaagcaaagatggtcttccatttcccccttgcccacaggttccctttattaacgtggtagaagcaccaaagcagatgggacaatatgtttcttttcttccatgttctattcttgttcattgcagcagggcatttgtcccaattttgctgtgaaccttgagattatttttgatatgcttttcaagtgtaatgaaatatttattccatcttgtttcctccatggttttacaaaaacaacaagaagaagctaattgcctatcatatacaaagaatgaatgccaatggacaataaaatctgtaattaggaacccacatggtgatttgatatagtattgtctcatagaagtcttctatctatagttatttattccaagcctctttttctttcccatcttgaagtatgttagaagagtctattccttaaggctaagtacctacaaaaaacaggctcttataatacagtaatcaagttttctcatgctttgggagtacacagagtatgcaagtacacatctggcaccaaaccaatattttgtttccttgatctttcttcattttacctagttcagagttgcacttcaaacaaattctacttgtcatacacttgaaaaggcagctgtaggtggtaggaagttaatattttctttgaacaacatacagaggtgcagacttttactttttgtatcaggttaatgagtgaacttctctcagggaaatccaggtgaacatcaaagttgagagcaggaagcattgatttcctacaaaacacattcacctgatatcttagtcagtttgtgttgctataatagaataccccagcctggataatctgtaaagaaaaagaagtttaaaattcggctctcagttttgcaggtggggaagtccaagaacatggtgctggcatctgttgaggacttccttgctgtatcatatatggtggaaggaagaagggcaagaggagggcaaaactgagttttataacaacccacttctcacaataacaaacccactccccccataatgatactaatttactcatgaggacagagactgaatcacctcttacaggacttacccctcaacattgttgcactggggattaagtttccaacacatgctttttaggggttacattcaaaccatagcagttggttactaaattagtgctacttgtaaaaaaatacatgtttgctgcattcagaatattacatgttttcttggagtttttggaaggacttagcgtaatccttgctgttcatgacttaggttggcaagctcaaagggccttaattccccatcagttctttaaagggggataggtcttaggatattagacagcagaagtcttctttttcttcccccattatgtactgctttttggcattgtattcatgacacacacacacataaaatgtcatattttctttaaagtcaaaatatacagtttaaaatggtttcagttgctttcataagagagtaaaacttaggtgttcagtttaatattggaagaccttgttcagttaatgtgtgccttccatgaatagttggtatttgttcacatcaaatcaaatgactgtcagttctcggggtaaaaaaagaccaaccttgactcaaaaagctatattcctgcaataaatatgaacatttatgtttaatatactgctgtgattaattatttatatcctagtgggaggtcaaatattcttcagataggaaggggtatgtaataaattttttaaatatctaaaatgacaaaaatagaggaaaaacataatcatccatcctattaagtctgtattcaaaggatgaactgatgattttaaattcaaatgtttccttaatttataggtcatgagagaatgggaagaggcagaacgtcaagcaaagaacttgcctaaagctgataagaaggcagttatccaggtaaaacctgaacccatttcctaccaaacatcacatgccagcagtctttttaatgagtgtctgcaggtttacctttatctgccagcacctcacgtgtgattgttcatagtcccatgaacttcagctgttcttaatcattgtctagtaattgagttgtttaaatcctgccttatatttgctcccaattatttcaagttataattcattgccataaaactcacattgtaacttgcaatctactttttattaataatacatcttttcagtcaccacaatcttctgctgcttctccctccagcaaaaattatgagaatttgaatttttgatggaaaccagtctcaaaattaccttaagtaaaccaacagtttattatggtcaattacttgaggtactgttagtaagagagggatttttttttaatatgtaatataagagtttcaagagtgcatatatttatccaaagttattagtcaaatcagccttcagagagcctatgaaatactgattttcaatgagaaagtaggtttgatgagggttggagagtgcaagaaagtgaagtaagctataaaacgtagaattagtcaatgttggaatgactatgcagtctttaggatactttttagcactagaagaaaatggaataggatgtcttttttagaagacttgaaattgctgcttcatcctacttattcagtccccatggacatatgtgtttatgatggcagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcgggtaggtctcgctgcagccgagttcacacttcaggtcacagcacagacagtaagggtggggcactgggaactggaagccatacaaaaagaatgaggagaaatgccttgagcactgttattcagaggttcaacccctgtccattccatcttgaaggtcaaagggtcacagggcagctacctccacaaggtcatctctacacagcaggtactcacacttccccacagagcagccagacaggactcccagggactcacattgcaagccctgacttgatttaatattcctgaggtggtgacaattgatggcatagttcaagttcgaacacaaaagctggaaggatgtcttccagtcccttcaggctgctgtaacagaataccatagactgggtaccttatcagcaacagaagtttatttctcacagttgcagaggctgagaagtccaagatcaagacatcagcatacttggttgtctggtgaaagtttgcttcctggttcatagaacttaaattttgtttatcttttatttattttatttttgtttttgtttgtattttgctgtatcctcatatggtggaatgggcaagagagctctctgggccctcttttataagggtactaatgtcaatcttgaaggctctgccctcatgcctaattgtctcccaaagctttcaccctcaaatagcatcacactgtggcttaggtttcaacatatgaattgggggtgggagagacacaaacattcagaccatggcaaaggattttagaagtaaccttgcccagccccctcatcatgtaagtataataatataactaaggtcaaggaaagttgagatttggctgagttgtaagaatttttagtggaccagaaagaattagaactcaaatttccatctcagagcagtggccatattctctgtggggaagcatttgtcagtcatgacattagcaatgatgagacttattcatggttttcagtgatcattgccccaaaaaggcatgttgagtggtacaggcatagatgtgttcaccggcattctgtgggagagcttacttggaatggaaggaactttctgagcatgagtgtgtaacttatgtccacttgttctttttttttttttttttttttaaatgagatggagtctcactctgtcacccaggctggagtgcagtggtgcaatctcagctcactgcaacttctgtctcctgggtttaagcaattctcatgcctcagcctcccaagtacctaggattacaggtgagccacgacacccagctaatttttgtattttagtagagacagggtttcaacatgttggccaggctgatctcgaactcctgacctcaggtgatccacccacctcggcctcccacagtgctgggattacaggcgtgaaccaccatgcccagccatttgttctttctgtttgtcattgagcaagggatattgaaagctctgcctgtgattgtggaccgggccatttttctaaacagtgctatcagtttttattttgtatagtttgaggctgtgtttttaggcgtgtaaatgttccaggctgttaggccctccttgagcctcttcttatgaaatgatctttttatcctttgtaatgttttgctccaaaatctactttaatattaataaagctattccagctttcttttgattaatattaatgtggtgcatttttccatccttatactcttattgaccttgtttgtctttatatttgaagtgtattttttgtaggcagcatgtaattgggtcttgcgttttcctttggattgggaagaccatgtacactgaatgaggttatggatatggttgtcactgaatctgtcatcttgatatttgtttgctatttgtccagtctgttccttgttcttcttttttcctcttttttttttggattaattagaaattttttatgattctattttatctcttttgttggtttgtaagttacaactagtttgttattttactggtttatctcctttgtgggtttataagttataactagtttgttattttactggttgctttaggcttatagaaaacattctaaacttgtcacgatgatgttatgtcagttcaaatgatattataccactccacagacagtactttacaaaagtacttccatttttcatctctctctctctctgtaagcggttgttgtcatactttttccttactacattgttgatgtttttctttaaacagttattttttaatgagttgtaaatttaaaaaaaaaacaacatgtttacccatgaagtcatcattttgggtgctgtaaactctttgcgcagatccagctggtatcattttccttctgcctgagagatttcctttaaaatgtcttagagactgggcttggtggcttgtacctgcaatcccagcaactcaggaggttgagataggagatcacttgaacccaggagttcgagactgcagtgagctgtgattgcatgactgtactcccatctgggtgacaaagggcatcctgtctctttaaaaaaaaaagtttgagtgcacataagttggtgatgaaatctttgtttggctttgaccaaaaatgtcccttttatacctttggttttgaaaaatatttttgctgggtaaaaaatactggattgaggtttctctttcactgcctgaaagatgatactctgctgtcttatttgtgttgatatctttatctttatttctctaaaaataacatgtcttttttttctgtttttacagttttctctatatcactggtttttaacaatttaattttgatatgccatggtatcattcagtctgcccttaattccatctggccgtttttcatcttatcacctcacatactgtagttgttgtatatagaaatttaacctgggctacccccaacacccataatatcgtctgtatcttaacttttgaacctatggaatacagttattatatgaaaactgtttaaatatccatgtttgctaattctaacatctctgtcaactttgaatcaattttgattgcttattattacatgtggcatttccctgtatcattttatgcctcataattttttattaaatgtcggacgctgaatttaaacctgttgaatgctaggtgtttttgtaatattcttgagcttttttctgtcacatagttgagttacttggaaacagtttgatccttttgagctttgcatttttagatttgttaggtgggatagaagcagttatcagcctaggactagttattccttactagtgaggcaagacccttctcagtcatctaccatgaatgttaagttttctcacctgactcgtgggaagaggcattattcctgtccctgtgtgaacaccaagcactgttaatgctaattctttttggtgattttccccctggcttcagtttctttcacatgcatgtacccatcattactcagctatattctcagggatcactctgcacatctctggattctcttttggagcaactctctcctcaccaataccctgtcctgtgaattacaactaccttgatctctccacacacttcatgtcctccactccacgagtcctctaggctctgtgtgggtttccccacctgaaaccatgacctggaaattcagggccatgagtttggcagctgtaagtcctcatttgtttccccttttcaggaatcagtgtccttcacctgatgtctaatgtcttgcagaaatgttgtttatgccaggtgtggtggctcacgcctgtaatcccagcactttgggaggccaaggcgggcggatcacgaggtcaagagatcaagaccatcctggccaacatggtgaaccccgtctctactaaaaatacaaaaaattagccaggtgtggtggcgcacacctgtagtcccagctactcaggaggctgaggcaggagaatcacctgaacctgggaggcagaggttgcagtgagccgagattgtgccactgcactccagtgtggtgacagagcaagactccatctcaaaaaaaaaaaagaaacgctgtttgtatatgaatatattgtccagttattggtggttcaggcaggagggtaaatccagttcttctcactcaatcttggctggaattgtttgtgcattttatgcaacttaaccagaaagtcacccatttgcaccgattttcctcaccattttgatttttctttctatggataaagttacagtgcacagaaagccatgaggagccgttaaagagctcgtggcattcttccaagacgtaagatactatttaccctgttctaccgatgatatcactgcaacctcgggagatggagcggctggttcagggtgacactgctgggaagggagagggtagagccgcgagtcactgtggtgccttctcactagaacgagcttcctctctaaacaaagagcttttgaaaaagaatattcatattagcgttttcattttctatttgccaaaattctctatcttagcaaggcacttgtcaggaaatttttttttatatctctgcagaacaaaagttccttggttcctctcttttttggagaacacgctttcaggagaggagccgtgtgatcatttgccagagaaggataggctggtgggtgctgagaccagaatctttatagcagaaacttgtgaagcgttccagctgaaagtccaatctctaaagtaagatctttgctaaatggacattgatgggactaaaaataagctgagggctgaatggaggctttaacaaatgtacatttaaactctcagtttagtctcatcagccatcacaggaaaggcagtggcaagaatgtttgccgaagtgtcatcttttatcaaaggggataattttagagtggttgtaatcgtgcttattgtgggcctccgcattgcttcatatggaattaggcaacatgttcatttgaaaaaaaaattaaaaactggattaggaagaaatattttactgtcaaaaatcagatcactgttgttgattgatctaaggattcctgttaaattcttaactcggatttatttatttatttatttattttctctctctctctctctttctctgtttctctttctctcctgccttccttccttccctccctccctccttccttcttcccttcctttcctttcctttccttttctcttcccctcccctcttctcttctctaatcttctcttttcttttctcttttcttttcttttcttttgttttcttttcttttcttttcagggtcttgctctttcacccaggctggcatgcagtggtccagtcgtagctcactgcagccccaacctcctgggctcaagcaatccttttgcctcagcctcatgagagttgggtctacagaagctccaccatgtgtggctaatttttaaaaaatttttgtaaagacagggtctcgctgtgttacccaggctagtcttgaacttctggcctcaagcgatcctttcacctcggctttttaaagtgctgatattacaggcatgagccaccatgactcacctttaaatctttcctttgtaataaaatgtggctgggtggtcacaggcctggagtctagggggcattacttgccatttttgttaagttaccatctttaggtgttcatttaagttttaatgttgtatggcttgacctggcacttaggtctgcactttggcaggcacaagaaaattgtaaataccttcttttttttttttttttttttttttttgagacagagtcttgctttgtcgcccaggctggagtgcagtggcgtgatcttggctcactgcaagctccgccttctgggttcacaccattctccagcctcagcctctcaagtagctgggactacaggcacccgccaccatgcctggctaatgtttttttttttttgtatttttagtagagacgggctttcaccgtgttagccaggttggtctggatctcctgacctcgtgatccgccagcctccgcctcccaaagtgctgagattgcaggcgtaattaaatgccttcttgaggacttttataccttcctcttgaagacataaatccagagcttactggtggttgtctgaaaaagatcacatcttgtaataggagtcatacataaatatttctgagatctccctcatacttaaccaccgtgcctggaaaatgcagacaactgtattcagtaaatggtatttgaatataagtaaatcaaaagtgtcgtttccatagattctgtagatgttaccacttagaataattacctgtctaacccttaacaaattctaccgtatattctcagactttgcaaacacgggattccaaaacaatgtggattgttttcagtttctttgaagctcagatttaaatcatacttttactcagtttttaaacctaaacgtggaagtgttatgagatgttgtttttgtgcagctagaattccttgttctactaatgaatttcatttcctgagtccctgtgtctgagtatactgtatgtttcaaatttctgatttagttatgtacctataggcaaatacactgaatggtcaacctaaaaatgcattgtttgactcatatttagagtcatacatatataggcacatgtatttaaaataaatactttaggctagatgtggtggctcacagctgtaatcccagcattttgggaggctgaggtgggaggatcccttgagcccaggaatttgagaccaccctaggcaacatagaaaggccctatctctacaaaaaaaaaaaaaaaaaaagatttttttaattagccaggcatgatggtgcgcgcctgtagtcgcagctactctggaggctgaggtgtgagtgtgaaccaggaagtcaaggctgcagtgagctgtgatggtgccactgcactccagcctggatgacaaagcaagacgctgttccaaaattaaaagttaaaaaatgagataaatacttgaaatattacaattattaaaatagttgttttccccatgggaaaaaaaagttgagcagagctgtgtctttgcactcagaaaaaactcttactctgttgaaggctagtgactcttctaaacaccaaccccttcacctttttactttccttagactttctttggaaaatcaaggagttagcactttttccccctggaagaattaaatgcctctccagaactgcagcacaggaggttaacttctaccacctttggaggcctgcaagctaccagattaaatacttaaagctaagcattcatcatttcttaacctgccctctacagtcatgcacttcctgctttttactttgtcagggctcgtactcaaactattcatagcttcatgaaacaggaactaaatgctgtctgagcctttccccttgtaactctcagcgttctgcttatccttcatagacagaagatgatgatatgtcctgactgttgaagagaaattagacttaatgtgatcctgtacagttaaaccaaaggccagatagggattttttatttcaatcaactttctattgtaggcctaggtttggaaaaccttagcaccccctaatttgtaagacttgaactgttttaaagataagggatacacagccaaagctttctttgtaattctgagatttttggtgtttttacagatacttagtctaaatctttcatcagacagttatcatgattattattctgacttcaacaactgcattttctaaagggaagtgagaattaggaaacattttcctgtacatctgtttatctatcccagttgtgttgtttggattttttcagtgtcttaacgattgaaaccatcttatccatactgaaattcatttcatgcttctatctgctgcttcacattcaggctatcagtttaattgagcaaaaaaaaaatgttggcagtttctatagcatcggcatccttctttggtttctcatttttgaatggttcagccacatgttaaattttgctgagatctctggaaacttcttcacataattctttgatagaacatgaattctgtgagactcttcattaaagagcacggtttcatcactaagttgagattttgctcaaggctatttggtaaattccagcagaagagcaataaatgttcaacaagaaacagccgtcattggagataacaagggtagacattaatgggaggtttgtcacaggtaaacaggtgatggcatgagaaggtttaaaccacctcataaataaatttttcgccactatatgatgtggattacaactaaaccaaactttaaccaatgtgggtgttggtgtacctttcttttctaaagcaaaaaagaaattgtggtactaaacttgagcttttatatgataccgctaattgcccttcacccaacaaaccaattatttctacccatcacttttcgttgcaaaagaaattatcttataaaatatatgtatatagcatatatctccaaagcaaatatgggacatctgtacctcaatgtaactgatagtaagttaaaaattatttgctgagttcttggtatgtggacagatgaatattataatgtttaaggcattccaccagcaggttttgaatttaaccaaaattttatgaagctaatcagaatggaattaaaagttgaaagactgccaggccaaaaagatcatctttcagaaaagtaaataattgctctatacttacatatttattatttatttgttcaccatccgtttatttttagttgagttctgtatacattggtgagcaaagggggactcattagcttgggcactcctaagggcaaacccaataacagatagtgaatgggaaggaaaatgagagaattagcatttagtgaacaaaatcttataaactatgctgaatggtatacatgctctttcagtaaagccactcagtactattacttccctaccttctgaatgtttaccaagttcgataaaatcttgctacatacaaatgggaagtatgtaacaagagatactagtggtttctgggtttaattaagttacatagaacattaagaagtatgttgggaaggcattagcttaaaagctgtcttttgccactgaggccatattcatcacccagattccctccacagtggagcactgctcagtcactgccttcagaatgcaaataggtcttcacaggatgctattaaaacccatagcaccgtatggttttacgctctgctttcttccagcaaacctacgtattttaatgttaatatatatgttcattttatctttgagaatgaaaatgtattatgaaaatataaatatttagtttttgtagacaaatagcacatttaaagatccagtagttattcacctgcctggtatgactgatagagaacagatgggtagcaaagaatcctcaccctggaaacatgtacgcatacacacgcaaacacaatctcgcataagctttaaggggttcattcattttgtgaaggttgaccatggataccagtttaaagatgccaggtgatgtttggcttttaagtatcatcaccttgagtttccttatataaatgttttaatatcaacaaaaaactcagaatttctaataggtcataacactgcctaggtatgggaagaggagagggtaaaattaaaagaaatgcaaatgtacttttctttgaataggctaaccagttagaggatgtatgaagcaggaccaccatagtccaagtatgaatatgcaaaattaaagcagattgctgtaaatctctcttaaaaagtcaagaatatgtcacagttggcatttttcggatgatcagaccgtttgacaaatctctgcaagggtccgagttagtcgaacatatcttttgcttatgatttcagcaacactgtgcgctttgccttctgggttgtattactaattctgttcatcattaagtgtacacatgtttcactgtttgactaaacaaagcttgaagacatacaagtccaaacattttctgtcctttctgtattcccaggtcagaagatcttagctctttatccattctgtgctagagtttgtctgaaagcatgaatcatggggttgtgttgggtgctaaatttcagttctatatgctgccttcactaatttatgactgtggtctgaacaaccaaaataaattatttctgagaactagcaaaataccctggaaaaaatttatgaaacctaccagatttgtaaaaaggtcagccaggcgtggtggctcatgcctgtaatcccagcactttgggaggccaaggcgggtggatcacctgaggtcgggagttcaagaccagccggaccaacatggagaaaccctgtctctactaaaaatacaaaattagctgggcatggtggcgcatgcctgtaatcccagctactcgggaggctgaggcaggagaatcgcttgaacccgggttgcagtgaggggagatcaccccattgtactccagcctgtataacaagagtgaaactccgtctcagaaaaaaaaaaaaaaaaaagaaaagaaaaaaggtcactaagtgatgagagaattaaaaccactcctattttctaccttttataacataaactcttgtctttacttcccaggtttgccatctttataacctgtaactaggagcaagtgatttaataaagggattcctcctattccgtctgcaaattggagttaatttttctcctctttctgcaatttctgttgttttagcttttgttagccataattagaaaatcaaaaggattcattgaaagttgagacaaaattgtgaagtaggaagatgtcttcctccgtaagaggagaaagattgttagaggaagtacatggaggctttggtgacacatccctatgaggaagtgatctgcttccacgggaaggagagaacagatgatagatgactcctagatgctcctgcgtcatgttcagtacaaaatcagagccgcatcagcaaaccggataccctgggaaggaacattgggataactccttggccataaacaaaagttttgctctggccttatgactaagtggattaatagagattagttgttcaatatccttccatctaatggatccatattgttaaaacttaagaaacaccaaagggggagagtgtgagaatctactgccattttctccattgcttcattcaagacttctgtgtccaccttggcatgggattatcatttgcccttttatactcaccttaaactacaaagatcatttgtttgcctttctgaagaaaggaaggtggataaattttgcaggagactttaaaaattcagctcaactcctggcattaaagacaataaaggtggtgattcagaggtccccatggcagtttccccaaatattacgtttaactaagatcatctcaagcacagagagacttaactctctaaagtgtgatttttaaacctgcacacggaaattttctacattttatatgctcaagatttcttgaaatccagggtgtaaaactgtgaaaataatagttctgaacctatttatgaaagcaggagagaattgctttgttcagttttggaaaccaccagggtgcaatttaatgctccaggagagggttttctgtgacctggaaaggttggggtgattcccgtacagtgagtcatttacttccttcttggcaggattatgcccagggcttttaaacgctcttgacacttcactgccttccataagaaacgggtttagggcacaagtgtctattatgataagatgccagatttctagatcaagcataaagctgtggagtgggacaataggacttaataattttaatacacttcccacgtgaactgtcaagtaataagctacatattcagacgtgactggcattgagtcatcctgccattagtcaaatgtatgagacttttcccaagtcccagcaacaccaaatttgagaaatcgtataaaagaaaaatgaaaaagtaaatacgtctaagaaatggggaaatcaataaaaaatgtactagcaccaggggcggtggctcacgcctgtaatcccagcactttgggaggccgaggcaggtggatcacctgaggtcaggagtttgagaccagcctatccaacatagtgaaacccttctctactaaaaatacaaaaattaggtcggtgtgagggtgcacgcctgtaatcccagctgaactcaagaggctggggcaagagaattgcttgaacccaggaggtggaggttgcagtgagttgaggtggaggtgcaccactgcactccagcctgggcgacagagtgagactccatctccaaaaccgtgttagcttttatcaggaaaggccattaaattttcttcaatattacctacatcaatttgacatcaaactataagaacacaatatgtgagatgttttgaaattaatgaaatgaatcttctctttgagacgagtttgtgcacgctatgaagtccattaaatattttctgggaagagttcccagttaaaactactttctaaagtacccaagcacactatatagatacatcaagtaggtttattaatgtcagtatttcggtgaaaattgggaaggccactattctgagtagagaggtgtttgttttctacttgaagactatgaaggatattctgcactactgggcctatttctgaaattgtatctagctgcaagttactcattaatttaattaacaaatatttattagcaaccaaattgtggcaggaactgtgcctagaggtagaaatacattacgctcttgtttccagagaggaaaatgagtaatgttctaaaatgtaaatcttatgtttctctttcctcttgctgtaaatgatgtctaagccatttcggtggtgcctgttcttaatcctttaataaattattttgaatgattttttaaaaggcagtgtgattgtgaggtgagtttagtacaaattaaaaaatgtcacatcagttgcaaacagacagcacataattatgagctaaacactgtttcagaaacagtagtgacaataaataggattcggtataattagtcctcctgggacagatctcttcctctgttctttgccatatattttaataatgagattagccatctaatgccaatataaccagactgtagtattaatacacagaaaaattattgctagtaacagaagatttaaagacatgagggtgatccggattatatgcctgcaggttaaaaaaaaaaaatgtttcttaaactctctttatcaatatccttttcgtgagccactgaattcttttgtttttctgatcatatgaataacccgtcttaagtatgaatattgtgacaatagtaatagcaattactttaagagaatatgtttctatagagataacagaggggtttttccctaccattcctgtatgaaggtttctgtgtttttaattaaaattttaagaaattcatctttgtcttagcataataaaataaacctcactagacaatcaggacaccttgttattcttagatacattattatagctcaagaaataacaaatgttaagcctggaggatttgtattttggccaaaagttgtgagggcaattatttatttatttgagtgaaatatgcaggaatcatatatcaaaaggagggaggtcttttttttaacctcaacaaaaatataaaagtatagggaactgttatctatgtgaacccagcaaatctgagacaggtctcagttaatttagaaagtttattttgccctggttgaggatgcacctgtgacacggcctcaggaagtcctgatgacatgtgcccaaagtggttggggcacagcttagtgttatgcatttttagggagacatgaggcatcagtcaatacatataagaagtacattggtttggtctggaaaggcgagacagcttgaaacaaaaagcaggaagactcgaagtggggaggggccttctaggtcagagataggtgagatgaacagttgcattcttttgagtttctgattagcctctccagggtagacaatcagggtgattggctctaagcagttcccagctagacttttccctttagcttagtgatttgggggccccaagaattattttctttttacacctgtgttttttctttcttaactaaaaatagtatttgtttctgagggatagagataactaccattttatttggtatgatttatttgcctaccgtccaacaaagaaggtaatctgattttgttttcatttccaagcatgattcctagagatgtgtttaggtggccaaatcaatgaagcagaaccctgcaaattcatttattgggagatatcctaaggtcttcctgaattatctgatgttcccaggctttgcttatccaataggcattaggaagaaaaggcttgtgtttatgtagatgcacacaaaatgcgcatctcagcttacataaacagtgaaggggtttcccaggtctcattatccctggaggagctgaggcctggggagtcctcagagcctcctttgctactctgttattgtggcaggccaaatctcactaatgcaggcctccgtaacagctgtttccgtactgactgagtggttcagttaaatactaaaagctaaaaaaaagccagcgcccttatacagaggctggaatgtaacaaaagcccaccaagagttttgcctaggcctttcctggcaaaaccaaggaattcttaacaggacccatttaggattaacaagttttactgggagtctgaagaaactctacaggcctccaagaacaagtttactggaggtctgcaggaactcctcaaacctccctgatttagcaggagacaagataagagtaatcaccccagcacctagacccatttagattaagtaaatttactgagactccagaggaaggtcttcaggactcagaccttagttatggattaaagaagttaatcacttctgtcatgagatgaatgcacacttacacgtagacatacagcttagaaggcatataagttctggaaactttgtaattttgagttggtctggcactaatttccaggccatctccctgtaaaaggttacaaaaataaaaactctcttcctcccagttcatctgcatctcgttattgggctgtgaggtatagtagcccaaccctcagttcagtccgggaacattattatcaaactctggggattttcaggataagttaggaatatattttgaaggagaatggcagtattaaatgagatcagcaattgaaaacatgtgaatacattcaccatttaattacccatctcaatagaagataaggatgaatttagcactagaaaccatgaatgtcacaaaagcctaactttaataccttgaatcactttccagtaatcctaatcctttatccacatggatagaggacattcctatcttgctctttcttctccccacacattagcttaataattagctaaataatttcatgcattagctaaggaataatggaactccagaaacctaggacagtaattgagagactgaacaagctaaatctgaaccttctgcacaattaaaggagctgatgttagcatggcccacagaatattacctgcacttgtaaaaactagaaagtcaggcaagtactcctggaacagactgtcttttctaaccatgcacttatttctttgtttttaactttgtttttgtttgtttgtttttgagacggagtctcgtcctgtcgcccaggctgtagtgcagtggtggaatctcaggtcactgcaagctccgcctcccaggttcaaacaattctcctgcctcagcctcccgagtagctgggactacaggcgcccaccaccatgccttgctaatttttgtatttttagtagagatggggtttcaccatgttggccaggctggtctggaactcctgacctcgtgacctgcccgcctcggcctcccaaagtgctgggattacaggcgtgagccacggcgcctggcctgtttttaacttcgtaagttgctttgtagaactatcttaaacttggtctattttagattctccttgaagggccagatcacaaagtgattaggagaaggaggctctggagccaagacagtgagactaacccattcttcaaatctaacactaactagctgtgtaaccattgggccagggatgtagcctatgtaaccacaatctcctttgtgcttcatgcctcaccaataaggtggggagagctgctatgactctgtcatagggctgagacgaagatttgatgagttaccagcaaaaagcatttaatatactgtctcattcagaagcagtcaatacacattgaactcttattacttagaggaaagaaaggggtcgatgatttatagataaggcctgagtgaaagaaataaatgtgtattgagtatcagcaatggactacgcatcattctgggtaactatcactgaacacatttaatctgcaaatactatctgaaagatactgccatctgaattgcataggtgggaaaagtcaaggcttttgtgcttatttttaaatgtatgttaacatgaataattaattggccgtgcactggtcgcacgcctgtaatcctagcactttgggaggccaaggcgggcagatcacgaggtcaggagatggagaccatcctggctaacatggcgaaaccccgtctctactaaaaatacaaaaaaattagctgagcatggtggcacgtgcctgtagtcccagctactcaggagactgaggcaggagaatcacttgaacccaggaggcggaggttgcagtgagccaagatcacgccactgcactccagcctgggagacagggcgagattccatctcaaagaataataataataataattagtgggaatatatgctgtttgttgtgataggagaagaaaaggggagatccagatgttggtcatcagaaatgacctgcatcttctctaaattgaaatttagcttgaagtagtaatgccaaggccagcctggggccatggcatcctggtgtacctgcttgttggtgacagtccttttaggacaaaatatcttacaaaaacattcatagatgactaactggaaaaaaagaagttatgatgaaaagcaattcttactgtttttgttttttgttttttgttttttttaattaaaagacaggcagttattctctttcccaggcttgagtacagtcgtgcaatcagggctcactacagcctcaacctcctgggttcaaataatcctcctgaccaagcttcccaagtagctggaactgcaggcatgtgtcactgcacctagctaatttttaaaattttcttgtagagacaggttcttgtaatgttgtccaggctggtctcaaactcttggctgcaagtgatcctcctgcctcagactcccaaagcaatccttacttttttaattgaagtgctttttttttttcttatttaaactcttaaaaattcatcccattttcttcaaaacaataaatcctttacaagggagaaaaatgaaatagagcaactaagaaagcatccatgaaggccaggaaggaacatcatggttaaatgcgattagtcacagccccttctgtctccagtcttggatgaataatggtaacagaactagagagatttctaagaccttggtgcttaataacaagcaagcatcgagggtggttgtgcaagatcctgatcacacaactctgcatatcaaatatccttcacaatcaacaagagtcagcctttaatctctgatgttttcaaatgttctttttttttaattcacatgtataacctatcctcttttcaatatatagagatatagcttatcagtcattacctcttttactacttttcttctgagatttaaaaggtcatcctctgatttgtaaattcaaccagatttcacagttcagatgagagttgtaataagttgccaagaactgttgcaagctgtgatggctgtctttctggggattatagagaagcacatttcttttaggaaaaaagaaggcagatgagcccaagttgttttatttttattgtgtatatgtatttgttttctcaagtacagtagcatggcctctgtggcaggcattcaccaagcaaatacagagcaccctctgggtcaagcactgtgttattggggttccaaataaagaaaaaagaccaaatccagccctgtggatctcacattaaagaggggagtaagaatcatagataatttaatgagttaggtacattaatggggatttgcacagtatttcataggagcacagaaaaagggtacctcgttccacctgagatggcagggagatggctcctaagttaactaagcaaaaggggaaggagaggcacatcccataggatatggataacacaacaaaagctccattaccacccgccccaccaccacacacaccatctctggaataaattttgtaccttccaactggttttcagggtactcatgacacatgggttaataatagctaggtttgcccaaatgaccaaaattctattcccaacatgaattttcacaactggtttttttgtttgtttgttttaatgaagaagaaatgttgtgtttttggaaatcatcttacaaaatcatgtagtggaatgcatatagtgatgaaattgttgaagaccagggaatctttgtccttagtttttattaggatggatgttttcaggtaaaactaaaataaaaatgaaatggaaccaattagaaagaaaataggagcacttaagacgaaatgtaattgttgctgaaagaattagaatctgatagcttagaaagaacttaattaaatttcagagtaaaataaatacagtttagcaatggcctttctcctagaaaaatataatgtctcttacagttcagttactccttggcttacaaaatagtggttaaatttcctgtttttctcattttgatttcgtatgttacatgttactggtgatgggattatctgtgtgacattacctctggtcttcaaaatgtacacgtagcaatgtagtctcacatatgccccttgggtgcgaaaagagaaggacctaacttaacctgacagacctcaaatctgttctctttctatagcagagtactcctatttaaaaagaaaatgttgagtaaggaggacagtcatcatatatgtcgagaatgttaaatgctcttgtggaagtgaaaagcagccgatctaagtaattacccattaagaaaggagtaggagaaatgtttagaaggcagacctgcaaaaaggtgactcacagtacgttcacatgaccggatgcattagtggaacctctaacccatcgccaatggaagaagcagtgttttgcacaaacttgaaaaagagtttttcattttcctcccacagcctcgtcacgtgttcaatatgctaaagaagtatgtccgcgcagaacagaaggacagacagcacaccctaaagcatttcgagcatgtgcgcatggtggatcccaagaaagccgctcagatccggtcccaggtaagcgtggggtataatcatcttctgcagctttgacaatccaggattcttgcttcttgggttggtgacaggatcccacgcattctccgagcacctataggtgttttccctatggcaagagctctttaacttctggtaaattctaaaagcttgtcttagagcaatatgtggtttggcattttctgagttatgaaaataacaaatgtaaaaatcttagtctactgctaactgattgctaaggaactgattaaaaccaatgactaaaatgagaaaaacaaaagttgtataaactaaaatcttgaaaactggttcaactgaagtatggcaggcaatcataaacctactggaatgttgagatttcatgtgagcctaaatgaagcccagtaataatgtattttatactccccggttcccaaaggctgaaaactgggatctaccttaggaaatgtgaaattttacattttcttgaatgaaagagttaaatatatatagcatgaaatagccaatcaaattgaaatacgttgttttctagggggaaatttgcaaagaaatgtatttaggcatgagggtcctgttaagagagcctcagtattaggaatcagaggtggcagaggtagcctttttaattggctgttataagcatacaagataatggtaagtttcaagatcaagtcttaaacctgagctcaaaaagttagagaacagaaataggagaacaagaaaaataaagttgccctttcagttcaacctttttaacagtagcagtgttttgtcagttttcttttggaagtttgtatctacctgcagtgcttggtaagaaaaataacctgggcgacagagcgagactccgtctcaaaaaaaaaagaaaaaaagaaagaaaaagaaccattcctacccccagacattgttgacctggagttgtcatcctttgatgcaggttatgacacacctccgtgtgatttatgagcgcatgaatcagtctctctccctgctctacaacgtgcctgcagtggccgaggagattcaggatgaagttggtaagtaagctgttcttttgatgctgcacatggacatgtattttcccccagaggaaaattgaggaagtgagttatctgtttgaggaatatggaagttaatagaacagcttgccttttacgatgaaactgtcaggctggaactagcttactagccttagcaattgaatcagttcttacctctccatacttgtaagactggaggaatgtcgcctgacttagggtatagttttgtaataattggacacttgttattgaattatacgtggcagcattttttctaatttgcccaatcttgttatggctctcaaattatgatgctttggtactttacaattaatggggaaaaaaactcattcttcatcttttgagtgtcacgattgttccatcttacaaatgaaaaagctcaaacttaaaatttgagtaacttactggcctgtgtcacatgcattataagtagcacagccaaaacacataactgtgacttgtgactttgaggttattgttatattcagtaagtcacaattctctttgatggctgagcagggaagccatgagtgttctctgaaccacatcattgtttaaacttaaaagatagtgggctattaaacttatattctgaaataattgtactctcacaagaacttggaaaaatagtacagagaggtctcatgcatccatcacccacatgcctcaccagtgacatcttacgtaactatagtccattatcaaaaccaggaaactgaccttgtgtgcccactgtcttttatgtccctagttttcctaatgactaatgatttcatttaagatctctcatttctgtttctcttggaaagagaatcaaataccacatgagtctctgtcagctgccaaaatgcccataactgtttaacttacttcacaaaggtacatagatgacgattatgttggtgcatctttcaatataagcatatgtacctatactggagatttagtgtgtagtctaccaacatataagcaatatgtatctggcatatccttccccataaagaatcctaccctttgttaattgggttggaaatagatacaggttatcatctggaatcagagcacccaggatgttgtagatggatgtacccattacagtaagtccgactttgaagtgcctgaaacaccgctcaagggaacagctggccctcactttttcccattttatgccagtgttattctgctcattctttcctgatttatgtggccttgtaaatattgtttgtattgctgtgaaatatttggcatgtgtcaagtgatgatgttctgttcagtactgcacattaatggacggaatctgaaattatactttgagctacataacttgcagaagaaacttcatatgtcagggaggtgcagtgtcccagaactttgggaggccaaggtgagcagatcacctgaggccaggagttcgaaaccagcctggccaacatggtgaaaccccctctctaatttaaaaaagaagagaaaaaaagcaacttcatagtgtgccaagcttttgttcagaaggagcaactcagtgttgagcaagaatttatgaagatagtggaggtggagtttttagtctacaccatgaattgagaacagagatctccaattaatttttcactgtatcaagtttctttagcaaacaaatatacttttcaggtatacttggagaatagacatcagtaaggaacctttaaaatactatgtattaaataagtgtaaagaagttctccaaaaagttgtagaaggtacaaaatgaaataatttatcagtcttaatgaactgaagctaaaatcttctagaaaacatggtttgtttgtttgtttgtttgttttttacagctatcttaagcctgttttcacatgtgacttcagttgaagtggcataggtaataactatagggaaataagccattacaaaggactaaaaagttgatgaccaatattccaccctcacccagcaattattgttgactctttataattaaattattagaactttttatttggcactatactactgaaaacattagccactaattagtcacacttccacagaacagatggtggttcagatatcttgaatattacgaaatgcccaagaatagggcagcacaataaacttcaaatctcagtaccagggaggatatttagataactagtatcatggaatctttttctgtaaaaagaaaaaaaaaaacagccattaggttggattaagaacatattagggggtgcagcacaccaacatggcacatgtatacatatgtaacagacctgcacgttgtgcacatgtaccctcaaacttaaagtataataaaaataaagaacatattacgtatgtgctcttaatgtgtgtgtgtgtgtgtgtgtgtgtgtgtgtgtgtatgtatgtaatatgctctcaatccacccattagttgggtttttttaagtaatgacatatatatcattctaaatataccagatgccacattctgaaagataatcttttttaaaaacctttcatttggaatcctgggtgcttccatttttaaatgactgttctctatagaaagagaagtttgcaataacgaaccctgtacactatctgagctgctagtgtcaaagctacagaagtggtttgctggggagaatagcttttctgggttgaacctagttgtcatctatagtcctttttcatttgaaggaacatttaaaacaatacgtccctttttagtaggaatatagttgaaacttcaagattggccaggggtgctaattgcttaagccaaatgttattagaggttcctcatgttattctttttacttttgtatgtgtttaaaatttctcataataaaaagtgttgcttttagcttactttctaaataggttcttcatagattttttttccaggttatgtcttgtagtcgttctaatattctctgattaaatgctttgtcattagtctgagttgcatttatttgctatttctgtatataatcaaattttagatttggagtcagttcagtacaaatagcatactaaaattcagagacactggcaaaaggagaaaattttaaagtccctaaaaatgcatgccttttgtatagaagtttgtgcttataaatacaagcaattaggatgtgcttaatgcagtcaaacatctgtagagagatacaatggtttaattaaataatggtaacagttgctttatggataagaggttttcaccagtctggggaactgccagtatctttagtgggagaggtatatctgcttcagtaaattatgcttccaattagaattctcttttatgggtgaaaatgaggaagaatggagtcatgctgagaagaaggaatgctatcggtgaaggggaaaattgacattaaatttgtgagcagtaaactgtgaccagagcatagggttcatcatgcttgtgagttctggccctggccttgtatcttccattgtgcttccctgaggcatcctgtctgttcatgctactttcccactaacttcagtgaatcccaaattcatgggtctagccacagccagcgtactgaatcccagtgccatattccagaccccaaatatcccatgtgcctctgaacctgcaatcaatgtgtataacactcatgtccttctggaaatttacttgttttacacactctattctgagttctcttggttggcctaaactatccatgatgccttcctctccctcacccattatttcttattagttgcaagttctgtatatttggccttctgtgttcatgtgaaatctctgcacatcccctacttacctgagatcctgtcctctcatcagtgtcattagactcccagccagtcaccctacctcttatctcacattggccccctcccacttcaattcctccttttcggaactatgcaagttcccttaaaaacaaaagacaaaacaaaaaaaacagctgcaggccggcggcagtggatcacctgaggtcaggagttcaagaccagcgtggccaacatggtgaaaccccatctctactaaaaatagaaagattagccaggcatggtggcaggcacctgtaattcccagttactcaggaggctgaggcaggagaatcacttgaactcgggaggcagaggttgcagtgagccaagattgcactattgcactccagcctgggcgtcaagagcaaaactccgtctccaaaaaaaaaaaaaaagaaaaaaaaaaagctgcttgtggtttttccttgcttgaaaagttgcacctttcaagattcaagtaaaatgcccaagtcctataaaccctttctaatttgcatagagaacggagaaaagagctggcatttgagtgacaaccatgtaaccaactaatgagtgcctctttgtgataatgctgatggtcacattctttctttatgaatgttccccgttgaggtacaggaccgccatatagagatgtggcagtggcaggtggtttagggtaagagtggagactgataattttattctacatcccactcaaggagccaagccctctggcattgtgcacacccactggaagtgggctgttcttgcatttcacacaaaggcagcatatgggctagtgaagatactcggaaattcagtgatagcatccccagtttacagttgaagaaattgaactttgaacctaagagagtttgccaacttgcccagcccagatagtaaagtgatgaaagtggggtggaagccaagacctgccagaagtccaaaatctgtgttctttctgctgcctattttcctctctgaagtggaattttcccctacttaacctcaatgtgaaagcactgggtatccacctccaataccatactctaacttggagtaaattatttgtttatatctctactaaccctccatcctgtgaactcttccagatatatgttggtcatccttgcctcctcaagatccagcaaggaccctggtactttttagatattagtaattataaatataaggttgaagccagtccccaggataaaaaggcagatgctccttaacttggactgatgaagtgggctcctccgtaaccctaaagacctgaccaaaactccttccttgacatctagcgtaagcaccacacaaaacaaatttctttcctaggccacagcatcctctaaagctgtgattgatcaagctaactaaatggatgtggttggccaagataacaaagcataaatcagggttctgaggacctttattaataaaattttacaacatggtccttctttgtaccatgaccctctgctatttggtcatctacttgatttaattgtgctgtcctgtttgaccgccaaccacagtctgaagctattcccatgacaaataagtgtgaattttcaagtgtcctgttgtgttataaaaggcccgtgttttattttataatttcatatttttttcattcaattttaattattatgtgcaagttcccaggctcagtcatttattaatgtttttattttttaaaaagttattttcaggcagattatgaagatttcttgcaaagaatggcctcaggcattgtttgtatagagataaaatctcctcttttaaatcactatgtaggtcttcattaatattttttcattcaacaaacgatggaatcattatttcagtgaaactatggctcctcttcagtgttcatattctgctttaattatttattaatccttcaaaagggcagaatcccttcatcctttcaacctttgctttgtcaaattgttacttaaaaatgatggtgttttctttgtcctttgtaatcttccttaaatctctgctaaaaggattctattctactctttgtattggcactgaggttatttatcaaagtgtgcttctctttgcagctacctcaaggctaaaaatgtagctcttttcagagatggctgttttacagaatgctataataaccaatttatgagaaaggcagtaggaaataaagagggaacaaaggcataattgtttcatacccattttttttcttctagcattcaaaattaataaaaacatgaattattataaaccagatgcgggaaagatctctggctgaataaatccatagtgaatggggcaggattgaaagatatggcggggaatcaagaaattcgcaataatgaaaggatgcttattttttattacaattttactgtggcctcttgataaaatgttccactcttcaggaaaaatgcagcatggttatttcaaagacagtgtttccatattctgtttctaataagcctgttgatttgggggtttagtgggtccaaagtagactaggtttaaaatagaatatttatagcacttcccagcccaacaaaatagaaatttaatgttattgagaatttcaagtatccttatgtgtaattgttagtcaaggatgaacatcaaaaaaaaaaaatcataacagcaaatagagagctgtcactttgttcttataagaacataggtgtgactttgtttttcataagaaatgacttttaaatgtatatagatttaatggtttacagctgacttaagtatttggacataagaactttttttttttttttttttttgagatggagtcttgctcagtcacccaggctggagtgcagtggcgtgatctcggctcactgcaacctccgcctcccgggttcaagtgattctcctgcctcagcctcccaagtagctgagactacagacgtgtgccaccacacctggctaatttttttgtatttttagtagagacagggtttcaccatgttggccaagctggtctcgaactcctgatctcaggtggtctgcccatcttggccccccaaaatgctgggaatacaggcgtgagcccccacatctggccaagaacatttatacatattatcaacaaccaaagccttgttcccccattggtttcataatagtctcagagctccttattcatattttccctagtgttattaagtaacagacatgaggcctgtaagtttagctgcctgtgtctaaatgtatatctttgacccaagacaatttctctttcaaaatagaaccactcatttataaatgaaaatgctttgtcaaatgaatggcaaagtacggtatactattaagacagcctcctagttttctgacatttggactgccatgttgaaatgtctccacacagcaagatacaaatgtgtgaatgccagtacacttcataccttaggtccccaactggtctgtctgcagacttgaggttacagacctgcaggtgtcgaactcaccccatagtggtggttagtagagtgctttgtatacgtttagctctctatgccttttttggttgtgatattaggtaagctctctttttcttgctttggagcttaattatggaatctgtaaatatagtgcatatgtcatccagatgatctgtggaagtcattgataatgacctttaaaaattcacatgtaatacagcatccgttttcctgaatgatttttatagttttaccattggcaaacagatactttcttttatgtatttctactccataattttaggaaaaagtaaatccttttagctacattaaaaagaatacactttctaggaataatgaatgtaaaaaaagtttccttttttaaaaaaatattgcgctctctaccattctggcagacaaattgttgaccttaatatgtataattcagtgcagtatattgatcttgtttattgtgctccaaatggacgctggcagaaatttgaggaattaaatatcaaccacagtgttttcttcatgcgattgcaatggtttgttatggattattttgttcagttcaaataaatgatgtacgttcagattctttgcccaattttttaattgggttgtttaccttactttcttaattttatttttatttttatttttatttttatttttggagacagagtcccgctctgtcacccgggctgaagtgaagcggcatgatctcagttcactgcaacctccgccttacggattcaagcgattctcgtgcctcagcctccccagtagttgggattacaggcacatgccaccacatccagctaatttttgtatttttagtagagacagggttttaccatgtttcccaggctggtctcaaactcctgagctcaggcaacctgcccacctcggccttccaaagtgctaggattacaggcgtgagccactgcactcggcctgctgttattattgagttgtaagaatttttatatattctggatactattaaattcttatcaaatacattatttggaaatattttacccattctgtaggttgtattttcacttccttaataggatccttttgacacacaaaaattttcaatttagaagtctattttatcagttttttcttttgtggcagttactatggtatggtatctaggaaaccattgtccgaaacaaggttacaacgatttatacctgttaccttctaagagttttgtagttttagctcttacagttaaatctttgattcattttaattttcatatatggtgtgaggtagaggtttgactttattcttttgcatataaatatccagtgtcccagcaccatttgttgaaaaagactattcttgccaggtgcagtggctcatgcctgtaattccagcattttgggaggccaaggtgggcagatgacttgagcccagaagttcaagaccagaatgggaaacatggcaagaccacatttctacaaaaaaattatccaggcatgataacatctatttgtagtcccagctactcaggaggctgtggtgggaggatctcctgagcctggggtggctgaggctgcagtgagccttgatcacgccacctgggcaatagagcaagaccctgtctcaaaaaaaggaagaaaaagactattatttcccccattgaatggtcttggcactattacacaaaatcaattgtccatagataatatgggtttatttcttaattcttagttcttttctttgatctgtgtgcctgtgcttactgtagtaccacactgttttgattattgtagctttgtagtaaattttgaaatcagcaagtgtgagtccgttatctttgttcttctttttcaatattactttgagtatccggagtctcttgcattacaacatgaattttaagatcttcctcaccccctgcccccactaccatttctgcaaaaaaaaaaaaaaaaaaaaaagtgcggattgcattggatctgtagatcattttggggaatatttatatataatgttaatgaggttttaaacaacacttttatctactagaactgatatttgcctcgaaatgattccaccttgagccccattcaggaacgttagtattttagaagtagcaagagaactgatttagcatgctatcatcttttactccaaattttggtgagattccagagaaattatttgtgagcttttccttaagccatgatgactgacatgtcagtagtttttgtctgtttttatgccattcttttatttttgtgcatgtgtgggcacctcaaccaaatggatggagcctgaggcatggtctcccacgccaaagtctgccgtttcttttgatgatccaatgcagttccaaagctattttctcaactcaccagctcgtttgtatggccctgtaatttgaaacaaaatctaaaaagaaatccaaattaaaatgattgggaagtgaagtcagaccacagtggtggctgtggaactacttcctggcatggaacactagagcagatgtgtcctgctttttttgtagagtcttgggaacaaccaatgattttagactggaacaagggctggttattctgtttttcacaagcatctgccttcaaggttcattttttaaaagaggaggggaaaaaaatcagtgatgtttgccaccctgagtatggagatgaatccaaattgagattaattttgaaatatgtttcccgaattttgagattaattttgaaatatgtttccacaaacactaactgggaatgacatgagtttgtttcatgtgagacggagtttggaagacatgttaatttgcttgttggtgttcaactcagaaacaaattttgcatgtattttctgagtaagtgccattattagttaacagttttcctgccgtatgcactatgggctgctttagaaagtaaatcatagaaggttagttataatatctcatcaaaaatagtggatccatctattaaccggttccaagttatgagacgtgtccaaaggaatgttgacttggtgtgcacctaaacactatttgtgaagaggctttggacttcgagcatgaaacggggtcagagggtcaaggccagggcgttacttaatttcagctcctgttgatggattcattgacttcagatagtgcgtttaaaaaagcctgtttaatgtgtttgtcttttgataattattatgtatttacatatgctcctaaaagcatgagttaaatcttatttctttgaaacagtgcttggcaacaacttgtattgtcttgtacatgccttttgtgtctttgcttttggttgggggggggcattgaaatttgtctgcccatcccctgagggcagatactgaggtgtggacctatgtcttcagcttcctgtatagcattgaagttgctatagctgcatgttctctaaaatttttatacatgcgatttaattgttaaagcttatgatttttaagtatagtattctgatagcaaatagattttgtcaaataaaaactaactttatcatcaccactgagatatcactaggtatatggcttagttctcagtcaccacttttatttgtaaggaagtcttagaagttttcacaagtgtatatgtgatagactgggcacagtggctcacgcctgtaatcccagcactttgggaggccgagtcaggcaggagttcgagaccagcctggccaacatggcaaaaccctgtctctactaaaaataaaaaaattagccgggcatggtggcacatgcctgtaatcccagctactcgggaggctgaggttggagaattgaacctaggaggcagaggctgtagtgagctgagatcctgccacttcaccctagccagggcaacagaacgagactccatctcaaaagagaaaaaaaaaaaaaagctgggtgcggtgctcacgcctgtaatcccagcactttgggaggccgaggtgggcagatcacctgaggtcaggagttcgagatcagcctcaacatggagaaaccccgtctctactaaaaatacaaaattatccaggcgtggtggtgcatgcctgtaatcccagctacttaggaggctgaggcaggagaattgcttgaacctgggaggcagaggttgtggtgagctgagatcgcgccattgcagtccagcctgggcaacaagagcgaaactccatctcaaaaaaaaagaagtatatatatgatgcttgctttagcagcacatatactaaaattagaatgattaaagagattaacctggcccctgcacaaggatgacatgcaaattttgtaaagtgttttatatggaactttacttacatgaggtacctagggtagtaaattcatagatacagaaagtagaatgctgattgccaggggctgagaggatgagggactgtggagttagtgtttaaagggcacagaggttcaatttggcaatattgaaaatagttcagtggggaagatggatggccgtgatgcatcgtccatcacattgcacagcatcgtgaaatatacttaataccctgagcgttacacttaaaatggttaaaacagtaagatttatgttatatatatttaccatgataggatatatatatgtatattcatatcattcgccattgtgaatagcaaggagattgaagaaataaggacacaggatcttcaataaatatgtattaaatgcttcccatgttccacattctttgtcattgccagacacgtcacaggtataagggcccaatctttgttactcaaataatttagaataatttagaatttgcttggggagactttggataaacagttaagaagctgtatccattaggtaaagggacattgcttgctgcgtctcgtagcatttggtagtctcgttagacagccgttcttactcctatcgtttgctgaaatttattagcaaaggaaagaatcttagttttcagctcgttcagccgcatgatttcataggctcaggaaaaccatgtaatatgcccaaggtcagacgtgatgaataagactaaagactgaatctattccagtgtctctgtgtccagtgatacttacttactgctgttgccaagttaaacaaaactgggtttgcagagctgtttattcaccttcagcatcttttcattgaaaatgcggtagatagaagaaatgtccctcttttagcatttgtacctcagtaataagatgcttcccaagctcaaagactccacacccttaaatatgtaagtcagtgttaagatatttaaagagaacatgaatatcagctacattgtgcatgttgcaggaattttatattattttgtatcttctgcttagaagtcataagccagcccatgaaccatgccaacaaacataccttcttccatgtgattattgcagccagatttttcagtgcaggacaagcaaagttttctctgaaagcatgagataataattctcagccaggtcttcgccatttgcaagggatcagacataagagtttttagctctgttgatgttaatagctagagcccctgttagcagatggcatgttagtggcctgttgctgctggctattaaatcatacacattggccaggcacggtcggtcacaccagtaatcccagcactttgggaggctgaggcgcacagatcacaaggtcaggagtttgagagcagcctgaccaacatggtgaaaccccatctctactaaaaataaaaaactaggcaggcatggcggcacatgcctataatcccagctactcaggaggctgaagcaggagaatcacttgagcccgggaggcgaaggttgcagtgagccgagatcacgccactgcactccagcctgggcgacagagcgagactccgtctcaaaaataaataaataaaaatattaaaagtaaatgatacacatcatgagatcatctcccaaggcagcagagaattttgcgtatttttttcttagtttatattgttgcaacagagtaattcttcttgatcacagactttgctttttttgttgttattgtttctaagtttcacttgatcctcaaaataacctgagacagacaaaacagataggttttcccattaatctccttcatgagtctgactttgccagccaggctgatcttatatccatagccctggatgcatttatttatgttattccatctgcctataatgcccatccccaatcttggcctattacatgtgtctaacattactttgatgtgtacatgtcagtgaattaccacttaactgattgttagctttttgagagtatgtatcaaatctcatgtgcatttgtgtgtcctgaaacaaattattaggtataaagtatgtattgcaaataccatgcccagggaatatttcagagctgtggttctccaagtgtggtccttagtctagcaacatcagcatcgcctggcatcacctgggaacttgttggaaaagcacattccatgatcccacccctgacctcctgaatcagcaaagctagggatggggctgagcaatctgtattttatcaagctgtccaggtgacattgataaatgctaaactttgcgaattactgatttagagactaatcaactcattaaaaagttaataaataggtgagaatccattgtagcaccccagtcacctatgctattatcttctaaattcacattatacaaattgtatgccacaaaaatgtttaaggtgaaaacccaaaggaattgatgcattggattaattcacaaaaatttaacagctgcaattcatagaaaaaaaatgcatgcatatctgataattagttgtagattaaaaggtgaacaacttcttattattaatacccaatgaaaatgtaattttttaaaaattatggccaattggtagttttttaaaaatctgttatgccttgtcatgataaggataggtgaaaaggaactttcataaactattgctaagtatatgggtgaaaccttttagactatttgggtctacctaataaattaaaatgttcagtcttttctaattttagcaccgtatttctacagatacactggcatatctgtggagaggggcacttctgcttgtcacctgatatcccttactactactaatatttttataagcattattacctttaaaatcatgttaaagggtaaatgatgtgcaacagactcagtttccccttctctgactcctgtttagtttacatctcttttctctctctcatttgggggctcaatacccttaagattatagagaaattgcagtaggtaatgttggctggaaatgctaacttgtaagaaacttgggtggaaatggtttaagccatcagcctaaacgatcagtgaaaatgtcagacattctccgctttcttcttccaagatactatactctactgtggccttagagttcttaattcctatataattctgattaaaaaggaaggagatgctcctaatataccttgctgttgtcccctcctgttcattagggtagctgatgtcagtcaaaataaagtctagaaattcttgagtactttttttttttgtaatatactgagaatgcttgaagcaaactaaccaaatttaagtgttttaaggatattaacagtaacaggagagcccctcgattttctgctaagaatcaacctgaacagtttccagactgctggttggcaatgtcagtatcatgagaaaaactgaaaaaataagaaactttagagaaaaagaggaatcagtgcaggctaggggaatgaacttggtaatccagtagtctttccctgccagcattttgattgataagctaagtgtctgctttttctcatcccttattttttggcaggaaaccagtattttgatacttgaatgccttctttctatcttaagggagaagccttcttaaacttcaagtgtgaagtagatcctttaggtataggatgaaaaattaagtgggactatttgtgaattactaaatcgcaaccattaaagaaactaatgccctaattagcctgtcttttgacagacatggcatccatatgtaagtctaatattgtatgcaaatgtgagacaggttttgctttctagatctctttgatgtgaaaggaaaagctggcttcctggcttccttcccatgttctgtatggacctgccttcatcctgctgccaacagctcctcaagggaacatgaaagtggaattaaaccctcctggttttcagattcagaatcctgggtgattaagtggcttgatcaaaatcagaaactgtgaaagtgctaaaaatggtcaaggcccaaggagttcctcatgtgtggtcaaaagcctttcagaaatcaaagtgtgtctttaaatacctggggtgcaaaaatcagatgttttttatttaaagaaaaataggtatattagagcagaactgggggccctctgtaggagcctgagtctgggtgtttactgtcgtctaaggccttgttaatgtgggcttgaggcttcggaatccacgaacaggggaagctagcgtctctcgctgactccagtcctagctcgagctttccatactgccctacgtgacttctgcacttttgtgccatcagccagtagactgagaaaagtatctggtatttgtcggtgtcttcggtttagctgctttaagtccttggatggaaaaggctgattgtcatcctgaaatcattgtctcctaatgtgtaattggggccaagtgaagcacagtaatttagaggaattctagttaaaaatacagattcttgggctctacctcagacctacagagatctgcatttttcttattcccccaggtgatcctttaaacattcagaggtttttaaaactcatgcttatatgcaataacaaaaatataaggattcttagaaacttgttatatttgtcttgaacatgggatgacagacattgtcattttcattgaaaaaaaaaatgaagcagagactcagaatgtatttagagacaactctaaatacattctggcagagcctaactactggattgcagagatcctccctgcttcccccagtccccatctataaactggccatgtgaatttctagtggtgttcacttccaaagtggtgattttataaaatattacaatgaagcattattaacctcaagaagttcagatgcatagagagtttatttgggtgtggcagcattagtggctaggttttatttatccaagataagacttcttattagctatgaaaagggattggcataaactgcttattaatgtccattaactgtatctaaagggaataaacagtataaaaggccaggcatggtggctcacacctgtaatcctagccctatgggaggctgaggcaggaggatcatttgagcccaggagtttaagaccagcctgggcaacatggggagaccctgtttctacaaaaagtcaaaaaaataataagctgggtttaggggtacacacctgtaatcccagctagtcaggaggctgtggttggaggatcacttgagcccaggagttggaggttgcagtgaggcctggcggcaccactacactccaggctgggtgacagagggacgaccttatctcaaaacaaacaaaaacaaaaaactgtataagcataagatgggcttgagtcacaaaattctgcctttcataactttcccccatttgctgtttgatttgctgtttcagaaattgtgaacctacttctctacttatagaacatttacctccctcttagattcttccctttctatcattgatactgacattctgcttaaagaattatccatttaggctctcttcaaatccaaagtgatattattttattttaataagagatgaaagggaaaattgacaaaatgtgcctggagttgtcagcattgtaaatgatggactgtaaaagcttcgtcttggaagattgtcagagagcctagtgccagctcaaaaagtgtggtctttttagagaattgacagaaatgttacaaagaaaacatgtgctgaataccaactccaggtgcgaatcctgagaagcaactcactctattctgcagtaggagatgaattttgcaggttatacagcagagcaatttaaactcccttgtttcttaaagtatatcctatagtgacttgggatggaagtcatatactatccccttttttgttccttcaaagtaaatacaccacagagctggagatgtcccagacacagggggaagctgtttggatttttcccactgcctgtaatgaagcaagatagggaggaagaacttgggaacttgcgtggcaaatggtcaagtaaagaaaaagaaaagctttttctttacttgaccatttgtcacatgtgctttcaaatcctgaactgagtcacatctgaaataaatcacttccttgtcatttccatgaaaggggcagtacaagaaattccttgcacatccctggtgagccccttgttactctcagattcagctattgccttcaggaaaaaaagaaaaagaagccgaagaaagaaagaaaacagaatcctctagaaaaagctattctgacctgtgctctttcttttgtctgagtgtgactagataaatgcattagtctgctagggctgccataacaaaataccaaaataccacagactgaatccttcaacaacagaaattatttttctcatgcttctggaggctggaagtccaagatcaaggtaccagcaggtcggtttctgaggaggcctctcttccttgatttacaaatggccacctacgcatcatgtcctcacatggtctttctcagagagaaagagaggtgtctcttcctcttcttcttttgttttgttgagacagagtttcgctcttgttgcccaggctggagtgcaatggcacgatcttggctcactgcaacctccacctcctgggttcaagcgattctcgtgcctcagcctcccgagtagctgagattacaggcatgcctggctaattttgtatttttagtagagacagggtttctccatgttggtcaggctggtctcgaactctcgacctcaggtgatccacctgccttgccctcccaaagttctgggattacaggcgtgagccccgcacccggcctcctccttttcttataaggacacctgtcctattggatcagagtcacacccccatgacctcatttaaccttaattatctctttaaaggtctgatctccaaaggtcccttgagggttagggctttgatacatgaattttcaggggcacacagttcgatccatagcagttggcttattcatactttgggtagatcctcttcatcgttcctcagctactgtctatttagtgcacagcttctttatcctaaagctagtcaggggaggcatggaaggtattatttctcctgtttctgcacctaaggcattaaaacgaggttgtggtctggcagcctaaactcggccttgatgaacttcaaatcctagacctccttggtatgtgaagtgcatacacttgtatggagggatttgacaggttttttttttttttttttttttgagacagagtctcactctgttgcccaggctggaatgcagtggcacgatctcggctcactacagcctccgcctcccggtttcaaatgattctcctgcctcagcctcctgagtagctgggattataggcgcgcaccaccacacccagctaatttttgtatttttagtagagacggggtttcagtatgttgatcgggctggtctcgaattcctgaccttgtaatccactcgcctcggcctcgcaaagtgttgggattacaggtgtgagccaccgcgcccagcccagatttttttaaattagtttttagtgtatccacactgttaaagcaggtggatactgctggatagtataacaaagtatagtgtgacaaacccataaccaagcattgttagctatattttctcatgtcttgagccaaaaggtcacagttaacctaatgttgctactaaacactcataccataaaacattctattccagtcttccagattttacacaagaataaaatgaaaacttttagactactggagatttttttttaaatacacaatttgttgaatgtgaacgctaaattgaaattgacatgctaagttttatagattacttcagctgccctgacttcagtaaatttagttgtgacacaggtacaacagctgacttctttctggcattagaggtggtgatgtttcgtcactctggagttagtaaaacctgaaacccgccaggcatttgacacacagtttgctgccagggacagctcagcttctggcttattggagaggaaatgacctaacctctgtgtttacaaagtgtttgaaactgaggcccttccacttccttgctgagtgtaagggttgacctggcatccaaaatccccatgatcttctcaggataagtcatgcagccattggtggtcaagaaaggatttgttccctgtggaagggcattggaagaggaacagagctgaaacacacgggtgttcagagggaaaacatctctgcacttaagagggctagattagactattagactattatcatagagctcgtgattcatctcctttgtttaattccagccttgagtaaaggactcactaagaaaccacattcggccgggtgttgtggctcacgcctgtaatcccaacactttgggagaccgaggcagatggatcacaaggtcaggagttcaagaccagcctggccaatatggtgaaaccccgtctcaactaaaaatacaaaaattagtcggggatggttgcgggcgcctgtagtctcagctactcgggaggctgaggcaggagaattgcttgaacctgggaggaagaggttacagtgagccgagatcacgctactgcattccagcctggtgactggagcgagactccatctcaaaaaaaaagaaaccacattctattccagtatatttattataaccagaaattcttgtcttatcagcaaatagctaatattcacttacatgggcagtgtattagattcctagggctgctggaaccagttaccacaaaccgcatggccgaaaccaacagaaatatactctcattatctcattattctagaggttagaagtcctaaagcaaggtgttgccagggccacactgcctcttacagctctaggggagaatctgtttcatgcctttcgcttgctctagtgctgctgccagcccttgatattcctttggttggtacatgcacccctccaatctccacctctgtcttcacatggtgttactccccatgtctctgtttctgtgtcttctctaatatgattaagggcccacccttctccattgtgacctcaatcttatatctgcaaagaccctgtttccaagtaaggctgtattcttaggtgtaggcattaggacttcaacatatattttgggggaaaattcaacccataatgcggttatgtttttattttccttgacttttaagaatgtgaactattgtcaaattttttcaaccactatcaaatttaaatctaagaactacatgtcttaaatttatgacctcttcattaaaaaaaaaataataacatttaattggtaccaaaacgtgaaagagtttaatgctttgcatttgcttgaggctgattaatgagtaaatgtgtcaactgtggctgttttaattagccagggttaattaatgtttaccacacttggaagatgaaaaggcatgtataaatgctctataatgtgaatcattttttaatttgttgttgaagcttatagtgtagccaagtaaggattgcttacatcatacttacaaagggaggttcattaatggatacaaatacacagtttgatagaagaagcaagacctagtatttaattatcagcaaggtgactaatttacagtaatctactgtatatttaaaaatagctaggagaacctaatttgaaagtttctagcgtaaagaagacaggctgggcacgatggcttacacctatagtcccagcacttgggaaggccgaggtgggaaaatcacttgaccccagcaattcaagactagccagggcatcatagtgagaccccatctctacaaaacaaatatttaaaaattagccaggtgtgatggtgcatgcctgtagacccagctgctctggaggctgaggtgagaagatcacttgagcccagcagttcaaggttcaagtgagctatgatcacatcactgcactccagcctgagtgacagagagagacccttcctcaaagcaaaaagaaaagaaaaacatttaagtgatggatatcccattatactgatttgatctttccaaattatatgaatatattaagttttctcatataatctgaaaatatgcacatctattatgtatcaataaaaaataaaattttaaaaatgtaaaaaacgatgttggagcactatggtacttaacgtagaggctctgacttttgaggtacattcattacctgttactgaagttgagtatgctttaatattgtatgtttagaaaaggtagttacatttttaaatctgattgtgtaagaagaaaagagtagaaatgtttggggccaagtcaataattatttagtatatcataatttgagatgaaatgtgtttttttttttcttttttagtttcaagttgaaacattttaagaacagataaggaaaggctaactgtgctgagatgaaaataacatttttaaagtaactatcttaaagaaaatacatttctagaattttgtgtaaattttcaacaaagactcagaagcctggatgaggtcttgtgccaagactggaaagtgtggggattagaaaaagtttgaagagattgttgtgagatcctctcgtatgaggcctgaagctgtacatccatggaaagcagttaccccaggcgggaaaacaggttatccattctcgagtgagtggccgggtgggaaatagtccatcatgtcgaatggatcgtactttccagcaccaagatttgtggttctctaggtgaataagatgacaccccttcacttcacttcacttcaccattgtctgcatggagtacgtatctaaaaacaaattagcaaaaaggttgggtctactttacttttcatctcctacactattttcttctttgttctcggcaggaaaagatgaagttgcattttattattatctcataggattcttattctaaaatgatttgatttaaaacccattgcactttatttggataaaaaatgcttcactctctagttcttaacaataacaccagcctgattttattacgttaagcactgcaattagacaatacaaagaatttgcagcaacaacatgttattttatcatgggtggaactgttcattgtctagcagaatttattaccagataaatcccttttggggaatctaatgtggatgtttttttcaaagcgcctgtggagttctgctagtattgggaacgtttccattttcattctgatgcttccctgtgggctgtttcagcatagaagtgagctctctgggtccatgtttccattggctccacccaatattctgaaagattgcaactcaacatgtgtaaacagaacctttctcccccatattttctcttttcgtagattaaactcaggtcaagaattagaagtttatcctccaccccgatagcatctcgccctatcccaaataccgccaccaaaatgcccactgtttttccttgggccagttcctgaaatatctttctggctgtatcactcttgtgtgcatccttatcggctatgcttttggccattttttactgctgactttgtctggttagtcttttataatctccaggcttctcctccatcctttttttacagtttacctgttcatgaagcgcaggtcacttgacccgtagaattcccataggctgggattttgctaattgcatactcatggtataggtcagtgtattcctctgccctctgtatcccacaaactgaaagcctcttggctaagctgcttcccttttcacttcccttgtaccacagcagccagaagagtcacagatcctgggcagcttgtcctctgtgcctcggtacatgcacttgtatctgctaggacaatgatctctttccctggggccccactgtaatatcactagtcacatccctaaagcccaggcttgtttctttgtcattctgtcggtatccctaccataacacttgtaatagtaattatttttatgcccatcttttcttctgaaccatgagcccttcaagtgaaaagaaattgatgaagttatttctagcactcccctggaaaaaggaaggcaggcagggagggaggggctcactagtttatgataaagtggtttgattttcttggttgcagattatttcttttaaagtgactcttttcagcttctctcactcctgtgcatcgtgaatgctctcctgaagacagacgctttccattcatttccaagccccccatgtgaagtgctttctgttgccaggcttggtttccccacacgttgttaaagaagttaaagaagcttgtgaataagctaaagcagaggagaaagaaccattgctatatctagaattgttcaccagaaagcaagttttgtgttaaagaaaacacgagtaggccactgtgcagaagtgcttacagggtttgtgtgctttttaaaatctgcataaataaacatttcaaatgagctatcatatatacaaggatgatgtgctgtctgtgggaaaaggacccaatcctgtgttgtgattttacatatatatatatatataatttgtgctattacacacttgcctgatccccttataattatctgcataagtctttttaggctctttgaatgaaattcactatgtatggatgttgaaaatcatcctttctaccatgagctcatcagacttgctaaccaattctttgcatcctttgaagtataaaattccatcaattcagcatgtatcaacatgaatcaattagaatgcactttcttcacagcatggcggagtttttgctggctgccgggagcggcactgtttcccaattcattcatttgcttgggcccgccccaggatccttcttatcccggtcagatagacttctctcatgagcttgagagacggggctggtcacacaggacacggctttagggacacgactcactgaaactacaaaatgagtgctgctcttagagttgtacccacagcctgcacagctgttatggcaatgctgtgggcaagtcttgctacggcccattctcctccctcccttgtcctgttggaaggttcaggttggagctgaaattcagtccagcacgcagctggacaagccatgtctcctgtatgtgactcactccccagagaaagcatcatgtggactagtaagggcttggctttatcagaagcaaaggagaggtttacatctcttcttgctctctcttagtgggttcagagctccagcagaaaaaatgggacactgtggattgatctccagaaaaccccaaaaccagcactgaaggctccaggagattatgcctttagattgtatacaggcaccagaatgacttttgcctttataaaaatgcgtgcaagagcatgaaaactaatatttgaggaatttttttttctcactgtactttctggtcctcactgaaagcattggcagttttaaaaaatgaaatgtgatactgttatagacgtagcaaacctcacgtttcattttttccaattcattctatatctaggaacttacttagaaggagaaacaaactgtacccagaaaaaggaatgaaactggagaattcttcctcatgattcctctgtgtcctcaggcctaattatcctcattccaaagactctccattgcctcaataacttcgaaaaatttactctccttaagcatagtcataaaaattgcatagtaaagtgataaagagacgaaaataatgctgtaagaataaaaccattaaaaaaaaaccactactcttgattatttctgctgttagaattgtaaatgaattctcattccttttttctacatttttggcatgaaatttatgttactcttataattagtaaaaacattatattttattaataaaagaagactcacagtgaagtcataacatatgctgcagaggttagactcttcaagtcagatttgaagagagaatttcacgtaacaatcaccttgaaatgtcttcaggtcagccatcaaagtacacatctctcaaccagccaggcagccagtttttcctccagcatgtgaagagtgaagagatagctgtttcttggcttaaagaccatatgaaataattgacttatgtttaaaagatatgagggatcaggccgggtgcagtggctcatgcctgtaatcccagcactttgggaggccgaggcgggtggatcacaaggttagaagatcgagaccatcccggccaacatggtgaaaccctgtctctactaaaaatacaaaaactagccgggcgtagtgatgcgcgcctgtaatcccagctactcaggaggccgaggttgccatgagccgagattacgcctctgcagtccaacctggtgacagagcaagactctgtctccagttaaaaaaaaaaaaaaggcatcaaaattacattcacttctatttttatttttgtttgtagaagacatgcagtttgtatcacattaatgggcatatgatgtaatttcactataaatcgaaccagaggaagacatccaaatatgtataaattaaagctgacaatgcctacagtactaaactttttgttttagtataattggagacttagaaaagttctaaaaatatatttaaaaatttttaaaggaagttgcagacataatggccctttacacctaaatacttcactgtacgtttcctcaaaacaagaagatatgtgtgtgtgtgtttgtgtgtgtgtgttgtaatcttaatacagcatctttcagagattatttctgtagtatataaacttggtttttttgtttgtttgtttgtttgtttgtttgttttgagatggagtctcgctctgtcgcccaggctggagtgcagtggcacaatcttggctcgctgcaagctccgcctcccaggttcacaccattctcctgcctcagcctcctgagtagctgggactacaggcgcctgcaccatgcccggctaatttttgtatttttagtagagacgcgatttcacctcgttagccaggatggtgtcgatctcctgacctcgtgaaccgcccacctcagcctcccaaaatgctgggattacaggcgtgagccaccgcgcctggccataaagttgttttttagtctctactttaagttcctgacaacccaggcaggaccaagactccctaatgtagttggaaatcagatcagcacattggcacaagttctacaggtaaacgggaaaaaggagccctacaatctgcttttagatgccagggagcctggctggtctttatcttgattactgactgatgccactggtaggtctaagttctttgatctatgatgtttgaaagccaaaaggaaatgtgttttaaaggaaaagggacttcttctgtaccctcagtctaagccagacaagtgatatttcaggaaaatgtccctagcaataaggaatattttcacaaatgatgtgaagctttgtttgtgtcccttccagggcagtttgttaccataaggggacaaagcatgcttcatttaaacatgtgtggccaggaaactgttatttttaaacaaaaataaaccccgatttagacatgtagtctgagttttaactctgggagattttcaagattgatagtgtagctaaaaataactgacctaattttctggaggtcaagataaaaactggatttatggtacagcatttactttgtgataatatgtgtataaattcttccccaagttggaaaatgtggccgcgtcatacagggctgtatctctcttgaccattgaagtgcctaggtcgctacactacaatttgacttagagttggtgaaacttctggccattcacgtagccatcgtattcatcctgagaaagggaaacctaaagacttgggagagtgttctgggaggaaagtgctattgagaatgggctgtccaagcaaagtgacaggcaggcctcacttagctagaagagggagcttttcagccctgctccaccatcaggaatctccgtgaccttggactactgctttaatctcttttatcctggcttctaaaagataccgtcgaattcattctttttcaaacctatcaaaaatttgaaatgtatgaaaaagagggtgcttgtgatgatagatgggtttatgcttccttttttttttttttttttttttttttgagacggagtctcactctgttgcccagactggagtgcagtggcgcgatctcggctcactgcaagctccgcctccggggttcacaccattctcctgcctcagcctcccgagtagctgggactacagtgccaccaccacatctggctaattttttttgtattttcagtaaagatggggtttcaccatgttagccaggatgctctcgatctcctgacctcatgattccaaagtgctgggattacaggcgtgatatgcttctttaatattagtttctaatccaagtaatatagccatgtcctctataaaatggaacttttcatggaacagtgagaaggttgcattaggaattgggatacttgggttcagatttcagttctgccacttgctagttttaagtatacttctcaggatggatgtcagtttgttcaactgtcagtgggagtagtgatcccactaaggaagattattgtggttatcaaatcagcagatgggtatgaaaaggccatgagtgcagttctcatggttatgaaggtgtcataactggcatagtcccatcacagtatggctttggcatcgcagatggctgtagcatatatgctaatcagaacatccctaccgtccttaaagatctcatgggttctctcccaaatcagctgctccaccgacctacaagtaagtccatgcttctttagtatatttactcactagtgaaggcccgtctgctaaaagtgtcttcagtaggggagagctgctcttttctgggaaacccagcaatgaagtttgtcctgtgatgccctctacacttactgacctgtttcctgagaacttcccgtaacaaatggtccagggaagaaggaagtctgtgaccagtctttcctttcctgtggctttctggttcagaaatgtgaccctgatgataagaaacttcaaggcatgactctggtgttttggcaatattcccctggcagtattgtttcttggctcacactcatctcatgccttgctttgtttgctttgtttttgagaattgtttttttgtttgttgtttgtttttggttttggcttgtgagacagagccttgctctgttgcccaggatggcgtgcagttggcacaatctcggctcactgcagcctccacctcccgggttcaagcaattctcctgcctcagcctcctgaataatggggattacaggcacacaccaccgtgcctggctaatttttgtatttttttagtagagacgggtttcaccatgttggccaggctggtcttgaactcctgacctcaagtgaaccacccacctcggcctcccaaagtgctggggttacaggcgtgagccaccacgcccagccattatgtttgtttttctttcatgaagggataaccgaggctgttgtgtggtggtgtcttggtacagctcgtggctgtggtcagtgtggtggagggtaggtcttctcagatggtaacatgcatgagagtcacctggggatcacattaacatgcagattctgattccctggatctgggtgggacctgaggttctgcatgtctaacaaactcccagagaacacccatgtagcaagagtacagacaaaaggattcaaagcgaagggactgcaagaaccatggaaccagcctcccacgattttgcggatgacgaacccgagccttggggagtttcagaggccatggagagatgaagtataataaacagcatagcagttgtatgttgagccaagaatcagacatgcacagaactgctgattggaaaattaaatctgtttctcatagccctggtgtggcaaaatgcctagctggaagtgggtgaccgcttgatttacagtgggcctcctgggtagggaaaattagtccatcccatttacagctacttccaatgcagctgtgtcaggggagagagaagaaaggacatggaagagagcgtagaggtatgatcttcagtcctttcaggcaggcacataagtaaatttttatttaaaagataagaattggtcatatgcgaatctcctgttagataagattaaaaacaaatttccattttgtggagaagttgcagataaatatttttccatgggatccaaaagaatcaaaaaggaaagaaaatagtaatagcagtggaggttcgatcctagacagaagaaaatccagcaagccgagggacttcatattctcactggtgtccaaatcagagctttttttttttttctttgtttactttaagttctgggatacatgtgctgagcatgcaggtttgttacataggtatacatgtgccttggtggtttgctgcacccatcaacccgtcatctaggttttaactccgcatgcattaggtatttgtcctaatgctctccctcccctttcaccccacccctggacaggccctggtgtatgatgtccccctccctgtgtccatgtgttctcattgttcagctcccacttatgagtgagaacatgagtgtttgggtttctggtcctatgttagtttgctgaggatgatggcctccagcttcatccatggccctgcaaaggacatgaactcaacctttttttttttccttttttttttttttttgtatttgcaaattcaaatctttttttttttttattatactttaagttttagggtacatgtgacattgtgcagcttagttacatatgtatacatgtgccatgctggcgctgcacccactaactcgtcatctagcattaggtatatctcccaatgctatccctcccccctccccccaccccaccacagtccccagagtgtgatattccccttcctgtgtccatgtgatctccattgttcaattcccacctatgagtgagaatatgcggtgtttggttttttgttcttgcgatagtttactgagaatgatggtttccagtttcatccatgtccctacaaaggacatgaactcatcattttttatggctgcatagtattccatggtgtatatgtgccacattttcttaatccagtctatcattgttggacatttgggttggttccaagtctttgctattgtgaataatgccgcaataaacatacgtgtgcatgtgtctttatagcagcatgatttatagtcctttgggtatatacccagtaatgggatggctgggtcaaatggtatttctagttctagatccctgaggaattgcgacactgacttccacaatggttgaactagtttacagtcccaccaacagtgtaaaagtgttcctatttctccacatcctctccagcacctgttgtttcctgactttttaatgatcgccattctaactggtgtgagatgatatctcatagtggttttgatttgcatttctctgatggccagtgatggtgagcattttttcatgtgttttttggctgcataaatgtcttcttttgagaagtgtctgttcatgtcctttgcccactttttgatggggttgtttgtttttttcttgtaaatttgtttgagttcattgtagattctggatattagccctttgtcagatgagtaggttgcgaaaattttctcccatgttgtaggttgcctgttcactctgatggtagtttcttttgctgtgcagaagctctttagtttaattagatcccatttgtccattttggcttttgttgccattgcttttggtgttttggacgtgaagtccttgcccacgcctatgtcctgaatggtaatgcctaggttttcttctagggtttttatggttttaggtctaacgtttaaatctttaatccatcttgaattgatttttgtataaggtgtaaggaagggatccagtttcagctttctacatatggctagccagttttcccagcaccatttattaaatagggaatcctttccccattgcttgtttttctcaggtttgtcaaagatcagatagttgtaggtatgcggcgttatttctgagggctctgttctgttccattgatctatatctctgttttggtaccagtaccatgctgtttcggttactgtagccttgtagtatagtttgaagtcaggtagtgtgatgcctccagcttcgttcttttggcttaggattgacttggcgatgcgggctcttttttggttccatatgaactttaaagtagttttttccaattctgtgaagaaagtcattggtagcttgatggggatggcattgaatctgtaaattaccttgggcagtatggccattttcacgatattgattcttcctacccatgagcatggaatgttcttccatttgtttgtgtcctcttttatttccttgagcagttggtttgtagttctcctttaagaggtccttcacatcccttgtaagttggattcctaggtattttattctctttgaagcaattgtgaatgggagttcactcatgatttggctctctgtttgtctgttgttggtgtataagaatgcttgtgatttttgtacattgattttgtatcctgagactttgctgaagttgcgtatcagcttaaggagattttgggctgagacaatggggttttctagataaacaatcatgtcgtctgcaaacagggacaatttgacttcctcttttcctaattgaataccttttatttccttctcctgcctgattgccctggccagaacttccaacactatgttgaataggagcggtgagagagggcatccctgtcttgtgccagttttcaaagggaatgcttccagtttttgcccattcagtatgatattggctgtgggtttgtcatagatagctcttattattttgaaatacgtcccatcaatacctaatttattgagagtttttagcatgaagggttgttgaattttgtcaaaggctttttctgcatctattgagataatcatgtggtttttgtctttggctctgtttatatgctggattacctttattgatttgcgtatattgaaccaagccttgcatcccagggatgaagcccacttgatcatggtggataagctttttgatgtgctgctggattcggtttgccagtattttattgaggatttttgcatcaatgttcatcaaggatattggtctaaaattctctttttttggttgtgtctctgcccggctttggtatcagaatgatgctggcctcataaaatgagttagggaggattccctctttttctattgattggaatagtttcagaaggaatggtaccagttcctccttgtacctctggtagaattcggctgtgaaccatctggtcctggactctttttggttggtaaactattgattattgccacaatttcagctcctgttattggtctattcagagattcaacttcttcctggtttagtcttgggagagtgtatgtgtcgaggaatgtatccatttcttctagattttctagtttatttgtgtagaggtttttgtagtattctctgatggtagtttgtatttctgtgggatcggtggtgatatcccctttatcattttttatcgtgtctatttgattctcctctttttctttattagtcttgctagcggtctatcaattttgttgatcctttcaaaaaaccagctcctggattcattgatttttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtgggcatttagtgctataaatttccctctacacactgctttgaatgcgtcccagagattctggtatgttgtgtctttgttctcgttggtttcaaagaacatctttatttctgccttcatttcgttatgtacccagtagtcattcaggagcaggttgttcagtttccatgtagttgagcggctttgagtgagattattaatcctgagttctagtttgattgcactgtggtcggagagatagtttgttataatttctgttcttttacatttgctgaggagagttttacttccaactatgtggtcaattttggaataggtgtggtgtggtgctgaaaaaaatgtatattctgttgatttggggtggagagttctgtagatgtctattaggtccgcttggtgcagagctgagttcaattcctgggtatccttgttgactttctgtctcgttgatctgtctaatgttgacagtggggtgttaaagtctcccattattaatgtgtgggagtctaagtctctttgtaggtcactcaggacttgctttatgaatctgggtgctcctgtattgggtgcatatatgtttaggatagttagctcctcttgttgaattgatccctttaccattatgtaatggccttctttgtctcttttgatctttgttggtttaacgtctgttttatcagagactaggattgcaacccctgcctttttttgttttccatttgcttggtagatcttcctccatccttttattttgagcctgtgtgtgtctctgcacatgagatgggtttcctgaatacagcacactgatgggtcttgactctttatccaacttgccagtctgtgtcttttaattgcagaatttagtccatttacatttaaagttaatactgttatgtgtgaatttgatcctgtcattatgatgttagctggtgattttgctcattagttgatgcagtttcttcctagtcttgatggtctttacattttggcatgattttgcagcggctggtaccggttgttcctttgcatgtttagtgcttccttcaggagctcttttagggcaggcctggtggtgacaaaatctctcagcatttgcttgtctgtaaagtattttatttctccttcacttatgaagcttagtttggctggatatgaaattctgggttgaaaattcttttctttaagaatgttgaatattggcccccactctcttctggcttgtagggtttctgccgagagatccgctgttagtctgatgggcttccctttgagggtaacccgacctttctctctggctgcccttaacattttttccttcatttcaactttggtgaatctgacaattacgtgtcttggagttgctcttctcgaggagtatctttgtggcgttctctgtatttcctgaatctgaacgttggcctgccttgctagattggggaagttctcctggataatatcctgcagagtgttttccaacttggttccattctccgcatcactttcaggtacaccaatcagacgtagatttggtcttttcacatagtcccatatttcttggaggctttgctcatttctttttattcgtttttctctaaacttcccttctcgcttcatttcattcatttcatcttccattgctgataccctttcttccagttgatcgcatcggctcctgaggcttctgcattcttcacgtagttctcgagccttggttttcagctccatcagctcctttaagcacttctctgtattggttattctagttatacattcttctaaatttttttcaaagttttcaacttctttgcctttggtttgaatgtcctcccgtagctcagagtaatttgatcgtctgaagccttcttctctcagctcgtcaaaatcattctccatccagctttgttccgttgctggtgaggaactgcgttctttggaggaggagaggcactctgcgttttagagtttccagtttttctgttctgttttttccccatctttgtggttttatctacttttggtctttgatgatggtgatgtacagatgggttttcggtgtggatgtcctttctgtttgttagttttccttctaacagacaggaccctcagctgcaggtctgttggaataccctgcagtgtgaggtgtcagtgtgcccctgctgggggtgcctcccagttaggctgctcgggggtcaggggtcagggaccacttgaggaggcagtctgccgttctcagatctccagctgcgtgctgggagaaccactgctctcttcaaagctgtcagacagggacatttaagtctgcagaggttactgctgtctttttgtttgtctgtgccctgcccccagaggtggagcctacagaggcaagcaggcctccttgagctgtggtgggctccacccagttcgagcttcccggctgctttgtttacctaagcaagcctgggcaatggcgggctcccctcccccagcctcgctgccgccttgcagtttgatctcagactgctgtgctagcaatcagcgcgattccgtgggcgtaggaccctccgagccaggtgtgggatatagtctcgtggtgcgccgtttcttaagccggtctgaaaagcgcaatattcgggtgggagtgacccgattttccaggtgcgtctgtcacccctttctttgactcagaaagggaactccctgaccccttgcgcttcccaggtgaggcaatgcctcgccctgcttcggctcgcgcacggtgcacgcacccactggcctgcgcccactgtctggcactccctagtgagatgaacccggtatctcagatggaaatgcggaaatcacccgtcttctgcgtcgctcacgctgggagctgtagaccggagctgttcctattcggccatcttggctcccacctcccgaactcatccttttttatggctgcatagtattccatggtgtatatgtgccacattttctttatccagtctatcgttgatggacattttggttggttccaagtctttgctattgtaaatagtgctacagtaaacatatgtgtgcatgtgtctttacagtagaatgatttataatcctttgggtatattcccagtaatgggattgctgggtcaaatggtatttctagttctatatccttgcagaatctcctcactgtcttccacaacggttgaactaacactctcaccaacagtgtaaaagcgtgcctatttctccgcatcctcgccagcatctgttgttgccagactttttaatgatcaccatcccaactggtgtgagatggtatctcattgtggttttgatttgcatttctctaatgaccagtgatgatgagctttttttttttttttcatatgtttgttggccacatacatgtcttgttttgagaagtgtctgttcatatcctttgcccactttttgatggggttgtttttcttttttttctggtaaatttgtttaagttccttgtagattctggatattagacctttgtcagatggatagattcagatcagagctttaacagccagactttacagggaaaaggctgagtggcacctcctttatatgaagaaaatgttcttctccaacccacttgtgttcagtggatggaagaagctgttcagtaattataactgacacttgtaacacataactgattgatttaatgcccataaattttattttctttaatcgtaagatgtgaccgttaaattcttagaagacatttggctagtggattaataattaaaagaatattggatgaattcatctgcttcctattcagcagttcttttatttataaggctatttttctcaaaatgaatgatcatgaatatctaagaaaaaacaaaatatttagctgtttgaagatgaaattattatcttttgatgttgttttctttaaagtgtttacaatgataactaagtggtagggcttcttccctctgttactctaagtatgcctcgtatacatcctcaaagcattttttttttttttttttttggagacagagtcttactcggttgcctaggctggagtgcagtggcatgatctcagctcactgcaacctctgtctcccaggtttaagcaattctcccgccttagcctcccaagtaggtgggattacaggcgtccgccaccatgcacggctaatttttgtatttttagtagagacggggtttcaccaagttagccaggctgctcttgaactcctgacctcgtgatccacccacctcggcctcccaaagtgctagattacagacgtgagccaccacgcctggccacctcgaagcatttgtaaaagagcaaggtgatggggttcattaggtgtttggtatttgaaattaaatacctctgttattgatgaagccatattttaaatattatcaccactaaactcagttaaatatttccactgttaactacaaggttcaaatgcttggatattatagaagatatttgacttcaaagataaacgctacaattttgggtcataacaaatagcttttagaaggggaaaaaataagtcttgggaggaaaataagcagtctcttagtttaatatagcggttggcaaatatagatgcttaaaagttctgaaaatccccattgcaggccatcagagtgttgtaattaagtattaaattaattcagataaaaggataatccatcagggccactaatgtagtggaagtgtggtttcggtgtgattagtctcaccatggaagtcaaagaaagtcccactcagaatgaacaatgaaagaatcatatttgtctctgggtattttgtcataggctcaattgcccagtgccagtgtgtaagccactggtaaaatcctgagcccttagtcatgtctacaggtagctggctgccttagacattttatgatgacgaaattgaattccacttctgataatggtaatccatcatgtctacctctttttttctcttaaaactacactgacggccaggcgcagtggctcccgcctcatcccagcactttgggaggccaaggcaggcagatcgcctgaggtcaggagtttgagaccagcctggccaacatggagaaactctgtctctactaaaaacataaaaactatccggtcatggtagtgtgtgcctgtaatcacagtactcgggactgaggcaggagaatcgcttgaacccaggaggcagaggttcgttgttctgaatgacatagccaggggtggttatttctaaattttctgtgttgaattaagataggaatgtggatttcttcctattttgaacaacgaccaaaaaaaaaaaacgaaggctaagtgtgtttcactctgctaaaaatgtaccttttcttgaatctgtcactttttcacttacattttctgaaaatgaatataattaaccaccatggggctcagcatgctggtggccatttggaggaacttttctaccaacagtgtaacagagaaaagagaaaagggtactacagcgtacctcaacctcccctgactgttctgtgaagtacaagtgtaagatttctcactgtgatcttttgctcctctgcctttttactgccaagggctaagatgggcacagggccctttccagctgcccctgcctgagaaatgcttatttgatgaccaaaagcaataaccattccataggactaacggtgattaagtcagcttacaaatccacaagggtggaagagactgctaaagatattggaacagacatgcttaataatgcctaatttctacagctacttttgtgataatacgagaggctgaaatagataggggctgaacatgggaaggctggtgttatgaagcccaggacttggatttcagcactggtgcttactggccattgggtcttgaagagattggttcctttctcccagcctcagtctccttgtcccatcagattaataatgcctgccttgggagaggagagtcatacaggagagggcatttgagctgggctctaaagagttcaaataacgtagatgagcagagatgggcttccaagatagcaggaatagaaaacaaaagtgtcagaatgggtatgcatggtcttttgtgggaataccccaagctatttagcctgagagagaggtgtggtgtagacagctggagctgtgagttccatggctagaaatacggggtgggcccagaccacagagggccttcagcatctggcaaaggagttggaattaattcggtagaaaatgaagacccactgaaacaaagagggagccattcttgtgtgtcaggaactaagatttcaggaaccaaacgtaaaataaaaaccagcagggagaccattgtctcagtcggtttgtgctgttatcatgataccagagactgggtaatttataaacaaacaatagaaatactacaattctggaggctgagaagcccaggatcaagccgggtgcagtggctcacgcctgtaatcccagcactttgggaagccgaggcgggcggatcacgaagtcaggagatggagaccatcctggctaacacggtgaaaccccgtctctactaaaactacaaaaaattagccgggcgtggtggcgggcgcctgtagtcccagctactcgggaggctgaggcaggagaatggcgtcaacccgggaggcggagcttgcagtgagccgagattgcgccactgcactccagcctgggcgacagagcaagactccgtctcaaaaaaaaaaaaaaaaaaaaaaggcgccttgtggctgcatcctccagaggcagcaaaaactgttctcacatggcagatgcacataaaggggcctgtgctggtttcctctagcccttttagaaggcagtagtccattcgggggcacggggtggaactgtcatgacttgatttccagaagaccccatctctcactactaccacagtggagatttaagtttcaacacatgactttgggggacattcagaccatagcaagcagtgaggagactatcgtaattatccaggcgaaaagtaatcatattcctaggggcggtgagggtggtgtggaaaagcaagcaggaaatggctgagagaattcagaaatagaatgcaaaaggatttgtcggtagtgtgagtactggacacgaagaaatacagggagtcctggatggctctgaaggattcaagcctgtgactgggagggagaatggttctgccatgattagaaaacaggaaaggtggaagggaaagccagggaccttgagtacagttgtgtacctaccaagtgcatggatcaggagggagccctaagttggtatctccagcaaactgttggaaatatacaaatttgagtgggagggagaagtcaaagctggggaagtacatttcagattcacccaaaggtaggagatctttagagtttcaagagtcgatgggatttccaaaggggagaattgtagaaagagatggaaagagatacaagaagaaaatgacagggatgaagagaggagaaaaaaagagtaaaaagagttattcagagatgtcacagggaagaagataagtactgtgtatgcgaaacagtgagaacatttccaaataaaggacatggtaagtgatagtagataccacagacccaagaacagcgtagcccctggggagttcaagagctttctcgctggcaaaggttatttcctgtcaagatgaatgtgtatgcaccaataattattagtattccagaatatcctctctttagcatagtaagtgcttaacagtcacaagcaaaagatttatataaaagaaatgctcatttaatttatttaatcagacattggctaagttattagccgctacctatggctttgtaaaacatgaaaaagttggaggaaatctaatgagggtcttttatctgtaagggaaaggtcatcatgggcatcaagtattgaattttagaaacggtgccacagtgaatagggatatattataactcagattagacttcaaaaggactaatttgcttgggacttccatcatttaaaatcacttgcaataaaactgactacatgaaaagatattaatcacaagctttaatgctattccatcaaattattttaatgggtatatttgaacagaattttaatttttcagatacattttttgtcaatagaaaagagaatttgaaagatgattgtaaaatgttacaaatcaaatttaaatcctgatcttattttcattttctctagcaataaaactaaccagtgatgtgtctgaaatatatactacagcaaactcttgacacagctatgattggaaaatgagacattttggttaatcaaatattattgttaatagtgcagtgctgtctgtcggtctctaatttgtaaaaatcagagaaaaacatggtgaatctttggtgacataagagaagactcacaaatcttcatatcctggggtcagtttttaaagcagtaattacacatgatacaagattatattctaagctctctttttccagtaagaaaagataagtcaatgatgttccataaagctgattcttgtttaatcaatttttaaagtgggggggtaggcggcactgacagcattctttggaaagggaagcaacagaggagaaaggcaggatatttggtgtctgctcacattgggaaatacggggagccaggggtggttaagtccagagaaggctggatgtctgtctgcagggctacatctggaagggcaagggaacagtttccttgttgcctcagagcataggtcatgcagcacaagaggagcatttaggaaatgcagattttttttcaaggcaagacagtgaaaactattcaagaaggtagcaaactactggccctttaatgcttttcttatccttgcaagtgtgcaaggcaagacaggagattgtctgttgtctttcagcttgaatgagattgtgcctcacttactgccgccacaccaaagcctggcgtttattcctgtgcgatcattgtatactagggacgtctaactgtgtcaggaggaaatggcagctgagcccctgtggcctttcacctctagcagtgccacgctgcagcacattggccggctgccatgctcctgcctgcagtcctgccagcagctcttgtgagctgcgactttgcataagtagcttgaatgccatgtgcctcagttttcacatctgtaaaagggagatgataatggtacctatgtcatggctctaaacgcgatcatgcacgtgaaagcagttgaagtcttgcctggcagaagtaaatggtggctgctgctgctgctgctgttgtgattgttgttactcaccaaagagatggttttgtttggtttagatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggtaagagtcccctgagccagcaagggcgttctgggaggtatatatacacatacataacgtgtgtgcaagagagagagttatcttttgtatgttcttgagtggtgaattttttttgatgtagtcataaatattttcattataattttactttaaaaagtaaatactaggagttactatgtcattaaaataatatatctgtgtaaattattctttatattttttatgctgcaaagcatgtaaactgccaattgttggaaaagacaacaactccattgtataggtggcaaaacaaaatggtaatgatttacataacatttgcaggtaaagggcctcttgtttcgcacatgtccaggtttgcattctagggaatattatccttcgagtgaacactcccggacgaaggggagccacagtgtattattgaagaggcagttcttcacaaggaaaatgtgtggcaataacattcttctaatttttattttccagttcctcttcttaatataaaatggtacattatataacaaaagtggatacataaagagcatgtactgtatatcatattattatgtttatgactttttcaaatgaggaaatggaggcaccaggagactaagttcttaagtgatgacttttggtaggaattttttagaaatgttttttaactgtgaggtatattgttccctctggaatggaagcacaaaattcaacttgtgtttatgacgtaatgacctagaatgccaccttccaaaaaagcagatagatagtgaaataaggaaaaattacagtttaccaacagagaggagttttgagggcctgcagagatgcagatttgacaggcaccaaagtagtcttcactggctacttattcttacatgaaatttcagattgaattaaatgttaaaggcaaagatagagaactacatataaataaaatttaattttgcttaagacttaatttgtgtttagaaaattaaatttaaggcagctgccaaaattaaggagcacattgctctaaatgttttttcctttggtacctcagagaagtaagattcttctctatcaacaaatttgagcagtagatggcatcctttttcacagggatttcttagcagaaacacctgttcaggcaagaatatctttcagcttcttttatctttttctacatgttcagaagtatacaattacacacaaacaccttgatttctagatacaaataacatgttttttaagaacacaacagcactttacctgattttctttatcaaagcagcaaatgttgatggagatttcatatttataatataccttataggccaggcatcgtgggtcacgcctgtaatcgcaggacttagggaggctgaggtgggcagatcgcaaggtcaggagatcgagaccatcctggctaacatggtgaaaccccgtctgtactaaaaatacaaaaaaaagttagccgggcgtggtggcgggcacctgtagtcccagctactcgggaggctgaggcaggagaatggcatgaacccgggaggcggacgtggccagtgagctgagatcacaccactgcactccagcctgggtgacagagcgagactttaaatttaaaaaaaataaataaataaagtaaaaaatatatataccttataacatgcaattagtactaaatctaaatataatattagaccttaagtgtactggacaagttttttctaaagaaccattgcagaaaatatatagactattttcacaatgtgaatcatcaccccaaatttgtttccttaatttgaaaaagggcaccttacagtaatagcaaacagcatgttactgacaatgtaaaaataattaaaactttaccatttatgaaaataattcttaatcacatagaagaaaacaattttagggacagttcattatcaaagacctggaaaactgtcctgttaaatgtattgagtgcagattgttttgtttctttacaggtagatagattttggtcattgaaggtcctcttcattcagtctctaatgagaatttttagtgaaaaatgtccgatctcattttgtaaattgaatgcacgctaaatttgctcttttactaacagcataccttgttgcatcagccttaggagaaaatctgcgtgcacacgtgtgaatgtacacacacacacatacactgtgaacccttggtaattgccaggatgtccatttgagcaaagcccttgattaagtacttctggatacttgacaaacacgcagctattacattgcctaggtctcatgaaaagagctgatattcttgttggatgattctgtttgaacagtagaatctggtataacaattttgggaggcaatagaacttgcagtgtcagagtcttgagttgcagtactcttcattatcagtcccaacattgacatatatatattgattatagcaaacaatttaaaaagccacttaagcaatttttttttttttttttttttttgagacggagtctcgctctgtcgcccaggctggctggagtgcagtggcgcgatctcagctcactgcaaactctgcctcccaggttcatgccattttcttgcttcagcctcccgagtagctgggactataggtacccgccaccacgcccggctaattttttgtatttttactagagatagggtttcaccgtgttagccaggatggtctcgatctcctgacctcgtgatccgcccgcctcagcctcccaaagtgctgggattacaggcatgagccacggcgcccggccccaattggtttttaaataaaatagacggccatcatctcataggatattaaatactaccccatacccctgaactcctgaatgatacagacccgagtttaaattccagccctgctatgtgcttctgagcaagttacttagcctctctggctgcttcctcgtctgtaaaataaggttgacaataattcctaactcagaggtatagtatgagcattaaataagaaggcataaaaaagctcttagcaaaattcctggaacgtagtaaatgtcagtggtagctttttttttttttaatggagccaggatcttattagagagactttatcagcctgtctcaagatgaatcaccttatggcctcaacaatgtatccttaaaataatgaatataataagccatataatgagatacagtcctaaagccgtctctcttctaacaccgtctcccacctctccccagaaacttgttagtctttctttgtacagacatgtgcacacacacacacatacacccaactactgaaaatatctaaaattctcaaaggatttccttttgttcttcatgcagtcctcattatcatagtttcaaatgcttaccaaaaatttgttgtttttcttaagaacaatcagtgaaatccagaggaggttctgatgcccattttttaaatttacattgaagaaaataagcatatcagagttttccaatgtgctgtgatttgccttaacaattttttattctttggttttcacatccacaaaacctaaatgaatggctaggctgaaatgaagctgttgttacagaattgtgctcacagatcgtgaaatgatctggctggaagcattagcgttgatgacatttgtgtttgttctactttttcctgactgcctgagaagggcagccacaaagagcagcatagcatgcagtgactgccagcaagtctttgtggccaacagtctgcctctttgtagataatacctagatataatatgtagataaaataatatttttaaagagacaaagcaggtcttataagacttgtatggatgtatttgcaaaatgtgaagctgccttctagtatatttttaactagctttatagaggcaatgaggaagccaatttaaatgttgaaatttagattaaataaatatttcattaaacttacaaaccagttcttgtcagcctcttatctatatgtaaaactggtgtactgctgattcctcattacctttggcctggtagagagtttgtacaaggcagcacggttaatagttgaggtttattacaaagaataccagaagtaacagactcaaaaatcccagaggtctgaccattctgtttctctcctcttacagcaaaggatttgattcaaagctactgagagcaagagaataactcatttctgcttacattttattttaactctattaaaagttaaagctaagataatgtatttcttgtttttaaattactaaacagccaatgtaagatgttttttagcacttcattgatgaagttactgtctggttaagtaaaggtcaattggaaaaattggaaagtatttgcagtggattgggaaataacacactactttgttttgttggtgtgtattcttttttttcggcaatgatatcataattgccttttaaaaaacttttaatgtctgctccctgagctacttattttttgagcattaatttatactaaatcagcttcaatcttttaaagcagattagggtttatgtcactgaaactagttttgtttaatcttcctaaaaaatagggttagggttttattattgtttttaaataatacatgcctatttaaaaaaatagaagaagaagaagaagagcggggagaggcagagaaacttataggacaatatcatttgtaagtttctttcacataagtatacgcatttgaaccctggaaaaaccatttactttgtgacacaactgaagttcagaaagaatgtgattagccccagatcacagagaaagtaagtggtgacattagcagtggcccgtgggactaccagctcctcagcctgcatttaaaccacaggtacacttaacttactgccccgagttgcatatacattttctaataacgtaactattgatgtatgtgctcaccaccacttcaaggtacgcgtttgccctcgtgcatgtggtctgttgtgttcttaactgcagttgaagcactattgggtccctcatcatcaggaaattctcacaaagtacaatgacctttcaactaactgcattttacaaggagtaatacagttactcattacaggttgctgcaaataggaaaaaaaatgaagtatagaaaaaccaaacctcattgtcgaaatgtatttgggagttgcagaaggaagtgaactatgtcttcctctaatgcactagtgaaatactagagaatagtcactctcgcatcagtagttactatggaatatttaatcataggatatttaatcagtgcagttctgtagtatctggtagggtctcaaacaggctttaaaggttacttatttgttcctcaccataattagaagatgcagatgttaacatttctgtttccatatggagaaacagccacagaagtggcgcatcccaaggccacagacattgtaaatgttgggcccaggcctgggattagatgcctcaccctccttttgactccaccatgctgccagtctctacatgagaaaggaccaaatttttatttcatccaaatatcctagtgctcccggaaatattttcttatattttggagacttggcttactgccactttttttctccctcactgtttctattaacatgtggcatccaaaatcgatttggactgagaatcccacctttgaatgcctggcctgtgtgcatcagtaagctggatggttttgctgaaattgttagaagccatttcatcgagcacaccgtgtctccttcagcacctgtgctcactttgtcttacctttttccctttacacttgccaccttaccaaagcagcttccccctgcacctacctccgtggcttcatttaccctatgaccattattaatcctaaaaacttcacttttgtccccattccttgtttcttcaaacgccagcgtaaaacaaggaagtattttctgagcatcagaagtgctttggaaaggtcagatgatggtgaggaatatcatgctgtccacactgctttctttgggtcggacccagaacacataaaaagggaaaattgtgaacaatggcaaatataaaatgaaatatcctcatttcacacgtttcagctgtatgtcatctgtcaaatggaagccagcctttttgaaaaagtgagaaaaagactgcggtttccctcttctgccctcatactaaggacaaatttgattcactcactaaatggcagattatatagtaaaactttgctctcctgataaattcatatttgcacttcccctggaaatttaacagagtccttttgtagttaattaactgtaaaataagataagattgggcagttgtaaaaggaagtatctttaaggttttctgctaggctgggaatattttatttctaatcaaaagccaagaaactatctctaaactagctcagagggaaatccaccctccccacccctcttctttgatgttgctgcgtatcctggtgagggcttttattagaaaactaaggagcaggactggcacatcaatagcgataagccatttaaagtggtgcccctcctggtcctcgatgctcgggattaactcactgtggttcttttctggctgctccttttttgtaacttgtttattgcatatgcttttttccctgctcgactatgtttgggagccacgacttaccatcttgatttgtcttgtttgctttctgtgtcccttgcttcctgtgcccagttgagcctgttgatgcccgccctgctgccgaccgaggactgaccactcgaccaggtatcagaaccgcttgacttgtgcctctccgcatcttgggcctaagctcgagcagacgttgtctcctttctgcattttaagcctctctcaattgtacttactgcagagtagaatttgaagtgaccttagaaaccaaacttttaaaaattccccttgactattccttactcttcatttcccaagttccgagtgaaatgattttttttgtttctgagctttccctttgaaacctaaaagagagcccttccaaatttgggtttttttttttcttttttctttaaaacaaacaaaacaaaacaaaaacagagacgattatatacagcctgtccttaaaaacagaagcccagatagatgtagactctcaggtgatgacttggataccagtgtgaacatctctcctttcttgtttggtgtccagatgggtaattttctaactgaactgggctcaaacttcaatcagttactgaagtagacatgatttcctgaaaataccagaattggtttctttccagagcccaaaatacatcttctgttttgtgggttgtatgtacttattctcaatgagtggctaaagtaacacaacagatgtggatgcataattttgcacataagccctttttttatgtattacttaaaaatcccattaacactgaaatagacacttctttttttacagtttcacaaacattaagaagaaacacatttttattttatgaataatgtcatagcacagcagaagcatatatgtgcctggtgtctactctatgaacaacctcacccaactgatagtagccagtgctttcagtgaatcaaaaagaggaagaaagttattgaattgacaaatttgtgtattccagggagattttaatcccaaataacacatacttttccatatttattttcttctttatcctcataatgtccccaggcgatagtcaaagaacagggaattaactttctttgaagatattaatacacagaggtcaacctttgctgcctggttcagatacagtcagcagcagaccaggagacccaccctgtccctcacacttccaccacaggctcgagccatgactgcctccaagcacggtcactgatgaagactgagagtctgcctgtggcatcttcatgcctttatgctgtgattaacagtggcagcagagacagtagtgtgacacgtcttgacagctggtctcgttgatttatgaatttctgtctgtaggcttttaaggggaacttaaattatagtgagtcagagacagtttcagttcttctaatgtcctccttattgtcaatgtcatgtaagggaatctcaaggagccaggagttgacattcctgttctgctcagccaacttgtaaggcctcgggctgagggtcagttcagttctaattgcaaccacagccagctcaggttggtcatttgccttgggacttttttcatggtgcttctctttaaaagacatgctcttcatctacaagcataaaatgtaacttagaaagcgctcattcgactcattcaattttatcaaatttctacattgggccaggagagcctatatgttactattgaaacatccaggaccaggtgacactaccagtcaatgtttttttcttttttaaaaaaattgttttttgattttttttttttttttttgagatggagtctcgctgtgtcccccaggctggagtggagtggcacgatctcaactcactgcaacctctgcctcgggggttcaagcaattcttgtgccccagcctctcaagtagctgggactacagatgcgccccaccatgcccagctaatttttttgtattttcagtagagacgggatttcaccatgttggccaggctggtctcgaactactgacctcaggtgatccacccacctcggcatcccaaagtgctgggattacaggcataagcaccatgcccggcctattcttttaaaaaaaattttttttaatctaaatctaacaaaaaggacacctccaaacgctgtcaaacaaaaacagtgtgttttaaaggaagagctagagaatagttaatggtattacactggtgggagaaaaccatccaaaacctttctcataacagtaagaatatgatgtcacttggggatttcacaaagatcaatccactaaattctttctaggtttgaaagaaattaaacggtagggtccctgctctcaggagcttgaggtataatagaaggaattttgcatatttggaaacatgagatggaatttggaagacaaggaaatacaaagcaacattaagtgctaacaagaatgttctgtgatgggaggagatggagtactaacccaggggcatgttaagtagagaacttactggagatgggcttccagagaggttatgaaagatttgaggcacctggattggcaaagaagaagggaacaattctattcagaagagagggtggcctaagccaagcttcctttcatacacagcgtaggcttgggttgcccagtaagatggctaaagttgagaaagaatgctcagatatgaagtgaggaagattctagttaaggtttgtctatgaatagcagtatttactgcacatgtattaagccatgcgtgtgttctgtttttgtgcttgtagtaagtttatcaaacatttgggctaatttccccgtgttttgtagcatagctttggttcttcttcacattttagagaccacccacttgagtctaatgtgccgaattatatctttagcctttccaaacttagaaagccttcccaagattgtcatgccgttcaagtgatggcattgtttttgaataatcactgttgtcctcagaaacaagttaaaaaaaggtttttgtttttcccccacccagtgctttataatcatgactgaggaaaatggcttctccttaactctcatatctaaaaaaacagaatatggtagcttccagcatgcagtgtcaagtagggaccattgtaataacagtggtgatttgggggcatagtggccattatagctcctcctcctggcttacttccaaagaaaaaatagactctgaaggactaattatacagtttgaccagaataaaaaagaaaaacacattagcgagctcttcacatagggtagaaagctaccctttctgttacaattcatcaccacccttctgcagtccaaataaagggaaaccaggtccttgacccgagccatgcaagaggtgtcatgaaggacatacctcccttagcccccacatgaacaaaaggctctgtccactcatcatgacttacaggaagagagagctggagtcaccagggcttagggtgaaatcagcacaactcaagaaagacctctaagtaagcctgcaaaccaaggtccctgccatttatttgaaactactcatcacattttattacatgcattatcaccttattatgaatatttccctatgtgccagcttactgattttgtcttttagtaagatttgcttaatgtaaaaagtcttccctagatgactattcatgctaaacaaacatgaactattatagcaagagctcttctgccaagagcttctcaaactattgagctgtaaacatgaatttatctgtcatgctgacttgataatatgaatttttttttttggaccaccaaaagtattcacagtatcatttcaaaagtagtgtgggcttgtaaagtttttagtggagagctcttggttttgttttttttttttttttttttttaaaacaggatttgtctctgtttccaggctggagaacagtggcatgataccagctcactgcaacccccatttcctgggctaaagctatcctcccacctaagtttcccatgcagctgggattacagggatgtgccaccacacctggctcatttttgtatttttagtagagacagagttttgccatattggctaggctggtctcgaactcctgagttcaaactgtttgcccgcctcagcctcccaaaggctgggattacaggcatgagtcactgcacctggccgagagctcattctttttttttttttttttgagatggagtctcactctgtcgcccaggctggagtgcagtggcacgaatctcggctcactgcaagctctgcctcctgggttcacaccattctcctgcctaagcctctagtagctgggactacaggtgcctgccaccacgtccggctaattttttgtatttttagtagagacggggtttcaccatggtcttgatctcttgacctcgtgatcctcccgcctcggcctcccaaagtgctgggattacaggtgtgagccactgtgcccggccagagctcattctttacttttatttttcagtataaatatttggctccaaaatctaaaagggaagactcttcaaactgttcaagaaagttttcttaaaaatctcagaaatatagttctaacctattgtgttgctagatcttttatttatttatttatttttggagacagagtctcattctgtcacccaggctggagtgcagtggcacaatctcagctcactgcaacctccacctgccgggttcaagcgattcttatgcctcagccttctggagtagctgggattacaggcacccgccaccacacctggctaatttttttttttttttttttttttttttttttgtatttttagtagagacagggtttcaccatgttgaccaggctggtctcaaactcctgacctcaagtgatccacccagcttggcctcccaaagtgctgggattacaggcgtgagccaccgtgcttggcacgttgccagatcttaataacagaaaaggagtgagctctaaagaaatcactggttatataaggatctcagatttaaaagggctatagaaagattaaaatatacagccagtgagaattaagagagttttagcatcactaccctataaaaataagtgtcagaaaggttcgtttcagcagaatgagttgaaacttaaccaaaaaatacttaacaatattaaagtaagaaaataaataagaatagagaactctgtgggtctcgtagcaagaagttgggggttgagggttaggtcattgagaggaccagagttaaagaatgagaaagggaaactgtcccacttccattgtgccttttatgtctatattaaagagaatgaacttctgatgaaaggaaattaaagcccgagatagatcaacatgagaaaagaaagccctagcccattagttacctattgctacgtagcaaataacgcgagaatttagcagcttaaaacaacaagcattagtttcttacagtttttataggtcaggatttgggatgtggtttagctggatggttctggcacagtgtccgtcatgtggttgcagttaagacactgcctgagctaaagtcatctgaaggtttgattggacctggtgggtctgtttccaacgtggctcactcacgtcactgttactaagagacctcagtatcttaccatgtgaactctgtaatgctgcttgagcgtccctacaacatggcatctcgcacccctgcaccaagaggtccaggaaagagattaaggaagttttagtgccttttttggcccagtctcagaagtcatacaccatcacttctatcatgctctgttcactagagcaagtcactacatcccgaccacattcgaaggaagggaatcaagttctccctcttgaaggaaggagtatcagcaaatttgtggatatattttaaactatgacatcttgctacccaagtccaagttcttatgacccagaccaactgcaggctaagagaacttgaaggggaagtagcaccattgcctgtggtctttgtggaaagctgggaaggtagggataggaaaatgaagttctgaatagcaaaaaggagagaagatagcttcacaatctgtaagccaggtatagagtgacattgattgtgggtctcttttcaaataggttatcaagtggaagctttttgaacaattggatgaggaaatggtgctaggtcacaaccagcgtggatttcctaggttttgtcagattatccttaccttctccttgtataggctgtgctcttagactggcacatcatatgctgtggacattgtgcttttcagtgtcagcaagatgtgtcacaagtttcccagaatctcgtagacaagattgaaagagtgattaggccaagagcaggaagtgaattcccctgggagccaaaggaattgggaatgtgtagcccaagtaagacaagaaccagcaggaacatgcctctccttagggtcgtgatacctgttcaaggttttaatgtggaagggaggattaggcttgctctgtgttgaatcaggctcaaaggatggaagttacagggaagctgattctggcttcatgtaaaaaaaggacagtttgggcaggcaaatctatcaaaaaatggagggaaattgatacattcctctatgttcaaacaggaactgacaatctgcccctgggtgggaacacggtagagaagatgacttcaaaagcccttttcatcctaaaattctgatgtttgataattaaatgttatagcatggacactgacatttacattttttacttatgtttttggtttttaaatgactctgcattttgttttaagcttcaaattattatttgaataatgaaattcatcagaacaattagtgttaagaatcatatagcaatttatagaaaaggaagagttcgtaggttataaattctgttagttgctaagaagcatttttaaaattatgtactatagctctttattcagcagacgaaccaattacaatctgtgtaactagaacacttgactaaaattatataatttttacaacgcttcactgcatagatacatgaacataatttatttgtaattggaacaaagccccaaagtagcagttttgttctaccaggtaattaatgctcatttttaaagccttttattattatttctgaagtaatgagtgcacatggaaaaagacacataataggctaaacaataagcccgtaagccaagccaacatattccaggaacaaatccttgccaacctctcaaccaggatttaacttctgcttttcccccattttcaaaaattatagcatgtatttaaaggcagcagaagccttactttcaggtttcccttaccctttcatttctttttgttcaaaataggtagtaattgaagttttaaatatagggtatcatttttctttaagagtcatttatcaattttcttctaacttcaggcctagaaagaagttttgggtaggctttgtcttacagtgttattatttatgagtaaaactaattggttgtcctgcatactttaattatgatgtaatacaggttctgggttgacaaatatcaagacggaggagatctctgaagtgaagatggatgcagaattccgacatgactcaggatatgaagttcatcatcaaaaattggtacgtaaaataatttacctctttccactactgtttgtcttgccaaatgacctattaactctggttcatcctgtgctagaaatcaaattaaggaaaagataaaaatacaatgcttgcctataggattaccatgaaaacatgaagaaaataaataggctaggctgagcgcagtggctcaagcctgtaatcccagcactttgggaggccaaggcgggtggatcacgaggtcagaaattcgagaccagcctggccaatatggtgaaaccccatctctactaaaaatacaaaaaagattagctgggtgtggtggcaaacacctgtagtcccagctgctggggaggctgacgcaggagacttgcttgaacccaggaggtggaggttgcagtgagctgagatcgtgcctaggcgacagagcgagactccatcccaaaaaaaaaaaagaaaagaaagaggctgtatgtatagttctttcagactacaaggcagcaaagttcgtgcatgactcgggacttaaagtggaattaatttcaatatagcagccactttgacttccactgtgttttctgggaaaataggtttacaataggtttatttgaaggatcaaacacatgcatacactgcttggttttacagaacactttatgtggcttaaattcacatccggaactgtcttcctttacccattcatttctcccccagctctttcttttcattccctcccctacctcccatgatttaacttctcttgcaagagtaagatcatggagtgagcaggaccccatgatgttcccgatagtgttattcatcaaaaggtttgtgcaaagaagacagcagcttccttttcagatgaaatcacttttcccccctaatgttagaattggagtaaatcaaaaagccacatctcctttgtggtcagctctagtagttatataaaatcctttaccaaaagcttagaaatggagataaatcaaatcgtggattatgttagggttccatcttatcagtaggtgcagtaagagggttaaattaatgaagacgacaattttatcacattcagtggtggacagaaaaatggtaagaaaatttccatagcaataatacttaaagttatctcaggcacttcttttgttttgttttgtgtgtgtgtgtgtgtgagtgttacttttttccaagcagaaaatgtcttttcaatattcataaagttgataaatcctagtattaatctctaaaagaaacacctccaaattattatttatgccttacttgactccaaataattgtagcaaataaaaaactgacttgggatttggatttgcattcttaactcccatagttctttttttgtagaaagacatttagctttttgaagcatggttttcattggcaagataatctagtatcagttgttataagatcagggtttctcttgatgaggctgttgctgaagaggttaataaaaactggggaaccactaaagagttgaagagttggtggggtagaaagctgacgattaatgtacagatttgcatttgtcggggcctggggcctgtgtcatataagcccatccccacaattacactaacgcctataatgcgacagtgactaatggcagcggcagcaattaggagaatcagctccctctactggactagttaagataatgtattataattagtgcaatgaatattacaaaattacagtattttcttaaaggcacaggcatatgtccagacttgtatttattcctgattacctcacactagtatattagctaattaatgatttgcttttcataaaaatgttgagctagcatatttgtttagtaaagggaataattatgaacaatttctcattttgttataaaccacgagtaaaacacttttagaagttgcttcatttgctatattttatattgcctttccagattgctagtatgttagtttcagcttagaaaaatcagtcatttgactaccttgaggctaaattgaaagaattttttagggaggtacaggcataccttggaaatttcagaacaccacaataaaacaaattttgcaacaaaatgtcacacaaattttttggcttcccagtacatataaaagttatgttggccaggcacggtggctcatgcctgtaatcccagcactttgggaggccaaggcaggtggatcacaaggtcaggaggtcgagagcatcctggccaacatggtgaaatgccatctctactaaaaatacaaaaattagctgcatatggtggcatgtgcctgtagtcccagctactcgggaggctgaggcaagagaatctcttgatcccaggaggcagaggttgcagtgagccgagatcgcactccagcctgggcaacaagagcaagactctgtctcaaaaaaaaaaaaaagtttgtatttacactatactctagtctgttacatgtgcaatagcattatgtctaaaaaaaaaaaaagccatgtatatactttacttttattagtaaaatattttattactaaaaatgctaatgatcatctgagtcttcagcaagtcctaatctttttgctcgtggagaatcttgccttgatgttcactgctgcaggctaatccaggtggtggctgctgaggtttgtggtggctgtggcaatttcctaaaataagacaataatgaagtttgctgcatcgattgactcttcctttcacaaaaagatgtctctgtagcatgtgatgctgtttgatagcattttgctcacagtagaacagctttcaaaattggagtcagtcttcgcaaatctagccactgcttcatcgagtttatgtgatatcctaaatcctttgttgtcatttcaacagtgttcatagcatcttcaccaagagtagattccattgcaagaaaccactttctttgctcatccataagaggtaactcctcatccattcaagtttgatcatgagatggtagcagttcagtcatgtctttaggctccacttctaattctagttctcttgttatttccaccacatctgctgtgacatcctttactgaagtcttgaactcctcaaagtcacccatgagagttggaatcattgtcttccaaactcccgttattgctgatgttttgatctcccatggatcacagatgttcttaatggcatctggaatggtgaatcctttttagaaggtttgcagtttactttgcccagatccattagaggaatcgctatctatggcagtgaatagccctacaaaatatatttgaaatatattttgaaataataagaggctgaaataataagacttgaaagttgatccacaggctgcagaatggatgttgtgttagcaggcatgaaaacaacagtaatctccctgtacctgtccttcagagctcttgggtgactaggtgctttgttagtgagcagtaatattttgaaatgagtcttttctgagcagtaggtctcaacagtgggtttaaaatattcagtaaaccatgctgttaagagatgtgctgtcatcccagttttgttccatttatagagcatgggcacagtagatttagaataattcttaagcgccctaggacttttacaatggtcagtcttggcttcaaaaacaaagtcgccagctgcattagcccctaacaagagagtcagcttgttctttgaagctttgaaggcaggacttctctctagtgatgaaagtcctagatggcattttcttccaaataagtctgtttcatctccactgaaaatctgttatttagtgtagccaccttcatcaggagtcttatctagatcttctggataacttattgcagcttctacatcagcacttgctgcttcaccttgcacttctgtgttatggagatggcttcttcccttaaacctcatgaaatcaacctctcccaacttttttttcttctgcagcttcttcacctctctcatccttcatagaattgaagacagttgtgggccttgttcttgattaggctttggcctaagagaatgttgtggttggtttgatcttccatccagacaactaaaagttttctccgtatcagcaataaggctgttttgctttatcatttgtgtgctcactggagtagcacttttaatctccttcaagagcttttctttgcattcacaacttggctaactggcacaagaatcttggctttcaatatgccttgcccactaagcttaattatttctagcttttgctttaaagtgagagacctgtgactcttcctttcacttgaatacttaagaggccactgtagggttatttactggcccactttcaatattgttgtctctcaagggatagggagcctgggagttgagggggtggagtggccagtttggggggcagacagaacacacacaacatttattgatttagtttgctgtctttatgggcatgtttcatgacactccaaaacaattaaaatagtaacatcaaaggccactgatcacagatcaccaaacagatataataatttttaatgtttgaatatttaaataattaccaaaatgtgacacagacatgaagtgaacctgtgttcactgttggaaaaatggcaccaatagacttgttcgatgcagggttaccagaaacctttttgttttttttttttaaatactatctgtaagcactactgtacaataaagctatgtatgcctgtattttcattaagttgcagagcaaacgtggtaatatttagctttagttttacttcatctggcataagatcaactccttatataacaagatgataaaagttgtggtgtgcttctataatttcattcaagtagataaagttgaaaaataatgacttgcttttataaacagtatgaagcaatgtagtgcagtaaatgaaattttattccttctttacaatgttctcaaaattatttttatgtttaatccaaataaagagcaagaataaagcaacatttcagattttggtttctggagacaatagttagaaagcatgagttatgagtgacttaaaattcttgttgcctgtacttcactttgaaataacattatgctttaaaaagcattacactgctaaaggttaattagaattctgcagaattactatagctaaaagtaggtaacaagatatctttttttctattgtttaactcctttgtttcagaatgcctattcctgtgcattaaaagtgtccctccaaggaaattaggacatctgcagagttgaaaaacacctaagtctcagtcacttagagtcacacatcagggctcagagtgctatgactaggaaaatgctgacctcctttcattagtatgatcgtgcctttccagcttttgatagatccaagcgctatcttcccaccactcaccaaatgttccacctgtcaaagggtttcaggtccctgcagacttcggttttgacctgtggggaaagtagacttcctcgaactggggaagccacatgttgtacatccttctataaactatgattatcattcttagtaggaaaatatgtgatttctttttttttttttttttttaaagtaagcatcaaatatttgaccaaccagttgggcagagaatatactgaaactttttatataacctcatccaaatgtcccctgcatttaagaaatgaaattcttctaattgcgtttataaattgtaaattatattgcatttagaaattaaaattctttttcttaatttgttttcaaggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggtaggtaaacttgactgcatgtttccaagtgggaattaagactatgagagaattaggcttagctttttgctaagaactagctaagtatctcttttaaaaaacgaatcagtgtgcttccatgatgcttgggttacagttgttctttcttgttttggttttcattcattgcaacttaccgtgaatattctgctcaaggtattgagagtgtgtgttgttatctcaacttacaatttgtgttgaagttatcaaaataatacaaatgataatgcatgactttaaaaaagcatgattactcttagacttttttttttcaaacacaagcaacaaaaaccagtacaagaagttaaaaataaaaatagaagtaggaagttgctttttccaaaaaaactaaacatgtttattcttggatttcttttgtcttaaatagtctggcctctcaatacaaccaggtaatagtaaataatgtgacttttcagtgcagactcttacctttaaagtttctcaggaaatcacactataccaaatggcccaccaaaacatacatgattgttatttgagagttctgcaacctattaccctgcacttccacctgaaatatcttagtttcaaggtgctatactaaaacaagaaagagagaaagaaaatttacgaataagttatttttatgcatttgaaatgccatttggatgggggaaaaagtgttaaaatctcacagtatatgcaactttttaggaaactgtcttgcagagagtatagtgtaaacataacttttatatgcacagggaaactgaaaaatttgtgtgacatttaattgcgatattttaagagttatggaatttttagagctgaaagatttattgatgatgaccaatcaaattgatcttctcatcttgtagatgagggaactgaggccgagacagaagggttcaaattatgcaaggtcaagctcagagtccagatttcccctcatattccccattgctttttttttttttttttgagacagtctcgcgctgttgcccaggctggatcttggctcactgcaacctctgcctcctgggttcaagcaattctcttgtctcagcttcctgagtagctgggactatgggcacacgccaccatgcccggctaatttttgtttttagtagagacagggtttcaccatattggtcaggctggtctcgaactcctgacctcaggtgatccacccgccttggcttcccaaagtgctgggattacaggcgtgagccaccgcgtctggcccccattgctttttccatcagagaaaagtccaggtgtttctagggagttgaagattccactatccccaaggaaatgggctctaaaccattcttgattagaagaggacaggcagagtttaaataaatgaaatttgtggtgaagccagaaatttcattttgctcagcaattaaaacctctgcagactacaacagaagctgctgatggaaattggggtggagcctcatttccccattcatactgacgtggatgagcagtgtgaggaaggaaaccaagagaacagcaggcagcagggtggaaaggaacggactgacccatagatcagagtgtgagcacataaaagcaatgaccccactgtcatgtttggccattgcaccttttgtgatagtatctaagcttatgtccctcaaaacagtgtaagataggttttcattagtatcagcttatttcatttctgctcaaattaatttttaaaaaaatttaattccatatttgagcaaataaagatgtcttcatacttgagtaattcaagaccccttttttgctgctgttgttgttttggtttttttcatggttttgttgttttcttttgttgttgttgtttctttgttttttgagatggagtcttgctctttcacccaggctggagtgcagtggcatgatctccgctcactgcaacctccacctcccaggttcaagcgattctcctacctcactctcagcctcctgagtaactgggattacaggggcataccaccacgcctggctaatttttgtatttttagtagagacagggttttgccatgttggccaggctggtcttgaattcctggcctcaagtgatccgccctccccaacctcccaaggtgctgggattacaggcatgagccaccatgcctggccttcaagagcactttttagtttatttaatataattctctttgttgccggtctgtccgttgaagttgccttctttgatgccagaggccaaagacttggcttcctcgggcttccataacaaagtaacacagatgggtggattaagcaacagaaatttaccctctcacatttctggagtgtaagatcaaggtatcagccaggttggttcctccccaggcatctctccttggtttgcagatgcctgtctgctccctgtatcttcacatgaccttcactctgtttttctgctgtgtcctaacctcttcctacaaggacaccagtcatagtggattaggatcaaccctgatgaccttgtttaactttaataacctcttcgaccctatctgcaaatacagtcacaggtggaggtactggcagcttaggacttcaacatatgaattctagggggacacgttttagcccgtaacagaccccagtgatgctgagcataaaagcaaggcctccacctggagctccttcccctggctgtgcgtgagaccaggtcctacctcagacctaaggaaaaagaatccccaaataggaggctgcgaatctgagtgttgaaaatttccccaaatgaatgagatgtgcagtcggatttgagaatccctgacacatcattttaacactccttccttttagttctccgcgcaagcctcagggagtggaaaatggtatctctcacctcccagataaccggttgccttttaagtgctcttccagctgcctgaattaaaacgctctttttttagtcagtttaccaagcagtgtctcattggacttaaaatgtcagaggagaagttgacactttgacccagattctgacattaatttttaagccaggaggtatccttgcaggagcaaaatcgggaaggatggctgttcagctcttctctcctcattttgctgtctactggagagcaactggaagcctttctagatgaggggctccacctcattcgtgtttgttttgtttatttcctctcagctccttcccctcaagacctttgtgactgtccttcctgaaagcagccctctgtgcagccccatagtgtgtctgtacagcaatgttgaaatagaccacattccgcttttctgtcctcaaaaaagatgtagtttagctgggatggtagaacctgaacacagggagttgcaacccagcctagggatgttcccaggtgagagagcacggtgggctttcagagcaccatttttcttagcattccagcgtctgcacccacagaagtgatgcttctgcaacatcctcttcctaccttaacaggcactctcaccctcaagtacatggaaatacctttatttccggctctggctaaccttcattaatcagtaaccctacttcagtaagaacaaaaatgagtacatcaatatttccagttggactcacctcagtcactcaggacagaataccattgtacttacataccatcgtgcctgaagaagtcatgatgaaagctttggtttccaggagattttttgtttttagagtgaaaaatgcccgcagaagatcacagagataggaaccctaggaaaaaaactcctgagaacaaatgtataaatgaaacaagcctagaacagtcacataccacttcacactggagagaacgttctgagaaatgtggcatggggcggtttgatcgctgtgcaaacatcataaagtacatgtacataaatctagatggtatagcctgctacacacctgggctacacacctacaactattgctcctaggctccacacctgtgcagcatattgctgcactgaatactatagacagttgtaacacactggcagggatttgcgtatctaaacaaactagaaaaggtacagtaaaaaatacggtattatggggccaccatcatatgtttggtctgtcgtggactaaaatgtcattatgtggcccataactgtatttttattggtagagttgttagcatttggaaatgagcacttgctagttgttgtttttttttttttttttttttttcaaataagcaggaaatattgacttactgaatttacttagccataaggccagagagtcactctgtgagccattggccacgctttcccaaggaggtaactgtgccttattggtgcttcaagcagtgaagcaatagccctttattattaccactccccaaatgtgccctccatgtcagaatagccctctggtggctggaaatctcagcactagctgaattaaccaccttgctcctgttttaggacgttcctgcaaagcccactaatcccaggatctgtttagtagcaagcggtacttttctacattcacatacatagacactggatatttaaaaatacgtattgtagctctttctgctactctccccagacatgtcacctttaataaggagaagttgggggcacagcttctctttattataggatgtgaaaaagttttcctctcacagatctgatcacttgaacaccagaagtctccccgcccccacctcctggttatctcattaatgatctaaataagaagagagttccttttatttggcttttatggaggtatggcatatgactttttctaagtaatatgctgtcacactggtgactgtataaaaagatgtccttagaggtcctttcttcccggccagaagctgacccaccaggatacagacgcactgacgttggcattgtagagtagttggagagagtggtcaggttgaatggaaagccacgataaggaaaagtgagggggggtgcagaacctaggagtggcttacagtgagcagcaggaagagaaattggaagatcaaaggaaagcatcaaaaatgatcacaggccaggcgtggtggctcacacctgtaatcccagcatttggggaggccaaggcaggtggatcacttgaggctaggagttcgagtccagcctggccaacatggtgagaccctgtctctaataaaaatacaaaaaattagccaggcgtggtggcatgcgcctgtagtcgcagctactcaggaggctgaggcaggagaatcgcttgaacccaggagatggaggctgcagtgagccaaaatcacgccactgcactccagcctgggtgacagagcaagactgtcttaaaaaaaaaaaaaattatgaagggatagcaaaggagttcctgaacctgcctgaagaattgcttgatgttggccataaacatagtcaactgccttctagttatatataactatagagagatggcatcaaaattaggaaaacaagcatctatacaactaggtagcaggcagttactgggaggttacgagggataagtcactagcttgaaaacttcctattttctttgcttttattttttttttccgcagacttgtggctcctttgatgtgtcacactattgttgtattgttcgccttagttcccttaccctcctttactcgtctggtccatctcaatgacaggattctgtcgtttggcctgactcagtcccttgtataaaaggacatctattaatccacatcctgctggttactgtacataataaaatgtaaacgtggtgtttcctgcctctcctccaagctcacacacccgcatgtaaatactctaggggcaataggaggagacctagaaactaaggtcacccaaatagaatcttgtccctgcaggcggatagctgagctctaaattactctggatacagtgaaggactaaatggttccaatgaatcaagttcagggagaaaagggtcagggggtatggaggttagccaaagagtgtcaaacgttctcctcaaagctattttagaacaaggctggtcaggctatttgggctcagctcttttcagcgtgtgtcataggcagcagccagttgaagccatgccaccatgcagtgcaggggtccaggtctcaggaaggtgcagcgtaccgagagtcacatgaagacctcagatcatttcctgctgctgtgtaatccaggcagtctgggaagagaatgaatattcagtggttgtatgtatgtgtgtcttcatcccagcaagtgacagatgttcctgcccagttggaaaatggaaattctgacttcattgtcttatcattccttccagaacaagcaccatgggctcaaaccctccctggctctgctttaagggaaataatattgtgtctgtgcagaggccctgtctgcacagggtcgtggtagggctggccttcacatggtggcccatgccactgctgtagcttagccatgaggaagagattggtgagcctgctggagaggaaggctacccttacttggtgctcctcctttatccatagacatgagtactcagattttcgcatctagctttcaaggggctggagattaaagatccaatccatactccttggatgcccttttgtcaagataatcccttcttctgtagctcctaagccctctaaggtctgagggccaagctgccccatggcaggaatggcaaaatgtagctgtcctctcagtggcaaaaggtaataggattatcaagtagcaaaacagaagccaagaaaagacaggagagttagaggtctggattgtgcaccagccttggagcctcgcatctctagcagggcactcttaagcgttaggctttggagtttgaagaggagataaacctatcaaattgccaggtgttcccctgcccttgatgcatgcaaatgattgttgacagccagtgtgatggtagatgtgcctcctggaagaaataaaagtataatattccaacaatcaagcagcattgtggcttttttctacaatcagaaacatagcagctttgtgcttgatctcctaagcaagataattttttttaacctgggcattctttgagttctcagtttcgaggtgatggggagcaaaaatgatgcccaagttcaaatggcgtactgctgcagataggctgtctcctggcacacagatttacagagtgaatgaaatctctcattcattggtgctaatctttaaccatggtttttaacttacaagttggaggctaattacacagttaccagttaaagccaggtgacctcatttgttgacaacagtgcacagcaaaggaaagaggagaggataaggaaaatatctgtgatttgctcccagaaggacagtgaagctgagcatccatttgagcagagtcctctgagtactggccttggtcagagcttgttggtgggagtgtgctaaggggagatgcctaggtctgtcattggagtaagctgagggtttggatgcaggggaactcaggcagctctgagccacagggtggtagatgggagggattcacagtggcgcacagtctcttccataaatggaaacgtaagaatcggggcacatggccatggcggctcatgcctgtagtcctagtgctactttaggaggccgaggtaggcggatcacctgaggtcagtttgagaccagcatggccaacatggtgaaaccccatctctactaaaaatacaaaaattagccaggcgtagtggcgggtgcctgtaatcccagctactcgggaggctgaggcaggagaatcacttgaacctgggaggcagaggttgcagtgagccgagattgcctgggcaacaagagcaagactccaactcaaaaaaaatgggcacataatggatggaacattctgtcccaagggatgagggagctgtatatctacctattccagctgccttgggattagccactggtagtcattccacctcactcccagactgaaaagagtcaatcacttcatgaagtggggctatccgaagggaccacaggggacctgattatactgacagatttatttttatttatttactttttttgagacagagtctccctctgttgcccaggccggagtgcagtggcacgatcttggctcactgcagcctcagcctcctgggttcaagcagttcttctgcctcagcctctggagtagctgggactacaggcacgtgctactgtgcctggctaaatttttttgtatttttgttaaagatggggtttcactatgttggccaggcgtgtctcaaactcccgacctcgtgatccacccgccttggcctcccaaaagggctgggattttgagccacctcacctggccttttttttcttggagatggagtctcactctgtctcccaggctggagtgcagtggcacaatcttggctcactgcaaactccaccccccaagttcaagccattctcctgcctccgcctgtggagcagctgggattacaggcgtgcaccaccacgcctggctaatttttgtatttttagtagagacaaggtttcaccatgttggccaggctggtctcaaactcctgacctcaagtgacgcacccacctcaggctcccaaagtgctgggattacaggagtgagccactgcacccggccatattgacagatcttttagtctgaattgttatcagccagtgtggtctagtcatgacatatctagctccatctcgagacctcatgtcttactgctgacactacatcctcgctggtactttttcttccttggcttcaggggcattgctgtgtaggctcagatccatgccagatggggtgtgtagggggaaaagctgcataagggaggaatgtcacttggcaacactcaacaagggggaatgggcctgcagtattctcccagagatgaatgagcagcccattctaggaaatatccctttgaaggaatatcttctctatggtacatccaaacttttctctggaaaacatgcctcttcattctgatgactttctatttattctcttcatcttaccatggttttacaaaagaggaattttaacatactcattcagcagagttaaggacctcctctcttctgcgaacggggagaggtataacaaatatagtaaaacaagaacagccactgcccacgttgtaggggtaagtaccctgcccaacctgcagacagtaaatgccacacacagctctaacagtgacagggggagaaaaacgtcttcagttactggcctgaccttagaggtaaagcaacatattaacagagagggtctgattttaaactttgagctgaacatacttctatagtatcttaattctcagtttttaaaaaattacatcatctagaatggcgtcgtccttgctcttccttttttcttgtgccaaataatagccccctttggtatatgttattgttttaattgattctcaaacctctccttaacattttaacaaacataaaatttgatagaaatgggctagactaaattcagcagtccaggaaatcgagtatttttctacacagatttgagtgtcacccaagatacagtatactttgctgaatctccctccatgatactcttgtggttttatctattttaccaaaacacttccacgtaaaacaggcttccgttgactctgtttgtcctttatggacccaacaggggaggaaaaaataccattgcactgggtttatccacttactgtggagttggggaaccagaaacaaccactggacacgttgcaagcgcgtcaaggtttccaccgccacctggtggctagatggagcacagcatctgtgtaactggtgggcaaaggcgttctgctccaagatgtcaaagtgggggagaaaattatgggtgttctgcaatcttggcaaaagaagagtactgtactccttatctttttactgcttctccatgttcacccttaaaagattgatttttattttttactcagctctcctcttgtttttcaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaccattgtgctggtgtgaatgattccatagtaacaatcttgaccatttactgacgtacagaccagtgagaagtcttcgcatgttgggtacccacacctgttgtgtcttaattgcaagtctgagtaggaagttggggccaacatgtgtctcccagtgctgggaaaatatttcatagacctaatttacagtctttacttgatctaaaacattttgctgccatattttggccctcaagtttgtcccaaatgagagacaaagggaaaagttccagggaaataaaaattaagacagctgattatctgtaaagcatggtttctcatcctgaacgctactaacattttgcagggaataattccttgttgaagggagttgtcctgaccagtgtaggatatttatttattttatttatgttttttgagagtctcgctctgtcacccaggctggagtgcagtggcacaatctcggctcactgcactccagcctgggagacagacgagactccgtctcaataaataaataaataaataaataaataaataaaaggagggcctggcacgaatgacatgcagggaaggcagtgagcaggtggaggtccctgtactcgttgtggtgccttatctaccaggcggttgagttgacgtctttgtggacagaattcgattacaaaggtgtccgaaagtctccaggtgggggagagttttgtagcgggcataatttgggttgtaaattgactgttgtctcctgagcagtctcctggtgagggagagtttcactggagctttgaaacacatgttagacagccttgccctgtagggagtgtctcgtgaaggagaggtaaacaattataattgcatttctaaagggctaagtaggacatgggaaactggaaaatggagaaaagaagagaaaaaaatagtaactcattctctttttcttagaaaaatgaggatacttggtcacatcctcacatggtgaagaataagatggtctcttcatttcccttcaacactagtcccatcgtgaggctctgccctcatgacctaatctaatcctaactgcctcccaaaggtcccacctccacatcccattattggggattagggtttcaacatagggatttgggggaaacacaaacactcagttcatagtgatatctgactgcgattccaaaggtctgaggacacattggccctgttgtttttgctgtgttgtccttggcctcaggtgaacattttagctttagattgcaacattttaagctaaaaacactaagctagaaaattaaacttatctcgtcacattctgcatggtgaacaaactcaaaactcacctgagtgtatcttgttttcatgagaaaaaatgaccggtcatctcccttgccctgttagggacagttagacatgactgctgtatgaagatatgtacatgttaacaattcatatgtacaagctactgttccattttgcctggcccaaggggaggtagctagctagttggggtagcagggcgaattaaccctaatataatagtacatttctaatgccaccaatgaaatgaaaaccagctctcaaacatggcactatatgggatggcattccaaggccaactcagttacagtaactacagctttaactttgatcattttgttaccagaaaggggtcccaatccagaccccaagacagggttcttggatctcatgcaagaaagaattcaaggcgggtccatagaggaaaaggaaatgaagttttaaagaaagtaggaaaataaagagtggttgctccataggcagagtgaacctaagggctgctggttgcccattttattgttatttcttaattatttactaaacaagggctgaattattcatgcctccccttggtagaccatatagggtaacttcctgatgttgccatggcatttgtaaaccgtcatggctctagtgggagtgtagcagtgaggacaaccagaggtcactctcattgctatcttggttttggtgggttttgaccggcttctttactgcaacttgttttatgagcaaggtctttatgacctgtatcttatgcagacctacaatctcatcctgtgacttagaatgacttaaccctctgggaatgcagcccagtaggtcccagccttattttacccagcccctattcaaaatggagttgctctggttcagacgcctctgacaattttaaagctaatgaaaaaaatgtctagccaaatgtatatatacatacatatataattatgaaaagataatgccttataatcttgattcttaaattgaggtatattgatagtattctcccaatggtctggggattaatcaataggtaatttatacaaaactgaagtgctgccaaatagaattataggagtagctgctgcgcaaaaaagattaatgagatttggggagactttgctgtggctaatatgatgggtcaggaagagtacaatctcagtacaattttaagttgtatttatcttatgaataattgggctattctttcatatgcccaagggccattttatagctctatatttcttttgtgttctgtttggggcttgaagtctctacttctgtatcgggattgcaatgcctgctgtcttactgtttccatttgtctggtaaattttcgttcattcctttatttttagctatttcaattgctgcattttcggtgtgtatcttgtgtgcagcacacagttgagtttttgctatacgagccaatccgaaaataatcttttttaataggcaaattaagccgatctagttttgatataactaccatgtttgatgtcaattctgtcatattttagattgtaattgctgaatgtattatatttactgtgtctatcctcttgcgttttcttttggcttatgaattttgtgtcttttatttatttatttatttatttatttatttatttatttagagacggggtctcactctgtcgcccaggctgcagtgcactggtgtgatctcggctcactgcaacctccacctcccggattcaagcgattctcccgagtagctgggactacaggcacgtgccaccacacccaactacttttttatttttagtagagacggggtttcaccatgttggccaggctggtctcaaactcctgacctcaggtgatccacctgcctcagcctcccaaagtgctggaattacaggtgtgagccaccgcgctgggtcttgtatctttattctaatgggttgcttttatactaatcttctgtagtgctcttactcccttttttttttttttttttttttttttaagacggaagtctggctctgtcgcccaggctggagtgcagtggcgcaatctcggctcactgtaagctctgcctcccgggttcacaccattctcgtgcctcagcctcccaagtaactgggactacaggcacccaccaccacgcccagctaatttttttgtatttttagtagagacggggtttcaccatgttagccaggatggtctcgatctcccgacctcatgatccacctgccgtggcctcccaaagtgctgggattacaggcatgagccactgcgcccggccccttttttctttcttaacctgttactatctgtcttttctagatgatactgattcccacctattacctatatgtcagtcaatggtgttactctacttttccccttttctctcccttctcccacctttttagttgtatttgttctactttatcagtatataatgtttacatatccatgtttttcattaacccatgaacgaactcttacattattctcagctctacagtcaaatatgtgaaatgatctctgttggtccttttgttcccagaaatattctggttgggtgaaactcaccctccagtaaattgctcagcaagagcacgtgtactgtattcctggaattctggtgagtttgaaactgattttctgtgaccttgaaacttgaagagcagcttgtttttccttaggtttcttgaaaatggtgttccagtgttttcttggtttgcatgttatttttgagaaatctgaggccaatctttaattctctttgtaaatattttttgggcctggaagccttgaggattttttttttataagtctaataattttccgagctatctcagaattatttgtggtcagttttcctatataccacactgggccctttcagtgtagagttgactcagttcttttatttctggaactttttcttggattagagttttaaatattctgttatattgtttttcttttcttcaggaacaccaataatacaaatatgcaacctcctttgtctggcttccatttcaactatcttctctggtcctttttattttatctccttttcattctcttggctatccttcttttattcaataccctttattaagtttttatttgaacccattctcttcggggcaccttgtaatctgttctttattactgataaagttttgtgattttcttctattttttttcctgaatttaatcagctctggcttcattttctcctattttttgttgatttctctacctagtttttatatttccgattcaacatattgatattttccatgttcccagatgcaggttggagaatatttcataaatattgagtgttgtcttagaattttcatcaagttcatggttatatcgaggagacatcaccagcagaaaaactttgatttgcaacttctgactttggcagttgttctctatagaagttggtccactccttcctcttcctgttcattctgtgggcaggagacctagttaggagcagcttcttccctcagttcaacaatgttctgtgcagttgcttctgtggctgatgttgacaggaaagatagaagagttgttgtgtctcctattttctgcaggatgcttaattttgttctgtctcctcttctgctgtgcctgtctcccagaagcaccactcctctccctgtgtctgattctccccaacaagcatggattctccatgtctggtctgcacactgtcaggccccactccggagcctgggctctgaactgccaagtgcccatcattgctcgttttttttggttttttttttttttttttttttttgagaccgagtttcactcttgttgccgaggctggagtgcaatggtgcaatcttggctcactgaaacctctacctcctgggttcaagcaattctcctgcctcagcctcctgagtagctgggattacagacgcacgccaccatacccggctaatgtttgtagctttagtagagacgaggtttcaccatgttggccaggaggtctcgaactcctgacctcagatgatccacccacctcggcctcccaaagtgctggaattacaggcgtgagccaccatgcccggccttgttcattctctttgtaaatattgtttcactttatcttttgggagtggctttgtctgttttttctctaagttctctgcttccctccacccttttccccaatctcttgagactcctctcccctgatttctgcacccataggtttgtggtggctgcttgctgcaggcccacagtgggcactctgttggatttttttttcttctatgcacagataattggaaaattgcagcttctcttatctcttagtcatgctgaaagagggacatttaccaccattatgaggaaatctgcacactggtatatttttaaagtgccccagatgattctaatacacagctgtggtggagaacatcttgttatatcatcataatgtgcttttagtacattttacatatattttcccaaaagtcccttggcttcccagccatgactgtctcatttttctcctacctgagtcctgtaggtaagagaagcactgtccttcacatctctgtttctcaaagaattcacctacatcagaatcacctatgagcctggccaaacattgcagttttccagatcccattctgggcctactgagatc

SEQ ID NO: 12 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 13 - DNA, 2638 bp, Homo sapiens
gaggaaaggggaaatgcggcccgctccccactcagtgccactctgtgccactccgtgccaggccctgagggcacccggttgctgcttccttccgtctttccccaaggactatcagagatgccagcgtgacccctgacacgtgtgtgcagcagcctgcagctgccccaagccatggctgaacactgactcccagctgtgggcttcaccattacagactccccagggcttcaaagacttctcagcttcgagcatggcttttggctgtcagggcagctgtacaatagtggatgtttgagacggaggcagatgagaagagggagatggccttggaggaagggaaggggcctggtgccgaggattccccacccagcaaggagccctctcctggccaggagcttcctccaggacaagaccttccacccaacaaggactccccttctgggcaggaacccgctcccagccaagaaccactgtccagcaaagactcagctacctctgaaggatcccctccaggcccagatgctccgcccagcaaggatgtgccaccatgccaggaaccccctccagcccaagacctctcaccctgccaggacctacctgctggtcaagaacccctgcctcaccaggaccctctactcaccaaagacctccctgccatccaggaatcccccacccgggaccttccaccctgtcaagatctgcctcctagccaggtctccctgccagccaaggcccttactgaggacaccatgagctccggggacctactagcagctactggggacccacctgcggcccccaggccagccttcgtgatccctgaggtccggctggatagcacctacagccagaaggcaggggcagagcagggctgctcgggagatgaggaggatgcagaagaggccgaggaggtggaggagggggaggaaggggaggaggacgaggatgaggacaccagcgatgacaactacggagagcgcagtgaggccaagcgcagcagcatgatcgagacgggccagggggctgagggtggcctctcactgcgtgtgcagaactcgctgcggcgccggacgcacagcgagggcagcctgctgcaggagccccgagggccctgctttgcctccgacaccaccttgcactgctcagacggtgagggcgccgcctccacctggggcatgccttcgcccagcaccctcaagaaagagctgggccgcaatggtggctccatgcaccacctttccctcttcttcacaggacacaggaagatgagcggggctgacaccgttggggatgatgacgaagcctcccggaagagaaagagcaaaaacctagccaaggacatgaagaacaagctggggatcttcagacggcggaatgagtcccctggagcccctcccgcgggcaaggcagacaaaatgatgaagtcattcaagcccacctcagaggaagccctcaagtggggcgagtccttggagaagctgctggttcacaaatacgggttagcagtgttccaagccttccttcgcactgagttcagtgaggagaatctggagttctggttggcttgtgaggacttcaagaaggtcaagtcacagtccaagatggcatccaaggccaagaagatctttgctgaatacatcgcgatccaggcatgcaaggaggtcaacctggactcctacacgcgggagcacaccaaggacaacctgcagagcgtcacgcggggctgcttcgacctggcacagaagcgcatcttcgggctcatggaaaaggactcgtaccctcgctttctccgttctgacctctacctggaccttattaaccagaagaagatgagtcccccgctttaggggccactggagtcgagctcagcgttcacaccaggcgggctgggtcccctgcccacctgcctccctgccccctgtgacggagggggcaagcaagcccccagaggccgtgtctctggacagacggatagacatacggaagcgaggcctggaccaagagaggcccaggctactggaggagtagaaggatgggccccgtggggtccccactgccccggtacgagggggcccaagaccctggcaggtcaggggccctggccaagccagatctggagctgctgctccctgctgcggagaccgcggaggcttcgcgttgaccaagttccttaaagaactggctgatggggcaggaggtccaggcctgggctctcgggccctcctagagggccattggagcttgcagctcagacccccactttgagttttatttatttaaatagtagttggatgcttggcacgtcgtcctgtaataggaaacccttgcctcatcagttttcctgatttacaagtgcaatattttagccaatgccttgggagaagctgccatgcaaaggtggacaccattctccagcttcaggggatatgctcgtcccgggcaccggtggcaggcagctggccttctggactaaggcagcctggggggacactgcagtctggctacacacagagatctggcaccccctgggtggagtgtccctcgggggctttgggaaagcatggcaccctcagaccacacagtagccaagttctggagcaaataaaaggcctgtgttatttcttgttcttgaaaaaaa

SEQ ID NO: 14 - protein, 519 aa, Homo sapiens
MFETEADEKREMALEEGKGPGAEDSPPSKEPSPGQELPPGQDLPPNKDSPSGQEPAPSQEPLSSKDSATSEGSPPGPDAPPSKDVPPCQEPPPAQDLSPCQDLPAGQEPLPHQDPLLTKDLPAIQESPTRDLPPCQDLPPSQVSLPAKALTEDTMSSGDLLAATGDPPAAPRPAFVIPEVRLDSTYSQKAGAEQGCSGDEEDAEEAEEVEEGEEGEEDEDEDTSDDNYGERSEAKRSSMIETGQGAEGGLSLRVQNSLRRRTHSEGSLLQEPRGPCFASDTTLHCSDGEGAASTWGMPSPSTLKKELGRNGGSMHHLSLFFTGHRKMSGADTVGDDDEASRKRKSKNLAKDMKNKLGIFRRRNESPGAPPAGKADKMMKSFKPTSEEALKWGESLEKLLVHKYGLAVFQAFLRTEFSEENLEFWLACEDFKKVKSQSKMASKAKKIFAEYIAIQACKEVNLDSYTREHTKDNLQSVTRGCFDLAQKRIFGLMEKDSYPRFLRSDLYLDLINQKKMSPPL

SEQ ID NO: 15 - DNA, 6608 bp, Homo sapiens
ccttcttttaaggagtttgccgcgagcgcgtctccttcattcgcaggctgggcgcgttcgcagtcggctggcggcgaaggaaggcgctctcgggacctcacgggcgcgcgtcttttggctcttgcccctgtccctgcggcttggggaaagcgtaacccggcggctaggcgcgggagaagtgcggaggagccatgggcgccgggagctccaccgagcagcgcagcccggagcagccgcccgaggggagctccacgccggctgagcccgagcccagcggcggcggcccctcggccgaggcggcgccagacaccaccgcggaccccgccatcgctgcctcggaccccgccaccaagctcctacagaagaatggtcagctgtccaccatcaatggcgtagctgagcaagatgagctcagcctccaggagggtgacctaaatggccagaaaggagccctgaacggtcaaggagccctaaacagccaggaggaagaagaagtcattgtcacggaggttggacagagagactctgaagatgtgagcgaaagagactccgataaagagatggctactaagtcagcggttgttcacgacatcacagatgatgggcaggaggagaaccgaaatatcgaacagattccttcttcagaaagcaatttagaagagctaacacaacccactgagtcccaggctaatgatattggatttaagaaggtgtttaagtttgttggctttaaattcactgtgaaaaaggataagacagagaagcctgacactgtccagctactcactgtgaagaaagatgaaggggagggagcagcaggggctggcgaccaccaggaccccagccttggggctggagaagcagcatccaaagaaagcgaacccaaacaatctacagagaaacccgaagagaccctgaagcgtgagcaaagccacgcagaaatttctcccccagccgaatctggccaagcagtggaggaatgcaaagaggaaggagaagagaaacaagaaaaagaacctagcaagtctgcagaatctccgactagtcccgtgaccagtgaaacaggatcaaccttcaaaaaattcttcactcaaggttgggccggctggcgcaaaaagaccagtttcaggaagccgaaggaggatgaagtggaagcttcagagaagaaaaaggaacaagagccagaaaaagtagacacagaagaagacggaaaggcagaggttgcctccgagaaactgaccgcctccgagcaagcccacccacaggagccggcagaaagtgcccacgagccccggttatcagctgaatatgagaaagttgagctgccctcagaggagcaagtcagtggctcgcagggaccttctgaagagaaacctgctccgttggcgacagaagtgtttgatgagaaaatagaagtccaccaagaagaggttgtggccgaagtccacgtcagcaccgtggaggagagaaccgaagagcagaaaacggaggtggaagaaacagcagggtctgtgccagctgaagaattggttggaatggatgcagaacctcaggaagccgaacctgccaaggagctggtgaagctcaaagaaacgtgtgtttccggagaggaccctacacagggagctgacctcagtcctgatgagaaggtgctgtccaaaccccccgaaggcgttgtgagtgaggtggaaatgctgtcatcacaggagagaatgaaggtgcagggaagtccactaaagaagctttttaccagcactggcttaaaaaagctttctggaaagaaacagaaagggaaaagaggaggaggagacgaggaatcaggggagcacactcaggttccagccgattctccggacagccaggaggagcaaaagggcgagagctctgcctcatcccctgaggagcccgaggagatcacgtgtctggaaaagggcttagccgaggtgcagcaggatggggaagctgaagaaggagctacttccgatggagagaaaaaaagagaaggtgtcactccctgggcatcattcaaaaagatggtgacgcccaagaagcgtgttagacggccttcggaaagtgataaagaagatgagctggacaaggtcaagagcgctaccttgtcttccaccgagagcacagcctctgaaatgcaagaagaaatgaaagggagcgtggaagagccaaagccggaagaaccaaagcgcaaggtggatacctcagtatcttgggaagctttaatttgtgtgggatcatccaagaaaagagcaaggagaaggtcctcttctgatgaggaagggggaccaaaagcaatgggaggagaccaccagaaagctgatgaggccggaaaagacaaagagacggggacagacgggatccttgctggttcccaagaacatgatccagggcagggaagttcctccccggagcaagctggaagccctaccgaaggggagggcgtttccacctgggagtcatttaaaaggttagtcacgccaagaaaaaaatcaaagtccaagctggaagagaaaagcgaagactccatagctgggtctggtgtagaacattccactccagacactgaacccggtaaagaagaatcctgggtctcaatcaagaagtttattcctggacgaaggaagaaaaggccagatgggaaacaagaacaagcccctgttgaagacgcagggccaacaggggccaacgaagatgactctgatgtcccggccgtggtccctctgtctgagtatgatgctgtagaaagggagaaaatggaggcacagcaagcccaaaaaggcgcagagcagcccgagcagaaggcagccactgaggtgtccaaggagctcagcgagagtcaggttcatatgatggcagcagctgtcgctgacgggacgagggcagctaccattattgaagaaaggtctccttcttggatatctgcttcagtgacagaacctcttgaacaagtagaagctgaagccgcactgttaactgaggaggtattggaaagagaagtaattgcagaagaagaaccccccacggttactgaacctctgccagagaacagagaggcccggggcgacacggtcgttagtgaggcggaattgacccccgaagctgtgacagctgcagaaactgcagggccattgggttccgaagaaggaaccgaagcatctgctgctgaagagaccacagaaatggtgtcagcagtctcccagttaaccgactccccagacaccacagaggaggccactccggtgcaggaggtggaaggtggcgtacctgacatagaagagcaagagaggcggactcaagaggtcctccaggcagtggcagaaaaagtgaaagaggaatcccagctgcctggcaccggtgggccagaagatgtgcttcagcctgtgcagagagcagaggcagaaagaccagaagagcaggctgaagcgtcgggtctgaagaaagagacggatgtagtgttgaaagtagatgctcaggaggcaaaaactgagccttttacacaagggaaggtggtggggcagaccaccccagaaagctttgaaaaagctcctcaagtcacagagagcatagagtccagtgagcttgtaaccacttgtcaagccgaaaccttagctggggtaaaatcacaggagatggtgatggaacaggctatcccccctgactcggtggaaacccctacagacagtgagactgatggaagcacccccgtagccgactttgacgcaccaggcacaacccagaaagacgagattgtggaaatccatgaggagaatgaggtcgcatctggtacccagtcagggggcacagaagcagaggcagttcctgcacagaaagagaggcctccagcaccttccagttttgtgttccaggaagaaactaaagaacaatcaaagatggaagacactctagagcatacagataaagaggtgtcagtggaaactgtatccattctgtcaaagactgaggggactcaagaggctgaccagtatgctgatgagaaaaccaaagacgtaccatttttcgaaggacttgaggggtctatagacacaggcataacagtcagtcgggaaaaggtcactgaagttgcccttaaaggtgaagggacagaagaagctgaatgtaaaaaggatgatgctcttgaactgcagagtcacgctaagtctcctccatcccccgtggagagagagatggtagttcaagtcgaaagggagaaaacagaagcagagccaacccatgtgaatgaagagaagcttgagcacgaaacagctgttaccgtatctgaagaggtcagtaagcagctcctccagacagtgaatgtgcccatcatagatggggcaaaggaagtcagcagtttggaaggaagccctcctccctgcctaggtcaagaggaggcagtatgcaccaaaattcaagttcagagctctgaggcatcattcactctaacagcggctgcagaggaggaaaaggtcttaggagaaactgccaacattttagaaacaggtgaaacgttggagcctgcaggtgcacatttagttctggaagagaaatcctctgaaaaaaatgaagactttgccgctcatccaggggaagatgctgtgcccacagggcccgactgtcaggcaaaatcgacaccagtgatagtatctgctactaccaagaaaggcttaagttccgacctggaaggagagaaaaccacatcactgaagtggaagtcagatgaagtcgatgagcaggttgcttgccaggaggtcaaagtgagtgtagcaattgaggatttagagcctgaaaatgggattttggaacttgagaccaaaagcagtaaacttgtccaaaacatcatccagacagccgttgaccagtttgtacgtacagaagaaacagccaccgaaatgttgacgtctgagttacagacacaagctcacgtgataaaagctgacagccaggacgctggacaggaaacggagaaagaaggagaggaacctcaggcctctgcacaggatgaaacaccaattacttcagccaaagaggagtcagagtcaaccgcagtgggacaagcacattctgatatttccaaagacatgagtgaagcctcagaaaagaccatgactgttgaggtagaaggttccactgtaaatgatcagcagctggaagaggtcgtcctcccatctgaggaagagggaggtggagctggaacaaagtctgtgccagaagatgatggtcatgccttgttagcagaaagaatagagaagtcactagttgaaccgaaagaagatgaaaaaggtgatgatgttgatgaccctgaaaaccagaactcagccctggctgatactgatgcctcaggaggcttaaccaaagagtccccagatacaaatggaccaaaacaaaaagagaaggaggatgcccaggaagtagaattgcaggaaggaaaagtgcacagtgaatcagataaagcgatcacaccccaagcacaggaggagttacagaaacaagagagagaatctgcaaagtcagaacttacagaatcttaaaacatcatgcagttaaactcattgtctgtttggaagaccagaatgtgaagacaagtagtagaagaaaatgaatgctgctgctgagactgaagaccagtatttcagaactttgagaattggagagcaggcacatcaactgatctcatttctagagagcccctgacaatcctgaggcttcatcaggagctagagccatttaacatttcctctttccaagaccaacctacaattttcccttgataaccatataaattctgatttaaggtcctaaattcttaacctggaactggagttggcaatacctagttctgcttctgaaactggagtatcattctttacatatttatatgtatgttttaagtagtcctcctgtatctattgtatatttttttcttaatgtttaaggaaatgtgcaggatactacatgctttttgtatcacacagtatatgatggggcatgtgccatagtgcaggcttggggagctttaagcctcagttatataacccacaaaaaacagagcctcctagatgtaacattcctgatcaaggtacaattctttaaaattcactaatgattgaggtccatatttagtggtactctgaaattggtcactttcctattacacggagtgtgccaaaactaaaaagcattttgaaacatacagaatgttctattgtcattgggaaattttgctttctaacccagtggaggttagaaagaagttatattctggtagcaaattaactttacatcctttttcctacttgttatggttgtttggaccgataagtgtgcttaatcctgaggcaaagtagtgaatatgttttatatgttatgaagaaaagaattgttgtaagtttttgattctactcttatatgctggactgcattcacacatggcatgaaataagtcaggttctttacaaatggtattttgatagatactggattgtgtttgtgccatatttgtgccattcctttaagaacaatgttgcaacacattcatttggataagttgtgatttgacgactgatttaaataaaatatttgcttcacttaaaaaaaaaaaa

SEQ ID NO: 16 - protein, 1781 aa, Homo sapiens [sequence omitted]

SEQ ID NO: 17 - DNA, 1103 bp, Homo sapiens
cttcggtcctgctgtagtgccttctgcgccaggcccggttcaatcagcggccacaactgtctagggctcagacaccaccagccaatgagggagggcacgtggagccgcgtctgggctcgcggctcctgaccaatggggaagtggcatgtgggagggcgccggggttccccccgccaatggggagctacggcgcgcggccgggacttggaggcggtgcggcgcggcgggtgcggttcagtcggtcggcggcggcagcggaggaggaggaggaggaggaggatgaggaggatgaggaggatgtgggccacgcaggggctggcggtgcgcgtggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcaccgaccgatttgtagtctgctcaatctctgttgcacctgagggggaaaaaacagttcaactgcttactcccaaaacagcctttttgtaatttattttttaagtgggctcctgacaatactgtatcagatgtgaagcctggagctttcctgatgatgctggccctacagtacccccatgaggggattcccttccttctgttgctggtgtactctaggacttcaaagtgtgtctgggatttttttattaaagaaaaaaaatttctagctgtcaaaaaaaaaaa

SEQ ID NO: 18 - DNA, 817 bp, Drosophila melanogaster
agtacagttaattaaagtacgctcaagcacagtcgaagtaataacaaaatgaagacgtggtacatggtggtagtgatcggcttcctggcgacgttggcccaaacgtcgctggccctgaaagaggaggactgcgaagtttgcgtcaagacggttaggcggttcgcagattcgctggacgactccaccaagaaggactacaaacagatcgaaacggccttcaaaaagttctgcaaagcgcagaaaaacaaggaacacagattctgttacctcggcggtctggaagaatccgccacgggcatccttaacgagctgagcaaacccctcagttggtccatgccagctgagaagatctgcgagaagctgaagaagaaggacgcacaaatctgcgaccttcgctatgagaaacaaatcgatctgaacagcgtggacctgaagaagctgaaggtacgcgacctgaagaaaatcctcaacgactgggacgagagctgtgacggttgcctggagaagggcgacttcatcaagcgtatcgaggagctgaagcccaagtactcgcgcagcgagctgtagagcgcagtcagttacttgtgtagttacatggaacacgcccacttagctaaaacaacaaccacatgaaacgccccctacacatccatatttttagttaatttacaattttgatagactttgagagaattgaatataactatatatgcctaaagataaaacctatattttgaaataaaaaacaacaaacacacaagacctcgtcctctcaaacggttgataaaaaaaaaaaaaaaaaaaaaaaaaaaaaaaa

SEQ ID NO: 19 - protein, 172 aa, Drosophila melanogaster
MKTWYMVVVIGFLATLAQTSLALKEEDCEVCVKTVRRFADSLDDSTKKDYKQIETAFKKFCKAQKNKEHRFCYLGGLEESATGILNELSKPLSWSMPAEKICEKLKKKDAQICDLRYEKQIDLNSVDLKKLKVRDLKKILNDWDESCDGCLEKGDFIKRIEELKPKYSRSEL

SEQ ID NO: 20 - DNA, 705 bp, Mus musculus
tccaagacaattttctttaataaaaaaatcccagacaaactttgaagtcctagagtaaaccagcaaagaaggaagtaaattgtccccacttggggatggggaaattgtccccactcggggatgggcaagagggaactgcagagccagcatcactaggaaagctccaggttcacatctcattgtcaggagcccgcttaaaaaataaattacaaaatggttatttggggaagagacagataaactgctttttccccttcaggtgcagcaggaattgggcagactacagatcagtccgtgcgctggctgccttgggggcgtatttaggcatcagttcatttatcttccggatatagtcagacttttctgcacagcctttgcacatctccccccagtcgtccaggatcttcttcagctctttcacccggagcttcttcaggtccactgtgctcaggtcaatctgcttgtcgtattttagttcacagatctggctgtctttcttcttcagcttctcacagatcttttccacagggatatggtgggccaggggcttcgacacctcattgatgatcttggtggcagcatcatctgtggctccaatgagtagcacaaccgattctctttgcctcttgcttcacgggcaaactttataagtcttcttcatagtggctggtgaaatgtgacatctctgtctttgaggtctgtaaa

SEQ ID NO: 21 - DNA, 890 bp, Mus musculus
ccgggtgcggttcattcgcgcggcatccggcggtggtggagacggctgaggaggatgtgggctacgcgcgggctggcggtacgctggccctgagcgtgctgcctgacagccgggcgctgcggccaggagactgtgaagtttgtatttcttatctgggacgattttaccaggacctcaaagacagagatgtcacattttcaccagccactattgaagaagaacttataaagttttgccgtgaagcaagaggcaaagagaatcggttgtgctactacattggagccacagatgatgctgccaccaagatcatcaatgaggtgtcgaagcccctggcccaccatatccctgtggaaaagatctgtgagaagctgaagaagaaagacagccagatctgtgaactaaaatacgacaagcagattgacctgagcacagtggacctgaagaagctccgggtgaaagagctgaagaagatcctggacgactggggggagatgtgcaaaggctgtgcagaaaagtctgactatatccggaagataaatgaactgatgcctaaatacgcccccaaggcagccagcgcacggactgatctgtagtctgcccaattcctgctgcacctgaaggggaaaaagcagtttatctgtctcttccccaaataaccattttgtaatttattttttaagcgggctcctgacaatgagatgtgaacctagagctttcctagtgatgctggttttgcagttccctcttgcccatccccgagtggggacaatttccccatccccaagtggggacaatttacttccttctttgctggtttactctaggacttcaaagtttgtctgggatttttttattaaaaaaaattgtctttggagagttaaaaaaaaaaa

SEQ ID NO: 22 - protein, 198 aa, Mus musculus
GCGSFARHPAVVETAEEDVGYARAGGTLALSVLPDSRALRPGDCEVCISYLGRFYQDLVEGFRDVTFSPATIEEELIKFCREARGKENRLCYYIGATDDAATKIINEVSKPLAHHIPVEKICEKLKKKDSQICELKYDKQIDLSTVDLKKLRVKELKKILDDWGEMCKGCAEKSDYIRKINELMPKYAPKAASARTDL

SEQ ID NO: 23 - DNA, 2173 bp, Homo sapiens
gaactatagttgaaggctgctgccaatacaacaccactgtgaaacagaatggtgtatgccaaatttgagtccctctttaggacgtatgacaaggacatcacctttcagtattttaagagcttcaaacgagtcagaataaacttcagcaaccccttctccgcagcagatgccaggctccagctgcataagactgagtttctgggaaaggaaatgaagttatattttgctcagaccttacacataggaagctcacacctggctccgccaaatccagacaagcagtttctgatctcccctcccgcctctccgccagtgggatggaaacaagtggaagatgcgaccccagtcataaactatgatctcttatatgccatctccaagctggggccaggggaaaagtatgaattgcacgcagcgactgacaccactcccagcgtggtggtccatgtatgtgagagtgatcaagagaaggaggaagaagaggaaatggaaagaatgaggagacctaagccaaaaattatccagaccaggaggccggagtacacgccgatccacctcagctgaactggcacgcgacgaggacgcattccaaatcatactcacgggaggaatcttttactgtggaggtggctggtcacgacttcttcggaggtggcagccgagatcggggtggcagaaatcccagttcatgttgctcagaagagaatcaaggccgtgtccccttgttctaatgctgcacaccagttactgttcatggcacccgggaatgacttgggccaatcactgagtttgtggtgatcgcacaaggacatttgggactgtcttgagaaaacagataatgatagtgttttgtacttgttcttttctggtaggttctgtctgtgccaagggcaggttgatcagtgagctcaggagagagcttcctgtttctaagtggcctgcaggggccactctctactggtaggaagaggtaccacaggaagccgcctagtgcagagaggttgtgaaaacagcagcaatgcaatgtggaaattgtagcgtttcctttcttccctcatgttctcatgtttgtgcatgtatattactgatttacaagactaacctttgttcgtatataaagttacaccgttgttgttttacatcttttgggaagccaggaaagcgtttggaaaacgtatcacctttcccagattctcggattctcgactctttgcaacagcacttgcttgcggaactcttcctggaatgcattcactcagcatccccaaccgtgcaacgtgtaacttgtgcttttgcaaaagaagttgatctgaaattcctctgtagaatttagcttatacaattcagagaatagcagtttcactgccaacttttagtgggtgagaaattttagtttaggtgtttgggatcggacctcagtttctgttgtttcttttatgtggtggtttctatacatgaatcatagccaaaaacttttttggaaactgttggttgagatagttggttcttttaccccacgaagacatcaagatacacttgtaaataaagctgatagcatatattcatacctgttgtacacttgggtgaaaagtatggcagtgggagactaagatgtattaacctacctgtgaatcatatgttgtaggaaaagctgttcccatgtctaacaggacttgaattcaaagcatgtcaagtggatagtagatctgtggcgatatgagagggatgcagtgcctttccccattcattcctgatggaattgttatactaggttaacatttgtaatttttttctagttgtaatgtgtatgtctggtaaataggtattatattttggccttacaataccgtaacaatgtttgtcattttgaaatacttaatgccaagtaacaatgcatgctttggaaatttggaagatggttttattctttgagaagcaaatatgtttgcattaaatgctttgattgttcatatcaagaaattgattgaacgttctcaaaccctgtttacggtacttggtaagagggagccggtttgggagagaccattgcatcgctgtccaagtgtttcttgttaagtgcttttaaactggagaggctaacctcaaaatactttttttaactgcattctataataaatgggcacagtatgctccttacaaaaaaaaaaaaaaa

SEQ ID NO: 24 - protein, 171 aa, Homo sapiens
MVYAKFESLFRTYDKDITFQYFKSFKRVRINFSNPFSAADARLQLHKTEFLGKEMKLYFAQTLHIGSSHLAPPNPDKQFLISPPASPPVGWKQVEDATPVINYDLLYAISKLGPGEKYELHAATDTTPSVVVHVCESDQEKEEEEEMERMRRPKPKIIQTRRPEYTPIHLS

SEQ ID NO: 25 - DNA, 4919 bp, Homo sapiens
ttgctgtcaggtgactctcccgtggcgccatggcggaagcagtggagcaggaaactgggtcccttgaagaatctacagatgagtctgaggaagaagagagcgaagaggaacccaagctgaagtatgaaaggctttccaatggggtaactgaaatacttcagaaggatgcagctagctgcatgacagtccatgacaagtttttggcattgggcacacattatggcaaggtttatttacttgatgtccaggggaacatcactcagaagtttgatgtaagtcctgtgaagataaatcagattagcttggatgaaagtggagagcacatgggtgtgtgttcagaggatggcaaggtgcaggtatttggactgtattctggagaagaatttcacgagacttttgactgtcccattaaaattattgctgtgcacccacatttcgtgagatccagttgcaagcagtttgtgaccggagggaagaagctgctactgtttgaacggtcttggatgaacagatggaagtctgctgttctgcatgaaggggaagggaacataaggagtgtgaagtggagaggccatctgattgcttgggccaataatatgggtgtgaagatttttgacatcatctcaaagcaaagaatcaccaatgtgccccgggatgatataagtcttcgcccagacatgtatccctgcagcctctgctggaaggacaatgtgacactgattattggctgggggacttctgtcaaggtgtgctcagtgaaggaacggcatgccagtgaaatgagggatttgccaagtcgatatgttgaaatagtgtctcagtttgaaactgaattctacatcagtggacttgcacctctctgtgatcagcttgttgtactttcgtatgtaaaggagatttcagaaaaaacggaaagagaatactgtgccaggcctagactggacatcatccagccactttctgagacttgtgaagagatctcttctgatgctttgacagtcagaggctttcaggagaatgaatgtagagattatcatttagaatactctgaaggggaatcacttttttacatcgtgagtccgagagatgttgtagtggccaaggaacgagaccaagatgatcacattgactggctccttgaaaagaagaaatatgaagaagcattgatggcagctgaaattagccaaaaaaatattaaaagacataagattctggatattggcttggcatatataaatcacctggtggagagaggagactatgacatagcagcacgcaaatgccagaaaattcttgggaaaaatgcagcactctgggaatatgaagtttataaatttaaagaaattggacagcttaaggctattagtccttatttgccaagaggtgatccagttctgaaaccactcatctatgaaatgatcttacatgaatttttggagagtgattatgagggttttgccacattgatccgagaatggcctggagatctgtataataattcagtcatagttcaagcagttcgggatcatttgaagaaagatagtcagaacaagactttacttaaaaccctggcagaattgtacacctatgacaagaactatggcaatgctctggaaatatacttaacattaagacataaagacgtttttcagttgatccacaagcataatcttttcagttctatcaaggataaaattgttttattaatggattttgattcagagaaagctgttgacatgcttttggacaatgaagataaaatttcaattaaaaaggtagtggaagaattggaagacagaccagagctacagcatgtgtatttgcataagcttttcaagagagaccaccataaggggcagcgttaccatgaaaaacagatcagtctttatgctgaatatgatcgaccaaacttacttccctttctccgagacagtacccattgcccacttgaaaaggctcttgagatctgtcaacagagaaactttgtagaagagacagtttatcttctgagccgaatgggtaatagccgaagtgccctgaagatgattatggaggaattacatgatgttgataaagcaatcgaatttgccaaggagcaagatgatggagagctgtgggaagatttgattttatattccattgacaaaccaccatttattactggcttgttaaacaacattggcacacatgttgacccaattctactgattcaccgtattaaggaaggaatggagatccccaatttgagagattccttggttaaaattctgcaagactacaatttgcaaattctgcttcgtgaaggctgcaagaagattctcgtagctgactctttgtccttactgaagaaaatgcaccgaactcaaatgaaaggtgttcttgttgatgaggagaacatctgtgagtcgtgcctttcccctattcttccatcagatgcagctaagcccttcagcgtggtggtcttccattgccggcacatgttccacaaggagtgcctgcccatgcccagcatgaactctgctgcacagttctgcaacatctgcagtgctaagaaccgtggaccaggaagtgcaattttggagatgaaaaaatagctcatttctccttgtcagtctccttgtcaccactctttttgagactgtttttgcaacaacaaaagcatttgttgacactcgtgctgttaagagatttgtttatgtttatattatactcaaaaacaatttcttcatctattcctgtactaatggtttctctttgcagttcacagagaatttggggctctcttcatgccttgaaattttggggtccatagtgaatattttgttatttatttgtttggctcattctttatatagtaatggaaacataagtctaggagttagaaatgaattttttagaccttagtaaaaccatttaaccataaaatggacaactgagaattctcccagctgcctgaaagcgtcgccaactgtggttatcctgcaagctgctacctgcaacttggacgttgtttccacgtgctctgctggctacgattcttgcattctgggtttggcttttttctgtgtcatcaactatggttatcctctaaataggcatttaatgaaacattgtacaaattgtcactcatttgatgacacctgggaataacattagcaggctgatgtcctgcaccattatgtttactaatcacatgttctgtgtgctgtgacgactgtcaaagagtatctggccatggcggacactcagcatttgttgattgaataaatgttagctcttctcattgtgaaggactcacttttactgggataaacaaatgcagttaagaattctggcacccttgtaaggaagaaaagagagttcaacaccttcgagtctgagcgcttgtggctagagtttgccaggagggaggaaaccagtgaccctgaaaactgagggtgcctcaggagcagtgggaccacctgatgctgaaggacggactaatgatgtttcctcttgccttctctggtgcctccattgccctccatggaacagagcatatcatagaaggagaaaaattccaacttgtaattgtgtcttacagttactggcttcatcttccttgggatatatggtcatcctctaatgagtgtaaaagtgcgcaaaacacatccttattgttcctgatctcttagtcccataaatgggaacaaatacagctttctgcttctttctttttggggaaaggacagggtgctagtgagtactgacagcatgccagctaccaaagtcacccagccattcccatgagcagcagttcatttaattgtcacagcgtcgccaggaagaagatctgataaacctaggtttacagataaagaaagcaaaatgtagagatgttgttgaggtcacagaggtgactgcctaacttcagagcagggcttctgatccctttaagaaattacagggccagccgggcatggtggctcacgcccgtaatcccagggctttgggaagccttggcgggtggatcacctgagatcgcacgttcgagaccggcctgaccaacatggagaaaccccatctctactaaaaacacaaattagccaggcatggtggtacatgcctgtaatcccggctactcaggaggctgaggcaggagaatcacttgaccccaggagacatatgttgtggtgagctgaggtctcgccattgcactccagcctgggcaacaagagcaaaactccgtctcaaaaaagaaaagaaaagaaaagaaatcatagggccaagttcaaaggaaatgcacagaacatatcttcacattagagttaagaattctctagcaaacaacagatttttttgttgttgttagtcacaaatacttagaactggaaggcgctttgttattattgaatgtacccctcagccttctcagcatttccttatcccaagactagtgtgctttctgctacactgctagttttcagttttgttcttacccaattgttttttcttttcaacattaccaatttacagattcagtttattacatttacattaatcctcacttatgatttgagcaagctcatttccagaaaagtttactttaagatcatcaataggatttgctaatttcagtgaagtcattttgcttcaggggtaaattatcctagttaccaagtcctatttggacataaagaaaatcctacttatagaaaaggagaaaataattaaacagtcttcatttttaagtaactgatttaaaaggaaaataataaaatatgttcgtttatcatttcagaaattgctgtaacacactggaaaattcctgaacaatatagattttatcgttaataaaaaacactagctttcgttccttagaatgtcttttcttttgaataaacagtattgggtgatttaaaaaaaaaaaaaaaaaaaaa

SEQ ID NO: 26 - protein, 854 aa, Homo sapiens
MAEAVEQETGSLEESTDESEEEESEEEPKLKYERLSNGVTEILQKDAASCMTVHDKFLALGTHYGKVYLLDVQGNITQKFDVSPVKINQISLDESGEHMGVCSEDGKVQVFGLYSGEEFHETFDCPIKIIAVHPHFVRSSCKQFVTGGKKLLLFERSWMNRWKSAVLHEGEGNIRSVKWRGHLIAWANNMGVKIFDIISKQRITNVPRDDISLRPDMYPCSLCWKDNVTLIIGWGTSVKVCSVKERHASEMRDLPSRYVEIVSQFETEFYISGLAPLCDQLVVLSYVKEISEKTEREYCARPRLDIIQPLSETCEEISSDALTVRGFQENECRDYHLEYSEGESLFYIVSPRDVVVAKERDQDDHIDWLLEKKKYEEALMAAEISQKNIKRHKILDIGLAYINHLVERGDYDIAARKCQKILGKNAALWEYEVYKFKEIGQLKAISPYLPRGDPVLKPLIYEMILHEFLESDYEGFATLIREWPGDLYNNSVIVQAVRDHLKKDSQNKTLLKTLAELYTYDKNYGNALEIYLTLRHKDVFQLIHKHNLFSSIKDKIVLLMDFDSEKAVDMLLDNEDKISIKKVVEELEDRPELQHVYLHKLFKRDHHKGQRYHEKQISLYAEYDRPNLLPFLRDSTHCPLEKALEICQQRNFVEETVYLLSRMGNSRSALKMIMEELHDVDKAIEFAKEQDDGELWEDLILYSIDKPPFITGLLNNIGTHVDPILLIHRIKEGMEIPNLRDSLVKILQDYNLQILLREGCKKILVADSLSLLKKMHRTQMKGVLVDEENICESCLSPILPSDAAKPFSVVVFHCRHMFHKECLPMPSMNSAAQFCNICSAKNRGPGSAILEMKK

SEQ ID NO: 27 - DNA, 2587 bp, Homo sapiens
aggcgctgcggccgtcccgggccgtgactcctcctttcccccgccccgcctccgttcggagagccggcgggcgggcgcctctcggccaggtacgcggccggctgggataggggtcgcgggcgggctttggtcgcgcagcagccggtcctccccggaggtaggcgggcgcgggccctgttgggtctttgggacgcgggtcccgctggggccggggatgctcctctctgtgagcgcggtcccgtccccccctgtccccgggcgggcgcacgggggtctaaccttggggggcggcgctcccgcctgtcccgggtgccggggttcgcgtcccgcggggccttcctcgctctttgtctcttctgagtgaacttgatgacccccttcttccaggaagcgcctcttggacgcgtgtgaccgatgcccagattgcacgaccacttctggagctgctcctgtgcgcacagcgcgaggcgccgaggccccccgcgagccagcaccgcggggctgccgcccaaggttggggagatgatcaacgtttccgtgtccgggccctccctgctggcggcccacggtgccccggacgctgaccccgcgcccaggggccgcagtgctgcgatgagcggccccgagcccggcagcccctaccccaacacctggcatcatcgcctgttgcagaggagcctcgtgctcttctcggttggggtggtcctaaccctggtgctcaacctgctgcagatccagaggaatgtcactctcttccccgaggaggtgatcgccaccatcttttcctccgcctggtgggtccctccctgctgcgggacagcagctgctgttgttggcctactgtacccctgtatcgacagtcacctcggagaaccccacaaatttaagagagaatgggccagtgtcatgcgctgcatagcaggttttggtggcattaaccacgccagtgctaaattggattttgccaataatgtccagctgtccttgactttagcagccctatctttgggcctttggtggacatttgatcgttccagaagtggccttgggctggggatcaccatagcttttctagctacgctgatcacgcagtttctcgtgtataatggtgtctatcagtatacgtccccagatttcctctatattcgttcttggctcccttgtatatttttctcaggaggcgtcacggtggggaacataggacgacagttagctatgggtgttcctgaaaagccccatagtgattgagtcttcaaaaccaccgattctgagagcaaggaagattttggaagaaaatctgactgtggattatgacaaagattatcttttttcttaagtaatctatttagatcgggctgactgtacaaatgactcctggaaaaaactcgtcacctatctagaaaagtcaagaatagggaggtggagaatgatgacttaccctgaagtcttcccttgctacccacactggcgcctgtctgtgccctggagcattctgcccagcctacgtgggttcagtcaggtgccaccttcccaagtattcgatttcattcatgtgattaaaacaagttgccatatttcaaagccttgagctaagactcaattaccaacccgcagttttgtgtcagtgcccaaaggagggaggttgatggtgcttaacaaacatgaagtatggtgtaataggaataatatttatccaaaagatttttaaaaatagggctgtgtttaaagaaggaatcaaaacaagaaaagcagcagtgattatagagaggtcacactctaagtggggtcgcggcgtggccacgcttcacggtcacgctcgtccgtcctgcagtggcgtgtttacatggtcacacgtgtgtgtatcaccagtgggtcaactgcttgtcattcctcccgcggcagtgttgtgtagacaatcttactgagcaaaaggcaatgaaaagtcttgggctcccacactgcgatatattggaattcccacctcagtttatgaagtttatttcgaaatccatagtcatctaagaatgaatacctgtctgccatgtatttcaatcttagtgagccaaaattgtttgtttgttactacagaatagagatgactgttttttggcacagccctatggaatttgcaatctgtgattgccttgtaaaaaggagagtgcatatggcactgcattaaacgtgtggtgtttctagtcaatgatattggtgagcacaatgtattcatttaatggcatagaccataccagacctaatttgcaagtattgggtcttcaaacttcaagtgcaatgtattatgaaaaccaatctgagccttgtatctcttaaatatttattccttctaacgtgtgagatgtcccgagagaagggttctccattcatttcagtgctgcctggaggaaactcggcaatgatttcttcagttgtgaagttcctttcgggttacaacctccactggaaccctcaaccttcgaaatactccagttttgggggttggggccatttacttataaatttaccgccgggtttttggaatctacatgtcttgggggcgggctcaaattcttcgaaagtggttggattaaaa

SEQ ID NO: 28 - protein, 277 aa, Homo sapiens
MPRLHDHFWSCSCAHSARRRGPPRASTAGLPPKVGEMINVSVSGPSLLAAHGAPDADPAPRGRSAAMSGPEPGSPYPNTWHHRLLQRSLVLFSVGVVLTLVLNLLQIQRNVTLFPEEVIATIFSSAWWVPPCCGTAAAVVGLLYPCIDSHLGEPHKFKREWASVMRCIAGFGGINHASAKLDFANNVQLSLTLAALSLGLWWTFDRSRSGLGLGITIAFLATLITQFLVYNGVYQYTSPDFLYIRSWLPCIFFSGGVTVGNIGRQLAMGVPEKPHSD

SEQ ID NO: 29 - DNA, 2114 bp, Homo sapiens
cacactgctcagggaagagcctgctacggtggactgtgagactcagtgcactgtcctcctcccagcgaccccacgctggaccccctgccggaccctccacccttcggcccccaagcttcccaggggcttcctttggactggactgtccctgctcatccattctcctgccacccccagacctcctcagctccaggttgccacctcctctcgccagagtgatgaggtcccggcttctgctctccgtggcccatctgcccacaattcgggagaccacggaggagatgctgcttgggggtcctggacaggagcccccaccctctcctagcctggatgactacgtgaggtctatatctcgactggcacagcccacctctgtgctggacaaggccacggcccagggccaacccaggccaccccacaggccagcccaggcctgccggaagggccgccctgctgtgtccctgcgagacatcaccgcacgtttcagtggccagcagcccacactgcccatggctgatactgtggaccccctggactggctttttggggagtcccaggaaaagcagccaagccagagggacctgccaaggaggactggcccctctgctggcctctggggtccacatagacagatggacagcagcaagcccatgggggcccccagagggaggctctgtgaagccaggatgcctgggcattccctggcaagaccaccgcaggatgggcagcagagctctgacctaagaagctggacttttgggcagtctgcccaagccatggcctcccgccaccgcccccgccccagcagtgtcctcagaacactctactcgcacctcccggtgatccatgaactctgacccctccccagtaaaggcttctgtagagagcatgctgggtctgcatctcctctcgtctcctccatggtggtcactgcccctggcaggtctctgaaagggaaatgcttttctgcagaggccccttcttgggcagttcacagttagacccaccccctctgaatatgataacagcctgtttcacatgaggagatgttaccaatcccgttcgctctgacccttgctggctgatcaccttgagcaacttacttaacatctgtgttcctcagtttctcatgggtaatatagggataattactggcacctgcctcccaggccattctgacgtgtaccgcatataggagcccactggctgagtagctaccatcatcgctggtggggaaactggtggtaggggtgtgagggtagtgggggtgtcagcccccaggtgtttcagaacaaggcctcgggcactcccaagtctgcctcttggctcccaccctcaaagcccatgttctgtgaggcccaagagaacacatggagtcttagcaaatgcactaatgtattccgggggactgtcacctggcaccactggggcactctgctggctacaactcatacgtcctgtggtggcattgggagagttcccccatgatgagggccaagatagaatctgtaccactcagtgctaccatccccacccctacaccacttccacacaggggcctcatggcatggtcagggtcccagctgtgggtgagagcagggcactgtccagctgtccactggggaagtcaagatgtcctaaggcccaggccagggcatctggagtctgaaggaccctagttcctagaggcatctggcagcaagaaggtgaggcatcagggaacgggaatcaggctgggactgatcagaggtgaagggacagagagaggagaggaggaagattgagctggggcaacagccaagctcacctggcaggtctctgccacctccttctctgtgagctgtcagtctaggttattctctttttttgtggctatttttaattgctttggatttgttaaatgttttctgtcttctgttaagtgtgttttctctggagatagaatgtaaaccatattaaaaggaaaaagtttcagacaagcaattacccagtttccttatctataaaatggggacatcagcaatgttcttcacaccctgcaagggctgggaattgcgcatgtgaacttggagctgcatttatgagcactgtagacaaatgtttgtatctgtcac

SEQ ID NO: 30 - protein, 212 aa, Homo sapiens
MRSRLLLSVAHLPTIRETTEEMLLGGPGQEPPPSPSLDDYVRSISRLAQPTSVLDKATAQGQPRPPHRPAQACRKGRPAVSLRDITARFSGQQPTLPMADTVDPLDWLFGESQEKQPSQRDLPRRTGPSAGLWGPHRQMDSSKPMGAPRGRLCEARMPGHSLARPPQDGQQSSDLRSWTFGQSAQAMASRHRPRPSSVLRTLYSHLPVIHEL

SEQ ID NO: 31 - DNA, 16571 bp, Homo sapiens
gatcacaggtctatcaccctattaaccactcacgggagctctccatgcatttggtattttcgtctggggggtgtgcacgcgatagcattgcgagacgctggagccggagcaccctatgtcgcagtatctgtctttgattcctgcctcattctattatttatcgcacctacgttcaatattacaggcgaacatacctactaaagtgtgttaattaattaatgcttgtaggacataataataacaattgaatgtctgcacagccgctttccacacagacatcataacaaaaaatttccaccaaacccccccctccccccgcttctggccacagcacttaaacacatctctgccaaaccccaaaaacaaagaaccctaacaccagcctaaccagatttcaaattttatctttaggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaagggacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactgagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagatcaaaacactgaactgacaattaacagcccaatatctacaatcaaccaacaagtcattattaccctcactgtcaacccaacacaggcatgctcataaggaaaggttaaaaaaagtaaaaggaactcggcaaaccttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcatgacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatggctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgcaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgatagccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcgcagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatggtggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaactaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgagccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaagcacataatttgaatcaacacaaccacccacagcctaattattagcatcatccccctactattttttaaccaaatcaacaacaacctatttagctgttccccaaccttttcctccgaccccctaacaacccccctcctaatactaactacctgactcctacccctcacaatcatggcaagccaacgccacttatccagcgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacggactcacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcattttcctaattaccatactaatcttagttaccgctaacaacctattccaactgttcatcggctgagagggcgtaggaattatatccttcttgctcatcagttgatgatacgcccgagcagatgccaacacagcagccattcaagcagtcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaaccactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcagtactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaattaaccccctaataaaattaattaaccactcattcatcgacctccccaccccatccaacatctccgcatgatgaaacttcggctcactccttggcgcctgcctgatcctccaaatcaccacaggactattcctagccatacactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttacccttcattattgcagccctagcagcactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagacgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaacccacatcaaaccccccccccccatgcttacaagcaagtacagcaatcaaccttcaactatcacacatcaactgcaactccaaagccacccctcacccactaggataccaacaaacctacccacccttaacagtacatagtacataaagtcatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggccataaagcctaaatagcccacacgttccccttaaataagacatcacgatg

SEQ ID NO: 32 - protein, 513 aa, Homo sapiens
MFADRWLFSTNHKDIGTLYLLFGAWAGVLGTALSLLIRAELGQPGNLLGNDHIYNVIVTAHAFVMIFFMVMPIMIGGFGNWLVPLMIGAPDMAFPRMNNMSFWLLPPSLLLLLASAMVEAGAGTGWTVYPPLAGNYSHPGASVDLTIFSLHLAGVSSILGAINFITTIINMKPPAMTQYQTPLFVWSVLITAVLLLLSLPVLAAGITMLLTDRNLNTTFFDPAGGGDPILYQHLFWFFGHPEVYILILPGFGMISHIVTYYSGKKEPFGYMGMVWAMMSIGFLGFIVWAHHMFTVGMDVDTRAYFTSATMIIAIPTGVKVFSWLATLHGSNMKWSAAVLWALGFIFLFTVGGLTGIVLANSSLDIVLHDTYYVVAHFHYVLSMGAVFAIMGGFIHWFPLFSGYTLDQTYAKIHFTIMFIGVNLTFFPQHFLGLSGMPRRYSDYPDAYTTWNILSSVGSFISLTAVMLMIFMIWEAFASKRKVLMVEEPSMNLEWLYGCPPPYHTFEEPVYMKS

SEQ ID NO: 33 - DNA, 957 bp, Homo sapiens
atacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaa

SEQ ID NO: 34 - protein, 318 aa, Homo sapiens
MPMANLLLLIVPILIAMAFLMLTERKILGYMQLRKGPNVVGPYGLLQPFADAMKLFTKEPLKPATSTITLYITAPTLALTIALLLWTPLPMPNPLVNLNLGLLFILATSSLAVYSILWSGWASNSNYALIGALRAVAQTISYEVTLAIILLSTLLMSGSFNLSTLITTQEHLWLLLPSWPLAMMWFISTLAETNRTPFDLAEGESELVSGFNIEYAAGPFALFFMAEYTNIIMMNTLTTTIFLGTTYDALSPELYTTYFVTKTLLLTSLFLWIRTAYPRFRYDQLMHLLWKNFLPLTLALLMWYVSMPITISSIPPQT

SEQ ID NO: 35 - DNA, 1380 bp, Homo sapiens
atgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgt

SEQ ID NO: 36 - protein, 459 aa, Homo sapiens
MLKLIVPTIMLLPLTWLSKKHMIWINTTTHSLIISIIPLLFFNQINNNLFSCSPTFSSDPLTTPLLMLTTWLLPLTIMASQRHLSSEPLSRKKLYLSMLISLQISLIMTFTATELIMFYIFFETTLIPTLAIITRWGNQPERLNAGTYFLFYTLVGSLPLLIALIYTHNTLGSLNILLLTLTAQELSNSWANNLMWLAYTMAFMVKMPLYGLHLWLPKAHVEAPIAGSMVLAAVLLKLGGYGMMRLTLILNPLTKHMAYPFLVLSLWGMIMTSSICLRQTDLKSLIAYSSISHMALVVTAILIQTPWSFTGAVILMIAHGLTSSLLFCLANSNYERTHSRIMILSQGLQTLLPLMAFWWLLASLANLALPPTINLLGELSVLVTTFSWSNITLLLTGLNMLVTALYSLYMFTTTQWGSLTHHINNMKPSFTRENTLMFMHLSPILLLSLNPDIITGFSS

SEQ ID NO: 37 - DNA, 2360 bp, Homo sapiens
gctacgcgggccacgctgctggctggcctgacctaggcgcgcggggtcgggcggccgcgcgggcgggctgagtgagcaagacaagacactcaagaagagcgagctgcgcctgggtcccggccaggcttgcacgcagaggcgggcggcagacggtgcccggcggaatctcctgagctccgccgcccagctctggtgccagcgcccagtggccgccgcttcgaaagtgactggtgcctcgccgcctcctctcggtgcgggaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccagtggaaaatcgcttatatacctatgaccacacaaccatcctggccgtggtggctgtggtgctgtcatctgtctgtctgctggtcatcgtggggcttctcatgtttaggtaccataggagaggaggttatgatgtggaaaatgaagagaaagtgaagttgggcatgactaattcccactgagagagacttgtgctcaaggaatcggctggggactgctacctctgagaagacacaaggtgatttcagactgcagaggggaaagacttccatctagtcacaaagactccttcgtccccagttgccgtctaggattgggcctcccataattgctttgccaaaataccagagccttcaagtgccaaacagagtatgtccgatggtatctgggtaagaagaaagcaaaagcaagggaccttcatgcccttctgattcccctccaccaaaccccacttcccctcataagtttgtttaaacacttatcttctggattagaatgccggttaaattccatatgctccaggatctttgactgaaaaaaaaaaagaagaagaagaaggagagcaagaaggaaagatttgtgaactggaagaaagcaacaaagattgagaagccatgtactcaagtaccaccaagggatctgccattgggaccctccagtgctggatttgatgagttaactgtgaaataccacaagcctgagaactgaattttgggacttctacccagatggaaaaataacaactatttttgttgttgttgtttgtaaatgcctcttaaattatatatttattttattctatgtatgttaatttatttagtttttaacaatctaacaataatatttcaagtgcctagactgttactttggcaatttcctggccctccactcctcatccccacaatctggcttagtgccacccacctttgccacaaagctaggatggttctgtgacccatctgtagtaatttattgtctgtctacatttctgcagatcttccgtggtcagagtgccactgcgggagctctgtatggtcaggatgtaggggttaacttggtcagagccactctatgagttggacttcagtcttgcctaggcgattttgtctaccatttgtgttttgaaagcccaaggtgctgatgtcaaagtgtaacagatatcagtgtctccccgtgtcctctccctgccaagtctcagaagaggttgggcttccatgcctgtagctttcctggtccctcacccccatggccccaggccacagcgtgggaactcactttcccttgtgtcaagacatttctctaactcctgccattcttctggtgctactccatgcaggggtcagtgcagcagaggacagtctggagaaggtattagcaaagcaaaaggctgagaaggaacagggaacattggagctgactgttcttggtaactgattacctgccaattgctaccgagaaggttggaggtggggaaggctttgtataatcccacccacctcaccaaaacgatgaaggtatgctgtcatggtcctttctggaagtttctggtgccatttctgaactgttacaacttgtatttccaaacctggttcatatttatactttgcaatccaaataaagataacccttattccataaaaaaaaaaaaaaaaaa

SEQ ID NO: 38 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 39 - DNA, 1471 bp, Homo sapiens
gtggccgcgcaggaggacggagccctaaccgcaacccgcgccgcgccgcgccgatttgatttgtatccactgtcaccagcactgctcacttaggactttctggatccagacccaggcagcgcacactggactcttgaggaagaaggagactctaattttggattccttggtggaggaaaataaaacactctggtcttgccgccaacgatgcaagtgtgactgctggcgtcttcatgagctccagaggtcacagcacgctaccaaggactctcatggcccctcggatgatttccgagggagacataggaggcattgctcaaatcacctcctctctattcctgggcagaggcagtgtggcctccaatcggcacctcctccaggctcgtggcatcacctgcattgttaatgctaccattgagatccctaatttcaactggccccaatttgagtatgttaaagtgcctctggctgacatgccgcatgcccccattggactgtactttgacaccgtggctgacaagatccacagtgtgagcaggaagcacggggccaccttggtgcactgtgctgcaggggtgagccgctcagccacgctgtgtatcgcgtacctgatgaaattccacaacgtgtgcctgctggaggcatacaactgggtgaaagcccggcgacctgtcatcaggcccaacgtaggcttctggaggcaactgatagactacgagcgccagctctttgggaagtcgacagttaaaatggtacagacaccttatggcatagttcccgacgtctatgagaaggagtcccgacacctgatgccttactgggggatttagtgccactgaagcctgcgtcagcagcccgagcggggccggcatctgctccccgccgtctgctccctctccactctcttctcaaatggctgacttctggttctccctcaagtgttttttacactgggtgttcaaatttattttaagagatagggagggaggggacataaagggaatgcatacattgctagtcacatttttaaaattaacattttggaatagtgtttatggaaatctttagcttttaatcatttttaccaatttgaacagtttaataaactggttctgctctcttctgaatctcatgcctttggcaccttggtaggtgcaggaggagctcagtgcaaaaatcactttggggcctcattaaccctttagagacaagctttgccccaggctgcggaccagacagatgcttagggaaggttgataccagcttcagtctctactggattagccctactctttcctttcccctccattatttagtgactctgtaagtaagttaaatacacccttattatttagctgttaagtaactataatgaaatctgctgcaaaatctctcttggaatccatgtgcccaggattatattagcattatttttaataaatctatatgcttaaaaaaaaaaaaaaaaaaa

SEQ ID NO: 40 - protein, 198 aa, Homo sapiens
MSSRGHSTLPRTLMAPRMISEGDIGGIAQITSSLFLGRGSVASNRHLLQARGITCIVNATIEIPNFNWPQFEYVKVPLADMPHAPIGLYFDTVADKIHSVSRKHGATLVHCAAGVSRSATLCIAYLMKFHNVCLLEAYNWVKARRPVIRPNVGFWRQLIDYERQLFGKSTVKMVQTPYGIVPDVYEKESRHLMPYWGI

SEQ ID NO: 41 - DNA, 4829 bp, Homo sapiens
atggagggggaccgggtggccgggcggccggtgctgtcgtcgttaccagtgctactgctgctgcagttgctaatgttgcgggccgcggcgctgcacccagacgagctcttcccacacggggagtcgtggtgggaccagctcctgcaggaaggcgacgacgtaaagctcagccgtggtgaagctggcgaatcccctgcacttcttacgaagcccgattcagcaacctctacgtgggcaccaacggcatcatctccactcaggacttccccagggaaacgcagtatgtggactatgatttccccaccgacttcccggccatcgccccttttctggcggacatcgacacgagccacggcagaggccgagtcctgtaccgagaggacacctcccccgcagtgctgggcctggccgcccgctatgtgcgcgctggcttcccgcgctctgcgcgctttttacccccacccacgccttcctggccacctgggagcaggtaggcgcttacgaggaggtcaaacgcgggcgctgccctcgggagagctgaacactttccaggcagttttggcatctgatgggtctgatagctacgccctctttctttatcctgccaacggcctgcagttccttggaacccgccccaaagagtcttacaatgtccagcttcagcttccagctcgggtgggcttctgccgaggggaggctgatgatctgaagtcagaaggaccatatttcagcttgactagcactgaacagtctgtgaaaaatctctatcaactaagcaacctggggatccctggagtgtgggctttccatatcggcagcacttccccgttggacaatgtcaggccagctgcagttggagacctttccgctgcccactcttctgttcccctgggacgttccttcagccatgctacagccctggaaagtgactataatgaggacaatttggattactacgatgtgaatgaggaggaagctgaataccttccgggtgaaccagaggaggcattgaatggccacagcagcattgatgtttccttccaatccaaagtggatacaaagcctttagaggaatcttccaccttggatcctcacaccaaagaaggaacatctctgggagaggtagggggcccagatttaaaaggccaagttgagccctgggatgagagagagaccagaagcccagctccaccagaggtagacagagattcactggctccttcctgggaaaccccaccaccgtaccccgaaaacggaagcatccagccctacccagatggagggccagtgccttcggaaatggatgttcccccagctcatcctgaagaagaaattgttcttcgaagttaccctgcttcaggtcacactacacccttaagtcgagggacgtatgaggtgggactggaagacaacataggttccaacaccgaggtcttcacgtataatgctgccaacaaggaaacctgtgaacacaaccacagacaatgctcccggcatgccttctgcacggactatgccactggcttctgctgccactgccaatccaagttttatggaaatgggaagcactgtctgcctgagggggcacctcaccgagtgaatgggaaagtgagtggccacctccacgtgggccatacacccgtgcacttcactgatgtggacctgcatgcgtatatcgtgggcaatgatggcagagcctacacggccatcagccacatcccacagccagcagcccaggccctcctccccctcacaccaattggaggcctgtttggctggctctttgctttagaaaaacctggctctgagaacggcttcagcctcgcaggtgctgcctttacccatgacatggaagttacattctacccgggagaggagacggttcgtatcactcaaactgctgagggacttgacccagagaactacctgagcattaagaccaacattcaaggccaggtgccttacgtcccagcaaatttcacagcccacatctctccctacaaggagctgtaccactactccgactccactgtgacctctacaagttccagagactactctctgacttttggtgcaatcaaccaaacatggtcctaccgcatccaccagaacatcacttaccaggtgtgcaggcacgcccccagacacccgtccttccccaccacccagcagctgaacgtggaccgggtctttgccttgtataatgatgaagaaagagtgcttagatttgctgtgaccaatcaaattggcccggtcaaagaagattcagaccccactccggtgaatccttgctatgatgggagccacatgtgtgacacaacagcacggtgccatccagggacaggtgtagattacacctgtgagtgcgcatctgggtaccagggagatggacggaactgtgtggatgaaaatgaatgtgcaactggctttcatcgctgtggccccaactctgtatgtatcaacttgcctggaagctacaggtgtgagtgccggagtggttatgagtttgcagatgaccggcatacttgcatcttgatcaccccacctgccaacccctgtgaggatggcagtcatacctgtgctcctgctgggcaggcccggtgtgttcaccatggaggcagcacgttcagctgtgcctgcctgcctggttatgccggcgatgggcaccagtgcactgatgtagatgaatgctcagaaaacagatgtcaccctgcagctacctgctacaatactcctggttccttctcctgccgttgtcaacccggatattatggggatggatttcagtgcatacctgactccacctcaagcctgacaccctgtgaacaacagcagcgccatgcccaggcccagtatgcctaccctggggcccggttccacatcccccaatgcgacgagcagggcaacttcctgcccctacagtgtcatggcagcactggtttctgctggtgcgtggaccctgatggtcatgaagttcctggtacccagactccacctggctccaccccgcctcactgtggaccatcaccagagcccacccagaggcccccgaccatctgtgagcgctggagggaaaacctgctggagcactacggtggcaccccccgagatgaccagtacgtgccccagtgcgatgacctgggccacttcatccccctgcagtgccacggaaagagcgacttctgctggtgtgtggacaaagatggcagagaggtgcagggcacccgctcccagccaggcaccacccctgcgtgtatacccaccgtcgctccacccatggtccggcccacgccccggccagatgtgacccctccatctgtgggcaccttcctgctctatactcagggccagcagattggctacttacccctcaatggcaccaggcttcagaaggatgcagctaagaccctgctgtctctgcatggctccataatcgtgggaattgattacgactgccgggagaggatggtgtactggacagatgttgctggacggacaatcagccgtgccggtctggaactgggagcagagcctgagacgatcgtgaattcaggtctgataagccctgaaggacttgccatagaccacatccgcagaacaatgtactggacggacagtgtcctggataagatagagagcgccctgctggatggctctgagcgcaaggtcctcttctacacagatctggtgaatccccgtgccatcgctgtggatccaatccgaggcaacttgtactggacagactggaatagagaagctcctaaaattgaaacgtcatctttagatggagaaaacagaagaattctgatcaatacagacattggattgcccaatggcttaacctttgaccctttctctaaactgctctgctgggcagatgcaggaaccaaaaaactggagtgtacactacctgatggaactggacggcgtgtcattcaaaacaacctcaagtaccccttcagcatcgtaagctatgcagatcacttctaccacacagactggaggagggatggtgttgtatcagtaaataaacatagtggccagtttactgatgagtatctcccagaacaacgatctcacctctacgggataactgcagtctacccctactgcccaacaggaagaaagtaagtacagtaatgtaaaggaagacttggagtttacaatcagaacctggaccctaaagaacagtgactgcaaaggcaaagaaagtaaaaaaggaattggccattagacgttcctgagcatccaagatgaacattttgtagtgcaaaaagacttttgtgaaaagctgatacctcaatctttactactgtatttttaaaaatgaaggttgttattgcaagtttaaaaaggtaacagaattttaactgttgcttattaaagcaacttcttgtaaacatttatcattaatatttaaaagatcaaattcattcaactaagaattagagtttaagactctaaacctgatttttgccatggattccttctggccaagaaattaaagcacatgtgatcaatataacaatataatcctaaaccttgacagttggagaagccaatgcagaactgatgggaaaggaccaattatttatagtttcccaacaaaagttctaagattttttacctctgcatcagtgcatttctatttatatcaaaaggtgctaaaatgattcaatttgcattttctgatcctgtagtgcctctatagaagtacccacagaaagtaaagtatcacatttataaataccaaagatgtaacaattttaaaattttctagattactccaataaagtgttttaagtttaaaaaaaaaaaaaaaaaaaaaa

SEQ ID NO: 42 - protein, 1376 aa, Homo sapiens
MEGDRVAGRPVLSSLPVLLLLQLLMLRAAALHPDELFPHGESWWDQLLQEGDDVKLSRGEAGESPALLTKPDSATSTWAPTASSPLRTSPGKRSMWTMISPPTSRPSPLFWRTSTRATAEAESCTERTPPPQCWAWPPAMCALASRALRAFYPHPRLPGHLGAGRRLRGGQTRALPSGELNTFQAVLASDGSDSYALFLYPANGLQFLGTRPKESYNVQLQLPARVGFCRGEADDLKSEGPYFSLTSTEQSVKNLYQLSNLGIPGVWAFHIGSTSPLDNVRPAAVGDLSAAHSSVPLGRSFSHATALESDYNEDNLDYYDVNEEEAEYLPGEPEEALNGHSSIDVSFQSKVDTKPLEESSTLDPHTKEGTSLGEVGGPDLKGQVEPWDERETRSPAPPEVDRDSLAPSWETPPPYPENGSIQPYPDGGPVPSEMDVPPAHPEEEIVLRSYPASGHTTPLSRGTYEVGLEDNIGSNTEVFTYNAANKETCEHNHRQCSRHAFCTDYATGFCCHCQSKFYGNGKHCLPEGAPHRVNGKVSGHLHVGHTPVHFTDVDLHAYIVGNDGRAYTAISHIPQPAAQALLPLTPIGGLFGWLFALEKPGSENGFSLAGAAFTHDMEVTFYPGEETVRITQTAEGLDPENYLSIKTNIQGQVPYVPANFTAHISPYKELYHYSDSTVTSTSSRDYSLTFGAINQTWSYRIHQNITYQVCRHAPRHPSFPTTQQLNVDRVFALYNDEERVLRFAVTNQIGPVKEDSDPTPVNPCYDGSHMCDTTARCHPGTGVDYTCECASGYQGDGRNCVDENECATGFHRCGPNSVCINLPGSYRCECRSGYEFADDRHTCILITPPANPCEDGSHTCAPAGQARCVHHGGSTFSCACLPGYAGDGHQCTDVDECSENRCHPAATCYNTPGSFSCRCQPGYYGDGFQCIPDSTSSLTPCEQQQRHAQAQYAYPGARFHIPQCDEQGNFLPLQCHGSTGFCWCVDPDGHEVPGTQTPPGSTPPHCGPSPEPTQRPPTICERWRENLLEHYGGTPRDDQYVPQCDDLGHFIPLQCHGKSDFCWCVDKDGREVQGTRSQPGTTPACIPTVAPPMVRPTPRPDVTPPSVGTFLLYTQGQQIGYLPLNGTRLQKDAAKTLLSLHGSIIVGIDYDCRERMVYWTDVAGRTISRAGLELGAEPETIVNSGLISPEGLAIDHIRRTMYWTDSVLDKIESALLDGSERKVLFYTDLVNPRAIAVDPIRGNLYWTDWNREAPKIETSSLDGENRRILINTDIGLPNGLTFDPFSKLLCWADAGTKKLECTLPDGTGRRVIQNNLKYPFSIVSYADHFYHTDWRRDGVVSVNKHSGQFTDEYLPEQRSHLYGITAVYPYCPTGRK

SEQ ID NO: 43 - DNA, 2312 bp, Homo sapiens
tccagtgacggagccgcccggccgacagccccgagacgacagcccggcgcgtcccggtccccacctccgaccaccgccagcgctccaggccccgcgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcttccccagacactggtttgaagaatgttaagacttgacagtggaactacattagtacacagcaccagaatgtatattaaggtgtggctttaggagcagtgggagggtaccagcagaaaggttagtatcatcagatagctcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttcctgtaacaagccagattttttaaaatttatattgtaaatattgtgtgtgtgtgtgtgtgtgtatatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctctcagatagaatgacagtccgtcaaaacagattgtttgcaaaggggaggcatcagtgtccttggcaggctgatttctaggtaggaaatgtggtagctcacgctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtatattttttctaaaaaaaaaaaaaaacgacagcaacggaattc

SEQ ID NO: 44 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 45 - DNA, 25 bp, Artificial Sequence
  Description of Artificial Sequence Synthetic primer
aaagttcccaaagtttgccggctgc

SEQ ID NO: 46 - DNA, 21 bp, Artificial Sequence
  Description of Artificial Sequence Synthetic primer
cgatgcttgctggaggataga

SEQ ID NO: 47 - DNA, 22 bp, Artificial Sequence
  Description of Artificial Sequence Synthetic primer
acactggtcgtccacactcact

SEQ ID NO: 48 - DNA, 25 bp, Artificial Sequence
  Description of Artificial Sequence Synthetic primer
ttcgcccagacatgtatccctgcag

SEQ ID NO: 49 - DNA, 20 bp, Artificial Sequence
  Description of Artificial Sequence Synthetic primer
atgtgccccgggatgatata

SEQ ID NO: 50 - DNA, 21 bp, Artificial Sequence
  Description of Artificial Sequence Synthetic primer
gtcccccagccaataatcagt

SEQ ID NO: 51 - DNA, 25 bp, Artificial Sequence
  Description of Artificial Sequence Synthetic primer
aggtatcaaagcctctggcccacca

SEQ ID NO: 52 - DNA, 21 bp, Artificial Sequence
  Description of Artificial Sequence Synthetic primer
gcagccaccaaaatcatcaat

SEQ ID NO: 53 - DNA, 23 bp, Artificial Sequence
  Description of Artificial Sequence Synthetic primer
tcacagatcttctccacagggat

SEQ ID NO: 54 - DNA, 29 bp, Artificial Sequence
  Description of Artificial Sequence Synthetic primer
aggttgtgaaaacagcagcaatgcaatgt

SEQ ID NO: 55 - DNA, 19 bp, Artificial Sequence
  Description of Artificial Sequence Synthetic primer
ccacaggaagccgcctagt

SEQ ID NO: 56 - DNA, 21 bp, Artificial Sequence
  Description of Artificial Sequence Synthetic primer
tgagggaagaaaggaaacgct

SEQ ID NO: 57 - DNA, 28 bp, Artificial Sequence
  Description of Artificial Sequence Synthetic primer
ctgaggcatcattcactctaacagcggc

SEQ ID NO: 58 - DNA, 21 bp, Artificial Sequence
  Description of Artificial Sequence Synthetic primer
gaggaggcagtatgcaccaaa

SEQ ID NO: 59 - DNA, 19 bp, Artificial Sequence
  Description of Artificial Sequence Synthetic primer
tgcaggctccaacgtttca

SEQ ID NO: 60 - DNA, 25 bp, Artificial Sequence
  Description of Artificial Sequence Synthetic primer
agaatgccgcgtgctttctcctgac

SEQ ID NO: 61 - DNA, 21 bp, Artificial Sequence
  Description of Artificial Sequence Synthetic primer
atgtaggacagaacgggcctt

SEQ ID NO: 62 - DNA, 20 bp, Artificial Sequence
  Description of Artificial Sequence Synthetic primer
gttttgaattgcattgcccc

SEQ ID NO: 63 - DNA, 24 bp, Artificial Sequence
  Description of Artificial Sequence Synthetic primer
aggacaacctgcagagcgtcacgc

SEQ ID NO: 64 - DNA, 19 bp, Artificial Sequence
  Description of Artificial Sequence Synthetic primer
aagatgcgcttctgtgcca

SEQ ID NO: 65 - DNA, 20 bp, Artificial Sequence
  Description of Artificial Sequence Synthetic primer
aacctggactcctacacgcg

SEQ ID NO: 66 - DNA, 33 bp, Artificial Sequence
  Description of Artificial Sequence Synthetic primer
tgtgaggaaatggaaggatacggacctcttaaa

SEQ ID NO: 67 - DNA, 20 bp, Artificial Sequence
  Description of Artificial Sequence Synthetic primer
gatgccacaaagcggttagg

SEQ ID NO: 68 - DNA, 20 bp, Artificial Sequence
  Description of Artificial Sequence Synthetic primer
gtgacggactcgaagaacgg

SEQ ID NO: 69 - DNA, 23 bp, Artificial Sequence
  Description of Artificial Sequence Synthetic primer
tacaactgggtgaaagcccggcg

SEQ ID NO: 70 - DNA, 19 bp, Artificial Sequence
  Description of Artificial Sequence Synthetic primer
acaacgtgtgcctgctgga

SEQ ID NO: 71 - DNA, 20 bp, Artificial Sequence
  Description of Artificial Sequence Synthetic primer
cctacgttgggcctgatgac

SEQ ID NO: 72 - DNA, 26 bp, Artificial Sequence
  Description of Artificial Sequence Synthetic primer
tgtgcccactgaggagtccaacatca

SEQ ID NO: 73 - DNA, 19 bp, Artificial Sequence
  Description of Artificial Sequence Synthetic primer
aatgacgagggcctggagt

SEQ ID NO: 74 - DNA, 21 bp, Artificial Sequence
  Description of Artificial Sequence Synthetic primer
ttgatccgcataatctgcatg

SEQ ID NO: 75 - DNA, 24 bp, Artificial Sequence
  Description of Artificial Sequence Synthetic primer
ctggctgcagttctctcggcactg

SEQ ID NO: 76 - DNA, 23 bp, Artificial Sequence
  Description of Artificial Sequence Synthetic primer
tgaacagtgaggtatgctgaact

SEQ ID NO: 77 - DNA, 19 bp, Artificial Sequence
  Description of Artificial Sequence Synthetic primer
ctccaggctctcgccagtc

SEQ ID NO: 78 - DNA, 24 bp, Artificial Sequence
  Description of Artificial Sequence Synthetic primer
accaaccagaagggctctgtggaa

SEQ ID NO: 79 - DNA, 22 bp, Artificial Sequence
  Description of Artificial Sequence Synthetic primer
aaggtgtctatcactgcaaagc

SEQ ID NO: 80 - DNA, 23 bp, Artificial Sequence
  Description of Artificial Sequence Synthetic primer
tgaacagtgaggtatgctgaact

SEQ ID NO: 81 - DNA, 20 bp, Artificial Sequence
  Description of Artificial Sequence Synthetic primer
agacaggtcgggtgagggcg

SEQ ID NO: 82 - DNA, 18 bp, Artificial Sequence
  Description of Artificial Sequence Synthetic primer
cgcctctgtgggtaagga

SEQ ID NO: 83 - DNA, 21 bp, Artificial Sequence
  Description of Artificial Sequence Synthetic primer
ccgagttagatctggctttca

SEQ ID NO: 84 - protein, 235 aa, Homo sapiens
PMGKWHVGGRRGSPRQWGATARGRDLEAVRRGGCGSVGRRRQRRRRRRRRMRRMRRMWATQGLAVRVALSVLPGSRALRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASAPTDL

(19) United States
(12) Patent Application Publication
(10) Pub. No.: US 2008/0166724 A1
(43) Pub. Date: Jul. 10, 2008

(54) VEGF-Modulated Genes and Methods Employing Them

(75) Inventors: Hans-Peter Gerber, San Francisco, CA (US); Luca Rastelli, Guilford, CT (US)

Correspondence Address: MERCHANT & GOULD PC, P.O. BOX 2903, MINNEAPOLIS, MN, 55402-0903, US

(73) Assignees: CuraGen Corporation, New Haven, CT (US); Genentech, Inc., South San Francisco, CA (US)

(21) Appl. No.: 11/932,133

(22) Filed: Oct. 31, 2007

Related U.S. Application Data
(63) Continuation of application No. 10/428,487, filed on May 2, 2003.
Application No. 10/428,487 is a division of application No. 09/815,153, filed on Mar. 21, 2001, now abandoned.
(60) Provisional application No. 60/191,201, filed on Mar. 21, 2000.

(51) Int. Cl. C12Q 1/68; C12Q 1/02; G01N 33/574

(52) U.S. Cl. 435/6; 435/29; 435/7.23

(57) ABSTRACT

The present invention provides methods for modulating angiogenesis and/or apoptosis comprising modulating the activity of at least one VEGF-modulated gene polypeptide. The invention also provides pharmaceutical compositions for modulating angiogenesis and apoptosis for the prevention or treatment of diseases associated with VEGF-modulated genes expression. The invention also provides diagnostic assays that use VEGF-modulated gene polynucleotides that hybridize with naturally occurring sequences encoding VEGF-modulated genes and antibodies that specifically bind to the protein.

The invention also provides novel human and mouse arginine-rich proteins (ARPs) and nucleotide sequences. The invention provides for genetically engineered expression vectors and host cells comprising the nucleic acid sequence encoding ARPs and for a method for producing the protein.

### RELATED APPLICATIONS

This application claims priority to U.S. provisional application Ser. No. 60/191,201 filed Mar. 21, 2000, which is incorporated herein by reference in its entirety.

### BACKGROUND

Cities have roads and alleys, plants have xylem and phloem, and people have arteries, veins and lymphatics. Without these byways, the vertebrate animal cells would starve or drown in their metabolic refuse. Not only do blood vessels deliver food and oxygen and carry away metabolic wastes, but they also transport signaling substances that apprise cells of situations remote to them but to which they need to respond. Hormonal messages are a common signal.

All blood vessels are ensheathed by a basal lamina and a delicate monolayer of remarkably plastic endothelial cells lining the luminal walls. Depending on location and function, smooth muscle and connective tissue may also be present.

Not only do healthy cells depend on the blood resources transported by the circulatory system, but so, too, unwanted cells: tumorigenic and malignant cells. These cells colonize and proliferate if they are able to divert bood resources to themselves. Angiogenesis, the type of blood vessel formation where new vessels emerge from the proliferation of preexisting vessels (Risau, 1995; Risau and Flamme, 1995), is exploited not only by usual processes, such as in wound healing or myocardial infarction repair, but also by tumors themselves and in cancers, diabetic retinopathy, macular degeneration, psoriasis, and rheumatoid arthritis. Regardless of the process, whether pathological or usual physiological, endothelial cells mediate angiogenesis in a multi-step fashion: (1) endothelia receive an extracellular cue, (2) the signaled cells breach the basal lamina sheath, abetted by proteases they secrete, (3) the cells then migrate to the signal and proliferate, and finally, (4) the cells form a tube, a morphogenic event (Alberts et al., 1994). The complexity of this process indicates complex changes in cellular physiology and morphology, gene expression, and signaling. Angiogenic accomplices that are cues include basic fibroblast growth factors (bFGF), angiopoietins (such as ANG1) and various forms of vascular endothelial growth factor (VEGF).

VEGF is a multifunctional mitogen that is secreted by many cells, including tumor cells (Ferrara, 1999b). Vascular endothelial cells (VECs) are responsive to VEGF, using two receptors: (1) kinase insert domain-containing receptor/fetal liver kinase 1 (KDR/Flk-1; VEGFR1), and (2) Fms-like tyrosine kinase 1 (FLT-1; VEGFR-2) (Warren et al., 1995). These receptors have different affinities for VEGF and appear to have different cellular responses (Athanassiades and Lala, 1998; Li et al., 1999). VEGFR1 and VEGFR-2 null mice die early during embryogenesis (Fong et al., 1995; Shalaby et al., 1995). From these knockout studies, VEGFR1 is necessary for blood island formation and the development of haematopoietic progenitors (Shalaby et al., 1995), while VEGFR-2 is required for organizing embryonic vasculature (Fong et al., 1995). Of these two receptors, VEGFR1 mediates the full spectrum of VEGF's biological effects, including mitogenesis, vasodilation, and tumor vascularization (Ferrara, 1999a), while VEGFR-2 promotes endothelial survival (Carmeliet et al., 1999).

The molecular events and the order in which they occur and the pathways that are required for this process are of fundamental importance to understand angiogenesis. In vitro models are useful for identifying alterations in gene expression that occur during angiogenesis. A particularly fruitful model systems involves the suspension in a three-dimensional type I collagen gel and various stimuli, such as phorbol myristate acetate (PMA), basic fibroblast growth factor (bFGF), and VEGF. The combination of the stimuli and the collagen gel results in the formation of a three-dimensional tubular network of endothelial cells with interconnecting lumen structures. In this model, endothelial differentiation into tubelike structures is completely blocked by inhibitors of new mRNA or protein synthesis. Furthermore, the cells progress through differentiation in a coordinated and synchronized manner, thus optimizing the profile of gene expression (Kahn et al., 2000; Yang et al., 1999).

VEGF and VEGFR-2 ensure endothelial cell survival. In the developing retina, capillaries disappear in response to hyperoxia (increase in oxygen/oxygen tension), correlating with an inhibition of VEGF secretion by neighboring cells. These vessels disappear by selective apoptosis of endothelial cells (Alon et al., 1995). Removing VEGF by using function-blocking anti-VEGF antibodies also causes blood vessels to regress, even tumor vasculature (Yuan et al., 1996). The mechanisms that mediate VEGF's ability to promote cell survival involve VEGFR-2. Ligation of VEGFR-2 induces a complex of vascular endothelial (VE)-cadherin, β-catenin, phosphoinositide-3-OH kinase (PI3-K), and VEGFR1. PI3-K phosphorylates and activates the serine/threonine protein kinase Akt (protein kinase B) (Carmeliet et al., 1999). Activated Akt is necessary and sufficient to mediate VEGF-dependent survival signal (Gerber et al., 1998).

Programmed cell death, apoptosis, and cell survival play crucial roles in development, homeostasis, stress, and various pathologies. Apoptosis (as opposed to necrosis) is mediated by caspases. Caspases reside in healthy cells as inactive proenzymes, which are activated in response to pro-apoptotic stimuli. Mitochondria activate caspases by releasing cytochrome c into the cytosol, binding the adaptor molecule Apaf-1 (apoptotic protease activating factor 1). Apaf1 oligomerizes and recruits and activates pro-caspase-9. Activated caspase-9 activates downstream caspases, and apoptosis has been initiated. Cytochrome c release may be released through mitochondrial permeability transition (PT) pores. Bcl-2, an anti-apoptosis inhibitor, prevents cytochrome c release by interacting with PT pores (Marzo et al., 1998). VEGF induces expression of Bcl-2 in VECs, indicating that regulation of the mitochondrial permeability is part of VEGF survival mechanism (Gerber et al., 1998).

Tumor cells exploit angiogenesis to facilitate tumor growth. Hypoxia—decreased levels of oxygen—induces tumor cells to secrete VEGF, promoting neovascularization. In addition to secreting VEGF, tumor cells, including hematopoietic cells (Bellamy et al., 1999), breast cancer cells (Speirs and Atkin, 1999), and Kaposi's sarcoma (Masood et al., 1997), express VEGFR1. VEGF can act both in a paracrine and autocrine fashion to stimulate endothelial proliferation and survival. The molecules that mediate neovascularization, in addition to VEGF and its receptors and that ultimately enable tumors to survive will be useful in diagnosis, characterization and ultimately in treatment of tumors.

Identifying genes that are modulated by VEGF is useful in not only understanding the complex endothelial responses, including cell differentiation, remodeling, etc., but also in a variety of diagnostic and therapeutic applications. For example, because mitochondrial cytochrome c release initiates apoptosis and the protective effect of VEGF in inhibiting such action, determining those genes that are modulated by VEGF is useful in controlling apoptosis therapeutically. Such genes and their proteins may be modulated, for example, by gene therapy methods, or the discovery of substances that act on the expression of the gene or the protein itself. Evaluating the expression of VEGF-modulated genes can be used to assess the metastatic potential of a tumor cell. Collections of endothelial-specific markers to assay for vascularization can be used to assay tumor growth. Various pathologies may be treated by exploiting VEGF-mediated angiogenesis.

### SUMMARY OF THE INVENTION

The present invention relates to several VEC genes that are differentially expressed in response to VEGF or related cytokines. These differentially expressed genes are collectedly referred to as “VEGF-modulated genes” (VEGFmg) and are:

1) glia-derived neurite promoting factor (GDNPF)/nexin

2) tissue factor pathway inhibitor-2 (TFPI2)/placental protein 5 (PP5)

3) heparin-binding EGF-like growth factor (HB-EGF)

4) regulator of G-protein signaling-3 (RGS3)

5) myasthenia gravis (MG) autoantigen/gravin

6) MKP-1 like protein tyrosine phosphatase

7) amyloid precursor-like protein 2 (APLP2)/CEI-box binding protein

8) osteonidogen (nidogen-2 precursor)

9) amyloid precursor protein (APP)

10) Human gene similar to yeast VPS41 (hVPS41p)

11) arginine-rich protein (ARP)

12) Down's syndrome critical region protein-1 (DSCR1)

13) insulin induced gene-1 (INSIG1)

14) decidual protein induced by progesterone (DEPP)

15) cytochrome oxidase subunit I (MTCO1)

16) NADH-ubiquinone oxidoreductase chain 1 (ND1)

17) NADH-ubiquinone oxidoreductase chain 4 (ND4)

18) connective tissue growth factor (CTGF)

In a first aspect, the present invention is an isolated polypeptide having at least 80% sequence identity to the sequence SEQ ID NO:3 or SEQ ID NO:22, polynucleotides encoding the same, and antibodies that specifically bind the same.

In a second aspect, the present invention is an isolated polynucleotide having at least 80% sequence identity to the sequence SEQ ID NO:2 or SEQ ID NO:21, or a complement thereof.

In a third aspect, the present invention is a transgenic non-human animal, having a disrupted arginine-rich protein (ARP) gene or a transgenic non-human animal expressing an exogenous polynucleotide having at least 80% sequence identity to the sequence SEQ ID NO:2 or SEQ ID NO:21, or a complement of said polynucleotide.

In a fourth aspect, the present invention is a method of screening a sample for an ARP mutation

In a fifth aspect, the present invention is a method of modulating angiogenesis comprising modulating the activity of at least one VEGF-modulated gene polypeptide.

In a sixth aspect, the present invention is a method of increasing, as well as decreasing angiogenesis, comprising modulating the activity of at least one VEGF-modulated gene polypeptide. Activity modulation of VEGF-modulated gene polypeptides may be over-expressing or eliminating expression of the gene, or impairing a VEGF-modulated gene polypeptide's function by contact with specific antagonists or agonists, such as antibodies or aptamers.

In a seventh aspect, the present invention is a method of treating various pathologies, including tumors, cancers, myocardial infarctions and the like.

In an eighth aspect, the present invention is a method of measuring a VEGF-modulated gene transcriptional and translational up-regulation or down-regulation activity of a compound. In some embodiments, the compounds are calcium channel regulators.

In a ninth aspect, the invention is a method of screening a tissue sample for tumorigenic potential.

In a tenth aspect, the invention is a method of modulating cell survival by modulating the activity of at least one VEGF-modulated gene polypeptide.

In an eleventh aspect, the invention is a method of increasing, as well as decreasing cell survival, comprising modulating the activity of at least one VEGF-modulated gene polypeptide. Activity modulation of VEGF-modulated gene polypeptides may be over-expressing or eliminating expression of the gene, or impairing a VEGF-modulated gene polypeptide's function by contact with specific antagonists or agonists, such as antibodies or aptamers.

In a twelfth aspect, the invention is a method of treating tumors and cancers comprising decreasing cell survival by modulating VEGF-modulated genes. In one embodiment, the modulated gene is DSCR1.

In a thirteenth aspect, the invention is a method of determining the clinical stage of tumor which compares the expression of at least one VEGF-modulated gene in a sample with expression of said at least one gene in control samples. In other embodiments, the VEGF-modulated gene is DSCR1 and/or ARP.

In a fourteenth aspect, the invention is a method of determining if a tumor has a potential for metastasis by determining the clinical stage of the tumor.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

### BRIEF DESCRIPTION OF THE DRAWING

FIG. 1 Survival of human umbilical cord endothelial cells after transfection with various genes related to the present invention.

### DETAILED DESCRIPTION OF THE INVENTION

Using amplification and an imaging approach called GeneCalling (Shimkets et al., 1999), genes that are differentially expressed in endothelial cells stimulated by VEGF were identified. This method provides a comprehensive sampling of cDNA populations in conjunction with the sensitive detection of quantitative differences in mRNA abundance for both known and novel genes (Shimkets et al., 1999). In the instant invention, 18 differentially expressed genes are disclosed. Identification and differential expression of these genes is confirmed by a second independent method employing real-time quantitative polymerase chain reaction (RT-PCR). In general, the present invention relates VEGF-modulated genes to angiogenesis and cell survival.

Definitions

Unless defined otherwise, all technical and scientific terms have the same meaning as is commonly understood by one of skill in the art to which this invention belongs. The definitions below are presented for clarity. All patents and publications referred to herein are, unless noted otherwise, incorporated by reference in their entirety.

The recommendations of (Demerec et al., 1966) where these are relevant to genetics are adapted herein. To distinguish between genes (and related nucleic acids) and the proteins that they encode, the abbreviations for genes are indicated by italicized (or underlined) text while abbreviations for the proteins start with a capital letter and are not italicized. Thus, arginine rich protein (ARP) or arginine rich protein (ARP) refers to the nucleotide sequence that encodes ARP. Likewise, VEGFmg represents the VEGF modulate genes nucleotide sequences and fragments, while VEGFmg refers to the encoded polypeptides and fragments.

“Isolated,” when referred to a molecule, refers to a molecule that has been identified and separated and/or recovered from a component of its natural environment. Contaminant components of its natural environment are materials that interfere with diagnostic or therapeutic use.

“Survival” is a cell remaining alive and maintaining all or most of its morphology and physiological activity, even under conditions of cellular stress, including serum starvation and hypoxia.

### Roles of VEGF-Modulated Genes in Cells

1. Apoptosis

Cell survival is impinged under stress, including oxidative stress and serum deprivation. VEGF stimulation appears to evoke a response similar to that of sub-lethal oxidative stress induced by reactive oxygen species (ROS). An important component of cell survival is mitochondrial respiration. Several VEGF-modulated genes of the instant invention, e.g. DSCR1, gravin, and HB-EGF, are also associated with ROS responses (Kayanoki et al., 1999). In addition, VEGF administration down-regulates several mitochondrial genes (e.g. cytochrome c oxidase subunits and NADH-ubiquinone reductase chains 1, 4 and 5; Examples) and inhibits respiration.

Several observations support the cell survival role of VEGF-modulated genes of the instant invention and their link to mitochondrial respiration. Oxidative stress causes a general, calcium-dependent degradation of mitochondrial polynucleotides in HA-1 fibroblasts (Crawford et al., 1998). When exposed to the anti-prostate cancer compound BMD188 apoptosis induction depends on the mitochondrial respiratory chain (Joshi et al., 1999). Finally, mitochondrial Raf-1 is activated in response to Akt, which counteracts apoptosis (Majewski et al., 1999).

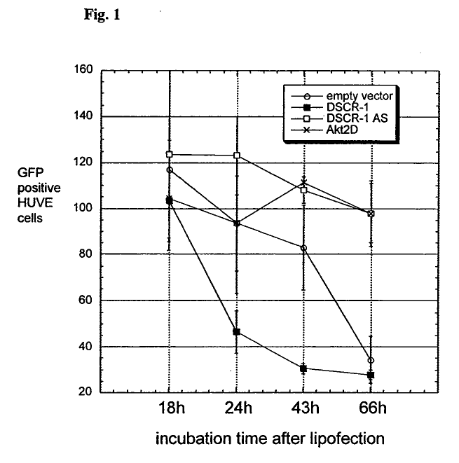

All the genes whose differential expression was confirmed in the present disclosure and that potentially localize in the mitochondria are important components in cell survival based on the experiments disclosed herein. These genes include DSCR1, ARP, INSIG1 and DEPP represent important therapeutic targets. Over expression of DSCR1 was able to hasten apoptosis in human umbilical vascular endothelial cells (HUVECs), while antisense DSCR1 expression promoted cell survival to similar levels as that of activated AKT expression (see FIG. 1).

Adherent cells that become detached from their substrates undergo apoptosis. If the substrate to which they bind, such as the medial and adventitial extracellular matrix layers of arterioles and venules, is defective or eliminated, cells die. These matrices are secreted in part by mesenchymal cells that are recruited by the endothelial cells during the course of angiogenesis. The growth factor, HB-EGF stimulates mesenchymal cell proliferation and migration, and, for example, promotes renal epithelial cell survival (Takemura et al., 1997).

Serpin activity may prevent cell death in endothelia. During angiogenesis when endothelial cells are invading new unvascularized tissues and stroma, serine proteases having thrombin-like activity will be present. Nexin, a serpin, promotes neurite outgrowth and survival by blocking thrombin activity, a multifunctional serine protease that is produced at sites of tissue injury. Thrombin acts via a cell surface protease-activated receptor (PAR-1) and increass in intracellular free calcium levels ([Ca2+]i) (Smith-Swintosky et al., 1995). The present invention demonstrates that serine protease inhibitors (serpins) nexin and placental protein 5 (PP5)/TFPI2 (TFPI2) are induced in response to VEGF. APP and APLP2 appear to play serpin-like roles since these membrane bound proteins can be processed endoproteolytically, yielding secreted forms with serpin-like properties.

### Description of Genes Differentially Expressed and Identified in the Present Invention

Several genes are differentially expressed in VECs when contacted with VEGF or related cytokines, and can be divided into four general classes (Table 1). These genes are collectively referred to as “VEGF-modulated genes” (VEGFmgs), “the set of VEGF-modulated genes” or “genes responsive to VEGF”. Furthermore, among the VEGF-modulated genes, a novel form of ARP is disclosed. The classes of Serpins, Regulators of G-protein-linked receptors and selected Mitochondrial proteins are especially preferred.

TABLE 1
VEGF modulated genes

| Class | Members |
|---|---|
| Serpins | 1) nexin/glia-derived neurite promoting factor |
| (serine | (GDNPF) |
| protease inhibitors) | 2) placental protein 5 (PP5)/tissue factor pathway |
| | inhibitor-2 (TFPI2) |
| | 3) amyloid precursor-like protein 2 (APLP2)/CEI- |
| | box binding protein |
| | 4) amyloid precursor protein (APP) |
| Regulators of | 5) regulator of G-protein signaling-3 (RGS3) |
| G-protein- | 6) gravin/myasthenia gravis (MG) autoantigen |
| linked receptors | |
| Mitochondrial | 7) arginine-rich protein (ARP) |
| proteins | 8) Down's syndrome critical region protein-1 |
| (selected group) | (DSCR1) |
| Others | 9) Human gene similar to yeast VPS41 (hVPS41p) |
| | 10) insulin induced gene-1 (INSIG1) |
| | 11) decidual protein induced by progesterone |
| | (DEPP) |
| | 12) cytochrome oxidase subunit I (MTCO1) |
| | 13) NADH-ubiquinone oxidoreductase chain 1 |
| | (ND1) |
| | 14) NADH-ubiquinone oxidoreductase chain 4 |
| | (ND4) |
| | 15) heparin-binding EGF-like growth factor (HB- |
| | EGF) |
| | 16) MKP-1 like protein tyrosine phosphatase |
| | 17) osteonidogen (nidogen-2 precursor) |
| | 18) connective tissue growth factor (CTGF) |

1. Serpins

Serpins are serine protease inhibitors; they may be secreted or membrane bound. VEGF-modulated serpins comprise nexin, PP5, APLP2, and APP.

(a) Nexin/Glia-Derived Neurite Promoting Factor (GDNPF)

Protease nexin I (PNI or PN1; GenBank A03911; SEQ ID NOS:4 and 5; (Monard et al., EP 233838, 1990)) promotes neurite outgrowth and survival in vitro from neurons and astrocytes by eliminating thrombin's neurite-inhibitory activity. Nexin regulates thrombin's proteolytic activity by forming post-translational, covalent complexes with thrombin (Smith-Swintosky et al., 1995). Thrombin, a multifunctional serine protease, is rapidly produced at sites of tissue injury and catalyzes the final steps in blood coagulation.

In the present invention GeneCalling™ analysis reveals that nexin is up-regulated in VEGF-stimulated endothelial cells at 24 hours (Example 1).

(b) Placental Protein 5 (PP5)/TFPI2 (TFPI2)

PP5/TFPI2 (SEQ ID NOS:6 and 7; GenBank NM_{—}006528, D29992) inhibits a number of blood coagulation and fibrinolysis serine proteases. In embryogenesis, PP5 is involved in trophoblast differentiation and helps maintain intervillous blood flow. PP5 is also frequently expressed in ovarian adenocarcinomas (Inaba et al., 1982). As PN1, PP5 acts by blocking thrombin's activity.

GeneCalling™ analysis found PP5 to be up-regulated in VEGF-stimulated endothelial cells at 24 hours (Example 1).

(c) Amyloid Precursor-like Protein 2 (APLP2)

The human amyloid precursor-like protein APLP2 (SEQ ID NOS:8 and 9; GenBank L27631) belongs to the Alzheimer peptide precursor (APP) family. While structurally related to amyloid precursor protein (APP), APLP2 functions differently. Like APP, APLP2 contains a transmembrane domain and a Kunitz type protease inhibitor domain; however, unlike APP, APLP2 binds DNA, recognizing the centromere DNA sequence element I (CDEI) motif (5′-GTCACATG-3′; SEQ ID NO:10) (Yang et al., 1996).

APLP2 is likely an important component of the cell survival pathway. APLP2 expression is increased in PC12 neuronal cells that undergo apoptosis (Araki and Wurtman, 1998) and is predicted to be a protease inhibitor.

APLP2 was up-regulated in VEGF-stimulated endothelial cells at 24 hours. This result was confirmed by Taqman™ analysis (See Examples).

(d) Amyloid Precursor Protein (APP)

Amyloid precursor protein (SEQ ID NOS:11 and 12; GenBank D87675) is a ubiquitously expressed, membrane spanning glycoprotein that is endoproteolytically processed yielding a secreted protein identical to protease nexin II (PN2) and an internalized 11.5 kDa, 100 residue C-terminal derivative (CTD). PN2 is an inhibitor of proteinases such as trypsin. APP is the source of the β-amyloid (Aβ), a 39-43 amino acid peptide that is the main component deposited in amyloid plaques in Alzheimer's Disease (AD). Neurons that express APP are protected from apoptosis (Xu et al., 1999), although over-expression of APP in endothelia is toxic (Jahroudi et al., 1998).

APP is down-regulated in VEGF-stimulated endothelial cells at 6 and 24 hours (Example 1).

2. Regulator of G-Protein Signaling Receptors

Two regulators of G-protein signaling receptors are VEGF-modulated, comprising RGS3 and gravin.

(a) Regulator of G-Protein Signaling 3 (RGS3, RGP3)

Prolonged stimulation of signal transduction pathways decreases responsiveness. This desensitization occurs because MAP kinase activation by G-protein-linked receptors becomes impaired. RGS3 (SEQ ID NOS:13 and 14; GenBank U27655) encodes a homologue of Sst2p, a yeast gene that mediates desensitization (Druey et al., 1996). RGS3 inhibits signal transduction by increasing the GTPase activity of G-protein α subunits, driving them to the inactive GDP-bound form.

GeneCalling™ analysis (Example 1) reveals that RGS3 is up-regulated in VEGF-stimulated endothelial cells at 24 hours. In situ hybridization analysis reveals high expression in tumors and sarcomas, as well as in adult muscle cells (See Examples). RGS3 expression correlates with VEGF and VEGFR1 expression in ovarian cancer, suggesting that signal transduction pathways are similar between endothelial and tumor cells.

(b) Gravin/Myasthenia Gravis (MG) Autoantigen/A Kinase-Anchoring Proteins (AKAP 250)

Gravin (SEQ ID NOS:15 and 16; GenBank U81607) belongs to the anchoring protein family and anchors both protein kinase A and C to their subcellular sites (Nauert et al., 1997). Gravin is induced by oxidative response (Sato et al., 1998), and mediates recovery from agonist-induced desensitization (Shih et al., 1999), as does RGS3.

GeneCalling analysis (Example 1) reveals that gravin is up-regulated in VEGF-stimulated endothelial cells at 6 and 24 hours. In situ hybridization analysis also demonstrates high expression in fetal tissues and non-vascular tumor components, and lower expression in adult tissue and tumor vascular components. Gravin expression correlates with VEGF expression in ovarian cancer (Examples).

3. Mitochondrial Proteins

(a) Arginine-Rich Protein (ARP)

The instant invention discloses novel arginine-rich protein nucleic acid and polypeptide sequences (SEQ ID NOS:2, 3, 21 and 22; Tables 2 and 3).

Previously described human ARP (SEQ ID NOS:1 (amino acid) and 17 (nucleotide); GenBank NM_{—}006010, M83751) maps to human chromosomal band 3p21, encoding a basic, 234 amino acid residue polypeptide. Highly conserved, ARP is found in all species examined, including hamster, rat, mouse, cow and yeast (Shridhar et al., 1996a; Shridhar et al., 1996b). ARP polymorphisms have been sometimes observed to correlate with neoplasia (Evron et al., 1997; Shridhar et al., 1996a; Shridhar et al., 1996b; Shridhar et al., 1997).

While Shridar (Shridhar et al., 1996a) was able to define a 1 kb mRNA clone for ARP, as well as a smaller form of about 850 bp. Genomic sequence analysis and 5′ RACE were used to establish the 5′ region of this clone. Contrary, the instant invention defines (CuraGen assembly No. 78893638) only a C-terminal fragment of 185 amino acid residues of the sequence deposited in GenBank. The novel nucleotide sequence (SEQ ID NO:2) and the translation of the encoded polypeptide (SEQ ID NO:3) are shown in Tables 2 and 3. Although SEQ ID NO:1 is a hydrophobic polypeptide, predicted by PSORT (Nakai and Horton, 1999) to enter the nucleus (see FIG. 2A), SEQ ID NO:3 is more hydrophilic and predicted to be nuclear localized (see FIG. 2B). Other ARP sequences include a Drosophila ARP-like protein (SEQ ID NOS:18 and 19; Genbank AF132912).

TABLE 2
Nucleotide sequence sequence of novel human ARP

(SEQ ID NO:2)

| | |
|---|---|
| atgaggagga tgaggaggat gtgggccacg caggggctgg cggtgcgcgt ggctctgagc | 60 |
| gtgctgccgg gcagccgggc gctgcggccg ggcgactgcg aagtttgtat ttcttatctg | 120 |
| ggaagatttt accaggacct caaagacaga gatgtcacat tctcaccagc cactattgaa | 180 |
| aacgaactta taaagttctg ccgggaagca agaggcaaag agaatcggtt gtgctactat | 240 |
| atcggggcca cagatgatgc agccaccaaa atcatcaatg aggtatcaaa gcctctggcc | 300 |
| caccacatcc ctgtggagaa gatctgtgag aagcttaaga agaaggacag ccagatatgt | 360 |
| gagcttaagt atgacaagca gatcgacctg agcacagtgg acctgaagaa gctccgagtt | 420 |
| aaagagctga agaagattct ggatgactgg ggggagacat gcaaaggctg tgcagaaaag | 480 |
| tctgactaca tccggaagat aaatgaactg atgcctaaat atgcccccaa ggcagccagt | 540 |
| gcaccgaccg atttgtagtc tgctcaatct ctgttgcacc tgagggggaa aaaacagttc | 600 |
| aactgcttac tcccaaaaca gcctttttgt aatttatttt ttaagtgggc tcctgacaat | 660 |
| actgtatcag atgtgaagcc tggagctttc ctgatgatgc tggccctaca gtacccccat | 720 |
| gaggggattc ccttccttct gttgctggtg tactctagga cttcaaagtg t | 771 |

TABLE 3
Amino acid sequence of novel human ARP

(SEQ ID NO:3)

```
Met Arg Arg Met Arg Arg Met Trp Ala Thr Gln Gly
1 5 10
Leu Ala Val Ala Leu Ala Leu Ser Val Leu Pro Gly
                15 20
Ser Arg Ala Leu Arg Pro Gly Asp Cys Glu Val Cys
25 30 35
Ile Ser Tyr Leu Gly Arg Phe Tyr Gln Asp Leu Val
                        40 45
Glu Gly Phe Arg Asp Val Thr Phe Ser Pro Ala Thr
        50 55 60
Ile Glu Asn Glu Leu Ile Lys Phe Cys Arg Glu Ala
                                65 70
Arg Gly Lys Glu Asn Arg Leu Cys Tyr Tyr Ile Gly
                75 80
Ala Thr Asp Asp Ala Ala Thr Lys Ile Ile Asn Glu
85 90 95
Val Ser Lys Pro Leu Ala His His Ile Pro Val Glu
                        100 105
Lys Ile Cys Glu Lys Leu Lys Lys Lys Asp Ser Gln
        110 115 120
Ile Cys Glu Leu Lys Tyr Asp Lys Gln Ile Asp Leu
                                125 130
Ser Thr Val Asp Leu Lys Lys Leu Arg Val Lys Glu
                135 140
Leu Lys Lys Ile Leu Asp Asp Trp Gly Glu Thr Cys
145 150 155
Lys Gly Cys Ala Glu Lys Ser Asp Tyr Ile Arg Lys
                        160 165
Ile Asn Glu Leu Met Pro Lys Tyr Ala Pro Lys Ala
        170 175 180
Ala Ser Ala Arg Thr Asp Leu
                                185
```

The present invention discloses a novel gene for murine ARP, assembled from EST sequences (SEQ ID NO:20; GenBank AI595930). The murine nucleotide sequence (SEQ ID NO:21) is shown in Table 4, and the translated polypeptide sequence it encodes (SEQ ID NO:22) is shown in Table 5.

TABLE 4
Nucleotide sequence of novel murine ARP

(SEQ ID NO:21)

| | |
|---|---|
| ccgggtgcgg ttcattcgcg cggcatccgg cggtggtgga gacggctgag gaggatgtgg | 60 |
| gctacgcgcg ggctggcggt acgctggccc tgagcgtgct gcctgacagc cgggcgctgc | 120 |
| ggccaggaga ctgtgaagtt tgtatttctt atctgggacg attttaccag gacctcaaag | 180 |
| acagagatgt cacattttca ccagccacta ttgaagaaga acttataaag ttttgccgtg | 240 |
| aagcaagagg caaagagaat cggttgtgct actacattgg agccacagat gatgctgcca | 300 |
| ccaagatcat caatgaggtg tcgaagcccc tggcccacca tatccctgtg gaaaagatct | 360 |
| gtgagaagct gaagaagaaa gacagccaga tctgtgaact aaaatacgac aagcagattg | 420 |
| acctgagcac agtggacctg aagaagctcc gggtgaaaga gctgaagaag atcctggacg | 480 |
| actgggggga gatgtgcaaa ggctgtgcag aaaagtctga ctatatccgg aagataaatg | 540 |
| aactgatgcc taaatacgcc cccaaggcag ccagcgcacg gactgatctg tagtctgccc | 600 |
| aattcctgct gcacctgaag gggaaaaagc agtttatctg tctcttcccc aaataaccat | 660 |
| tttgtaattt attttttaag cgggctcctg acaatgagat gtgaacctag agctttccta | 720 |
| gtgatgctgg ttttgcagtt ccctcttgcc catccccgag tggggacaat ttccccatcc | 780 |
| ccaagtgggg acaatttact tccttctttg ctggtttact ctaggacttc aaagtttgtc | 840 |
| tgggattttt ttattaaaaa aaattgtctt tggagagtta aaaaaaaaaa | 890 |

TABLE 5
Amino acid sequence novel murine ARP

(SEQ ID NO:22)

```
Gly Cys Gly Ser Phe Ala Arg His Pro Ala Val Val
1 5 10
Glu Thr Ala Glu Glu Asp Val Gly Tyr Ala Arg Ala
                15 20
Gly Gly Thr Leu Ala Leu Ser Val Leu Pro Asp Ser
25 30 35
Arg Ala Leu Arg Pro Gly Asp Cys Glu Val Cys Ile
                        40 45
Ser Tyr Leu Gly Arg Phe Tyr Gln Asp Leu Val Glu
        50 55 60
Gly Phe Arg Asp Val Thr Phe Ser Pro Ala Thr Ile
                                65 70
Glu Glu Glu Leu Ile Lys Phe Cys Arg Glu Ala Arg
                75 80
Gly Lys Glu Asn Arg Leu Cys Tyr Tyr Ile Gly Ala
85 90 95
Thr Asp Asp Ala Ala Thr Lys Ile Ile Asn Glu Val
                        100 105
Ser Lys Pro Leu Ala His His Ile Pro Val Glu Lys
        110 115 120
Ile Cys Glu Lys Leu Lys Lys Lys Asp Ser Gln Ile
                                125 130
Cys Glu Leu Lys Tyr Asp Lys Gln Ile Asp Leu Ser
                135 140
Thr Val Asp Leu Lys Lys Leu Arg Val Lys Glu Leu
145 150 155
Lys Lys Ile Leu Asp Asp Trp Gly Glu Met Cys Lys
                        160 165
Gly Cys Ala Glu Lys Ser Asp Tyr Ile Arg Lys Ile
        170 175 180
Asn Glu Leu Met Pro Lys Tyr Ala Pro Lys Ala Ala
                                  185 190
Ser Ala Arg Thr Asp Leu
                195
```

Table 6 shows the alignment of the novel human ARP of the instant invention (Curagen assembly 78893608; SEQ ID NO:3), a published human sequence (Shridar et al. (1996b); gbh_m83751), mouse (AI595930_EXT), and Drosophila melanogaster (AAD32615) using ClustalW alignment. Only the protein described by Shridar et al. (1996b) has the longer N-terminal sequence; while that of the instant invention is truncated at the N-terminus.

GeneCalling™ analysis (Example 1) reveals that ARP is up-regulated in VEGF-stimulated endothelial cells during the first 6 hours. In situ hybridization analysis reveals high expression in fetal and non-vascularized tumor components. Over-expression of ARP correlates with ovarian cancer.

(b) Down's Syndrome Critical Region Protein 1 (DSCR1)

DSCR1 (SEQ ID NOS:23 and 24; GenBank NM_{—}004414, U28833) is a member of the minimal candidate region for the Down syndrome phenotype. DSCR1 has an acidic domain, a serine-proline motif, a putative DNA binding domain and a proline-rich region, much like SH3 domain ligands (Fuentes et al., 1995). The hamster homologue, adapt78, is related to Gpr78, a glucose-regulated protein (Leahy et al., 1999) and is oxidant- and calcium-inducible. PSORT (Nakai and Horton, 1999) predicts mitochondrial localization. DSCR1's structural and functional features suggest roles in transcriptional regulation and/or signal transduction.

GeneCalling™ analysis (Example 1) demonstrated that DSCR1 is up-regulated in VEGF-stimulated endothelial cells during the first 6 hours. Taqman™ analysis revealed that DSCR1 is up-regulated in an in vitro model of endothelial tube formation. In situ hybridization analysis reveals high expression in fetal tissues, but lower levels in adult and tumor non-vascular tissues. Over-expression of DSCR1 correlates with clinical stage of ovarian cancer. Elimination of DSCR1 by antisense experiments increases endothelial cell survival.

4. Other VEGF-Modulated Genes

(a) Human Gene Similar to Yeast VPS41 (hVSP41p)

hVSP41p (SEQ ID NOS:25 and 26; GenBank U87309) in yeast n(VSP41) is required for vacuolar traffic (Radisky et al., 1997) and is involved in endocytosis (Singer-Kruger and Ferro-Novick, 1997).

In the present invention, GeneCalling analysis (Example 1) reveals that hVPS41 is down-regulated in VEGF-stimulated endothelial cells at 24 hours. In situ hybridization analysis localised expression to non-vascularized regions of tumors. Expression of hVSP41 correlates with ovarian cancer (Examples).

(b) Insulin Induced gene 1 (INSIG1)

INSIG1 (SEQ ID NOS:27 and 28; GenBank 5031800, U96876) expression is transcriptionally up-regulated in rat regenerating livers, and is induced in murine adipocyte differentiation, suggesting that INSIG1 may play a role in growth and differentiation of tissues involved in metabolic control (Peng et al., 1997). INSIG1 is also expressed by monocytes in a model of atherogenesis, as are oxidized lipoprotein HB-EGF and gravin (Falb, WO9730065, 1997). Hydrophobicity analysis predicts a transmembrane localization. The protein is homologous to sodium channels and to G-protein coupled receptors. PSORT (Nakai and Horton, 1999) predicts localization to the mitochondrial inner membrane.

GeneCalling analysis (Example 1) demonstrated that INSIG1 was up-regulated in VEGF-stimulated endothelial cells at 24 hours and in an in vitro model of endothelial tube formation.

(c) Decidual Protein Induced by Progesterone (DEPP)

DEPP (SEQ ID NOS:29 and 30; GenBank AB022718) is published only in the database. SEQ ID NO:29 comprises a 2114 bp transcript encoding a putative 212 amino acid peptide that is induced by the steroid progesterone. Steroid hormones play vital roles in angiogenesis, especially in the female reproductive tract (Hyder and Stancel, 1999).

GeneCalling analysis (Example 1) reveals that DEPP was up-regulated in VEGF-stimulated endothelial cells at 6 hours.

(d) Cytochrome Oxidase Subunit I (MTCO1)

Cytochrome c oxidase subunit I (MTCO1, SEQ ID NO:31 (nucleotide sequence extracted from the complete human mitochondrial genome sequence, GenBank NC_{—}001807) and SEQ ID NO:32 (amino acid; GenBank NP_{—}008344) is 1 of 3 mitochondrial DNA encoded subunits of respiratory Complex IV. Complex IV localizes to the mitochondrial inner membrane and mediates the final step in the electron transport chain of oxidative phosphorylation. Complex IV collects electrons from reduced cytochrome c and transfers them to oxygen, producing energy and water. The released energy is used to transport protons across the mitochondrial inner membrane.

(e) NADH-Ubiquinone Oxidoreductase Chain 1 (ND1 or DNHUN1) and

(f) NADH-Ubiquinone Oxidoreductase Chain 4 (ND4 or DNHUN4)

The proton-translocating NADH:ubiquinone oxidoreductase or complex I chain 1 (SEQ ID NOS:33, GenBank NC_{—}001807 and 34; GenBank DUNHUN1) and chain 4 (SEQ ID NOS:35, GenBank NC_{—}001807 and 36; GenBank DUNHUN4) are located in the inner membranes of mitochondria. Complex I is the site for electrons entering the respiratory chain and important in conserving cell energy. The complex I-catalyzed oxidation of NADH is coupled to proton membrane translocation.

(g) Heparin-Binding EGF-like Growth Factor (HB-EGF)

HB-EGF (SEQ ID NOS:37 and 38; GenBank NM_{—}001945) is an EGF family member that ligates EGF receptors 1 (HER-1) or 4 (HER4) to induce mitogenic and/or chemotactic activities. HB-EGF is expressed by numerous cell types, including leukemia cells (Vinante et al., 1999), and does not directly induce endothelial cell mitosis, but does induce these cells to migrate and induces the vascular smooth muscle cells to release factors that induce endothelia mitosis (Morita et al., 1993). While previously observed to be induced by VEGF (Arkonac et al., 1998), no specific role in endothelial cell survival has been proposed.

In addition to VEGF, reactive oxygen species and calcium induce HB-EGF expression (Kayanoki et al., 1999) as they do for DSCR1. Membrane-bound HB-EGF retains growth activity, adhesion capabilities and promotes renal epithelial cells survival (Takemura et al., 1997). ProHB-EGF forms a complex in the plasma membrane with the tetraspanin CD9 that also increases the survival activity of HB-EGF expression (Takemura et al., 1999).

In the instant invention (Examples), HB-EGF was found to be up-regulated in VEGF-stimulated endothelial cells at 24 hours. In situ hybridization analysis reveals expression in non vascular component in tumors, fetal and adult tissue, and high expression in endothelial cells of the appendix.

(h) MKP-1 like Protein Tyrosine Phosphatase (SEQ ID NOS:39 and 40; GenBank AF038844)

The protein sequence is 58% similar to Mitogen-activated protein (MAP) kinase phosphatase-1 (MKP-1), a dual-specificity protein tyrosine phosphatase. Homology for the catalytic domain is very high, although no specific substrate has yet been described for MKP-1 like protein tyrosine phosphatase. MAP kinase cascades play critical roles in inhibiting apoptosis, phosphorylating Bcl-2 (Deng et al., 2000). MAP kinases are activated by tyrosine and threonine phosphorylation and inactivated by dephosphorylation (Wilkinson and Millar, 2000). MKP-1 increases cell survival Winter et al., 1998), and is induced by elevated calcium (Scimeca et al., 1997). Because of its similarly to MKP-1, the MKP-1-like protein tyrosine phosphatase may regulate one or more MAP kinases involved in cell survival.

(i) Osteonidogen (Nidogen-2 Precursor)

Nidogen-2 (SEQ ID NOS:41 and 42; GenBank D86425) is 46% identical, and has a similar domain structure with the basement membrane (basal lamina) protein nidogen-1/enactin. Nidogens 1 and 2 have similar but distinct binding and adhesive properties for basement membrane components (Lohi et al., 1998). The complex laminin-entactin can stimulate and inhibit angiogenesis in a dose-dependent fashion (Nicosia et al., 1994).

In the present invention, GeneCalling analysis (Example 1) reveals that nidogen-2 is up-regulated in VEGF-stimulated endothelial cells at 6 and 24 hours. In situ hybridization analysis demonstrates expression in fetal tissues, inflamed appendix and vascular and non-vascular component of peritumoral stroma. (Oivula et al., 1999) also report expression by the endothelial basal lamina and stroma in carcinomas.

(j) Connective Tissue Growth Factor (CTGF)

CTGF (connective tissue growth factor; SEQ ID NOS: 43 and 44, GenBank X78947) is a member of a family of secreted proteins that includes CYR61, Nov, Elm-1, Cop-1/WISP-2, WISP-3 and the mouse CTGF homolog, Fisp12. CTGF stimulates fibroblast migration and promotes adhesion and mitogenesis in both fibroblasts and endothelial cells through the integrin receptor αvβ3. In addition, the presence of CTGF promotes endothelial cell survival. In vivo, CTGF induces neovascularization in rat corneal micropocket implants.

In the instant invention, CTGF is up-regulated in VEGF-stimulated endothelial cells at 6 and 24 hours. In situ hybridization analysis reveals that CTGF is expressed in most tested tissues, which the highest expression in fetal tissues. These observations, with the localization of CTGF in angiogenic tissues and in atherosclerotic plaques, suggest a possible role for CTGF in the regulation of vessel growth during development, wound healing, and vascular disease.

### VEGFmg Polynucleotides

One aspect of the invention pertains to isolated nucleic acid molecules that encode VEGFmg or biologically-active portions thereof. Also included in the invention are nucleic acid fragments sufficient for use as hybridization probes to identify VEGFmg-encoding nucleic acids (e.g., VEGFmg mRNAs) and fragments for use as polymerase chain reaction (PCR) primers for the amplification and/or mutation of VEGFmg molecules. A “nucleic acid molecule” includes DNA molecules (e.g., cDNA or genomic DNA), RNA molecules (e.g., mRNA), analogs of the DNA or RNA generated using nucleotide analogs, and derivatives, fragments and homologs. The nucleic acid molecule may be single-stranded or double-stranded, but preferably comprises double-stranded DNA.

1. Control Sequences

Control sequence are DNA sequences that enable the expression of an operably-linked coding sequence in a particular host organism. Prokaryotic control sequences include promoters, operator sequences, and ribosome binding sites. Eukaryotic cells utilize promoters, polyadenylation signals, and enhancers.

2. Operably-Linked

Nucleic acid is operably-linked when it is placed into a functional relationship with another nucleic acid sequence. For example, a promoter or enhancer is operably-linked to a coding sequence if it affects the transcription of the sequence, or a ribosome-binding site is operably-linked to a coding sequence if positioned to facilitate translation. Generally, “operably-linked” means that the DNA sequences being linked are contiguous, and, in the case of a secretory leader, contiguous and in reading phase. However, enhancers do not have to be contiguous. Linking is accomplished by conventional recombinant DNA methods.

3. Isolated Nucleic Acids

An isolated nucleic acid molecule is purified from the setting in which it is found in nature and is separated from at least one contaminant nucleic acid molecule. Isolated ARP molecules are distinguished from the specific ARP molecules, as they exist in cells. However, an isolated ARP molecule includes ARP molecules contained in cells that ordinarily express the ARP where, for example, the nucleic acid molecule is in a chromosomal location different from that of natural cells.

4. Probes

Probes are nucleic acid sequences of variable length, preferably between at least about 10 nucleotides (nt), 100 nt, or many (e.g., 6,000 nt) depending on the specific use. Probes are used to detect identical, similar, or complementary nucleic acid sequences. Longer length probes can be obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies. Probes are substantially purified oligonucleotides that will hybridize under stringent conditions to at least optimally 12, 25, 50, 100, 150, 200, 250, 300, 350 or 400 consecutive sense strand nucleotide sequence; or an anti-sense strand nucleotide sequence; or of a naturally occurring mutant of the VEGFmg sequence of interest.

The full- or partial length native sequence VEGFmg may be used to “pull out” similar (homologous) sequences (Ausubel et al., 1987; Sambrook, 1989), such as: (1) full-length or fragments of VEGFmg cDNA from a cDNA library from any species (e.g. human, murine, feline, canine, bacterial, viral, retroviral, yeast), (2) from cells or tissues, (3) variants within a species, and (4) homologues and variants from other species. To find related sequences that may encode related genes, the probe may be designed to encode unique sequences or degenerate sequences. Sequences may also be genomic sequences including promoters, enhancer elements and introns of native sequence VEGFmg.

For example, VEGFmg coding region in another species may be isolated using such probes. A probe of about 40 bases is designed, based on VEGFmg, and made. To detect hybridizations, probes are labeled using, for example, radionuclides such as ³²P or ³⁵S, or enzymatic labels such as alkaline phosphatase coupled to the probe via avidin-biotin systems. Labeled probes are used to detect nucleic acids having a complementary sequence to that of VEGFmg in libraries of cDNA, genomic DNA or mRNA of a desired species.

Such probes can be used as a part of a diagnostic test kit for identifying cells or tissues which mis-express a VEGFmg, such as by measuring a level of a VEGFmg in a sample of cells from a subject e.g., detecting VEGFmg mRNA levels or determining whether a genomic VEGFmg has been mutated or deleted.

5. Isolated Nucleic Acid

An isolated nucleic acid molecule is separated from other nucleic acid molecules which are present in the natural source of the nucleic acid. Preferably, an isolated nucleic acid is free of sequences that naturally flank the nucleic acid (i.e., sequences located at the 5′- and 3′-termini of the nucleic acid) in the genomic DNA of the organism from which the nucleic acid is derived. For example, in various embodiments, isolated VEGFmg molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an isolated nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a VEGFmg nucleic acid molecule, or a complement of this aforementioned nucleotide sequence, can be isolated using standard molecular biology techniques and the provided sequence information. Using all or a portion of a VEGFmg nucleic acid sequence of interest as a hybridization probe, VEGFmg molecules can be isolated using standard hybridization and cloning techniques (Ausubel et al., 1987; Sambrook, 1989).

PCR amplification techniques can be used to amplify VEGFmg using cDNA, mRNA or alternatively, genomic DNA, as a template and appropriate oligonucleotide primers. Such nucleic acids can be cloned into an appropriate vector and characterized by DNA sequence analysis. Furthermore, oligonucleotides corresponding to VEGFmg sequences can be prepared by standard synthetic techniques, e.g., an automated DNA synthesizer.

6. Oligonucleotide

An oligonucleotide comprises a series of linked nucleotide residues, which oligonucleotide has a sufficient number of nucleotide bases to be used in a PCR reaction or other application. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of a VEGFmg sequence of interest, or a complement thereof. Oligonucleotides may be chemically synthesized and may also be used as probes.

7. Complementary Nucleic Acid Sequences; Binding

In another embodiment, an isolated nucleic acid molecule comprises a nucleic acid molecule that is a complement of a VEGFmg nucleotide sequence of the invention, or a portion of this nucleotide sequence (e.g., a fragment that can be used as a probe or primer or a fragment encoding a biologically-active portion of a VEGFmg). A nucleic acid molecule that is complementary to a VEGFmg nucleotide sequence of interest, is one that is sufficiently complementary to that nucleotide sequence such that it can hydrogen bond with little or no mismatches, forming a stable duplex.

“Complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Nucleic acid fragments are at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full-length sequence. Fragments may be derived from any contiguous portion of a nucleic acid or amino acid sequence of choice.

8. Derivatives, and Analogs

Derivatives are nucleic acid sequences or amino acid sequences formed from the native compounds either directly or by modification or partial substitution. Analogs are nucleic acid sequences or amino acid sequences that have a structure similar to, but not identical to, the native compound but differ from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions (Ausubel et al., 1987).

9. Open Reading Frames

The open reading frame (ORF) of a VEGFmg gene encodes VEGFmg. An ORF is a nucleotide sequence that has a start codon (ATG) and terminates with one of the three “stop” codons (TAA, TAG, or TGA). In this invention, however, an ORF may be any part of a coding sequence that may or may not comprise a start codon and a stop codon. To achieve a unique sequence, preferable VEGFmg ORFs encode at least 50 amino acids.

10. Homology

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of VEGFmg. Isoforms can be expressed in different tissues of the same organism as a result of, for example, alternative splicing of RNA. Alternatively, different genes can encode isoforms. In the invention, homologous nucleotide sequences include nucleotide sequences encoding for a VEGFmg of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding a human VEGFmg. Homologous nucleic acid sequences include those nucleic acid sequences that encode conservative amino acid substitutions in a VEGFmg sequence of interest, as well as a polypeptide possessing VEGFmg biological activity. Various biological activities of the VEGFmg are described below.

11. Sequence Identity

“Percent (%) nucleic acid sequence identity” with respect to a VEGFmg is defined as the percentage of nucleotides in a candidate sequence that are identical with the nucleotides in that particular VEGFmg, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

%_{nucleic acid sequence identity}=W/Z·100

where

W is the number of nucleotides cored as identical matches by the sequence alignment program's or algorithm's alignment of C and D

And

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

12. Stringency

Homologs (i.e., nucleic acids encoding VEGF-modulated molecules derived from species other than human) or other related sequences (e.g., paralogs) can be obtained by low, moderate or high stringency hybridization with all or a portion of the particular human sequence as a probe using methods well known in the art for nucleic acid hybridization and cloning.

The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach is to vary the temperature: higher relative temperatures result in more stringent reaction conditions. (Ausubel et al., 1987) provide an excellent explanation of stringency of hybridization reactions.

To hybridize under “stringent conditions” describes hybridization protocols in which nucleotide sequences at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium

(a) High Stringency

“Stringent hybridization conditions” conditions enable a probe, primer or oligonucleotide to hybridize only to its target sequence. Stringent conditions are sequence-dependent and will differ. Stringent conditions comprise: (1) low ionic strength and high temperature washes (e.g. 15 mM sodium chloride, 1.5 mM sodium citrate, 0.1% sodium dodecyl sulfate at 50° C.); (2) a denaturing agent during hybridization (e.g. 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate at 42° C.); or (3) 50% formamide. Washes typically also comprise 5×SSC (0.75 M NaCl, 75 mM sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% homologous to each other typically remain hybridized to each other. These conditions are presented as examples and are not meant to be limiting.

(b) Moderate Stringency

“Moderately stringent conditions” use washing solutions and hybridization conditions that are less stringent (Sambrook, 1989), such that a polynucleotide will hybridize to the entire, fragments, derivatives or analogs of a target VEGFmg target sequence. One example comprises hybridization in 6× SSC, 5× Denhardt's solution, 0.5% SDS and 100 mg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions are described in (Ausubel et al., 1987; Kriegler, 1990).

(c) Low Stringency

“Low stringent conditions” use washing solutions and hybridization conditions that are less stringent than those for moderate stringency (Sambrook, 1989), such that a polynucleotide will hybridize to the entire, fragments, derivatives or analogs of a target VEGFmg target sequence. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency, such as those for cross-species hybridizations are described in (Ausubel et al., 1987; Kriegler, 1990; Shilo and Weinberg, 1981).

13. Conservative Mutations

In addition to naturally-occurring allelic variants of VEGFmg, changes can be introduced by mutation into VEGFmg sequences that incur alterations in the amino acid sequences of the encoded VEGF-modulated molecules that do not alter VEGF-modulated molecules function. For example, nucleotide substitutions leading to amino acid substitutions at “non-essential” amino acid residues can be made in the sequence of a VEGFmg polypeptide. A “non-essential” amino acid residue is a residue that can be altered from the wild-type sequences of VEGFmg without altering their biological activity, whereas an “essential” amino acid residue is required for such biological activity. For example, amino acid residues that are conserved among the VEGFmg molecules of the invention are predicted to be particularly non-amenable to alteration. Amino acids for which conservative substitutions can be made are well-known in the art.

Useful conservative substitutions are shown in Table A, “Preferred substitutions.” Conservative substitutions whereby an amino acid of one class is replaced with another amino acid of the same type fall within the scope of the subject invention so long as the substitution does not materially alter the biological activity of the compound. If such substitutions result in a change in biological activity, then more substantial changes, indicated in Table B as exemplary are introduced and the products screened for VEGFmg polypeptide biological activity.

TABLE A
Preferred substitutions

| | | Preferred |
|---|---|---|
| Original residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, Ala, | Ile |
| | Phe | |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | Norleucine | |

Non-conservative substitutions that effect (1) the structure of the polypeptide backbone, such as a β-sheet or α-helical conformation, (2) the charge or (3) hydrophobicity, or (4) the bulk of the side chain of the target site can modify VEGFmg function or immunological identity. Residues are divided into groups based on common side-chain properties as denoted in Table B. Non-conservative substitutions entail exchanging a member of one of these classes for another class. Substitutions may be introduced into conservative substitution sites or more preferably into non-conserved sites.

TABLE B
Amino acid classes

| Class | Amino acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce the VEGFmg variant DNA (Ausubel et al., 1987; Sambrook, 1989).

In one embodiment, the isolated nucleic acid molecule comprises a nucleotide sequence encoding a protein, wherein the protein comprises an amino acid sequence at least about 45%, preferably 60%, more preferably 70%, 80%, 90%, and most preferably about 95% homologous to that of a VEGFmg of interest.

A mutant VEGFmg can be assayed for modulating cell survival and/or angiogenesis in vitro.

14. VEGFmg Variant Polynucleotides, Genes and Recombinant Genes

The invention further encompasses nucleic acid molecules that differ from the nucleotide sequences due to degeneracy of the genetic code and thus encode the same VEGFmg as that encoded by, for example, the ARP nucleotide sequences shown in SEQ ID NO NOS:2 or 21. An isolated nucleic acid molecule of the invention has a nucleotide sequence encoding, for example, an ARP protein having an amino acid sequence shown in SEQ ID NOS:3 or 22.

In addition sequence polymorphisms that change the amino acid sequences of the VEGFmg may exist within a population. For example, allelic variation among individuals will exhibit genetic polymorphism in VEGFmg. The terms “gene” and “recombinant gene” refer to nucleic acid molecules comprising an open reading frame (ORF) encoding VEGFmg, preferably a vertebrate VEGFmg. Such natural allelic variations can typically result in 1-5% variance in VEGFmg. Any and all such nucleotide variations and resulting amino acid polymorphisms in the VEGFmg, which are the result of natural allelic variation and that do not alter the functional activity of the VEGFmg are within the scope of the invention.

Moreover, VEGFmg from other species that have a nucleotide sequence that differs from the human sequence of VEGFmgs are contemplated. Nucleic acid molecules corresponding to natural allelic variants and homologues of VEGFmg cDNAs of the invention can be isolated based on their homology to VEGFmg using cDNA-derived probes to hybridize to homologous VEGFmg sequences under stringent conditions.

“VEGFmg variant polynucleotide” or “VEGFmg variant nucleic acid sequence” means a nucleic acid molecule which encodes an active VEGFmg that (1) has at least about 80% nucleic acid sequence identity with a nucleotide acid sequence encoding a full-length native VEGFmg, (2) a full-length native VEGFmg lacking the signal peptide, (3) an extracellular domain of a VEGFmg, with or without the signal peptide, or (4) any other fragment of a fill-length VEGFmg. Ordinarily, a VEGFmg variant polynucleotide will have at least about 80% nucleic acid sequence identity, more preferably at least about 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with the nucleic acid sequence encoding a full-length native VEGFmg. A VEGFmg variant polynucleotide may encode full-length native VEGFmg lacking the signal peptide, an extracellular domain of a VEGFmg, with or without the signal sequence, or any other fragment of a full-length VEGFmg. Variants do not encompass the native nucleotide sequence.

Ordinarily, VEGFmg variant polynucleotides are at least about 30 nucleotides in length, often at least about 60, 90, 120, 150, 180, 210, 240, 270, 300, 450, 600 nucleotides in length, more often at least about 900 nucleotides in length, or more.

### VEGFmg Polypeptides

1. Mature

A VEGFmg can encode a mature VEGFmg. A “mature” form of a polypeptide or protein disclosed in the present invention is the product of a naturally occurring polypeptide or precursor form or proproten. The naturally occurring polypeptide, precursor or proprotein includes, by way of nonlimiting example, the full-length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

2. Isolated VEGFmg Polypeptide

An “isolated” or “purified” polypeptide, protein or biologically active fragment is separated and/or recovered from a component of its natural environment. Contaminant components include materials that would typically interfere with diagnostic or therapeutic uses for the polypeptide, and may include enzymes, hormones, and other proteinaceous or non-proteinaceous materials. Preferably, the polypeptide is purified to a sufficient degree to obtain at least 15 residues of N-terminal or internal amino acid sequence. To be substantially isolated, preparations having less than 30% by dry weight of non-VEGFmg contaminating material (contaminants), more preferably less than 20%, 10% and most preferably less than 5% contaminants. An isolated, recombinantly-produced VEGFmg or biologically active portion is preferably substantially free of culture medium, i.e., culture medium represents less than 20%, more preferably less than about 10%, and most preferably less than about 5% of the volume of the VEGFmg preparation. Examples of contaminants include cell debris, culture media, and substances used and produced during in vitro synthesis of VEGFmg.

When the molecule is a purified polypeptide, the polypeptide will be purified (1) to obtain at least 15 residues of N-terminal or internal amino acid sequence using a sequenator, or (2) to homogeneity by SDS-PAGE under non-reducing or reducing conditions using Coomassie blue or silver stain. Isolated polypeptides include those expressed heterologously in genetically-engineered cells or expressed in vitro, since at least one component of the VEGFmg's natural environment will not be present. Ordinarily, isolated polypeptides are prepared by at least one purification step.

3. Biologically Active

Biologically active portions of VEGFmgs include peptides comprising amino acid sequences sufficiently homologous to or derived from VEGFmg amino acid sequences that include fewer amino acids than the full-length VEGFmg, and exhibit at least one activity of a VEGFmg. Biologically active portions comprise a domain or motif with at least one activity of native VEGFmg. A biologically active portion of a VEGFmg can be a polypeptide that is, for example, 10, 25, 50, 100 or more amino acid residues in length. Other biologically active portions, in which other regions of the protein are deleted, can be prepared by recombinant techniques and evaluated for one or more of the functional activities of a native VEGFmg.

Biologically active portions of VEGFmg may retain the functional activity of the protein, yet differs in amino acid sequence due to natural allelic variation or mutagenesis.

4. anti-VEGFmg Abs

Antibody may be single anti-VEGFmg monoclonal Abs (mAbs; including agonist, antagonist, and neutralizing Abs), anti-VEGFmg antibody compositions with polyepitopic specificity, single chain anti-VEGFmg Abs, and fragments of anti-VEGFmg Abs. A “monoclonal antibody” refers to an antibody obtained from a population of substantially homogeneous Abs, i.e., the individual Abs comprising the population are identical except for naturally-occurring mutations that may be present in minor amounts.

5. Epitope Tags

An epitope tagged polypeptide refers to a chimeric polypeptide fused to a “tag polypeptide”. Such tags provide epitopes against which Abs can be made or are available, but do not interfere with polypeptide activity. To reduce anti-tag antibody reactivity with endogenous epitopes, the tag polypeptide is preferably unique. Suitable tag polypeptides generally have at least six amino acid residues and usually between about 8 and 50 amino acid residues, preferably between 8 and 20 amino acid residues). Examples of epitope tag sequences include HA from Influenza A virus and FLAG.

6. Variant VEGFmg Polypeptides

In general, a VEGFmg variant that preserves VEGFmg-like function and includes any variant in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above. “VEGFmg polypeptide variant” means an active VEGFmg polypeptide having at least: (1) about 80% amino acid sequence identity with a full-length native sequence VEGFmg polypeptide sequence, (2) a VEGFmg polypeptide sequence lacking the signal peptide, (3) an extracellular domain of a VEGFmg polypeptide, with or without the signal peptide, or (4) any other fragment of a full-length VEGFmg polypeptide sequence. For example, VEGFmg polypeptide variants include VEGFmg polypeptides wherein one or more amino acid residues are added or deleted at the N— or C-terminus of the full-length native amino acid sequence. A VEGFmg polypeptide variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence VEGFmg polypeptide sequence. A VEGFmg polypeptide variant may have a sequence lacking the signal peptide, an extracellular domain of a VEGFmg polypeptide, with or without the signal peptide, or any other fragment of a full-length VEGFmg polypeptide sequence. Ordinarily, VEGFmg variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in the disclosed VEGFmg polypeptide sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

%_{amino acid sequence identity}=X/Y·100

where
X is the number of amino acid residues scored as identical matches by the sequence alignment program's or algorithm's alignment of A and B and
Y is the total number of amino acid residues in B.

If the length of amino acid sequence A is not equal to the length of amino acid sequence B, the % amino acid sequence identity of A to B will not equal the % amino acid sequence identity of B to A.

7. Determining Homology between Two or More Sequences

“VEGFmg variant” means an active VEGFmg having at least: (1) about 80% amino acid sequence identity with a full-length native sequence VEGFmg sequence, (2) a VEGFmg sequence lacking the signal peptide, (3) an extracellular domain of a VEGFmg, with or without the signal peptide, or (4) any other fragment of a full-length VEGFmg sequence. For example, VEGFmg variants include VEGFmg wherein one or more amino acid residues are added or deleted at the N— or C-terminus of the full-length native amino acid sequence. A VEGFmg variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence VEGFmg sequence. A VEGFmg variant may have a sequence lacking the signal peptide, an extracellular domain of a VEGFmg, with or without the signal peptide, or any other fragment of a full-length VEGFmg sequence. Ordinarily, VEGFmg variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in the disclosed VEGFmg sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

%_{amino acid sequence identity}=X/Y·100

where
X is the number of amino acid residues scored as identical matches by the sequence alignment program's or algorithm's alignment of A and B and
Y is the total number of amino acid residues in B.

If the length of amino acid sequence A is not equal to the length of amino acid sequence B, the % amino acid sequence identity of A to B will not equal the % amino acid sequence identity of B to A.

8. Chimeric and Fusion Proteins

Fusion polypeptides are useful in expression studies, cell-localization, bioassays, and VEGFmg purification. A VEGFmg “chimeric protein” or “fusion protein” comprises VEGFmg fused to a non-VEGFmg polypeptide. A VEGFmg fusion protein may include any portion to the entire VEGFmg, including any number of the biologically active portions. VEGFmg may be fused to the C-terminus of the GST (glutathione S-transferase) sequences. Such fusion proteins facilitate the purification of recombinant VEGFmg. In certain host cells, (e.g. mammalian), heterologous signal sequences fusions may ameliorate VEGFmg expression and/or secretion. Additional exemplary fusions are presented in Table C.

Other fusion partners can adapt VEGFmg therapeutically. Fusions with members of the immunoglobulin (Ig) protein family are useful in therapies that inhibit VEGFmg ligand or substrate interactions, consequently suppressing VEGFmg-mediated signal transduction in vivo. Such fusions, incorporated into pharmaceutical compositions, may be used to treat proliferative and differentiation disorders, as well as modulating cell survival. VEGFmg-Ig fusion polypeptides can also be used as immunogens to produce anti-VEGFmg Abs in a subject, to purify VEGFmg ligands, and to screen for molecules that inhibit interactions of VEGFmg with other molecules.

Fusion proteins can be easily created using recombinant methods. A nucleic acid encoding VEGFmg can be fused in-frame with a non-VEGFmg encoding nucleic acid, to the VEGFmg NH₂— or COO— —terminus, or internally. Fusion genes may also be synthesized by conventional techniques, including automated DNA synthesizers. PCR amplification using anchor primers that give rise to complementary overhangs between two consecutive gene fragments that can subsequently be annealed and reamplified to generate a chimeric gene sequence (Ausubel et al., 1987) is also useful. Many vectors are commercially available that facilitate sub-cloning VEGFmg in-frame to a fusion moiety.

TABLE C
Useful non-VEGFmg fusion polypeptides

| Reporter | in vitro | in vivo | Notes | Reference |
|---|---|---|---|---|
| Human growth | Radioimmunoassay | none | Expensive, | (Selden et al., |
| hormone (hGH) | | | insensitive, | 1986) |
| | | | narrow linear | |
| | | | range. | |
| β-glucuronidase | Colorimetric, | colorimetric | sensitive, | (Gallagher, |
| (GUS) | fluorescent, or | (histo-chemical | broad linear | 1992) |
| | chemi- | staining with X- | range, non- | |
| | luminescent | gluc) | iostopic. | |
| Green | Fluorescent | fluorescent | can be used in | (Chalfie et al., |
| fluorescent | | | live cells; | 1994) |
| protein (GFP) | | | resists photo- | |
| and related | | | bleaching | |
| molecules (RFP, | | | | |
| BFP, VEGFmg, | | | | |
| etc.) | | | | |
| Luciferase | bioluminsecent | Bioluminescent | protein is | (de Wet et al., |
| (firefly) | | | unstable, | 1987) |
| | | | difficult to | |
| | | | reproduce, | |
| | | | signal is brief | |
| Chloramphenicoal | Chromatography, | none | Expensive | (Gorman et al., |
| acetyltransferase | differential | | radioactive | 1982) |
| (CAT) | extraction, | | substrates, | |
| | fluorescent, or | | time- | |
| | immunoassay | | consuming, | |
| | | | insensitive, | |
| | | | narrow linear | |
| | | | range | |
| β-galacto-sidase | colorimetric, | colorimetric | sensitive, | (Alam and |
| | fluorescence, | (histochemical | broad linear | Cook, 1990) |
| | chemi- | staining with X- | range; some | |
| | luminscence | gal), bioluminescent | cells have high | |
| | | in | endogenous | |
| | | live cells | activity | |
| Secrete alkaline | colorimetric, | none | Chemiluminscence | (Berger et al., |
| phosphatase | bioluminescent, | | assay is | 1988) |
| (SEAP) | chemi- | | sensitive and | |
| | luminescent | | broad linear | |
| | | | range; some | |
| | | | cells have | |
| | | | endogenouse | |
| | | | alkaline | |
| | | | phosphatase | |
| | | | activity | |

9. VEGFmg Recombinant Expression Vectors and Host Cells

Vectors are tools used to shuttle DNA between host cells or as a means to express a nucleotide sequence. Some vectors function only in prokaryotes, while others function in both prokaryotes and eukaryotes, enabling large-scale DNA preparation from prokaryotes for expression in eukaryotes. Inserting the DNA of interest, such as VEGFmg nucleotide sequence or a fragment, is accomplished by ligation techniques and/or mating protocols well-known to the skilled artisan. Such DNA is inserted such that its integration does not disrupt any necessary components of the vector. In the case of vectors that are used to express the inserted DNA protein, the introduced DNA is operably-linked to the vector elements that govern its transcription and translation.

Vectors can be divided into two general classes: Cloning vectors are replicating plasmid or phage with regions that are non-essential for propagation in an appropriate host cell, and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably-linked to elements, such as promoters, that signal to the host cell to transcribe the inserted DNA. Some promoters are exceptionally useful, such as inducible promoters that control gene transcription in response to specific factors. Operably-linking VEGFmg or anti-sense construct to an inducible promoter can control the expression of VEGFmg or fragments, or anti-sense constructs. Examples of classic inducible promoters include those that are responsive to α-interferon, heat-shock, heavy metal ions, and steroids such as glucocorticoids (Kaufman, 1990) and tetracycline. Other desirable inducible promoters include those that are not endogenous to the cells in which the construct is being introduced, but, however, is responsive in those cells when the induction agent is exogenously supplied.

Vectors have many difference manifestations. A “plasmid” is a circular double stranded DNA molecule into which additional DNA segments can be introduced. Viral vectors can accept additional DNA segments into the viral genome. Certain vectors are capable of autonomous replication in a host cell (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. In general, useful expression vectors are often plasmids. However, other forms of expression vectors, such as viral vectors (e.g., replication defective retroviruses, adenoviruses and adeno-associated viruses) are contemplated. Recombinant expression vectors that comprise VEGFmg (or fragments) regulate VEGFmg transcription by exploiting one or more host cell-responsive (or that can be manipulated in vitro) regulatory sequences that is operably-linked to VEGFmg. “Operably-linked” indicates that a nucleotide sequence of interest is linked to regulatory sequences such that expression of the nucleotide sequence is achieved.

Vectors can be introduced in a variety of organisms and/or cells (Table D). Alternatively, the vectors can be transcribed and translated in vitro, for example using T7 promoter regulatory sequences and T7 polymerase.

TABLE D
Examples of hosts for cloning or expression

| Organisms | Examples | Sources and References* |
|---|---|---|
| Prokaryotes | | |
| Enterobacteriaceae | E. coli | |
| | K 12 strain MM294 | ATCC 31,446 |
| | X1776 | ATCC 31,537 |
| | W3110 | ATCC 27,325 |
| | K5 772 | ATCC 53,635 |
| | Enterobacter | |
| | Erwinia | |
| | Klebsiella | |
| | Proteus | |
| | Salmonella (S. tyhpimurium) | |
| | Serratia (S. marcescans) | |
| | Shigella | |
| | Bacilli (B. subtilis and B. licheniformis) | |
| | Pseudomonas (P. aeruginosa) | |
| | Streptomyces | |
| Eukaryotes | | |
| Yeasts | Saccharomyces cerevisiae | |
| | Schizosaccharomyces pombe | |
| | Kluyveromyces | (Fleer et al., 1991) |
| | K. lactis MW98-8C, | (de Louvencourt et al., 1983) |
| | CBS683, CBS4574 | |
| | K. fragilis | ATCC 12,424 |
| | K. bulgaricus | ATCC 16,045 |
| | K. wickeramii | ATCC 24,178 |
| | K. waltii | ATCC 56,500 |
| | K. drosophilarum | ATCC 36,906 |
| | K. thermotolerans | |
| | K. marxianus; yarrowia | (EPO 402226, 1990) |
| | Pichia pastoris | (Sreekrishna et al., 1988) |
| | Candida | |
| | Trichoderma reesia | |
| | Neurospora crassa | (Case et al., 1979) |
| | Torulopsis | |
| | Rhodotorula | |
| | Schwanniomyces (S. occidentalis) | |
| Filamentous Fungi | Neurospora | |
| | Penicillium | |
| | Tolypocladium | (WO 91/00357, 1991) |
| | Aspergillus (A. nidulans and | (Kelly and Hynes, 1985; |
| | A. niger) | Tilburn et al., 1983; Yelton et |
| | | al., 1984) |
| Invertebrate cells | Drosophila S2 | |
| | Spodoptera Sf9 | |
| Vertebrate cells | Chinese Hamster Ovary | |
| | (CHO) | |
| | simian COS | ATCC CRL 1651 |
| | COS-7 | |
| | HEK 293 | |

*Unreferenced cells are generally available from American Type Culture Collection (Manassas, VA).

Vector choice is dictated by the organism or cells being used and the desired fate of the vector. Vectors may replicate once in the target cells, or may be “suicide” vectors. In general, vectors comprise signal sequences, origins of replication, marker genes, enhancer elements, promoters, and transcription termination sequences. The choice of these elements depends on the organisms in which the vector will be used and are easily determined. Some of these elements may be conditional, such as an inducible or conditional promoter that is turned “on” when conditions are appropriate. Examples of inducible promoters include those that are tissue-specific, which relegate expression to certain cell types, steroid-responsive, or heat-shock reactive. Some bacterial repression systems, such as the lac operon, have been exploited in mammalian cells and transgenic animals (Fieck et al., 1992; Wyborski et al., 1996; Wyborski and Short, 1991). Vectors often use a selectable marker to facilitate identifying those cells that have incorporated the vector. Many selectable markers are well known in the art for the use with prokaryotes, usually antibiotic-resistance genes or the use of autotrophy and auxotrophy mutants.

Using antisense and sense VEGFmg oligonucleotides can prevent VEGFmg polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target VEGFmg mRNA (sense) or VEGFmg DNA (antisense) sequences. According to the present invention, antisense or sense oligonucleotides comprise a fragment of the VEGFmg DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Among others, (Stein and Cohen, 1988; van der Krol et al., 1988a) describe methods to derive antisense or a sense oligonucleotides from a given cDNA sequence.

Modifications of antisense and sense oligonucleotides can augment their effectiveness. Modified sugar-phosphodiester bonds or other sugar linkages (WO 91/06629, 1991), increase in vivo stability by conferring resistance to endogenous nucleases without disrupting binding specificity to target sequences. Other modifications can increase the affinities of the oligonucleotides for their targets, such as covalently linked organic moieties (WO 90/10448, 1990) or poly-(L)-lysine. Other attachments modify binding specificities of the oligonucleotides for their targets, including metal complexes or intercalating (e.g. ellipticine) and alkylating agents.

To introduce antisense or sense oligonucleotides into target cells (cells containing the target nucleic acid sequence), any gene transfer method may be used and are well known to those of skill in the art. Examples of gene transfer methods include 1) biological, such as gene transfer vectors like Epstein-Barr virus or conjugating the exogenous DNA to a ligand-binding molecule (WO 91/04753, 1991), 2) physical, such as electroporation, and 3) chemical, such as CaPO₄ precipitation and oligonucleotide-lipid complexes (WO 90/10448, 1990).

The terms “host cell” and “recombinant host cell” are used interchangeably. Such terms refer not only to a particular subject cell but also to the progeny or potential progeny of such a cell. Because certain modifications may occur in succeeding generations due to either mutation or environmental influences, such progeny may not, in fact, be identical to the parent cell, but are still included within the scope of the term.

Methods of eukaryotic cell transfection and prokaryotic cell transformation are well known in the art. The choice of host cell will dictate the preferred technique for introducing the nucleic acid of interest. Table E which is not meant to be limiting, summarizes many of the known techniques in the art. Introduction of nucleic acids into an organism may also be done with ex vivo techniques that use an in vitro method of transfection, as well as established genetic techniques, if any, for that particular organism.

TABLE E
Methods to introduce nucleic acid into cells

| Cells | Methods | References | Notes |
|---|---|---|---|
| Prokaryotes | Calcium chloride | (Cohen et al., 1972; | |
| (bacteria) | | Hanahan, 1983; Mandel | |
| | | and Higa, 1970) | |
| | Electroporation | (Shigekawa and Dower, | |
| | | 1988) | |
| Eukaryotes | Calcium | N-(2- | Cells may be |
| Mammalian cells | phosphate | Hydroxyethyl)piperazine- | “shocked” with |
| | transfection | N′-(2-ethanesulfonic acid | glycerol or |
| | | (HEPES) buffered saline | dimethylsulfoxide |
| | | solution (Chen and | (DMSO) to |
| | | Okayama, 1988; Graham | increase |
| | | and van der Eb, 1973; | transfection |
| | | Wigler et al., 1978) | efficiency |
| | | BES (N,N-bis(2- | (Ausubel et al., |
| | | hydroxyethyl)-2- | 1987). |
| | | aminoethanesulfonic acid) | |
| | | buffered solution (Ishiura | |
| | | et al., 1982) | |
| | Diethylaminoethyl | (Fujita et al., 1986; Lopata | Most useful for |
| | (DEAE)-Dextran | et al., 1984; Selden et al., | transient, but not |
| | transfections. | 1986) | stable, |
| | | | transfections. |
| | | | Chloroquine can |
| | | | be used to increase |
| | | | efficiency. |
| | Electroporation | (Neumann el al., 1982; | Especially useful |
| | | Potter, 1988; Potter et al., | for hard-to- |
| | | 1984; Wong and | transfect |
| | | Neumann, 1982) | lymphocytes. |
| | Cationic lipid | (Elroy-Stein and Moss, | Applicable to both |
| | reagent | 1990; Felgner et al., 1987; | in vivo and in vitro |
| | transfection | Rose et al., 1991; Whitt et | transfection. |
| | | al., 1990) | |
| | Retroviral | Production exemplified by | Lengthy process, |
| | | (Cepko et al., 1984; Miller | many packaging |
| | | and Buttimore, 1986; Pear | lines available at |
| | | et al., 1993) | ATCC. |
| | | Infection in vitro and in | Applicable to both |
| | | vivo: (Austin and Cepko, | in vivo and in vitro |
| | | 1990; Bodine et al., 1991; | transfection. |
| | | Fekete and Cepko, 1993; | |
| | | Lemischka et al., 1986; | |
| | | Turner et al., 1990; | |
| | | Williams et al., 1984) | |
| | Polybrene | (Chaney et al., 1986; | |
| | | Kawai and Nishizawa, | |
| | | 1984) | |
| | Microinjection | (Capecchi, 1980) | Can be used to |
| | | | establish cell lines |
| | | | carrying integrated |
| | | | copies of |
| | | | VEGFmg DNA |
| | | | sequences. |
| | Protoplast fusion | (Rassoulzadegan et al., | |
| | | 1982; Sandri-Goldin et al., | |
| | | 1981; Schaffner, 1980) | |
| Insect cells | Baculovirus | (Luckow, 1991; Miller, | Useful for in vitro |
| (in vitro) | systems | 1988; O'Reilly et al., 1992) | production of |
| | | | proteins with |
| | | | eukaryotic |
| | | | modifications. |
| Yeast | Electroporation | (Becker and Guarente, | |
| | | 1991) | |
| | Lithium acetate | (Gietz et al., 1998; Ito et | |
| | | al., 1983) | |
| | Spheroplast fusion | (Beggs, 1978; Hinnen et | Laborious, can |
| | | al., 1978) | produce |
| | | | aneuploids. |
| Plant cells | Agrobacterium | (Bechtold and Pelletier, | |
| (general | transformation | 1998; Escudero and Hohn, | |
| reference: | | 1997; Hansen and Chilton, | |
| (Hansen and | | 1999; Touraev and al., | |
| Wright, | | 1997) | |
| 1999)) | Biolistics | (Finer et al., 1999; Hansen | |
| | (microprojectiles) | and Chilton, 1999; Shillito, | |
| | | 1999) | |
| | Electroporation | (Fromm et al., 1985; Ou- | |
| | (protoplasts) | Lee et al., 1986; Rhodes et | |
| | | al., 1988; Saunders et al., | |
| | | 1989) | |
| | | May be combined with | |
| | | liposomes (Trick and al., | |
| | | 1997) | |
| | Polyethylene | (Shillito, 1999) | |
| | glycol (PEG) | | |
| | treatment | | |
| | Liposomes | May be combined with | |
| | | electroporation (Trick and | |
| | | al., 1997) | |
| | in planta | (Leduc and al., 1996; Zhou | |
| | microinjection | and al., 1983) | |
| | Seed imbibition | (Trick and al., 1997) | |
| | Laser beam | (Hoffman, 1996) | |
| | Silicon carbide | (Thompson and al., 1995) | |
| | whiskers | | |

Vectors often use a selectable marker to facilitate identifying those cells that have incorporated the vector. Many selectable markers are well known in the art for the use with prokaryotes, usually antibiotic-resistance genes or the use of autotrophy and auxotrophy mutants. Table F lists often-used selectable markers for mammalian cell transfection.

TABLE F
Useful selectable markers for eukaryote cell transfection

| Selectable | | | |
|---|---|---|---|
| Marker | Selection | Action | Reference |
| Adenosine | Media includes 9-β-D- | Conversion of Xyl-A | (Kaufman et |
| deaminase (ADA) | xylofuranosyl adenine | to Xyl-ATP, which | al., 1986) |
| | (Xyl-A) | incorporates into | |
| | | nucleic acids, killing | |
| | | cells. ADA detoxifies | |
| Dihydrofolate | Methotrexate (MTX) | MTX competitive | (Simonsen |
| reductase (DHFR) | and dialyzed serum | inhibitor of DHFR. In | and |
| | (purine-free media) | absence of exogenous | Levinson, |
| | | purines, cells require | 1983) |
| | | DHFR, a necessary | |
| | | enzyme in purine | |
| | | biosynthesis. | |
| Aminoglycoside | G418 | G418, an | (Southern |
| phosphotransferase | | aminoglycoside | and Berg, |
| (“APH”, “neo”, “G418”) | | detoxified by APH, | 1982) |
| | | interferes with | |
| | | ribosomal function | |
| | | and consequently, | |
| | | translation. | |
| Hygromycin-B- | hygromycin-B | Hygromycin-B, an | (Palmer et |
| phosphotransferase | | aminocyclitol | al., 1987) |
| (HPH) | | detoxified by HPH, | |
| | | disrupts protein | |
| | | translocation and | |
| | | promotes | |
| | | mistranslation. | |
| Thymidine kinase | Forward selection | Forward: | (Littlefield, |
| (TK) | (TK+): Media (HAT) | Aminopterin forces | 1964) |
| | incorporates | cells to synthesze | |
| | aminopterin. | dTTP from thymidine, | |
| | Reverse selection | a pathway requiring | |
| | (TK−): Media | TK. | |
| | incorporates 5- | Reverse: TK | |
| | bromodeoxyuridine | phosphorylates BrdU, | |
| | (BrdU). | which incorporates | |
| | | into nucleic acids, | |
| | | killing cells. | |

A host cell of the invention, such as a prokaryotic or eukaryotic host cell in culture, can be used to produce VEGFmg. Accordingly, the invention provides methods for producing VEGFmg using the host cells of the invention. In one embodiment, the method comprises culturing the host cell of the invention (into which a recombinant expression vector encoding VEGFmg has been introduced) in a suitable medium, such that VEGFmg is produced. In another embodiment, the method further comprises isolating VEGFmg from the medium or the host cell.

### Transgenic VEGFmg Animals

Transgenic animals are useful for studying the function and/or activity of VEGFmg and for identifying and/or evaluating modulators of VEGFmg activity. “Transgenic animals” are non-human animals, preferably mammals, more preferably rodents such as rats or mice, in which one or more of the cells include a transgene. Other transgenic animals include primates, sheep, dogs, cows, goats, chickens, amphibians, etc. A “transgene” is exogenous DNA that is integrated into the genome of a cell from which a transgenic animal develops, and that remains in the genome of the mature animal. Transgenes preferably direct the expression of an encoded gene product in one or more cell types or tissues of the transgenic animal with the purpose of preventing expression of a naturally encoded gene product in one or more cell types or tissues (a “knockout” transgenic animal), or serving as a marker or indicator of an integration, chromosomal location, or region of recombination (e.g. cre/loxP mice). A “homologous recombinant animal” is a non-human animal, such as a rodent, in which endogenous VEGFmg has been altered by an exogenous DNA molecule that recombines homologously with endogenous VEGFmg in a (e.g. embryonic) cell prior to development the animal. Host cells with exogenous VEGFmg can be used to produce non-human transgenic animals, such as fertilized oocytes or embryonic stem cells into which VEGFmg-coding sequences have been introduced. Such host cells can then be used to create non-human transgenic animals or homologous recombinant animals.

1. Approaches to Transgenic Animal Production

A transgenic animal can be created by introducing VEGFmg into the male pronuclei of a fertilized oocyte (e.g., by microinjection, retroviral infection) and allowing the oocyte to develop in a pseudopregnant female foster animal (pffa). The VEGFmg cDNA sequences can be introduced as a transgene into the genome of a non-human animal. Alternatively, a homologue of VEGFmg can be used as a transgene. Intronic sequences and polyadenylation signals can also be included in the transgene to increase transgene expression. Tissue-specific regulatory sequences can be operably-linked to the VEGFmg transgene to direct expression of VEGFmg to particular cells. Methods for generating transgenic animals via embryo manipulation and microinjection, particularly animals such as mice, have become conventional in the art, e.g. (Evans et al., U.S. Pat. No. 4,870,009, 1989; Hogan, 0879693843, 1994; Leder and Stewart, U.S. Pat. No. 4,736,866, 1988; Wagner and Hoppe, U.S. Pat. No. 4,873,191, 1989). Other non-mice transgenic animals may be made by similar methods. A transgenic founder animal, which can be used to breed additional transgenic animals, can be identified based upon the presence of the transgene in its genome and/or expression of the transgene mRNA in tissues or cells of the animals. Transgenic (e.g. VEGFmg) animals can be bred to other transgenic animals carrying other transgenes.

2. Vectors for Transgenic Animal Production

To create a homologous recombinant animal, a vector containing at least a portion of VEGFmg into which a deletion, addition or substitution has been introduced to thereby alter, e.g., functionally disrupt, VEGFmg. VEGFmg can be a murine gene or other VEGFmg homologue, such as the naturally occurring variant. In one approach, a knockout vector functionally disrupts the endogenous VEGFmg gene upon homologous recombination, and thus a non-functional VEGFmg protein, if any, is expressed.

Alternatively, the vector can be designed such that, upon homologous recombination, the endogenous VEGFmg is mutated or otherwise altered but still encodes functional protein (e.g., the upstream regulatory region can be altered to alter the expression of endogenous VEGFmg). In this type of homologous recombination vector, the altered portion of the VEGFmg is flanked at its 5′- and 3′-termini by additional nucleic acid of the VEGFmg to allow for homologous recombination to occur between the exogenous VEGFmg carried by the vector and an endogenous VEGFmg in an embryonic stem cell. The additional flanking VEGFmg nucleic acid is sufficient to engender homologous recombination with endogenous VEGFmg. Typically, several kilobases of flanking DNA (both at the 5′- and 3′-termini) are included in the vector (Thomas and Capecchi, 1987). The vector is then introduced into an embryonic stem cell line (e.g., by electroporation), and cells in which the introduced VEGFmg has homologously-recombined with the endogenous VEGFmg are selected (Li et al., 1992).

3. Introduction of VEGFmg Transgene Cells during Development

Selected cells are then injected into a blastocyst of an animal (e.g., a mouse) to form aggregation chimeras (Bradley, 1987). A chimeric embryo can then be implanted into a suitable pffa and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described (Berns et al., WO 93/04169, 1993; Bradley, 1991; Kucherlapati et al., WO 91/01140, 1991; Le Mouellic and Brullet, WO 90/11354, 1990).

Alternatively, transgenic animals that contain selected systems that allow for regulated expression of the transgene can be produced. An example of such a system is the cre/loxP recombinase system of bacteriophage P1 (Lakso et al., 1992). Another recombinase system is the FLP recombinase system of Saccharomyces cerevisiae (O'Gorman et al., 1991). If a cre/loxP recombinase system is used to regulate expression of the transgene, animals containing transgenes encoding both the Cre recombinase and a selected protein are required. Such animals can be produced as “double” transgenic animals, by mating an animal containing a transgene encoding a selected protein to another containing a transgene encoding a recombinase.

Clones of transgenic animals can also be produced (Wilmut et al., 1997). In brief, a cell from a transgenic animal can be isolated and induced to exit the growth cycle and enter G₀ phase. The quiescent cell can then be fused to an enucleated oocyte from an animal of the same species from which the quiescent cell is isolated. The reconstructed oocyte is then cultured to develop to a morula or blastocyte and then transferred to a pffa. The offspring borne of this female foster animal will be a clone of the “parent” transgenic animal.

### Anti-VEGFmg Abs

The invention encompasses Abs and antibody fragments, such as F_{ab} or (F_{ab})₂, that bind immunospecifically to any VEGFmg epitopes.

“Antibody” (Ab) comprises single Abs directed against VEGFmg (anti-VEGFmg Ab; including agonist, antagonist, and neutralizing Abs), anti-VEGFmg Ab compositions with poly-epitope specificity, single chain anti-VEGFmg Abs, and fragments of anti-VEGFmg Abs. A “monoclonal antibody” is obtained from a population of substantially homogeneous Abs, i.e., the individual Abs comprising the population are identical except for possible naturally-occurring mutations that may be present in minor amount. Exemplary Abs include polyclonal (pAb), monoclonal (mAb), humanized, bi-specific (bsAb), and heteroconjugate Abs.

1. Polyclonal Abs (pAbs)

Polyclonal Abs can be raised in a mammalian host, for example, by one or more injections of an immunogen and, if desired, an adjuvant. Typically, the immunogen and/or adjuvant are injected in the mammal by multiple subcutaneous or intraperitoneal injections. The immunogen may include VEGFmg or a fusion protein. Examples of adjuvants include Freund's complete and monophosphoryl Lipid A synthetic-trehalose dicorynomycolate (MPL-TDM). To improve the immune response, an immunogen may be conjugated to a protein that is immunogenic in the VEGF host, such as keyhole limpet hemocyanin (KLH), serum albumin, bovine thyroglobulin, and soybean trypsin inhibitor. Protocols for antibody production are described by (Ausubel et al., 1987; Harlow and Lane, 1988). Alternatively, pAbs may be made in chickens, producing IgY molecules (Schade et al., 1996).

2. Monoclonal Abs (mAbs)

Anti-VEGFmg mAbs may be prepared using hybridoma methods (Milstein and Cuello, 1983). Hybridoma methods comprise at least four steps: (1) immunizing a host, or lymphocytes from a host; (2) harvesting the mAb secreting (or potentially secreting) lymphocytes, (3) fusing the lymphocytes to immortalized cells, and (4) selecting those cells that secrete the desired (anti-VEGFmg) mAb.

A mouse, rat, guinea pig, hamster, or other appropriate host is immunized to elicit lymphocytes that produce or are capable of producing Abs that will specifically bind to the immunogen. Alternatively, the lymphocytes may be immunized in vitro. If human cells are desired, peripheral blood lymphocytes (PBLs) are generally used; however, spleen cells or lymphocytes from other mammalian sources are preferred. The immunogen typically includes VEGFmg or a fusion protein.

The lymphocytes are then fused with an immortalized cell line to form hybridoma cells, facilitated by a fusing agent such as polyethylene glycol (Goding, 1996). Rodent, bovine, or human myeloma cells immortalized by transformation may be used, or rat or mouse myeloma cell lines. Because pure populations of hybridoma cells and not unfused immortalized cells are preferred, the cells after fusion are grown in a suitable medium that contains one or more substances that inhibit the growth or survival of unfused, immortalized cells. A common technique uses parental cells that lack the enzyme hypoxanthine guanine phosphoribosyl transferase (HGPRT or HPRT). In this case, hypoxanthine, aminopterin and thymidine are added to the medium (HAT medium) to prevent the growth of HGPRT-deficient cells while permitting hybridomas to grow.

Preferred immortalized cells fuse efficiently, can be isolated from mixed populations by selecting in a medium such as HAT, and support stable and high-level expression of antibody after fusion. Preferred immortalized cell lines are murine myeloma lines, available from the American Type Culture Collection (Manassas, Va.). Human myeloma and mouse-human heteromyeloma cell lines also have been described for the production of human mAbs (Kozbor et al., 1984; Schook, 1987).

Because hybridoma cells secrete antibody extracellularly, the culture media can be assayed for the presence of mAbs directed against VEGFmg (anti-VEGFmg mAbs). Immunoprecipitation or in vitro binding assays, such as radio immunoassay (RIA) or enzyme-linked immunoabsorbent assay (ELISA), measure the binding specificity of mAbs (Harlow and Lane, 1988; Harlow and Lane, 1999), including Scatchard analysis (Munson and Rodbard, 1980).

Anti-VEGFmg mAb secreting hybridoma cells may be isolated as single clones by limiting dilution procedures and sub-cultured (Goding, 1996). Suitable culture media include Dulbecco's Modified Eagle's Medium, RPMI-1640, or if desired, a protein-free or -reduced or serum-free medium (e.g., Ultra DOMA PF or HL-1; Biowhittaker; Walkersville, Md.). The hybridoma cells may also be grown in vivo as ascites.

The mAbs may be isolated or purified from the culture medium or ascites fluid by conventional Ig purification procedures such as protein A-Sepharose, hydroxylapatite chromatography, gel electrophoresis, dialysis, ammonium sulfate precipitation or affinity chromatography (Harlow and Lane, 1988; Harlow and Lane, 1999).

The mAbs may also be made by recombinant methods (U.S. Pat. No. 4,166,452, 1979). DNA encoding anti-VEGFmg mAbs can be readily isolated and sequenced using conventional procedures, e.g., using oligonucleotide probes that specifically bind to murine heavy and light antibody chain genes, to probe preferably DNA isolated from anti-VEGFmg-secreting mAb hybridoma cell lines. Once isolated, the isolated DNA fragments are sub-cloned into expression vectors that are then transfected into host cells such as simian COS-7 cells, Chinese hamster ovary (CHO) cells, or myeloma cells that do not otherwise produce Ig protein, to express mAbs. The isolated DNA fragments can be modified, for example, by substituting the coding sequence for human heavy and light chain constant domains in place of the homologous murine sequences (U.S. Pat. No. 4,816,567, 1989; Morrison et al., 1987), or by fusing the Ig coding sequence to all or part of the coding sequence for a non-Ig polypeptide. Such a non-Ig polypeptide can be substituted for the constant domains of an antibody, or can be substituted for the variable domains of one antigen-combining site to create a chimeric bivalent antibody.

3. Monovalent Abs

The Abs may be monovalent Abs that consequently do not cross-link with each other. For example, one method involves recombinant expression of Ig light chain and modified heavy chain. Heavy chain truncations generally at any point in the F_{c} region will prevent heavy chain cross-linking. Alternatively, the relevant cysteine residues are substituted with another amino acid residue or are deleted, preventing crosslinking. In vitro methods are also suitable for preparing monovalent Abs. Abs can be digested to produce fragments, such as F_{ab} fragments (Harlow and Lane, 1988; Harlow and Lane, 1999).

4. Humanized and Human Abs

Anti-VEGFmg Abs may further comprise humanized or human Abs. Humanized forms of non-human Abs are chimeric Igs, Ig chains or fragments (such as Fᵥ, F_{ab}, F_{ab′}, F_{(ab′)2} or other antigen-binding subsequences of Abs) that contain minimal sequence derived from non-human Ig.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Jones et al., 1986; Riechmann et al., 1988; Verhoeyen et al., 1988). Such “humanized” Abs are chimeric Abs (U.S. Pat. No. 4,816,567, 1989), wherein substantially less than an intact human variable domain has been substituted by the corresponding sequence from a non-human species. In practice, humanized Abs are typically human Abs in which some CDR residues and possibly some FR residues are substituted by residues from analogous sites in rodent Abs. Humanized Abs include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fᵥ framework residues of the human Ig. Humanized Abs may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (F_{c}), typically that of a human Ig (Jones et al., 1986; Presta, 1992; Riechmann et al., 1988).

Human Abs can also be produced using various techniques, including phage display libraries (Hoogenboom et al., 1991; Marks et al., 1991) and the preparation of human mAbs (Boerner et al., 1991; Reisfeld and Sell, 1985). Similarly, introducing human Ig genes into transgenic animals in which the endogenous Ig genes have been partially or completely inactivated can be exploited to synthesize human Abs. Upon challenge, human antibody production is observed, which closely resembles that seen in humans in all respects, including gene rearrangement, assembly, and antibody repertoire (U.S. Pat. No. 5,545,807, 1996; U.S. Pat. No. 5,545,806, 1996; U.S. Pat. No. 5,569,825, 1996; U.S. Pat. No. 5,633,425, 1997; U.S. Pat. No. 5,661,016, 1997; U.S. Pat. No. 5,625,126, 1997; Fishwild et al., 1996; Lonberg and Huszar, 1995; Lonberg et al., 1994; Marks et al., 1992).

5. Bi-Specific mAbs

Bi-specific Abs are monoclonal, preferably human or humanized, that have binding specificities for at least two different antigens. For example, a binding specificity is VEGFmg; the other is for any antigen of choice, preferably a cell-surface protein or receptor or receptor subunit.

Traditionally, the recombinant production of bi-specific Abs is based on the co-expression of two Ig heavy-chain/light-chain pairs, where the two heavy chains have different specificities (Milstein and Cuello, 1983). Because of the random assortment of Ig heavy and light chains, the resulting hybridomas (quadromas) produce a potential mixture of ten different antibody molecules, of which only one has the desired bi-specific structure. The desired antibody can be purified using affinity chromatography or other techniques (WO 93/08829, 1993; Traunecker et al., 1991).

To manufacture a bi-specific antibody (Suresh et al., 1986), variable domains with the desired antibody-antigen combining sites are fused to Ig constant domain sequences. The fusion is preferably with an Ig heavy-chain constant domain, comprising at least part of the hinge, CH2, and CH3 regions. Preferably, the first heavy-chain constant region (CH1) containing the site necessary for light-chain binding is in at least one of the fusions. DNAs encoding the Ig heavy-chain fusions and, if desired, the Ig light chain, are inserted into separate expression vectors and are co-transfected into a suitable host organism.

The interface between a pair of antibody molecules can be engineered to maximize the percentage of heterodimers that are recovered from recombinant cell culture (WO 96/27011, 1996). The preferred interface comprises at least part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This mechanism increases the yield of the heterodimer over unwanted end products such as homodimers.

Bi-specific Abs can be prepared as full length Abs or antibody fragments (e.g. F_{(ab′)2} bi-specific Abs). One technique to generate bi-specific Abs exploits chemical linkage. Intact Abs can be proteolytically cleaved to generate F_{(ab′)2} fragments (Brennan et al., 1985). Fragments are reduced with a dithiol complexing agent, such as sodium arsenite, to stabilize vicinal dithiols and prevent intermolecular disulfide formation. The generated F_{ab′} fragments are then converted to thionitrobenzoate (TNB) derivatives. One of the F_{ab′}-TNB derivatives is then reconverted to the F_{ab′}-thiol by reduction with mercaptoethylamine and is mixed with an equimolar amount of the other F_{ab′}-TNB derivative to form the bi-specific antibody. The produced bi-specific Abs can be used as agents for the selective immobilization of enzymes.

F_{ab′} fragments may be directly recovered from E. coli and chemically coupled to form bi-specific Abs. For example, fully humanized bi-specific F_{(ab′)2} Abs can be produced (Shalaby et al., 1992). Each F_{ab′} fragment is separately secreted from E. coli and directly coupled chemically in vitro, forming the bi-specific antibody.

Various techniques for making and isolating bi-specific antibody fragments directly from recombinant cell culture have also been described. For example, leucine zipper motifs can be exploited (Kostelny et al., 1992). Peptides from the Fos and Jun proteins are linked to the F_{ab′} portions of two different Abs by gene fusion. The antibody homodimers are reduced at the hinge region to form monomers and then re-oxidized to form antibody heterodimers. This method can also produce antibody homodimers. The “diabody” technology (Holliger et al., 1993) provides an alternative method to generate bi-specific antibody fragments. The fragments comprise a heavy-chain variable domain (V_{H}) connected to a light-chain variable domain (V_{L}) by a linker that is too short to allow pairing between the two domains on the same chain. The V_{H} and V_{L} domains of one fragment are forced to pair with the complementary V_{L} and V_{H} domains of another fragment, forming two antigen-binding sites. Another strategy for making bi-specific antibody fragments is the use of single-chain Fᵥ (sFᵥ) dimers (Gruber et al., 1994). Abs with more than two valencies are also contemplated, such as tri-specific Abs (Tutt et al., 1991).

Exemplary bi-specific Abs may bind to two different epitopes on a given VEGFmg. Alternatively, cellular defense mechanisms can be restricted to a particular cell expressing the particular VEGFmg: an anti-VEGFmg arm may be combined with an arm that binds to a leukocyte triggering molecule, such as a T-cell receptor molecule (e.g. CD2, CD3, CD28, or B7), or to F_{c} receptors for IgG (F_{c}γR), such as F_{c}γRI (CD64), F_{c}γRII (CD32) and F_{c}γRIII (CD16). Bi-specific Abs may also be used to target cytotoxic agents to cells that express a particular VEGFmg. These Abs possess a VEGFmg-binding arm and an arm that binds a cytotoxic agent or a radionuclide chelator.

6. Heteroconjugate Abs

Heteroconjugate Abs, consisting of two covalently joined Abs, have been proposed to target immune system cells to unwanted cells (U.S. Pat. No. 4,676,980, 1987) and for treatment of human immunodeficiency virus (HIV) infection (WO 91/00360, 1991; WO 92/20373, 1992). Abs prepared in vitro using synthetic protein chemistry methods, including those involving cross-linking agents, are contemplated. For example, immunotoxins may be constructed using a disulfide exchange reaction or by forming a thioether bond. Examples of suitable reagents include iminothiolate and methyl-4-mercaptobutyrimidate (U.S. Pat. No. 4,676,980, 1987).

7. Immunoconjugates

Immunoconjugates may comprise an antibody conjugated to a cytotoxic agent such as a chemotherapeutic agent, toxin (e.g., an enzymatically active toxin or fragment of bacterial, fungal, plant, or animal origin), or a radioactive isotope (i.e., a radioconjugate).

Useful enzymatically-active toxins and fragments include Diphtheria A chain, non-binding active fragments of Diphtheria toxin, exotoxin A chain from Pseudomonas aeruginosa, ricin A chain, abrin A chain, modeccin A chain, α-sarcin, Aleurites fordii proteins, Dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated Abs, such as ²¹²Bi, ¹³¹I, ¹³¹In, ⁹⁰Y, and ¹⁸⁶Re.

Conjugates of the antibody and cytotoxic agent are made using a variety of bi-functional protein-coupling agents, such as N-succinimidyl-3-(2-pyridyldithiol)propionate (SPDP), iminothiolane (IT), bi-functional derivatives of imidoesters (such as dimethyl adipimidate HCl), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared (Vitetta et al., 1987). ¹⁴C-labeled 1-isothiocyanatobenzyl-3-methyldiethylene triaminepentaacetic acid (MX-DTPA) is an exemplary chelating agent for conjugating radionuclide to antibody (WO 94/11026, 1994).

In another embodiment, the antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a streptavidin “ligand” (e.g., biotin) that is conjugated to a cytotoxic agent (e.g., a radionuclide).

8. Effector Function Engineering

The antibody can be modified to enhance its effectiveness in treating a disease, such as cancer. For example, cysteine residue(s) may be introduced into the F_{c} region, thereby allowing interchain disulfide bond formation in this region. Such homodimeric Abs may have improved internalization capability and/or increased complement-mediated cell killing and antibody-dependent cellular cytotoxicity (ADCC) (Caron et al., 1992; Shopes, 1992). Homodimeric Abs with enhanced anti-tumor activity can be prepared using hetero-bifunctional cross-linkers (Wolff et al., 1993). Alternatively, an antibody engineered with dual F_{c} regions may have enhanced complement lysis (Stevenson et al., 1989).

9. Immunoliposomes

Liposomes containing the antibody may also be formulated (U.S. Pat. No. 4,485,045, 1984; U.S. Pat. No. 4,544,545, 1985; U.S. Pat. No. 5,013,556, 1991; Eppstein et al., 1985; Hwang et al., 1980). Useful liposomes can be generated by a reverse-phase evaporation method with a lipid composition comprising phosphatidylcholine, cholesterol, and PEG-derivatized phosphatidylethanolamine (PEG-PE). Such preparations are extruded through filters of defined pore size to yield liposomes with a desired diameter. F_{ab′} fragments of the antibody can be conjugated to the liposomes (Martin and Papahadjopoulos, 1982) via a disulfide-interchange reaction. A chemotherapeutic agent, such as Doxorubicin, may also be contained in the liposome (Gabizon et al., 1989). Other useful liposomes with different compositions are contemplated.

10. Diagnostic Applications of Abs Directed against VEGFmg

Anti-VEGFmg Abs can be used to localize and/or quantitate VEGFmg (e.g., for use in measuring levels of VEGFmg within tissue samples or for use in diagnostic methods, etc.). Anti-VEGFmg epitope Abs can be utilized as pharmacologically-active compounds.

Anti-VEGFmg Abs can be used to isolate a VEGFmg of choice by standard techniques, such as immunoaffinity chromatography or immunoprecipitation. These approaches facilitate purifying endogenous VEGFmg antigen-containing polypeptides from cells and tissues. These approaches, as well as others, can be used to detect a VEGFmg in a sample to evaluate the abundance and pattern of expression of the antigenic protein. Anti-VEGFmg Abs can be used to monitor protein levels in tissues as part of a clinical testing procedure; for example, to determine the efficacy of a given treatment regimen. Coupling the antibody to a detectable substance (label) allows detection of Ab-antigen complexes. Classes of labels include fluorescent, luminescent, bioluminescent, and radioactive materials, enzymes and prosthetic groups. Useful labels include horseradish peroxidase, alkaline phosphatase, β-galactosidase, acetylcholinesterase, streptavidin/biotin, avidin/biotin, umbelliferone, fluorescein, fluorescein isothiocyanate, rhodamine, dichlorotriazinylamine fluorescein, dansyl chloride, phycoerythrin, luminol, luciferase, luciferin, aequorin, and ¹²⁵I, ¹³¹I, ³⁵S or ³H.

11. Antibody Therapeutics

Abs of the invention, including polyclonal, monoclonal, humanized and fully human Abs, can be used therapeutically. Such agents will generally be employed to treat or prevent a disease or pathology in a subject. An antibody preparation, preferably one having high antigen specificity and affinity generally mediates an effect by binding the target epitope(s). Generally, administration of such Abs may mediate one of two effects: (1) the antibody may prevent ligand binding, eliminating endogenous ligand binding and subsequent signal transduction, or (2) the antibody elicits a physiological result by binding an effector site on the target molecule, initiating signal transduction.

A therapeutically effective amount of an antibody relates generally to the amount needed to achieve a therapeutic objective, epitope binding affinity, administration rate, and depletion rate of the antibody from a subject. Common ranges for therapeutically effective doses may be, as a nonlimiting example, from about 0.1 mg/kg body weight to about 50 mg/kg body weight. Dosing frequencies may range, for example, from twice daily to once a week.

12. Pharmaceutical Compositions of Abs

Anti-VEGFmg Abs, as well as other VEGFmg interacting molecules (such as aptamers) identified in other assays, can be administered in pharmaceutical compositions to treat various disorders. Principles and considerations involved in preparing such compositions, as well as guidance in the choice of components can be found in (de Boer, 1994; Gennaro, 2000; Lee, 1990).

Because many VEGFmgs are intracellular, Abs that are internalized are preferred when whole Abs are used as inhibitors to these molecules. Otherwise, Abs that are not internalized are preferred, such as anti-osteonidogen Abs. Liposomes may also be used as a delivery vehicle for intracellular introduction. Where antibody fragments are used, the smallest inhibitory fragment that specifically binds to the epitope is preferred. For example, peptide molecules can be designed that bind a preferred epitope based on the variable-region sequences of a useful antibody. Such peptides can be synthesized chemically and/or produced by recombinant DNA technology (Marasco et al., 1993). Formulations may also contain more than one active compound for a particular treatment, preferably those with activities that do not adversely affect each other. The composition may comprise an agent that enhances function, such as a cytotoxic agent, cytokine, chemotherapeutic agent, or growth-inhibitory agent.

The active ingredients can also be entrapped in microcapsules prepared by coacervation techniques or by interfacial polymerization; for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration are highly preferred to be sterile. This is readily accomplished by filtration through sterile filtration membranes or any of a number of techniques.

Sustained-release preparations may also be prepared, such as semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (Boswell and Scribner, U.S. Pat. No. 3,773,919, 1973), copolymers of L-glutamic acid and γ ethyl-L-glutamate, non-degradable ethylene-vinyl acetate, degradable lactic acid-glycolic acid copolymers such as injectable microspheres composed of lactic acid-glycolic acid copolymer, and poly-D-(−)-3-hydroxybutyric acid. While polymers such as ethylene-vinyl acetate and lactic acid-glycolic acid enable release of molecules for over 100 days, certain hydrogels release proteins for shorter time periods and may be preferred.

### Therapeutic Applications of VEGFmg

1. Pathology-Related Utilities

The polynucleotides and proteins of the invention are useful in potential therapeutic applications implicated in tumors and neoplasias, hamangiomas, rheumatoid arthritis, atherosclerosis, idiopathic pulmonary fibrosis, vascular restenosis, arteriovenous malformations, meningioma, neovascular glaucoma, psoriasis, agniofibroma, hemophilic joints, hypertrophic scars, Osler-Weber syndrome, pyogenic gtranuloma retrolental fibroplasias, scleroderma, trachoma, vascular adhesion pathologies, synovitis, dermatitis, enometriosis, pterygium, diabetic retinopathy, newovascularization associated with corneal injury or grafts, wound, sore, and ulcers (skin, gastric and duodenal) healing. For example, a cDNA encoding ARP may be useful in gene therapy, and ARP protein may be useful when administered to a subject in need thereof. The novel nucleic acid encoding ARP, and the ARP protein of the invention, or fragments thereof, may further be useful in diagnostic applications, wherein the presence or amount of the nucleic acid or the protein are to be assessed. These materials are further useful in the generation of Abs that bind immunospecifically to the novel substances of the invention for use in therapeutic or diagnostic methods.

In addition, the instant invention may be used to determine the clinical state or pathology of a sample, such as a biopsy of cells taken from a patient. A clinical state of a growth, such as a tumor or cancer, is a classification system recognized by those of skill in the art to categorize, for example, the metastatic aggressiveness of a cancer.

2. Agonists and Antagonists

“Antagonist” includes any molecule that partially or fully blocks, inhibits, or neutralizes a biological activity of endogenous VEGFmg. Similarly, “agonist” includes any molecule that mimics a biological activity of endogenous VEGFmg. Molecules that can act as agonists or antagonists include Abs or antibody fragments, fragments or variants of endogenous VEGFmg, peptides, antisense oligonucleotides, small organic molecules, etc.

3. Identifying Antagonists and Agonists

To assay for antagonists, VEGFmg is added to, or expressed in, a cell along with the compound to be screened for a particular activity. If the compound inhibits the activity of interest in the presence of the VEGFmg, that compound is an antagonist to the VEGFmg; if VEGFmg activity is enhanced, the compound is an agonist.

(a) Specific Examples of Potential Antagonists and Agonist

Any molecule that alters VEGFmg cellular effects, such as angiogenesis or cell survival, is a candidate antagonist or agonist. Screening techniques well known to those skilled in the art can identify these molecules. Examples of antagonists and agonists include: (1) small organic and inorganic compounds, (2) small peptides, (3) Abs and derivatives, (4) polypeptides closely related to VEGFmg, (5) antisense DNA and RNA, (6) ribozymes, (7) triple DNA helices and (8) nucleic acid aptamers.

Small molecules that bind to the VEGFmg active site or other relevant part of the polypeptide and inhibit the biological activity of the VEGFmg are antagonists. Examples of small molecule antagonists include small peptides, peptide-like molecules, preferably soluble, and synthetic non-peptidyl organic or inorganic compounds. These same molecules, if they enhance VEGFmg activity, are examples of agonists.

Almost any antibody that affects a VEGFmg's function is a candidate antagonist, and occasionally, agonist. Examples of antibody antagonists include polyclonal, monoclonal, single-chain, anti-idiotypic, chimeric Abs, or humanized versions of such Abs or fragments. Abs may be from any species in which an immune response can be raised Humanized Abs are also contemplated.

Alternatively, a potential antagonist or agonist may be a closely related protein, for example, a mutated form of the VEGFmg that recognizes a VEGFmg-interacting protein but imparts no effect, competitively inhibiting VEGFmg action. Alternatively, a mutated VEGFmg may be constitutively activated and may act as an agonist.

Antisense RNA or DNA constructs can be effective antagonist. Antisense RNA or DNA molecules block function by inhibiting translation by hybridizing to targeted mRNA. Antisense technology can be used to control gene expression through triple-helix formation or antisense DNA or RNA, both of which depend on polynucleotide binding to DNA or RNA. For example, the 5′ coding portion of the VEGFmg sequence is used to design an antisense RNA oligonucleotide of from about 10 to 40 base pairs in length. A DNA oligonucleotide is designed to be complementary to a region of the gene involved in transcription (triple helix) (Beal and Dervan, 1991; Cooney et al., 1988; Lee et al., 1979), preventing transcription and the production of the VEGFmg. The antisense RNA oligonucleotide hybridizes to the mRNA in vivo and blocks translation of the mRNA molecule into the VEGFmg (antisense) (Cohen, 1989; Okano et al., 1991). These oligonucleotides can also be delivered to cells such that the antisense RNA or DNA may be expressed in vivo to inhibit production of the VEGFmg. When antisense DNA is used, oligodeoxyribonucleotides derived from the translation-initiation site, e.g., between about −10 and +10 positions of the target gene nucleotide sequence, are preferred.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques (WO 97/33551, 1997; Rossi, 1994).

To inhibit transcription, triple-helix nucleic acids that are single-stranded and comprise deoxynucleotides are useful antagonists. These oligonucleotides are designed such that triple-helix formation via Hoogsteen base-pairing rules is promoted, generally requiring stretches of purines or pyrimidines (WO 97/33551, 1997).

Because a VEGFmg activity may include nucleic acid binding, molecules that compete for VEGFmg nucleic acid binding site(s) can be effective intracellular competitors. Aptamers are short oligonucleotide sequences that can be used to recognize and specifically bind almost any molecule. The systematic evolution of ligands by exponential enrichment (SELEX) process (Ausubel et al., 1987; Ellington and Szostak, 1990; Tuerk and Gold, 1990) is powerful and can be used to find such aptamers. Aptamers have many diagnostic and clinical uses; almost any use in which an antibody has been used clinically or diagnostically, aptamers too may be used. In addition, are cheaper to make once they have been identified, and can be easily applied in a variety of formats, including administration in pharmaceutical compositions, in bioassays, and diagnostic tests (Jayasena, 1999).

### Pharmaceutical Compositions

The VEGFmg nucleic acid molecules, VEGFmg polypeptides, and anti-VEGFmg Abs (active compounds) of the invention, and derivatives, fragments, analogs and homologs thereof, can be incorporated into pharmaceutical compositions. Such compositions typically comprise the nucleic acid molecule, protein, or antibody and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (Gennaro, 2000). Preferred examples of such carriers or diluents include, but are not limited to, water, saline, finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

1. General Considerations

A pharmaceutical composition of the invention is formulated to be compatible with its intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

2. Injectable Formulations

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents, for example, parabens, chlorobutanol, phenol, ascorbic acid, and thimerosal, can contain microorganism contamination. Isotonic agents, for example, sugars, polyalcohols such as manitol, sorbitol, and sodium chloride can be included in the composition. Compositions that can delay absorption include agents such as aluminum monostearate and gelatin.

Sterile injectable solutions can be prepared by incorporating the active compound (e.g., VEGFmg or anti-VEGFmg antibody) in the required amount in an appropriate solvent with one or a combination of ingredients as required, followed by sterilization. Generally, dispersions are prepared by incorporating the active compound into a sterile vehicle that contains a basic dispersion medium, and the other required ingredients as discussed. Sterile powders for the preparation of sterile injectable solutions, methods of preparation include vacuum drying and freeze-drying that yield a powder containing the active ingredient and any desired ingredient from a sterile solutions.

3. Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

4. Compositions for Inhalation

For administration by inhalation, the compounds are delivered as an aerosol spray from a a nebulizer or a pressurized container that contains a suitable propellant, e.g., a gas such as carbon dioxide.

5. Systemic Administration

Systemic administration can also be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams.

The compounds can also be prepared in the form of suppositories (e.g., with bases such as cocoa butter and other glycerides) or retention enemas for rectal delivery.

6. Carriers

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art. Liposomal suspensions can also be used as pharmaceutically acceptable carriers. These can be prepared according to methods known to those skilled in the art, such as in (Eppstein et al., U.S. Pat. No. 4,522,811, 1985).

7. Unit Dosage

Oral formulations or parenteral compositions in unit dosage form can be created to facilitate administration and dosage uniformity. Unit dosage form refers to physically discrete units suited as single dosages for the subject to be treated, containing a therapeutically effective quantity of active compound in association with the required pharmaceutical carrier. The specification for the unit dosage forms of the invention are dictated by, and directly dependent on, the unique characteristics of the active compound and the particular desired therapeutic effect, and the inherent limitations of compounding the active compound.

8. Gene Therapy Compositions

The nucleic acid molecules of the invention can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

9. Kits for Pharmaceutical Compositions

The pharmaceutical compositions can be included in a kit, container, pack, or dispenser together with instructions for administration. When the invention is supplied as a kit, the different components of the composition may be packaged in separate containers and admixed immediately before use. Such packaging of the components separately may permit long-term storage without losing the active components' functions.

Kits may also include reagents in separate containers that facilitate the execution of a specific test, such as diagnostic tests or tissue typing. For example, VEGFmg DNA templates and suitable primers may be supplied for internal controls.

(a) Containers or Vessels

The reagents included in the kits can be supplied in containers of any sort such that the life of the different components are preserved, and are not adsorbed or altered by the materials of the container. For example, sealed glass ampules may contain lyophilized luciferase or buffer that have been packaged under a neutral, non-reacting gas, such as nitrogen. Ampoules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, video tape, audio tape, etc. Detailed instructions may not be physically associated with the kit; instead, a user may be directed to an internet web site specified by the manufacturer or distributor of the kit, or supplied as electronic mail.

### Screening and Detection Methods

Isolated nucleic acid molecules can be used to express VEGFmg (e.g., via a recombinant expression vector in a host cell in gene therapy applications), to detect VEGFmg mRNA (e.g., in a biological sample) or a genetic lesion in a VEGFmg, and to modulate VEGFmg activity, as described below. In addition, VEGFmg polypeptides can be used to screen drugs or compounds that modulate VEGFmg activity or expression as well as to treat disorders characterized by insufficient or excessive production of VEGFmg or production of VEGFmg forms that have decreased or aberrant activity compared to VEGFmg wild-type protein, or modulate biological function that involve VEGFmg (e.g. angiogenesis). In addition, the anti-VEGFmg Abs of the invention can be used to detect and isolate VEGFmg and modulate VEGFmg activity.

To modulate cell survival means to decrease or increase probability that a cell will die in the future over a period of time as compared to cells prior to modulation.

1. Screening Assays

The invention provides a method (screening assay) for identifying modalities, i.e., candidate or test compounds or agents (e.g., peptides, peptidomimetics, small molecules or other drugs), foods, dosing regimens, combinations thereof, etc., that effect VEGFmg, a stimulatory or inhibitory effect, including translation, transcription, activity or copies of the gene in cells. The invention also includes compounds identified in screening assays.

Testing for compounds that increase or decrease VEGFmg activity are desirable. A compound may modulate VEGFmg activity by affecting: (1) the number of copies of the gene in the cell (amplifiers and deamplifiers); (2) increasing or decreasing transcription of the VEGFmg (transcription up-regulators and down-regulators); (3) by increasing or decreasing the translation of VEGFmg mRNA into protein (translation up-regulators and down-regulators); or (4) by increasing or decreasing the activity of VEGFmg itself (agonists and antagonists).

(a) Effects of Compounds

To identify compounds that affect VEGFmg at the DNA, RNA and protein levels, cells or organisms are contacted with a candidate compound and the corresponding change in VEGFmg DNA, RNA or protein is assessed (Ausubel et al., 1987). For DNA amplifiers and deamplifiers, the amount of VEGFmg DNA is measured, for those compounds that are transcription up-regulators and down-regulators the amount of VEGFmg mRNA is determined; for translational up- and down-regulators, the amount of VEGFmg polypeptides is measured. Compounds that are agonists or antagonists may be identified by contacting cells or organisms with the compound, and then measuring, for example, angiogenesis or cell survival in vitro.

In one embodiment, many assays for screening candidate or test compounds that bind to or modulate the activity of VEGFmg or polypeptide or biologically-active portion are available. Test compounds can be obtained using any of the numerous approaches in combinatorial library methods, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptides, while the other four approaches encompass peptide, non-peptide oligomer or small molecule libraries of compounds (Lam, 1997).

(b) Small Molecules

A “small molecule” refers to a composition that has a molecular weight of less than about 5 kD and most preferably less than about 4 kD, even more preferably less than 0.6 kD. Small molecules can be, nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994).

Libraries of compounds may be presented in solution (Houghten et al., 1992) or on beads (Lam et al., 1991), on chips (Fodor et al., 1993), bacteria, spores (Ladner et al., U.S. Pat. No. 5,223,409, 1993), plasmids (Cull et al., 1992) or on phage (Cwirla et al., 1990; Devlin et al., 1990; Felici et al., 1991; Ladner et al., U.S. Pat. No. 5,223,409, 1993; Scott and Smith, 1990). A cell-free assay comprises contacting VEGFmg or biologically-active fragment with a known compound that binds VEGFmg to form an assay mixture, contacting the assay mixture with a test compound, and determining the ability of the test compound to interact with VEGFmg, where determining the ability of the test compound to interact with VEGFmg comprises determining the ability of the VEGFmg to preferentially bind to or modulate the activity of a VEGFmg target molecule.

(c) Cell-Free Assays

The cell-free assays of the invention may be used with both soluble or a membrane-bound forms of VEGFmg. In the case of cell-free assays comprising the membrane-bound form, a solubilizing agent to maintain VEGFmg in solution. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON® X-100 and others from the TRITON® series, THESIT®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

(d) Immobilization of Target Molecules to Facilitate Screening

In more than one embodiment of the assay methods, immobilizing either VEGFmg or a partner molecule can facilitate separation of complexed from uncomplexed forms of one or both of the proteins, as well as to accommodate high throughput assays. Binding of a test compound to VEGFmg, or interaction of VEGFmg with a target molecule in the presence and absence of a candidate compound, can be accomplished in any vessel suitable for containing the reactants, such as microtiter plates, test tubes, and micro-centrifuge tubes. A fusion protein can be provided that adds a domain that allows one or both of the proteins to be bound to a matrix. For example, GST-VEGFmg fusion proteins or GST-target fusion proteins can be adsorbed onto glutathione sepharose beads (Sigma Chemical; St. Louis, Mo.) or glutathione derivatized microtiter plates that are then combined with the test compound or the test compound and either the non-adsorbed target protein or VEGFmg, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described. Alternatively, the complexes can be dissociated from the matrix, and the level of VEGFmg binding or activity determined using standard techniques.

Other techniques for immobilizing proteins on matrices can also be used in screening assays. Either VEGFmg or a target molecule can be immobilized using biotin-avidin or biotin-streptavidin systems. Biotinylation can be accomplished using many reagents, such as biotin-NHS (N-hydroxy-succinimide; PIERCE Chemicals, Rockford, Ill.), and immobilized in wells of streptavidin-coated 96 well plates (PIERCE Chemical). Alternatively, Abs reactive with VEGFmg or target molecules, but which do not interfere with binding of the VEGFmg to its target molecule, can be derivatized to the wells of the plate, and unbound target or VEGFmg trapped in the wells by antibody conjugation. Methods for detecting such complexes, in addition to those described for the GST-immobilized complexes, include immunodetection of complexes using Abs reactive with VEGFmg or its target, as well as enzyme-linked assays that rely on detecting an enzymatic activity associated with the VEGFmg or target molecule.

(e) Screens to Identify Modulators

Modulators of VEGFmg expression can be identified in a method where a cell is contacted with a candidate compound and the expression of VEGFmg mRNA or protein in the cell is determined. The expression level of VEGFmg mRNA or protein in the presence of the candidate compound is compared to VEGFmg mRNA or protein levels in the absence of the candidate compound. The candidate compound can then be identified as a modulator of VEGFmg mRNA or protein expression based upon this comparison. For example, when expression of VEGFmg mRNA or protein is greater (i.e., statistically significant) in the presence of the candidate compound than in its absence, the candidate compound is identified as a stimulator of VEGFmg mRNA or protein expression. Alternatively, when expression of VEGFmg mRNA or protein is less (statistically significant) in the presence of the candidate compound than in its absence, the candidate compound is identified as an inhibitor of VEGFmg mRNA or protein expression. The level of VEGFmg mRNA or protein expression in the cells can be determined by methods described for detecting VEGFmg mRNA or protein.

(f) Hybrid Assays

In yet another aspect of the invention, VEGFmg can be used as “bait” in two-hybrid or three hybrid assays (Bartel et al., 1993; Brent et al., WO94/10300, 1994; Iwabuchi et al., 1993; Madura et al., 1993; Saifer et al., U.S. Pat. No. 5,283,317, 1994; Zervos et al., 1993) to identify other proteins that bind or interact with VEGFmg (VEGFmg-binding proteins (VEGFmg-bps)) and modulate VEGFmg activity. Such VEGFmg-bps are also likely to be involved in the propagation of signals by the VEGFmg as, for example, upstream or downstream elements of a VEGFmg pathway.

The two-hybrid system is based on the modular nature of most transcription factors, which consist of separable DNA-binding and activation domains. Briefly, the assay utilizes two different DNA constructs. In one construct, the gene that codes for VEGFmg is fused to a gene encoding the DNA binding domain of a known transcription factor (e.g., GAL4). The other construct, a DNA sequence from a library of DNA sequences that encodes an unidentified protein (“prey” or “sample”) is fused to a gene that codes for the activation domain of the known transcription factor. If the “bait” and the “prey” proteins are able to interact in vivo, forming a VEGFmg-dependent complex, the DNA-binding and activation domains of the transcription factor are brought into close proximity. This proximity allows transcription of a reporter gene (e.g., LacZ) that is operably-linked to a transcriptional regulatory site responsive to the transcription factor. Expression of the reporter gene can be detected, and cell colonies containing the functional transcription factor can be isolated and used to obtain the cloned gene that encodes the VEGFmg-interacting protein.

(g) Calcium Channel Regulators

Several classes of calcium channel blocker are known and may be effective antagonists and agonists. For example, Mak et al. (Mak et al., 1995) report the activity of the lipophilic calcium channel blockers, nicardipine, nifedipine, verapamil, and diltiazem as anti-oxidants and protectants for endothelial cells. Calcium channels may play a significant role in the cell survival in which the genes identified herein are differentially expressed. Among the VEGFmgs that are significant in calcium regulation are DSCR1 and nexin. For example, those agents that stimulate the expression of DSCR1 or nexin and reduce the activity of the mitochondrial respiratory chain will promote survival and are useful to treat angiogenesis-related diseases, that is, diseases in which angiogenesis is repressed or insufficient. Agents that reduce the expression of e.g. DSCR1 or nexin and that increase the activity of the mitochondrial respiratory chain will induce or promote apoptosis and therefore are useful to treat diseases where the angiogenesis is stimulated.

The invention further pertains to novel agents identified by the aforementioned screening assays and uses thereof for treatments as described herein.

2. Detection Assays

Portions or fragments of VEGFmg cDNA sequences identified herein (and the complete VEGFmg gene sequences) are useful in themselves. By way of non-limiting example, these sequences can be used to: (1) identify an individual from a minute biological sample (tissue typing); and (2) aid in forensic identification of a biological sample.

(a) Tissue Typing

The VEGFmg sequences of the invention can be used to identify individuals from minute biological samples. In this technique, an individual's genomic DNA is digested with one or more restriction enzymes and probed on a Southern blot to yield unique bands. The sequences of the invention are useful as additional DNA markers for “restriction fragment length polymorphisms” (RFLP; (Smulson et al., U.S. Pat. No. 5,272,057, 1993)).

Furthermore, the VEGFmg sequences can be used to determine the actual base-by-base DNA sequence of targeted portions of an individual's genome. VEGFmg sequences can be used to prepare two PCR primers from the 5′- and 3′-termini of the sequences that can then be used to amplify an the corresponding sequences from an individual's genome and then sequence the amplified fragment.

Panels of corresponding DNA sequences from individuals can provide unique individual identifications, as each individual will have a unique set of such DNA sequences due to allelic differences. The sequences of the invention can be used to obtain such identification sequences from individuals and from tissue. The VEGFmg sequences of the invention uniquely represent portions of an individual's genome. Allelic variation occurs to some degree in the coding regions of these sequences, and to a greater degree in the noncoding regions. The allelic variation between individual humans occurs with a frequency of about once ever 500 bases. Much of the allelic variation is due to single nucleotide polymorphisms (SNPs), which include RFLPs.

Each of the sequences described herein can, to some degree, be used as a standard against which DNA from an individual can be compared for identification purposes. Because greater numbers of polymorphisms occur in noncoding regions, fewer sequences are necessary to differentiate individuals. Noncoding sequences can positively identify individuals with a panel of 10 to 1,000 primers that each yield a noncoding amplified sequence of 100 bases.

3. Assaying VEGF-Modulated Genes using Oligonucleotide Arrays

In addition to using the nucleotide probes, antibodies, etc., described above, other methods are available to identify VEGFmg expression.

The invention provides for the use of the genes identified as differentially expressed in methods directed to screen for compounds that affect survival of endothelial cells, such as HUVECs. The simultaneous analysis of VEGFmg expression levels with appropriate controls can assess drugs, proteins, or other compounds and formulations. Assessing the extent of differential expression of VEGFmgs can be accomplished using an array or similar device containing oligonucleotides complementary to and capable of binding or hybridizing to the mRNAs corresponding to VEGFmgs. For example, such an array can measure mRNA levels in endothelial cells treated with, for example, a compound, and compared to mRNA levels in untreated cells. One example of this device is GeneChip™ (Affymetrix, CITY, CA), a miniaturized, high-density array of oligonucleotides complementary to and capable of binding or hybridizing to a set of mRNAs. The technical implementation of this strategy is described in detail (Lipshutz et al., 1999).

### Predictive Medicine

The invention also pertains to the field of predictive medicine in which diagnostic assays, prognostic assays, pharmacogenomics, and monitoring clinical trials are used for prognostic (predictive) purposes to treat an individual prophylactically. Accordingly, one aspect of the invention relates to diagnostic assays for determining VEGFmg and/or nucleic acid expression as well as VEGFmg activity, in the context of a biological sample (e.g., blood, serum, cells, tissue) to determine whether an individual is afflicted with a disease or disorder, or is at risk of developing a disorder, associated with aberrant VEGFmg expression or activity, including angiogenesis and cell survival. The invention also provides for prognostic (or predictive) assays for determining whether an individual is at risk of developing a disorder associated with VEGFmg, nucleic acid expression or activity. For example, mutations in VEGFmg can be assayed in a biological sample. Such assays can be used for prognostic or predictive purpose to prophylactically treat an individual prior to the onset of a disorder characterized by or associated with VEGFmg, nucleic acid expression, or biological activity.

Another aspect of the invention provides methods for determining VEGFmg activity, or nucleic acid expression, in an individual to select appropriate therapeutic or prophylactic agents for that individual (referred to herein as “pharmacogenomics”). Pharmacogenomics allows for the selection of modalities (e.g., drugs, foods) for therapeutic or prophylactic treatment of an individual based on the individual's genotype (e.g., the individual's genotype to determine the individual's ability to respond to a particular agent). Another aspect of the invention pertains to monitoring the influence of modalities (e.g., drugs, foods) on the expression or activity of VEGFmg in clinical trials.

1. Diagnostic Assays

An exemplary method for detecting the presence or absence of VEGFmg in a biological sample involves obtaining a biological sample from a subject and contacting the biological sample with a compound or an agent capable of detecting VEGFmg or VEGFmg nucleic acids (e.g., mRNA, genomic DNA) such that the presence of a VEGFmg is confirmed in the sample. An agent for detecting VEGFmg mRNA or genomic DNA is a labeled nucleic acid probe that can hybridize to VEGFmg mRNA or genomic DNA. The nucleic acid probe can be, for example, a full-length VEGFmg nucleic acid or a portion thereof, such as an oligonucleotide of at least 15, 30, 50, 100, 250 or 500 nucleotides in length and sufficient to specifically hybridize under stringent conditions to VEGFmg mRNA or genomic DNA.

An agent for detecting VEGFmg polypeptide is an antibody capable of binding to a VEGFmg, preferably an antibody with a detectable label. Abs can be polyclonal, or more preferably, monoclonal. An intact antibody, or a fragment (e.g., F_{ab} or F(ab′)₂) can be used. A labeled probe or antibody is coupled (i.e., physically linking) to a detectable substance, as well as indirect detection of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” includes tissues, cells and biological fluids isolated from a subject, as well as tissues, cells and fluids present within a subject. The detection method of the invention can be used to detect VEGFmg mRNA, protein, or genomic DNA in a biological sample in vitro as well as in vivo. For example, in vitro techniques for detection of VEGFmg mRNA include Northern hybridizations and in situ hybridizations. In vitro techniques for detection of VEGFmg polypeptide include enzyme linked immunosorbent assays (ELISAs), Western blots, immunoprecipitations, and immunofluorescence. In vitro techniques for detection of VEGFmg genomic DNA include Southern hybridizations and fluorescence in situ hybridization (FISH). Furthermore, in vivo techniques for detecting VEGFmg include introducing into a subject a labeled anti-VEGFmg antibody. For example, the antibody can be labeled with a radioactive marker whose presence and location in a subject can be detected by standard imaging techniques.

In one embodiment, the biological sample from the subject contains protein molecules, and/or mRNA molecules, and/or genomic DNA molecules. A preferred biological sample is blood.

In another embodiment, the methods further involve obtaining a biological sample from a subject to provide a control, contacting the sample with a compound or agent to detect VEGFmg, mRNA, or genomic DNA, and comparing the presence of VEGFmg, mRNA or genomic DNA in the control sample with the presence of VEGFmg, mRNA or genomic DNA in the test sample.

The invention also encompasses kits for detecting VEGFmg in a biological sample. For example, the kit can comprise: a labeled compound or agent capable of detecting VEGFmg or VEGFmg mRNA in a sample; reagent and/or equipment for determining the amount of VEGFmg in the sample; and reagent and/or equipment for comparing the amount of VEGFmg in the sample with a standard. The compound or agent can be packaged in a suitable container. The kit can further comprise instructions for using the kit to detect VEGFmg or nucleic acid.

2. Prognostic Assays

The diagnostic methods described herein can furthermore be utilized to identify subjects having or at risk of developing a disease or disorder associated with aberrant VEGFmg expression or activity. For example, the assays described herein, can be used to identify a subject having or at risk of developing a disorder associated with VEGFmg, nucleic acid expression or activity. Alternatively, the prognostic assays can be used to identify a subject having or at risk for developing a disease or disorder. The invention provides a method for identifying a disease or disorder associated with aberrant VEGFmg expression or activity in which a test sample is obtained from a subject and VEGFmg or nucleic acid (e.g., mRNA, genomic DNA) is detected. A test sample is a biological sample obtained from a subject. For example, a test sample can be a biological fluid (e.g., serum), cell sample, or tissue.

Prognostic assays can be used to determine whether a subject can be administered a modality (e.g., an agonist, antagonist, peptidomimetic, protein, peptide, nucleic acid, small molecule, food, etc.) to treat a disease or disorder associated with aberrant VEGFmg expression or activity. Such methods can be used to determine whether a subject can be effectively treated with an agent for a disorder. The invention provides methods for determining whether a subject can be effectively treated with an agent for a disorder associated with aberrant VEGFmg expression or activity in which a test sample is obtained and VEGFmg or nucleic acid is detected (e.g., where the presence of VEGFmg or nucleic acid is diagnostic for a subject that can be administered the agent to treat a disorder associated with aberrant VEGFmg expression or activity).

The methods of the invention can also be used to detect genetic lesions in a VEGFmg to determine if a subject with the genetic lesion is at risk for a disorder characterized by aberrant cell proliferation or differentiation. Methods include detecting, in a sample from the subject, the presence or absence of a genetic lesion characterized by at an alteration affecting the integrity of a gene encoding a VEGFmg polypeptide, or the mis-expression of VEGFmg. Such genetic lesions can be detected by ascertaining: (1) a deletion of one or more nucleotides from VEGFmg, (2) an addition of one or more nucleotides to a VEGFmg, (3) a substitution of one or more nucleotides in a VEGFmg, (4) a chromosomal rearrangement of a VEGFmg gene; (5) an alteration in the level of a VEGFmg mRNA transcripts, (6) aberrant modification of a VEGFmg, such as a change genomic DNA methylation, (7) the presence of a non-wild-type splicing pattern of a VEGFmg mRNA transcript, (8) a non-wild-type level of a VEGFmg, (9) allelic loss of VEGFmg, and/or (10) inappropriate post-translational modification of VEGFmg polypeptide. There are a large number of known assay techniques that can be used to detect lesions in a VEGFmg. Any biological sample containing nucleated cells may be used.

In certain embodiments, lesion detection may use a probe/primer in a polymerase chain reaction (PCR) (e.g., (Mullis, U.S. Pat. No. 4,683,202, 1987; Mullis et al., U.S. Pat. No. 4,683,195, 1987), such as anchor PCR or rapid amplification of cDNA ends (RACE) PCR, or, alternatively, in a ligation chain reaction (LCR) (e.g., (Landegren et al., 1988; Nakazawa et al., 1994), the latter is particularly useful for detecting point mutations in VEGFmg-genes (Abravaya et al., 1995). This method may include collecting a sample from a patient, isolating nucleic acids from the sample, contacting the nucleic acids with one or more primers that specifically hybridize to a VEGFmg under conditions such that hybridization and amplification of the VEGFmg (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli et al., 1990), transcriptional amplification system (Kwoh et al., 1989); Qβ Replicase (Lizardi et al., 1988), or any other nucleic acid amplification method, followed by the detection of the amplified molecules using techniques well known to those of skill in the art. These detection schemes are especially useful for the detection of nucleic acid molecules present in low abundance.

Mutations in a VEGFmg from a sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes can be used to score for the presence of specific mutations by development or loss of a ribozyme cleavage site.

Hybridizing a sample and control nucleic acids, e.g., DNA or RNA, to high-density arrays containing hundreds or thousands of oligonucleotides probes, can identify genetic mutations in VEGFmg (Cronin et al., 1996; Kozal et al., 1996). For example, genetic mutations in VEGFmg can be identified in two-dimensional arrays containing light-generated DNA probes (Cronin, et al., 1996). Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the VEGFmg of interest and detect mutations by comparing the sequence of the sample VEGFmg-with the corresponding wild-type (control) sequence. Examples of sequencing reactions include those based on classic techniques (Maxam and Gilbert, 1977; Sanger et al., 1977). Any of a variety of automated sequencing procedures can be used when performing diagnostic assays (Naeve et al., 1995) including sequencing by mass spectrometry (Cohen et al., 1996; Griffin and Griffin, 1993; Koster, WO94/16101, 1994).

Other methods for detecting mutations in a VEGFmg include those in which protection from cleavage agents is used to detect mismatched bases in RNA/RNA or RNA/DNA heteroduplexes (Myers et al., 1985). In general, the technique of “mismatch cleavage” starts by providing heteroduplexes formed by hybridizing (labeled) RNA or DNA containing the wild-type VEGFmg sequence with potentially mutant RNA or DNA obtained from a sample. The double-stranded duplexes are treated with an agent that cleaves single-stranded regions of the duplex such as those that arise from base pair mismatches between the control and sample strands. For instance, RNA/DNA duplexes can be treated with RNase and DNA/DNA hybrids treated with S₁ nuclease to enzymatically digest the mismatched regions. In other embodiments, either DNA/DNA or RNA/DNA duplexes can be treated with hydroxylamine or osmium tetroxide and with piperidine in order to digest mismatched regions. The digested material is then separated by size on denaturing polyacrylamide gels to determine the mutation site (Grompe et al., 1989; Saleeba and Cotton, 1993). The control DNA or RNA can be labeled for detection.

Mismatch cleavage reactions may employ one or more proteins that recognize mismatched base pairs in double-stranded DNA (DNA mismatch repair) in defined systems for detecting and mapping point mutations in VEGFmg cDNAs obtained from samples of cells. For example, the mutY enzyme of E. coli cleaves A at G/A mismatches and the thymidine DNA glycosylase from HeLa cells cleaves T at G/T mismatches (Hsu et al., 1994). According to an exemplary embodiment, a probe based on a wild-type VEGFmg sequence is hybridized to a cDNA or other DNA product from a test cell(s). The duplex is treated with a DNA mismatch repair enzyme, and the cleavage products, if any, can be detected from electrophoresis protocols or the like (Modrich et al., U.S. Pat. No. 5,459,039, 1995).

Electrophoretic mobility alterations can be used to identify mutations in VEGFmg. For example, single strand conformation polymorphism (SSCP) may be used to detect differences in electrophoretic mobility between mutant and wild type nucleic acids (Cotton, 1993; Hayashi, 1992; Orita et al., 1989). Single-stranded DNA fragments of sample and control VEGFmg nucleic acids are denatured and then renatured. The secondary structure of single-stranded nucleic acids varies according to sequence; the resulting alteration in electrophoretic mobility allows detection of even a single base change. The DNA fragments may be labeled or detected with labeled probes. The sensitivity of the assay may be enhanced by using RNA (rather than DNA), in which the secondary structure is more sensitive to a sequence changes. The subject method may use heteroduplex analysis to separate double stranded heteroduplex molecules on the basis of changes in electrophoretic mobility (Keen et al., 1991).

The migration of mutant or wild-type fragments can be assayed using denaturing gradient gel electrophoresis (DGGE; (Myers et al., 1985). In DGGE, DNA is modified to prevent complete denaturation, for example by adding a GC clamp of approximately 40 bp of high-melting GC-rich DNA by PCR. A temperature gradient may also be used in place of a denaturing gradient to identify differences in the mobility of control and sample DNA (Rossiter and Caskey, 1990).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al., 1986; Saiki et al., 1989). Such allele-specific oligonucleotides are hybridized to PCR-amplified target DNA or a number of different mutations when the oligonucleotides are attached to the hybridizing membrane and hybridized with labeled target DNA.

Alternatively, allele specific amplification technology that depends on selective PCR amplification may be used. Oligonucleotide primers for specific amplifications may carry the mutation of interest in the center of the molecule (so that amplification depends on differential hybridization (Gibbs et al., 1989)) or at the extreme 3′-terminus of one primer where, under appropriate conditions, mismatch can prevent, or reduce polymerase extension (Prosser, 1993). Novel restriction site in the region of the mutation may be introduced to create cleavage-based detection (Gasparini et al., 1992). Certain amplification may also be performed using Taq ligase for amplification (Barany, 1991). In such cases, ligation occurs only if there is a perfect match at the 3′-terminus of the 5′ sequence, allowing detection of a known mutation by scoring for amplification.

The described methods may be performed, for example, by using pre-packaged kits comprising at least one probe (nucleic acid or antibody) that may be conveniently used, for example, in clinical settings to diagnose patients exhibiting symptoms or family history of a disease or illness involving VEGFmg.

Furthermore, any cell type or tissue in which VEGFmg is expressed may be utilized in the prognostic assays described herein.

3. Pharmacogenomics

Agents, or modulators that have a stimulatory or inhibitory effect on VEGFmg activity or expression, as identified by a screening assay can be administered to individuals to treat, prophylactically or therapeutically, disorders, including insufficient blood supply or improper cell survival. In conjunction with such treatment, the pharmacogenomics (i.e., the study of the relationship between a subject's genotype and the subject's response to a foreign modality, such as a food, compound or drug) may be considered. Metabolic differences of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Pharmacogenomics can further be used to determine appropriate dosages and therapeutic regimens. Accordingly, the activity of VEGFmg, expression of VEGFmg nucleic acid, or VEGFmg mutation(s) in an individual can be determined to guide the selection of appropriate agent(s) for therapeutic or prophylactic treatment.

Pharmacogenomics deals with clinically significant hereditary variations in the response to modalities due to altered modality disposition and abnormal action in affected persons (Eichelbaum and Evert, 1996; Linder et al., 1997). In general, two pharmacogenetic conditions can be differentiated: (1) genetic conditions transmitted as a single factor altering the interaction of a modality with the body (altered drug action) or (2) genetic conditions transmitted as single factors altering the way the body acts on a modality (altered drug metabolism). These pharmacogenetic conditions can occur either as rare defects or as nucleic acid polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) explains the phenomena of some patients who show exaggerated drug response and/or serious toxicity after taking the standard and safe dose of a drug. These polymorphisms are expressed in two phenotypes in the population, the extensive metabolizer (EM) and poor metabolizer (PM). The prevalence of PM is different among different populations. For example, the CYP2D6 gene is highly polymorphic and several mutations have been identified in PM, which all lead to the absence of functional CYP2D6. Poor metabolizers due to mutant CYP2D6 and CYP2C19 frequently experience exaggerated drug responses and side effects when they receive standard doses. If a metabolite is the active therapeutic moiety, PM shows no therapeutic response, as demonstrated for the analgesic effect of codeine mediated by its CYP2D6-formed metabolite morphine. At the other extreme are the so-called ultra-rapid metabolizers who are unresponsive to standard doses. Recently, the molecular basis of ultra-rapid metabolism has been identified to be due to CYP2D6 gene amplification.

The activity of VEGFmg, expression of VEGFmg nucleic acid, or mutation content of VEGFmg in an individual can be determined to select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a VEGFmg modulator, such as a modulator identified by one of the described exemplary screening assays.

4. Monitoring Effects during Clinical Trials

Monitoring the influence of agents (e.g., drugs, compounds) on the expression r activity of VEGFmg (e.g., the ability to modulate aberrant cell proliferation and/or differentiation) can be applied not only in basic drug screening, but also in clinical trials. For example, the effectiveness of an agent determined by a screening assay to increase VEGFmg expression, protein levels, or up-regulate VEGFmg activity can be monitored in clinical trails of subjects exhibiting decreased VEGFmg expression, protein levels, or down-regulated VEGFmg activity. Alternatively, the effectiveness of an agent determined to decrease VEGFmg expression, protein levels, or down-regulate VEGFmg activity, can be monitored in clinical trails of subjects exhibiting increased VEGFmg expression, protein levels, or up-regulated VEGFmg activity. In such clinical trials, the expression or activity of VEGFmg and, preferably, other genes that have been implicated in, for example, angiogenesis or apoptosis, can be used as a “read out” or markers for a particular cell's responsiveness.

For example, genes, including VEGFmg, that are modulated in cells by treatment with a modality (e.g., food, compound, drug or small molecule) can be identified. To study the effect of agents on cellular proliferation disorders, for example, in a clinical trial, cells can be isolated and RNA prepared and analyzed for the levels of expression of VEGFmg and other genes implicated in the disorder. The gene expression pattern can be quantified by Northern blot analysis, nuclear run-on or RT-PCR experiments, or by measuring the amount of protein, or by measuring the activity level of VEGFmg or other gene products. In this manner, the gene expression pattern itself can serve as a marker, indicative of the cellular physiological response to the agent. Accordingly, this response state may be determined before, and at various points during, treatment of the individual with the agent.

The invention provides a method for monitoring the effectiveness of treatment of a subject with an agent (e.g., an agonist, antagonist, protein, peptide, peptidomimetic, nucleic acid, small molecule, food or other drug candidate identified by the screening assays described herein) comprising the steps of (1) obtaining a pre-administration sample from a subject; (2) detecting the level of expression of a VEGFmg, mRNA, or genomic DNA in the preadministration sample; (3) obtaining one or more post-administration samples from the subject; (4) detecting the level of expression or activity of the VEGFmg, mRNA, or genomic DNA in the post-administration samples; (5) comparing the level of expression or activity of the VEGFmg, mRNA, or genomic DNA in the pre-administration sample with the VEGFmg, mRNA, or genomic DNA in the post administration sample or samples; and (6) altering the administration of the agent to the subject accordingly. For example, increased administration of the agent may be desirable to increase the expression or activity of VEGFmg to higher levels than detected, i.e., to increase the effectiveness of the agent. Alternatively, decreased administration of the agent may be desirable to decrease expression or activity of VEGFmg to lower levels than detected, i.e., to decrease the effectiveness of the agent.

5. Methods of Treatment

The invention provides for both prophylactic and therapeutic methods of treating a subject at risk of (or susceptible to) a disorder or having a disorder associated with aberrant VEGFmg expression or activity.

6. Diseases and Disorders

Diseases and disorders that are characterized by increased VEGFmg levels or biological activity may be treated with therapeutics that antagonize (i.e., reduce or inhibit) activity. Antognists may be administered in a therapeutic or prophylactic manner. Therapeutics that may be used include: (1) VEGFmg peptides, or analogs, derivatives, fragments or homologs thereof; (2) Abs to a VEGFmg peptide; (3) VEGFmg nucleic acids; (4) administration of antisense nucleic acid and nucleic acids that are “dysfunctional” (i.e., due to a heterologous insertion within the coding sequences) that are used to eliminate endogenous function of by homologous recombination (Capecchi, 1989); or (5) modulators (i.e., inhibitors, agonists and antagonists, including additional peptide mimetic of the invention or Abs specific to VEGFmg) that alter the interaction between VEGFmg and its binding partner.

Diseases and disorders that are characterized by decreased VEGFmg levels or biological activity may be treated with therapeutics that increase (i.e., are agonists to) activity. Therapeutics that upregulate activity may be administered therapeutically or prophylactically. Therapeutics that may be used include peptides, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying in vitro for RNA or peptide levels, structure and/or activity of the expressed peptides (or VEGFmg mRNAs). Methods include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

7. Prophylactic Methods

The invention provides a method for preventing, in a subject, a disease or condition associated with an aberrant VEGFmg expression or activity, by administering an agent that modulates VEGFmg expression or at least one VEGFmg activity. Subjects at risk for a disease that is caused or contributed to by aberrant VEGFmg expression or activity, such as tumorigenesis or metastasis, can be identified by, for example, any or a combination of diagnostic or prognostic assays. Administration of a prophylactic agent can occur prior to the manifestation of symptoms characteristic of the VEGFmg aberrancy, such that a disease or disorder is prevented or, alternatively, delayed in its progression. Depending on the type of VEGFmg aberrancy, for example, a VEGFmg agonist or VEGFmg antagonist can be used to treat the subject. The appropriate agent can be determined based on screening assays.

VEGFmg nucleic acids, or fragments, may also be useful in diagnostic applications, wherein the presence or amount of the nucleic acid or the protein is to be assessed. A further use could be as an anti-bacterial molecule (i.e., some peptides have been found to possess anti-bacterial properties). These materials are further useful in the generation of Abs that immunospecifically bind to the novel substances of the invention for use in therapeutic or diagnostic methods.

8. Therapeutic Methods

Another aspect of the invention pertains to methods of modulating VEGFmg expression or activity for therapeutic purposes. The modulatory method of the invention involves contacting a cell with an agent that modulates one or more of the activities of VEGFmg activity associated with the cell. An agent that modulates VEGFmg activity can be a nucleic acid or a protein, a naturally occurring cognate ligand of VEGFmg, a peptide, a VEGFmg peptidomimetic, or other small molecule. The agent may stimulate VEGFmg activity. Examples of such stimulatory agents include active VEGFmg and a VEGFmg nucleic acid molecule that has been introduced into the cell. In another embodiment, the agent inhibits VEGFmg activity. Examples of inhibitory agents include antisense VEGFmg nucleic acids and anti-VEGFmg Abs. Modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a VEGFmg or nucleic acid molecule. In one embodiment, the method involves administering an agent (e.g., an agent identified by a screening assay), or combination of agents that modulates (e.g., up-regulates or down-regulates) VEGFmg expression or activity. In another embodiment, the method involves administering a VEGFmg or nucleic acid molecule as therapy to compensate for reduced or aberrant VEGFmg expression or activity.

Stimulation of VEGFmg activity is desirable in situations in which VEGFmg is abnormally down-regulated and/or in which increased VEGFmg activity is likely to have a beneficial effect. One example of such a situation is where a subject has a disorder characterized by aberrant cell proliferation and/or differentiation (e.g., cancer or immune associated disorders).

9. Determination of the Biological Effect of the Therapeutic

Suitable in vitro or in vivo assays can be performed to determine the effect of a specific therapeutic and whether its administration is indicated for treatment of the affected tissue.

In various specific embodiments, in vitro assays may be performed with representative cells of the type(s) involved in the patient's disorder, to determine if a given therapeutic exerts the desired effect upon the cell type(s). Modalities for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects. Various assays directed at measuring angiogenesis and cell survival may be used.

10. Anti-sense Nucleic Acids

Using antisense and sense VEGFmg oligonucleotides can prevent VEGFmg polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target VEGFmg mRNA (sense) or VEGFmg DNA (antisense) sequences. Anti-sense nucleic acids can be designed according to Watson and Crick or Hoogsteen base pairing rules. The anti-sense nucleic acid molecule can be complementary to the entire coding region of VEGFmg mRNA, but more preferably, to only a portion of the coding or noncoding region of VEGFmg mRNA. For example, the anti-sense oligonucleotide can be complementary to the region surrounding the translation start site of VEGFmg mRNA. Antisense or sense oligonucleotides may comprise a fragment of the VEGFmg DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Among others, (Stein and Cohen, 1988; van der Krol et al., 1988a) describe methods to derive antisense or a sense oligonucleotides from a given cDNA sequence.

Examples of modified nucleotides that can be used to generate the anti-sense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the anti-sense nucleic acid can be produced biologically using an expression vector into which a nucleic acid has been sub-cloned in an anti-sense orientation such that the transcribed RNA will be complementary to a target nucleic acid of interest.

To introduce antisense or sense oligonucleotides into target cells (cells containing the target nucleic acid sequence), any gene transfer method may be used. Examples of gene transfer methods include (1) biological, such as gene transfer vectors like Epstein-Barr virus or conjugating the exogenous DNA to a ligand-binding molecule, (2) physical, such as electroporation and injection, and (3) chemical, such as CaPO₄ precipitation and oligonucleotide-lipid complexes.

An antisense or sense oligonucleotide is inserted into a suitable gene transfer retroviral vector. A cell containing the target nucleic acid sequence is contacted with the recombinant retroviral vector, either in vivo or ex vivo. Examples of suitable retroviral vectors include those derived from the murine retrovirus M-MuLV, N2 (a retrovirus derived from M-MuLV), or the double copy vectors designated DCT5A, DCT5B and DCT5C (WO 90/13641, 1990). To achieve sufficient nucleic acid molecule transcription, vector constructs in which the transcription of the anti-sense nucleic acid molecule is controlled by a strong pol II or pol III promoter are preferred.

To specify target cells in a mixed population of cells cell surface receptors that are specific to the target cells can be exploited. Antisense and sense oligonucleotides are conjugated to a ligand-binding molecule, as described in (WO 91/04753, 1991). Ligands are chosen for receptors that are specific to the target cells. Examples of suitable ligand-binding molecules include cell surface receptors, growth factors, cytokines, or other ligands that bind to cell surface receptors or molecules. Preferably, conjugation of the ligand-binding molecule does not substantially interfere with the ability of the receptors or molecule to bind the ligand-binding molecule conjugate, or block entry of the sense or antisense oligonucleotide or its conjugated version into the cell.

Liposomes efficiently transfer sense or an antisense oligonucleotide to cells (WO 90/10448, 1990). The sense or antisense oligonucleotide-lipid complex is preferably dissociated within the cell by an endogenous lipase.

The anti-sense nucleic acid molecule of the invention may be an α-anomeric nucleic acid molecule. An α-anomeric nucleic acid molecule forms specific double-stranded hybrids with complementary RNA in which, contrary to the usual α-units, the strands run parallel to each other (Gautier et al., 1987). The anti-sense nucleic acid molecule can also comprise a 2′-o-methylribonucleotide (Inoue et al., 1987a) or a chimeric RNA-DNA analogue (Inoue et al., 1987b).

In one embodiment, an anti-sense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes, such as hammerhead ribozymes (Haseloff and Gerlach, 1988) can be used to catalytically cleave VEGFmg mRNA transcripts and thus inhibit translation. A ribozyme specific for a VEGFmg-encoding nucleic acid can be designed based on the nucleotide sequence of a VEGFmg cDNA. For example, a derivative of a Tetrahymena L-19 IVS RNA can be constructed in which the nucleotide sequence of the active site is complementary to the nucleotide sequence to be cleaved in a VEGFmg-encoding mRNA (Cech et al., U.S. Pat. No. 5,116,742, 1992; Cech et al., U.S. Pat. No. 4,987,071, 1991). VEGFmg mRNA can also be used to select a catalytic RNA having a specific ribonuclease activity from a pool of RNA molecules (Bartel and Szostak, 1993).

Alternatively, VEGFmg expression can be inhibited by targeting nucleotide sequences complementary to the regulatory region of the VEGFmg (e.g., the VEGFmg promoter and/or enhancers) to form triple helical structures that prevent transcription of the VEGFmg in target cells (Helene, 1991; Helene et al., 1992; Maher, 1992).

Modifications of antisense and sense oligonucleotides can augment their effectiveness. Modified sugar-phosphodiester bonds or other sugar linkages (WO 91/06629, 1991), increase in vivo stability by conferring resistance to endogenous nucleases without disrupting binding specificity to target sequences. Other modifications can increase the affinities of the oligonucleotides for their targets, such as covalently linked organic moieties (WO 90/10448, 1990) or poly-(L)-lysine. Other attachments modify binding specificities of the oligonucleotides for their targets, including metal complexes or intercalating (e.g. ellipticine) and alkylating agents. For example, the deoxyribose phosphate backbone of the nucleic acids can be modified to generate peptide nucleic acids (Hyrup and Nielsen, 1996). “Peptide nucleic acids” or “PNAs” refer to nucleic acid mimics (e.g., DNA mimics) in that the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols (Hyrup and Nielsen, 1996; Perry-O'Keefe et al., 1996). PNAs of VEGFmg can be used in therapeutic and diagnostic applications. For example, PNAs can be used as anti-sense or antigene agents for sequence-specific modulation of gene expression by inducing transcription or translation arrest or inhibiting replication. VEGFmg PNAs may also be used in the analysis of single base pair mutations (e.g., PNA directed PCR clamping; as artificial restriction enzymes when used in combination with other enzymes, e.g., S₁ nucleases (Hyrup and Nielsen, 1996); or as probes or primers for DNA sequence and hybridization (Hyrup and Nielsen, 1996; Perry-O'Keefe et al., 1996).

PNAs of VEGFmg can be modified to enhance their stability or cellular uptake. Lipophilic or other helper groups may be attached to PNAs, PNA-DNA dimmers formed, or the use of liposomes or other drug delivery techniques. For example, PNA-DNA chimeras can be generated that may combine the advantageous properties of PNA and DNA. Such chimeras allow DNA recognition enzymes (e.g., RNase H and DNA polymerases) to interact with the DNA portion while the PNA portion provides high binding affinity and specificity. PNA-DNA chimeras can be linked using linkers of appropriate lengths selected in terms of base stacking, number of bonds between the nucleobases, and orientation (Hyrup and Nielsen, 1996). The synthesis of PNA-DNA chimeras can be performed (Finn et al., 1996; Hyrup and Nielsen, 1996). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Finn et al., 1996; Hyrup and Nielsen, 1996). PNA monomers are then coupled in a stepwise manner to produce a chimeric molecule with a 5′ PNA segment and a 3′ DNA segment (Finn et al., 1996). Alternatively, chimeric molecules can be synthesized with a 5′ DNA segment and a 3′ PNA segment (Petersen et al., 1976).

The oligonucleotide may include other appended groups such as peptides (e.g., for targeting host cell receptors in vivo), or agents facilitating transport across the cell membrane (Lemaitre et al., 1987; Letsinger et al., 1989) or PCT Publication No. WO88/09810) or the blood-brain barrier (e.g., PCT Publication No. WO 89/10134). In addition, oligonucleotides can be modified with hybridization-triggered cleavage agents (van der Krol et al., 1988b) or intercalating agents (Zon, 1988). The oligonucleotide may be conjugated to another molecule, e.g., a peptide, a hybridization triggered cross-linking agent, a transport agent, a hybridization-triggered cleavage agent, and the like.

The following examples illustrate by way of non-limiting example various aspects of the invention.

### EXAMPLES

### Example 1

### Differential Gene Expression in Human Umbilical Cord Endothelial Cells (HUVECs)

1. Background

To obtain a comprehensive profile of those genes whose expression is modulated during VEGF-dependent, or mutant VEGFR1-dependent, survival pathway, GeneCalling™^{s} technology (Rothberg et al., U.S. Pat. No. 5,871,697, 1999; Shimkets et al., 1999), was applied to serum-starved human umbilical cord endothelial cells treated with a set of growth factors and to reference HUVEC cells grown in the presence of 10% serum. Cells grown in the absence of both any growth factor and serum served as the negative control. GeneCalling™ technology relies on Quantitative Expression Analysis to generate the gene expression profile of a given sample and then generates differential expression analysis of pairwise comparison of these profiles to controls containing no addition. Polynucleotides exhibiting differential expression are confirmed by conducting a PCR reaction according to the GeneCalling™ protocol with the addition of a competing unlabelled primer that prevents the amplification from being detected.

2. Growth Factors Used

(a) VEGF

A principal growth factor employed in this example is VEGF, which binds to both VEGFR1 and VEGFR2. In addition, a mutant of VEGF that binds only VEGFR1 (VEGFR1s) was used. The other growth factors used in this study bind to receptors other than VEFGR1 and have different angiogenic potential. They are included as positive (VEGF, VEGFR1s) and negative (PlGF, bFGF, HGS/SF) controls to focus the analysis on the VEGFR1 pathway.

(b) bFGF

Basic fibroblast growth factor (bFGF) is expressed in vascular endothelium during tumor neovascularization and angioproliferative diseases. VEGF and bFGF are potently synergistic in their combined mitogenic activity. A possible explanation for this synergism is the evidence that bFGF induces the expression of VEGF receptor VEGFR1 and of VEGF itself (Hata et al., 1999). Treatment with bFGF will modulate a set of genes overlapping with those modulated by VEGF and VEGFR1s.

(c) HGF/SF

Hepatocyte growth factor/scatter factor (HGF/SF) is a pleiotropic growth factor that stimulates proliferation and migration of endothelial cells. Similarly to bFGF, HGF and VEGF are synergistic in their combined angiogenic activity (Van Belle et al., 1997). HGF induces VEGF expression (Gille et al., 1998). Therefore it could be expected that treatment with bFGF will modulate a set of genes overlapping with those modulated by VEGF.

(d) PlGF

Placenta growth factor (PlGF) belongs to the family of VEGFs (VEGFs). Three PlGF isoforms are produced by alternative splicing and all induce migration of endothelial cells while having no effect on cell proliferation (Migdal et al., 1998). They ligate VEGFR2 receptor but not to VEGFR1 that is thought to mediate most of the angiogenic and proliferative effects of VEGF. Treatment with PlGF will modulate a set of genes overlapping with those modulated by VEGF but not with those modulated by VEGFR1s. This observation allows for the identification of the set of genes specifically modulated by VEGF via the VEGFR1 receptor.

3. Genes Analysed and Corresponding GenBank Accession

Table E1 provides the GenBank Accession numbers for the genes whose expression was analyzed in this example.

TABLE E1
GenBank Accessions for analysed genes

| Gene Name | GenBank Accession |
|---|---|
| Nexin/Glia derived neurite promoting factor | A03911 |
| (GDNPF) | |
| placental protein 5 (PP5)/tissue factor pathway | 5730090 |
| inhibitor 2 | D29992 |
| heparin-binding EGF-like growth factor (HB-EGF) | 4503412 |
| Regulator of G-protein signaling 3 (RGS3) | U27655 |
| Gravin/myasthenia gravis autoantigen | U81607 |
| MKP-1 like protein tyrosine phosphatase | AF038844 |
| (MKP1LPTP) | |
| amyloid precursor-like protein 2 (APLP2) | L27631 |
| Osteonidogen, nidogen-2 precursor | D86425 |
| amyloid precursor protein (APP) | D87675 |
| hVPS41p | U87309 |
| arginine-rich protein (ARP) | 5174392/M83751 |
| Down's syndrome critical region protein 1 (DSCR1) | 4758195/U28833 |
| insulin induced protein 1 (INSIG1) | 5031800/U96876 |
| cytochrome oxidase subunit I (MTCO1) | AF035429 |
| NADH-ubiquinone oxidoreductase chain 1 (NH1) | DNHUN1 |
| NADH-ubiquinone oxidoreductase chain 4 (NH4) | DNHUN4 |
| decidual protein induced by progesterone (DEPP) | AB022718 |
| connective tissue growth factor (CTGF) | X78947 |

4. Results

HUVECs were treated with various growth factors, or none, and harvested after 6 or 24 hours. This permits distinguishing between those genes that are more directly regulated by growth factor treatment (after 6 hours) vs. those that may be indirectly regulated, and so appear to be modulated only after 24 hours.

The results of this analysis are summarized in Table E2.

The serum-starved HUVECs represent a valid in vitro model because 30% of the cells undergo apoptosis after serum deprivation, representing a 6 fold increase over non-serum starved controls. VEGF or VEGFR1s addition strongly decreases the number of apoptotic cells, while PlGF addition does not stimulate survival (Gerber et al., 1998). These results show that signaling via VEGFR1 and not via the PlGF receptor is important for VEGF activity.

TABLE E2
GeneCalling ™ results

| | | Treatment | | | | | | | | | | | | | | | |
|---|---|---|---|---|---|---|---|---|---|---|---|---|---|---|---|---|---|
| | | Serum | | VEGF | | Serum | | VEGF | | VEGFR1s | | BFGF | | HGF | | P1GF | |
| | | Time (hours): | | | | | | | | | | | | | | | |
| Gene | GeneCalling bands | 6 | 24 | 6 | 24 | 6 | 24 | 6 | 24 | 6 | 24 | 6 | 24 | 6 | 24 | 6 | 24 |
| Nexin | f0n0-178.8 | — | — | +1.2 | +2.7 | — | — | — | — | — | — | — | — | — | — | — | — |
| PP5 | b1i0-190.7 | −1.6 | −1.4 | +1.9 | +2.7 | — | — | +2.4 | +2.4 | +2.1 | +2 | +2.3 | — | — | — | — | +1.4 |
| | d0l0-227.9 | | | | | | | | | | | | | | | | |
| | i0u0-108.1 | | | | | | | | | | | | | | | | |
| HB-EGF | u0f0-157.6 | — | +3.9 | +3.5 | +4.1 | +3 | −2.6 | +2.3 | — | — | — | — | +2.1 | — | — | — | — |
| RGS33 | b1i0-75.5 | — | −2.3 | +2.2 | +2.6 | — | −2.9 | — | — | — | +2.2 | — | — | — | — | — | — |
| gravin | d0y0-108.1 | +1.4 | +5.7 | +1.6 | +5.7 | +4.1 | — | — | +2.7 | — | — | — | — | — | — | — | — |
| | y0h0-123.3 | | | | | | | | | | | | | | | | |
| MKP1LPTP | l1c0-184.5 | +3 | — | +2.1 | +1.6 | — | — | — | — | — | — | — | — | — | — | — | — |
| APLP2 | d0v0-324.8 | — | — | −1.2 | — | −1.4 | +1.9 | +1.2 | +3.2 | +1.3 | — | — | — | −1.5 | +2.3 | — | — |
| Osteo- | h0a0-166.1 | — | −2.2 | +2.1 | +2.4 | — | — | — | — | — | — | — | — | — | — | — | — |
| nidogen | | | | | | | | | | | | | | | | | |
| APP | n0s0-112.8 | −2 | +2.8 | — | −1.9 | −2 | +6.5 | — | — | — | +5.6 | — | — | — | — | — | — |
| hVPS41p | i0r0-152.3 | +1.6 | +2.8 | −2 | −2.1 | — | +3.7 | — | — | — | — | — | — | — | — | — | — |
| | w0c0-259 | | | | | | | | | | | | | | | | |
| ARP | i0c0-224.3 | — | +2.1 | — | — | +2.2 | — | +2.3 | — | +2.8 | +2.4 | +1.6 | — | +1.3 | — | — | — |
| DSCR1 | h0a0-78.1 | +1.2 | −1.1 | +6.3 | +4.8 | — | — | +3 | +2.5 | +3.1 | — | — | +1.8 | — | — | — | — |
| | i0n0-136.2 | | | | | | | | | | | | | | | | |
| | i0n0-136.3 | | | | | | | | | | | | | | | | |
| INSIG1 | g1n0-43.2 | — | — | — | +2.7 | — | — | — | — | — | — | — | — | — | — | — | — |
| MTCO1 | l0r0-215.7 | — | −1.4 | — | −2.3 | −1.5 | −1.6 | — | — | −6.2 | −1.8 | — | — | −1.4 | −1.3 | −1.5 | +6.3 |
| | r0y0-360.5 | | | | | | | | | | | | | | | | |
| NH1 | | +2.5 | +11.5 | +1.9 | +4.2 | +8.2 | +2.8 | — | −3.8 | +2 | −7 | +2.2 | −6.8 | — | −2.3 | — | −7.1 |
| NH4 | u0w0-153.5 | — | — | — | −1.7 | — | — | −2.3 | −2.6 | — | −2 | — | −1.9 | — | −1.4 | — | — |
| DEPP | s0h0-217.2 | — | — | — | — | — | — | +4.7 | — | — | — | — | — | +3.2 | — | — | −2.8 |
| CTGF | m0a0-399.6 | +3.4 | — | +1.4 | +1.6 | — | +2.5 | — | +3.1 | +1.8 | +5.1 | — | — | −2.8 | — | −4.4 | +2.6 |

For each gene, this Table lists the GeneCalling generated cDNA fragments (bands) that were positively associated with that gene by confirmation and the modulation levels observed in each GeneCalling job for those bands.

### Example 2

### TaqMan™ Analysis of Differential Gene Expression in HUVECs

Genes that were shown to be modulated in the GeneCalling analysis were then subjected to Taqman™ analysis (TaqMan™ polymerase chain reaction detection; Perkin Elmer, Applied Biosystems Division, Foster City, Calif.).

100 ng of total RNA was added to a 50 μl RT-PCR reaction (PCR-Access, Promega). Primers and probes for real time PCR analysis were designed using the Oligo Version 4.0 program (National Bioscience, Plymouth, Minn.) (Heid et al., 1996). RT-PCR reactions and the resulting relative increase in reporter fluorescent dye emission were monitored in real time with the 7700 Sequence Detector (Perkin Elmer, Foster City, Calif.). Signals were analyzed using the sequence detector 1.0 program (PE). Conditions were as follows: 1 cycle 48° C. for 45 min., 1 cycle 94° C. for 2 min., 40 cycles 94° C., 30 sec., 60° C., 1 min., 68° C., 2 min.

The results are shown in Table E3.

TABLE E3
TaqMan ™ analysis results.

| | | SERUM | | | VEGF | | | |
|---|---|---|---|---|---|---|---|---|
| Gene | Bands | 6h | 18 h | 24 h | 6h | 18 h | 24 h | 32 h |
| PP5 | b1i0-190.7 | nd | nd | nd | nd | 5 | nd | 6 |
| | d0l0-227.9 | | | | | | | |
| | i0u0-108.1 | | | | | | | |
| HB-EGF | U0f0-157.6 | nd | nd | 4 | 4 | nd | 4.9 | nd |
| RGS3 | B1i0-75.5 | nd | nd | nd | 4 | 4 | nd | Nd |
| Gravin | D0y0-108.1 | nd | nd | nd | 5 | 4 | nd | 4.5 |
| | y0h0-123.3 | | | | | | | |
| MKP1LPTP | L1c0-184.5 | 1 | 2 | nd | 3 | 2 | nd | Nd |
| APLP2 | D0v0-324.8 | 1 | nd | nd | 1.6 | nd | 5 | Nd |
| Osteo- | H0a0-166.1 | nd | nd | 4 | 3 | 3 | 5.3 | Nd |
| nidogen | | | | | | | | |
| hVPS41p | I0r0-152.3 | nd | nd | nd | nd | 2 | nd | 3 |
| | w0c0-259 | | | | | | | |
| ARP | I0c0-224.3 | nd | nd | nd | 3 | 2 | nd | 3 |
| DSCR1 | H0a0-78.1 | nd | nd | nd | 4 | 6 | nd | 5 |
| | i0n0-136.2 | | | | | | | |
| | i0n0-136.3 | | | | | | | |
| Nexin | F0n0-178.8 | 1 | nd | 1 | 1.5 | nd | nd | 2.4 |
| INSIG1 | G1n0-43.2 | 1 | nd | 1 | 1.5 | nd | nd | 3.2 |
| CTGF | M0a0-399.6 | nd | nd | nd | nd | 1.7 | nd | 6 |

For each gene, the GeneCalling generated cDNA fragments (bands) that were positively associated with that gene by confirmation and the modulation levels observed by TaqMan analysis.

TABLE E7
Probe Primer sets used for Real-time RT-PCR analysis.

| | | | | | |
|---|---|---|---|---|---|
| Probe sequence | # | Forward primer | # | Reverse primer | # |

| | | | | | | |
|---|---|---|---|---|---|---|
| HSPP5.P/#43 | aaagttcccaaagtttgccggctgc | 45 | cgatgcttgctggaggataga | 46 | acactggtcgtccacactcact | 47 |
| HVPS41 | ttcgcccagacatgtatccctgcag | 48 | atgtgccccgggatgatata | 49 | gtcccccagccaataatcagt | 50 |
| 1667.FP/#50 | | | | | | |
| HSARP | aggtatcaaagcctctggcccacca | 51 | gcagccaccaaaatcatcaat | 52 | tcacagatcttctccacagggat | 53 |
| 560.FP/#51 | | | | | | |
| HSDSCR1 | aggttgtgaaaacagcagcaatgcaatgt | 54 | ccacaggaagccgcctagt | 55 | tgagggaagaaaggaaacgct | 56 |
| 1113.FP/#52 | | | | | | |
| HSGRAVIN | ctgaggcatcattcactctaacagcggc | 57 | gaggaggcagtatgcaccaaa | 58 | tgcaggctccaacgtttca | 59 |
| 4118.FP/#53 | | | | | | |
| HSDOCK | agaatgccgcgtgctttctcctgac | 60 | atgtaggacagaacgggcctt | 61 | gttttgaattgcattgcccc | 62 |
| 180hlg.259.FP# | | | | | | |
| HSRGS3 | aggacaacctgcagagcgtcacgc | 63 | aagatgcgcttctgtgcca | 64 | aacctggactcctacacgcg | 65 |
| 1696.FP/#55 | | | | | | |
| HSPDK-1 | tgtgaggaaatggaaggatacggacctct | 66 | gatgccacaaagcggttagg | 67 | gtgacggactcgaagaacgg | 68 |
| 1059.FP | taaa | | | | | |
| HSPTPLC100hlg | tacaactgggtgaaagcccggcg | 69 | acaacgtgtgcctgctgga | 70 | cctacgttgggcctgatgac | 71 |
| 183.FP/#57 | | | | | | |
| HSVEGF.294.FP/ | tgtgcccactgaggagtccaacatca | 72 | aatgacgagggcctggagt | 73 | ttgatccgcataatctgcatg | 74 |
| #92 | | | | | | |
| HSHB-EGF.300. | ctggctgcagttctctcggcactg | 75 | tgaacagtgaggtatgctgaact | 76 | ctccaggctctcgccagtc | 77 |
| FP/ | | | | | | |
| HSFlt-1-2449T/ | accaaccagaagggctctgtggaa | 78 | aaggtgtctatcactgcaaagc | 79 | tgaacagtgaggtatgctgaact | 80 |
| #175 | | | | | | |
| HSKDR.1180.RP/ | agacaggtcgggtgagggcg | 81 | cgcctctgtgggtaagga | 82 | ccgagttagatctggctttca | 83 |
| #93 | | | | | | |

#, SEQ ID NO:.

### Example 3

### ³³P-Hybridization Analysis of Differential Gene Expression

Formalin fixed, paraffin-embedded human tissues were investigated for in situ mRNA expression. Tissues included first trimester (14-15 week) placenta, adult adrenal cortex, aorta, muscular artery with atherosclerosis, brain, gall bladder, heart, pancreas, prostate, stomach, eye with age related macular degeneration (AMD), and inflamed appendix, pulmonary adenocarcinoma, ductal mammary adenocarcinoma, kidney with renal cell carcinoma, hepatocellular carcinoma, squamous cell carcinoma, osteosarcoma, and chondrosarcoma. In vitro transcription and [³³P] labeling of sense and anti-sense riboprobes was performed as follows: Sequences for the genes to be analyzed were PCR-amplified from plasmid DNA using gene-specific primers that encoded T3 or T7 RNA polymerase initiation sites. Sense and antisense riboprobes were prepared by in vitro transcription from the PCR-amplified templates and diluted in hybridization buffer to a specific activity of 1×10⁶ cpm/ml. Tissue sections 5 micrometers thick were deparaffinized, deproteinated in 4 μg/ml of proteinase K for 30 minutes at 37° C., hybridized at 55° C. overnight, then washed at high stringency (55° C. in 0.1×SSC for 2 hours). Glass slides were dipped in NBT2 nuclear track emulsion (Eastman Kodak), exposed in sealed plastic slide boxes containing dessicant for 4 weeks at 4° C., developed and counterstained with hematoxylin and eosin.

The results of the in-situ hybridization experiments are shown in Table E4.

TABLE E4
In situ hybridization analysis

| | | | | | | | | MKP1- | | | Osteo- |
|---|---|---|---|---|---|---|---|---|---|---|---|
| | DSCR1 | PP5 | RGS3 | ARP | hVPS41p | HB-EGF | Gravin | LPTP | CTGF | nexin | nidogen |
| HUVEC: ct values | 23.1 | 18.7 | 21.7 | 20.5 | 22.4 | 25.9 | 19.3 | 23 | 21 | − | 22 |
| tumor: vascular | − | − | ++ | − | − | − | (+) | − | ++ | − | ++ |
| tumor: non vascular | + | − | +++ | ++ | ++ | ++ | ++ | +/++ | stromal | +/++ | ++ |
| fetal: vascular | ++ | +++ | − | − | − | − | ++ | − | +++ | − | ++ |
| fetal: non vascular | ++ | + | + | ++ | − | +/++ | ++ | | +++ | +/++ | ++ |
| adult vascular | − | − | − | − | − | − | (+) | − | ++ | − | + |
| adult non vascular | + | − | + | − | − | + | − | − | ++ | (+) | + |
| Inflammation | − | − | + | +++ | − | +++ | ? | − | ++ | | ++ |

+ weak expression,
++ moderate expression,
+++ strong expression

The results in Table E4 show that in fetal vascular tissue certain of the differentially expressed genes identified by GeneCalling are also differentially identified by in-situ hybridization. In adult vascular tissue, however, only pathological states, such as presence of a tumor or of inflammation, lead to significant modulation of genes among the set of differentially expressed genes.

### Example 4

### Clinical Stage Correlation of Ovarian Tumors with Differential Expression of VEGF-Modulated Genes

In order to test, whether the correlation between VEGF stimulation and DSCR1 expression observed in tissue culture conditions in vitro did also translate in vivo in tumors associated with high VEGF expression, we have analyzed 3 matched sets of RNA derived from ovarian tumors and control tissues from the same patients (Clonetech) by real-time RT-PCR. VEGF overespression is thought to play a major role in the progression of ovarian cancer by promoting the neovascularization and subsequent growth of solid intraperitoneal tumors and by inducing ascites formation by increasing the permeability of the tumor vasculature (Mesiano et al, Am. J. Pathol, 153, p 1249, 1998). VEGF mRNA levels in ovarian carcinomas are significantly higher than in normal ovaries. The average levels of VEGF expression in normal versus tumor tissues was increased 3.2 fold and correlated with the 2.7 fold increase in DSCR1 expression in the tumor RNA.

Two thirds of patients with epithelial ovarian carcinomas have advanced disease at diagnosis and have poor prognosis because of the presence of highly invasive carcinoma cells (CA) and rapidly accumulating ascites fluid. One third of patients with low metastatic epithelial adenocarcinomas (low malignant potential=LMPs), have extremely favorable long term outcomes. Previous studies indicated not only a correlation between disease and VEGF expression, but identified VEGF as key regulator of angiogenesis and ascites formation in ovarian cancer. (Fujimoto et al, Cancer, 83, p. 2532, 1998.)

A series of total RNAs isolated from 12 patients with LMPs and 9 patients with CAs for expression of VEGF were tested, VEGF receptors and DSCR1 by real-time RT-PCR. Expression levels were normalized to the levels of GAPDH or β-actin (data not shown). Based on these expression levels, statistical analysis using StatView statistical analysis software program, lead to the identification of a correlation between VEGF, VEGF receptors and the expression levels of DSCR1 (Table E5). In addition, a correlation between clinical stage (R═), KDR (R=0.834) and VEGF (R═) expression. These findings indicate that gene profiling experiments in endothelial cells grown in tumor like conditions mimicked by the presence of VEGF, might be instrumental in the search of novel VEGF target genes that are specifically upregulated in tumors or the tumor vasculature. Moreover, the correlation between with clinical stages of tumor development and DSCR1 levels opens the question whether DSCR can serve as a predictive marker for tumor progression in ovarian tumor patients and in other indications.

Total RNA was isolated from tumor biopsies of 12 patients with Low Malignant Potential (LMP) ovarian tumor and from 9 patients with the more malignant Cystoadeno Carcinoma (CA) ovarian tumor. The RNA was analyzed for the expression of VEGF, VEGF receptors and VEGF target genes by TaqMan™ as described above. RNA was run in triplicate, a standard curve with HUVE cell RNA was generated for each probe and relative expression levels were calculated using as a standard the housekeeping gene β-glucuronidase (GUS) and the endothelial marker CD31 to correct for the amount of endothelial cells present. The results are summarized in Table E5. The first row reports the results of ANOVA analysis between the expression of a given gene and grouping the tumor samples based on the clinical stage, LMP vs CA. The second and third rows report the correlation between expression of a given gene and the expression of VEGF or VEGFR1 receptor by the tumor samples. They indicate that there is a positive correlation between high metastatic potential and increased expression level for DSCR1 and ARG rich genes.

TABLE E5
Ovarian tumor clinical stage correlation analysis

| | Ovarian tumor RNA | | | | | | | | | | |
|---|---|---|---|---|---|---|---|---|---|---|---|
| Correlation | | | | | | | | MKP1LP | | | Osteo- |
| with: | DSCR1 | PP5 | RGS3 | ARP | HVPS41 | HB-EGF | Gravin | TP | CTGF | Nexin | nidogen |
| clinical stage | p = | — | — | p = 0.0157 | — | nd | — | — | nd | nd | nd |
| (LMP/CA) | 0.0157 | | | | | | | | | | |
| VEGF | — | R = | R = 0.590 | — | R = 0.665 | nd | R = 0.956 | — | nd | nd | nd |
| expression | | 0.949 | p = 0.0049 | | p = 0.001 | | p = 0.0001 | | | | |
| | | p = | | | | | | | | | |
| | | 0.0001 | | | | | | | | | |
| VEGFR1 | R = | — | R = 0.667 | R = 0.799 | R = 0.662 | nd | — | R = 0.662 | nd | nd | nd |
| expression: | 0.834 | | p = 0.0009 | p = 0.0001 | p = 0.0011 | | | p = 0.0034 | | | |
| | p < | | | | | | | | | | |
| | 0.0001 | | | | | | | | | | |

### Example 5

### Survival of Endothelial Cells Transfected with VEGFmgs

In order to study whether DSCR1 directly regulates endothelial cell survival, we transiently cotransfected epitope tagged version of DSCR1 with an expression vectors for EGFP and quantified the ratio between EGFP positive and healthy and apoptotic endothelial cells by fluorescenz micropscopy. As shown in FIG. 1, transient overexpression of epitope tagged version of DSCR1 (DSCR1-FLAG) led to a modest decrease in cell viability. Overexpression of the antisense construct, in contrast, increased survival to similar extends as observed for a constitutive active form of Akt (Akt 179). These findings excluded a direct survival effect of DSCR1 when overexpressed in endothelial cells and suggested a decrease in viability under serum starvation conditions. However, no such decrease in viability was observed in cells grown in 5% serum conditions (FIG. 1).

It is seen that in the control, DSCR1 removal induces apoptosis; at 66 hours only about 25% of the cells are alive. On the other hand, about 80% of the cells transfected with Akt2D survive. Cells transfected with DSCR1 have a survival rate similar to Akt2d while transfection with the sense strand of DSCR1, presumably leading to higher expression, induces faster cell death.

Experimental Details:

Expression in HUVECs of sense and antisense polynucleotides corresponding to genes in this invention was carried out as follows:

a) Cells:

HUVEC, p6 (Cell system) in 6 cm tissue culture dish (Falcon 3802, primaria, surface modified polystyrene). grown on gelatin coated plastic.

6 cm dishes were coated for >20 min with 0.2% gelatin in PBS, before applying the cells.

Cells were coated at a density of 140,000 cells per 6 cm dish, i.e., ca. 5000 cells/cm2

Cells should attain at least 60% confluency, since otherwise increased toxicity was observed. At high density, low transfection efficiency was observed.

For microvascular cells, other DNA/lipofectin ratios have to be determined, otherwise increased toxicity is found.

Control Samples:

| # | VEGF (50 ng/ml) | GFP | Annexin-PE |
|---|---|---|---|
| 1 | + | − | − |
| 2 | + | + | − |
| 3 | + | − | + |
| 4 | − | − | − |
| 5 | − | + | − |
| 6 | − | − | − |

DNA: 3.0 μg total DNA/6 cm dish: 2.0 μg test, 1.0 μg Green Fluorescent Protein (GFP)

F1: 4 μl/6 cm dish

OPTIMEM: 1.3 ml per 6 cm dish

Use Falcon clear tubes (polystyrene)

For HMVE cells: 2 μg DNA+4 μl F1

b) Procedure:

Day 1: Split cells 24 hours before Lipofectin,

Day 2: 4 pm to 6 pm: Vortex Lipofectin (Life Technologies, Inc., Rockville, Md.) thoroughly in clear Falcon tubes for 20 sec before using.

First add 1.35 ml/sample of OPTIMEM (Gibco BRL Cat No. 31985). Next add 3 μg total DNA per sample and mix well by vortexing. Then add 4 μl of F1 per sample and mix well by vortexing. Mix DNA+Lipofectin+OPTIMEM and incubate in a water bath at 37 C for 20 to 30 min; then wash the cells twice with OPTIMEM. Add 1.35 ml of the transfection mix and incubate for 2 h at 37 C. After 2 h, add 3 ml of complete medium and incubate 16 to 19 hours.

Day 3: 10 am: replace media next morning to 10% serum-containing medium, but do not wash the cells. Alternatively, leave the transfection mix for another 24 hours; this will lead to a higher transfection efficiency but also lead to increased cell death.

If apoptosis is being determined:

Day 3: evening: The cells are washed with 2× PBS and the medium is changed to serum starvation, then GF+WM are added.

Day 4: late afternoon: The cells are analyzed by using FACS set to detect annexin-PE and FITC channels for % apoptotic cells (30 h time point). Up to 32% transfection efficiency after 72 h was observed when Green Lantern was transfected.

If survival is being studied:

Day 4, morning: The cells are washed with 2× PBS and the medium is changed to serum starvation, then GF+WM are added.

Day 4, evening: count GFP positive cells and compare apoptotic/healthy

Day 5 (24 h later): The cells are harvested for FACS analysis.

c) FACS Analysis:

1. The supernatant (3 ml) is pulled off and added to prelabelled 5 ml Falcon tubes with a filter on top at 0° C., and the tubes were spun down at 2000 rpm for 3 min. In the meantime:

2. The cells were washed carefully with 3 ml PBS.

3. 0.5 ml 2× Trypsin was added, and the mixture was incubated for 3 min. in the 37 C incubator

4. After 3 min, 3 ml of medium was added, containing 10% serum, to stop the digestion.

5. The supernatant from step 1 was drawn off by aspiration and 3.5 ml from step 4 were added to the tubes containing the cell pellets.

6. The cells were pelleted at 2000 rpm for 3 min.

7. The pellets were washed 1× with 2 ml of 1× Ca binding buffer.

8. The cells were pelleted at 2000 rpm for 3 min, and the supernatant was aspirated off.

9. The pellet was taken up in 0.5 ml Ca-binding buffer (generate pool containing Annexin-PE, or simple 1× Ca-binding buffer for control samples), and the cells were disaggregated by pipetting up and down 6 times.

10. Add 10 μl of Annexin-PE to the control samples, or 1 μl of the BioVison annexin-Cy3 stock solution.

11. The tubes were kept on ice and submitted to the FACS lab for analysis.

d) Materials

F1: targeting systems, Targfect F-1 (2 mg/ml), Cat No #001 (1 ml) or #002 (4×1 ml).

Growth Factors: for 5 ml medium in 6 cm dishes

VEGF: 10 μl of 0.1 mg/ml stock+650 μl serum-free medium. 100 μl of this stock was added to 5 ml medium present in 6 cm dish to give a 30 ng/ml final concentration.

Wortmannin (a potent, irreversible inhibitor of phosphatidylinositol 3-kinase; BIOMOL, #ST-415; Catalogue Number 1232, Tocris Cookson, United Kingdom)

The contents of the vial (5 mg) were taken up in 500 μl dimethylsulfoxide (stock: 10 mg/ml stock; 23.3 mM). 4.3 μl of the 10 mg/ml stock solution was diluted in 1 ml medium to give a 100 μM solution. 10 μl of this stock was diluted in 650 μl serum-free medium, and 100 μl was added to the 5 ml medium present in the 6 cm dishes.

e) DNA

| | | |
|---|---|---|
| empty vector: pRLCMV, | 1.3 μg/μl | 2.7 μl/dish |
| GreenLantern ™: | 0.7 μg/μl | 1.5 μl/dish |

f) FACS:

Use Annexin-Cy3, GFP and Pi (works well)

Annexin-PE (R&D), add 10 μl of stock, undiluted, to the cells. Rest as before

Annexin-Cy3, BioVision, 1002-1000

Opti-MEM-1 Gibco, BRL Cat No. 31985, 0.5 l

CSC medium, Cat. No. 4Z0-500,

noGF. no serum Cat. no 4Z3-500-S,

Endothelial cells were transfected with pRLCMV (empty vector, negative control) or with pRLCMV further containing nucleotide sequences expressing either DSCR1 in the sense direction (DSCR1), or DSCR1 in the anti-sense direction (DSCR1 AS), or the activated mutant of AKT (Akt2D, a positive control that induces cell survival) as outlined above. The cells were co-transfected with Green Lantern expressing Green Fluorescent Protein that gives an indication of the efficiency of transfection and provides a visible marker for surviving cells. 18 hours after transfection, serum was removed from the media.

### Example 6

### Further Analysis of DSCR1

1. Introduction

Down's Syndrome induces mental retardation and congenital heart malformations. The open reading frame encoding DSCR1 was one of several located within the minimal region on chromosome 21 capable to induces the down syndrome phenotype (Fuentes et al, Hum Mol Genet October 1995;4(10):1935-44). More recently, DSCR1 was found to interact physically and functionally with calcineurin A, the catalytic subunit of the Ca(2+)/calmodulin-dependent protein phosphatase PP2B. Transient overexpression of DSCR1 blocked calcineurin-dependent gene transcription through the inhibition of the nuclear translocation of nuclear factor of activated T cells (NFAT). (Fuentes J J, Hum Mol Genet Jul. 1, 2000;9(11):1681-90).

NFAT was originally described as transcription factor that supported the activation of cytokine gene expression in T-cells and as the primary target of the immunoregulatory effects of cyclosporin A (CsA) and FK506. Elevated levels of NFAT in activated endothelial cells were first observed by Cockerill et al ( Blood Oct. 1, 1995;86(7):2689-98) and interference with NFAT activity by CsA resulted in a 40% reduction of E-selection expression on endothelial cells stimulated with TNF-α as well as a 29% decrease in neutrophil adhesion. These findings suggested a biological role of DSCR1 to regulate NFAT activity and the expression of cell adhesion molecules on activated endothelial cells.

2. Materials and Methods

(a) Cells

Human umbilical vein endothelial cells (HUVECs) were purchased from Cell Systems and were grown in endothelial growth medium (CS-C medium, Cell Systems)) complemented to a final concentration of 5% serum. Cells were split at a cell density of 19,000 cell/cm^{2,} and experiments were run in triplicates. 24 hours after seeding, the cells were washed three times with phosphate buffered saline (PBS) and media, 0.1% BSA or 0.1% BSA and VEGF (10 ng/ml) or 5% serum.

(b) RNA Harvest and Real Time RT-PCR Analysis

Medium was aspirated from the cultures, and 10 ml of Trizol (Gibco) was added to 1×10⁶ cells. The tissue culture flasks were incubated on vertical shaker for 10 min. RNA isolation and cDNA synthesis and data analysis were as described elsewhere (Kahn et al., 2000). For tissues, RNA was isolated from frozen tumor tissue harvested at necropsy from five specimens of each treatment group using the STAT 60 method (TEL-TEST “B”; Friendswood, Tex.), and purified on RNeasy Quick spin columns (Qiagen; Valencia, Calif.). One hundred ng of total RNA/reaction was analyzed using the RT-PCR kit from Perkin Elmer, following the manufacturer's instructions (PE Applied Biosystems, Foster City, Calif.). Reactions were run in 96 well plates in a Model 7700 Sequence Detector (PE Applied Biosystems, Foster City, Calif.) and results were analyzed using Sequence Detection Software (PE Applied Biosystems, Foster City, Calif.). RT-PCR conditions were 30 min at 48° C., 10 min at 95° C., and 40 cycles of 30 seconds at 95° C., 90 seconds at 60° C. Relative RNA equivalents for each sample were obtained by standardizing to GAPDH levels. Each of the five samples per group was run in duplicates to determine sample reproducibility, and the average relative RNA equivalents per sample pair was used for further analysis. Statistical analysis was performed using ANOVA software (Abacus Concepts, Inc., Berkeley, Calif.). Species specificity of the probe primer sets was verified by testing total RNA derived from human epithelial cells or mouse kidney RNA (data not shown). Expression levels were standardized to the probe/primer sets specific for human or murine GAPDH, respectively.

(c) Transient Transfection of Primary Human Endothelial Cells

Used HUVE cells before they reach passage 6 and HMVEC before reaching passage 4.

Use Falcon primaria 6 well dishes uncoated. (coating with gelatin is not recommended).

Harvest cells by incubation with 2× trypsin at rt for 3 to 5 min, dilute trypsinized cells in 3 volumes complete medium (do not trypsinize too long).

Count 10 μl of mix on the hemocytometer

Spin cells 5 min at 2 krp in the meantime.

Remove sn and dilute cells with complete medium to 0.5×10e5 cells in 3 ml of complete medium, make a pool

Add 3 ml of cells from the pool to each well (50000 cells/well, (5000 cells/cm2)

Cells should not be <60% confluent, otherwise increased toxicity might be observed. At cell densities >80%, lower transfection efficiency was observed.

Lipofection:

For HUVE and HMVE Cells:

The following amounts were calculated for transfection of 3 wells. It is advisable to generate a pool of 3 transfections in order to have duplicate or triplicates for each gene tested.

Pipette DNA into 15 ml Falcon clear tubes (polystyrene), best results when DNA conentration measured immediately prior to experiment:

11.25 μg of expression vector (pRKN driven)

3.75 μg of luciferase reporter

1.0 μg of SV-renilla refference reporters

Add 4.5 l ml of Optimem (Serum Free)

9.) Vortex F1 targefectin solution for 30 sec and add 14 μl of F1 to the mix.

10.) Mix the lipofection mix by inversion and incubate samples in 37 C water-bath for 20 to 30 minutes

10.) Wash cells once with PBS, remove PBS and add 1.5 ml of lipofection mix using 5 ml plastic pipette per dish.

11.) Incubate cells for 2.5 hours in CO2 incubator,

12.) Add 3 ml of complete medium and incubate overnight (12 to 16 hours). The effects of prolonged incubation are not determined yet.

13.) Wash cells 1× PBS

14.) Add 3 ml of complete medium, wait for 24 hours before dosing.

15.) harvest cells after 36 hours after lipfection or 6 to 9 hours after dosing.

Serum Starvation (0.5% FCS):

1.) Next morning: wash cells 1× with 3 ml PBS

2.) Add 3 ml of 0.5% FCS medium, 0.2% BSA, Pen/Step, fungizone

Up to 32% transfection efficiency after 72 h was observed when EGFP was transfected.\

Cell Harvest and Luciferase Measurement:

Remove medium by aspiration, wash carefully 1× with PBS and add 300 μl 1× passive lysis buffer, sample can be stored at −20 C at this point, however activity might decrease up to 50%.

Luminometer:

Prefill tube with 100 μl luciferast substrate solution

Add 30 μl extract

Add 100 μl STOP and GLOW

Additional Materials

Materials: F1: targeting systems, Targfect F-1 (2 mg/ml), Cat No #001 (1 ml) or #002 (4×1 ml), (Targeting systems, Tel 619 562 15 18, Rhumpia)

Culture dishes: 60 mm cell culture dishes, Falcon 3802, primaria, surface modified polystyrene.

Cells: HUVEC: Cell systems, 2VO-C75

HDMEC, Cell Systems, 2M1-C75

Medium:

5% serum containing:

Opti-MEM-1 Gibco, BRL Cat No. 31985, 0.5 l

CSC medium, Cat. no. 4Z0-500, 110$

noGF. no serum Cat. no 4Z3-500-S, 90$

3. DSCR1 is Expressed in Tumor Vasculature and in Neoplastic Cells

In order to study the cellular localization of DSCR1 expression within various human tumors and other malignancies, in situ hybridization experiments including a series of different human tumors as well on sections prepared from a variety of healthy human organs were performed. During fetal development in humans, DSCR1 was found to be expressed in the fetal liver and in dorsal root ganglia, in cells of the atrio-ventricular junction near the A-V valve insertions and focally in the cells within the subendocardial layer of the left ventricular septum and right ventricular apex. There was weak expression in embryonic large hepatic vein endothelium and small vessel endothelium. In the embryonic spinal cord, there was expression in neurons. When studied in adult chimpansees, DSCR1 expression was further detected in myoepithelial cells surrounding normal mammary ducts and in normal chimp parathyroid. In adult liver, expression was localized to hepatocytes and bile duct epithelium of cirrhotic, but not normal liver. There was focal expression within human adenocarcinomas of the mammary gland as well as in renal cell carcinoma and. Sense control were run on all samples and revealed no background signals (data not shown). These findings might reflect some degree of redundancy in the signal transduction pathways regulating DSCR1 expression on endothelial cells and transformed tumor cells. Alternatively, upregulation of VEGF receptors on tumors cells and stimulation of the VEGF specific signal transduction pathways could help to explain our findings. In summary, we found DSCR1 gene expression in fetal vasculature during normal ontogeny as well as in neoplastic tumor cells in adults and therefore identified DSCR1 as a member of the oncofetal family of genes.

4. Functional Analysis of DSCR1 by Transient Transfection of Primary Human Endothelial Cells

Recently it was shown in yeast two hybrid experiments, that DSCR1 interacts physically and functionally with calcineurin A, the catalytic subunit of the Ca2+/calmodulin-dependent protein phosphatase PP2B. In studies in T-cells, transient overexpression of DSCR1 inhibited the transcriptional activation of the interleukin 2 promoter in response to PMA/calcium stimulation. In DSCR1 transfected cells, NFAT was unable to accumulate in the nucleus after stimulation with calcium ionophores such as ionomycine.

Overexpression of DSCR in primary human endothelial cells had any effect on the NFAT activation after stimulating the cells with PMA and the calcium Ionophore A23187 was tested. Transient cotransfection experiment of expression vector encoding DSCR1-FLAG and a luciferase reporter construct containing three NFAT binding sites (NFAT-Luc) revealed complete ablation of NFAT activity in response to PMA and ionophore after 6 hours of stimulation. Enforced expression of DSCR in endothelial cells leads to a significant downregulatin of calcineurin regulated signal transduction pathways, presumably via interference with calcineurin regulated signal transduction pathways.

Equivalents

Although particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

### REFERENCES

U.S. Pat. No. 4,166,452. Apparatus for testing human responses to stimuli. 1979.
U.S. Pat. No. 4,485,045. Synthetic phosphatidyl cholines useful in forming liposomes. 1984.
U.S. Pat. No. 4,544,545. Liposomes containing modified cholesterol for organ targeting. 1985.
U.S. Pat. No. 4,676,980. Target specific cross-linked heteroantibodies. 1987.
U.S. Pat. No. 4,816,567. Recombinant immunoglobin preparations. 1989.
WO 90/10448. Covalent conjugates of lipid and oligonucleotide. 1990.
WO 90/13641. Stably transformed eucaryotic cells comprising a foreign transcribable DNA under the control of a pol III promoter. 1990.
EPO 402226. Transformation vectors for yeast Yarrowia. 1990.
WO 91/00360. Bispecific reagents for AIDS therapy. 1991.
WO 91/04753. Conjugates of antisense oligonucleotides and therapeutic uses thereof. 1991.
U.S. Pat. No. 5,013,556. Liposomes with enhanced circulation time. 1991.
WO 91/06629. Oligonucleotide analogs with novel linkages. 1991.
WO 92/20373. Heteroconjugate antibodies for treatment of HIV infection. 1992.
WO 93/08829. Compositions that mediate killing of HIV-infected cells. 1993.
WO 94/11026. Therapeutic application of chimeric and radiolabeled antibodies to human B lymphocyte restricted differentiation antigen for treatment of B cells. 1994.
WO 96/27011. A method for making heteromultimeric polypeptides. 1996.
U.S. Pat. No. 5,545,807. Production of antibodies from transgenic animals. 1996.
U.S. Pat. No. 5,545,806. Ransgenic <sic> non-human animals for producing heterologous antibodies. 1996.
U.S. Pat. No. 5,569,825. Transgenic non-human animals capable of producing heterologous antibodies of various isotypes. 1996.
WO 97/33551. Compositions and methods for the diagnosis, prevention, and treatment of neoplastic cell growth and proliferation. 1997.
U.S. Pat. No. 5,633,425. Transgenic non-human animals capable of producing heterologous antibodies. 1997.
U.S. Pat. No. 5,661,016. Transgenic non-human animals capable of producing heterologous antibodies of various isotypes. 1997.
U.S. Pat. No. 5,625,126. Transgenic non-human animals for producing heterologous antibodies. 1997.
Abravaya, K., J. J. Carrino, S. Muldoon, and H. H. Lee. 1995. Detection of point mutations with a modified ligase chain reaction (Gap-LCR). Nucleic Acids Res. 23:675-82.
Alam, J., and J. L. Cook. 1990. Reporter genes: Application to the study of mammalian gene transcription. Anal. Biochem. 188:245-254.
Alberts, B., D. Bray, J. Lewis, M. Raff, et al. 1994. Molecular Biology of the Cell. Garland Publishing, Inc., New York, N.Y. 1294 pp.
Alon, T., I. Hemo, A. Itin, J. Pe'er, et al. 1995. Vascular endothelial growth factor acts as a survival factor for newly formed retinal vessels and has implications for retinopathy of prematurity. Nat Med. 1:1024-8.
Araki, W., and R. J. Wurtman. 1998. Increased expression of amyloid precursor protein and amyloid precursor—like protein 2 during trophic factor withdrawal—induced death of neuronal PC12 cells. Brain Res Mol Brain Res. 56:169-77.
Arkonac, B. M., L. C. Foster, N. E. Sibinga, C. Patterson, et al. 1998. Vascular endothelial growth factor induces heparin-binding epidermal growth factor—like growth factor in vascular endothelial cells. J Biol Chem. 273:4400-5.
Athanassiades, A., and P. K. Lala. 1998. Role of placenta growth factor (PlGF) in human extravillous trophoblast proliferation, migration and invasiveness. Placenta. 19:465-73.
Austin, C. P., and C. L. Cepko. 1990. Cellular migration patterns in the developing mouse cerebral cortex. Development. 110:713-732.
Ausubel, F. M., R. Brent, R. E. Kingston, D. D. Moore, et al. 1987. Current protocols in molecular biology. John Wiley & Sons, New York.
Barany, F. 1991. Genetic disease detection and DNA amplification using cloned thermostable ligase. Proc Natl Acad Sci USA. 88:189-93.
Bartel, D. P., and J. W. Szostak. 1993. Isolation of new ribozymes from a large pool of random sequences [see comment]. Science. 261:1411-8.
Bartel, P., C. T. Chien, R. Sternglanz, and S. Fields. 1993. Elimination of false positives that arise in using the two-hybrid system. Biotechniques. 14:920-4.
Beal, P. A., and P. B. Dervan. 1991. Second structural motif for recognition of DNA by oligonucleotide—directed triple-helix formation. Science. 251:1360-3.
Bechtold, N., and G. Pelletier. 1998. In planta Agrobacterium-mediated transformation of adult Arabidopsis thaliana plants by vacuum infiltration. Methods Mol Biol. 82:259-66.
Becker, D. M., and L. Guarente. 1991. High-efficiency transformation of yeast by electroporation. Methods Enzymol. 194:182-187.
Beggs, J. D. 1978. Transformation of yeast by a replicating hybrid plasmid. Nature. 275:104-109.
Bellamy, W. T., L. Richter, Y. Frutiger, and T. M. Grogan. 1999. Expression of vascular endothelial growth factor and its receptors in hematopoietic malignancies. Cancer Res. 59:728-33.
Berger, J., J. Hauber, R. Hauber, R. Geiger, et al. 1988. Secreted placental alkaline phosphatase: A powerful new quantitative indicator of gene expression in eukaryotic cells. Gene. 66:1-10.
WO 93/04169. GENE TARGETING IN ANIMAL CELLS USING ISOGENIC DNA CONSTRUCTS. 1993.
Bodine, D. M., K. T. McDonagh, N. E. Seidel, and A. W. Nienhuis. 1991. Survival and retrovirus infection of murine hematopoietic stem cells in vitro: effects of 5-FU and method of infection. Exp. Hematol. 19:206-212.
Boerner, P., R. Lafond, W. Z. Lu, P. Brams, et al. 1991. Production of antigen-specific human monoclonal antibodies from in vitro-primed human splenocytes. J Immunol. 147:86-95.
U.S. Pat. No. 3,773,919. Polylactide-drug mixtures. 1973.
Bradley. 1987. Teratocarcinomas and Embryonic Stem Cells: A Practical Approach. Oxford University Press, Inc., Oxford. 268 pp.
Bradley, A. 1991. Modifying the mammalian genome by gene targeting. Curr Opin Biotechnol. 2:823-9.
Brennan, M., P. F. Davison, and H. Paulus. 1985. Preparation of bispecific antibodies by chemical recombination of monoclonal immunoglobulin G1 fragments. Science. 229:81-3.
WO94/10300. INTERACTION TRAP SYSTEM FOR ISOLATING NOVEL PROTEINS. 1994.
Capecchi, M. R. 1980. High efficiency transformation by direct microinjection of DNA into cultured mammalian cells. Cell. 22:479.
Capecchi, M. R. 1989. Altering the genome by homologous recombination. Science. 244:1288-92.
Carell, T., E. A. Wintner, and J. Rebek Jr. 1994a. A novel procedure for the synthesis of libraries containing small organic molecules. Angewandte Chemie International Edition. 33:2059-2061.
Carell, T., E. A. Wintner, and J. Rebek Jr. 1994b. A solution phase screening procedure for the isolation of active compounds from a molecular library. Angewandte Chemie International Edition. 33:2061-2064.
Carmeliet, P., M. G. Lampugnani, L. Moons, F. Breviario, et al. 1999. Targeted deficiency or cytosolic truncation of the VE-cadherin gene in mice impairs VEGF-mediated endothelial survival and angiogenesis. Cell. 98:147-57.
Caron, P. C., W. Laird, M. S. Co, N. M. Avdalovic, et al. 1992. Engineered humanized dimeric forms of IgG are more effective antibodies. J Exp Med. 176:1191-5.
Carter, P. 1986. Site-directed mutagenesis. Biochem J. 237:1-7.
Case, M. E., M. Schweizer, S. R. Kushner, and N. H. Giles. 1979. Efficient transformation of Neurospora crassa by utilizing hybrid plasmid DNA. Proc Natl Acad Sci USA. 76:5259-63.
U.S. Pat. No. 5,116,742. RNA ribozyme restriction endoribonucleases and methods. 1992.
U.S. Pat. No. 4,987,071. RNA ribozyme polymerases, dephosphorylases, restriction endoribonucleases and methods. 1991.
Cepko, C. L., B. E. Roberts, and R. E. Mulligan. 1984. Construction and applications of a highly transmissible murine retrovirus shuttle vector. Cell. 37:1053-1062.
Chalfie, M., Y. tu, G. Euskirchen, W. W. Ward, et al. 1994. Green fluorescent protein as a marker for gene expression. Science. 263:802-805.
Chaney, W. G., D. R. Howard, J. W. Pollard, S. Sallustio, et al. 1986. High-frequency transfection of CHO cells using Polybrene. Somatic Cell Mol. Genet. 12:237.
Chen, C., and H. Okayama. 1988. Calcium phosphate-mediated gene transfer: A highly efficient system for stably transforming cells with plasmid DNA. BioTechniques. 6:632-638.
Chen, S. H., H. D. Shine, J. C. Goodman, R. G. Grossman, et al. 1994. Gene therapy for brain tumors: regression of experimental gliomas by adenovirus-mediated gene transfer in vivo. Proc Natl Acad Sci USA. 91:3054-7.
Cho, C. Y., E. J. Moran, S. R. Cherry, J. C. Stephans, et al. 1993. An unnatural biopolymer. Science. 261:1303-5.
Cohen, A. S., D. L. Smisek, and B. H. Wang. 1996. Emerging technologies for sequencing antisense oligonucleotides: capillary electrophoresis and mass spectrometry. Adv Chromatogr. 36:127-62.
Cohen, J. S. 1989. Oligodeoxynucleotides: Antisense inhibitors of gene expression. CRC Press, Boca Raton, Fla. 255 pp.
Cohen, S. M. N., A. C. Y. Chang, and L. Hsu. 1972. Nonchromosomal antibiotic resistance in bacteria: Genetic transformation of Escherichia coli by R-factor DNA. Proc. Natl. Acad Sci. USA. 69:2110.
Cooney, M., G. Czernuszewicz, E. H. Postel, S. J. Flint, et al. 1988. Site-specific oligonucleotide binding represses transcription of the human c-myc gene in vitro. Science. 241:456-9.
Cotton, R. G. 1993. Current methods of mutation detection. Mutat Res. 285:125-44.
Crawford, D. R., N. E. Abramova, and K. J. Davies. 1998. Oxidative stress causes a general, calcium-dependent degradation of mitochondrial polynucleotides. Free Radic Biol Med. 25:1106-11.
Cronin, M. T., R. V. Fucini, S. M. Kim, R. S. Masino, et al. 1996. Cystic fibrosis mutation detection by hybridization to light-generated DNA probe arrays. Hum Mutat. 7:244-55.
Cull, M. G., J. F. Miller, and P. J. Schatz. 1992. Screening for receptor ligands using large libraries of peptides linked to the C terminus of the lac repressor. Proc Natl Acad Sci USA. 89:1865-9.
Cwirla, S. E., E. A. Peters, R. W. Barrett, and W. J. Dower. 1990. Peptides on phage: a vast library of peptides for identifying ligands. Proc Natl Acad Sci USA. 87:6378-82.
de Boer, A. G. 1994. Drug absorption enhancement: Concepts, possibilities, limitations and trends. Harwood Academic Publishers, Langhorne, Pa.
de Louvencourt, L., H. Fukuhara, H. Heslot, and M. Wesolowski. 1983. Transformation of Kluyveromyces lactis by killer plasmid DNA. J Bacteriol. 154:737-42.
de Wet, J. R., K. V. Wood, M. DeLuca, D. R. Helinski, et al. 1987. Structure and expression in mammalian cells. Mol. Cell Biol. 7:725-737.
Demerec, M., E. A. Adelberg, A. J. Clark, and P. E. Hartman. 1966. A proposal for a uniform nomenclature in bacterial genetics. Genetics. 54:61-76.
Deng, X., P. Ruvolo, B. Carr, and W. S. May. 2000. Survival function of ERK1/2 as IL-3-activated, staurosporine-resistant Bcl2 kinases. Proc Natl Acad Sci USA. 97:1578-83.
Devlin, J. J., L. C. Panganiban, and P. E. Devlin. 1990. Random peptide libraries: a source of specific protein binding molecules. Science. 249:404-6.
DeWitt, S. H., J. S. Kiely, C. J. Stankovic, M. C. Schroeder, et al. 1993. “Diversomers”: an approach to nonpeptide, nonoligomeric chemical diversity. Proc Natl Acad Sci USA. 90:6909-13.
Druey, K. M., K. J. Blumer, V. H. Kang, and J. H. Kehrl. 1996. Inhibition of G-protein-mediated MAP kinase activation by a new mammalian gene family. Nature. 379:742-6.
Eichelbaum, M., and B. Evert. 1996. Influence of pharmacogenetics on drug disposition and response. Clin Exp Pharmacol Physiol. 23:983-5.
Ellington, A. D., and J. W. Szostak. 1990. In vitro selection of RNA molecules that bind specific ligands. Nature. 346:818-22.
Elroy-Stein, O., and B. Moss. 1990. Cytoplasmic expression system based on constitutive synthesis of bacteriophage T7 RNA polymerase in mammalian cells. Proc. Natl. Acad. Sci. USA. 87:6743-6747.
U.S. Pat. No. 4,522,811. Serial injection of muramyldipeptides and liposomes enhances the anti-infective activity of muramyldipeptides Serial injection of muramyldipeptides and liposomes enhances the anti-infective activity of muramyldipeptides. 1985.
Eppstein, D. A., Y. V. Marsh, M. van der Pas, P. L. Feigner, et al. 1985. Biological activity of liposome-encapsulated murine interferon gamma is mediated by a cell membrane receptor. Proc Natl Acad Sci USA. 82:3688-92.
Escudero, J., and B. Hohn. 1997. Transfer and integration of T-DNA without cell injury in the host plant. Plant Cell. 9:2135-2142.
U.S. Pat. No. 4,870,009. Method of obtaining gene product through the generation of transgenic animals. 1989.
Evron, E., P. Cairns, N. Halachmi, S. A. Ahrendt, et al. 1997. Normal polymorphism in the incomplete trinucleotide repeat of the arginine-rich protein gene. Cancer Res. 57:2888-9.
WO9730065. COMPOSITIONS AND METHODS FOR THE TREATMENT AND DIAGNOSIS OF CARDIOVASCULAR DISEASE. 1997.
Fekete, D. M., and C. L. Cepko. 1993. Retroviral infection coupled with tissue transplantation limits gene transfer in the chick embryo. Proc. Natl. Acad. Sci. USA. 90:2350-2354.
Feigner, P. L., T. R. Gadek, M. Holm, R. Roman, et al. 1987. Lipofectin: A highly efficient, lipid-mediated DNA/transfection procedure. Proc. Natl. Acad. Sci. USA. 84:7413-7417.
Felici, F., L. Castagnoli, A. Musacchio, R. Jappelli, et al. 1991. Selection of antibody ligands from a large library of oligopeptides expressed on a multivalent exposition vector. J Mol Biol. 222:301-10.
Ferrara, N. 1999a. Molecular and biological properties of vascular endothelial growth factor. J Mol Med. 77:527-43.
Ferrara, N. 1999b. Vascular endothelial growth factor: molecular and biological aspects. Curr Top Microbiol Immunol. 237:1-30.
Fieck, A., D. L. Wyborski, and J. M. Short. 1992. Modifications of the E. coli Lac repressor for expression in eukaryotic cells: effects of nuclear signal sequences on protein activity and nuclear accumulation. Nucleic Acids Res. 20:1785-91.
Finer, J. J., K. R. Finer, and T. Ponappa. 1999. Particle bombardment-mediated transformation. Current Topics in microbiology and immunology. 240:59-80.
Finn, P. J., N. J. Gibson, R. Fallon, A. Hamilton, et al. 1996. Synthesis and properties of DNA-PNA chimeric oligomers. Nucleic Acids Res. 24:3357-63.
Fishwild, D. M., S. L. O'Donnell, T. Bengoechea, D. V. Hudson, et al. 1996. High-avidity human IgG kappa monoclonal antibodies from a novel strain of minilocus transgenic mice [see comments]. Nat Biotechnol. 14:845-51.
Fleer, R., P. Yeh, N. Amellal, I. Maury, et al. 1991. Stable multicopy vectors for high-level secretion of recombinant human serum albumin by Kluyveromyces yeasts. Biotechnology (NY). 9:968-75.
Fodor, S. P., R. P. Rava, X. C. Huang, A. C. Pease, et al. 1993. Multiplexed biochemical assays with biological chips. Nature. 364:555-6.
Fong, G. H., J. Rossant, M. Gertsenstein, and M. L. Breitman. 1995. Role of the Flt-1 receptor tyrosine kinase in regulating the assembly of vascular endothelium. Nature. 376:66-70.
Fromm, M., L. P. Taylor, and V. Walbot. 1985. Expression of genes transferred into monocot and dicot plant cells by electroporation. Proc. Natl. Acad. Sci. USA. 82:5824-5828.
Fuentes, J. J., M. A. Pritchard, A. M. Planas, A. Bosch, et al. 1995. A new human gene from the Down syndrome critical region encodes a proline-rich protein highly expressed in fetal brain and heart. Hum Mol Genet. 4:1935-44.
Fujita, T., H. Shubiya, T. Ohashi, K. Yamanishi, et al. 1986. Regulation of human interleukin-2 gene: Functional DNA sequences in the 5′ flanking region for the gene expression in activated T lymphocytes. Cell. 46:401-407.
Gabizon, A., R. Shiota, and D. Papahadjopoulos. 1989. Pharmacokinetics and tissue distribution of doxorubicin encapsulated in stable liposomes with long circulation times. J Natl Cancer Inst. 81:1484-8.
Gallagher, S. R. 1992. GUS protocols: Using the GUS gene as a reporter of gene expression. Academic Press, San Diego, Calif.
Gallop, M. A., R. W. Barrett, W. J. Dower, S. P. Fodor, et al. 1994. Applications of combinatorial technologies to drug discovery. 1. Background and peptide combinatorial libraries. J Med Chem. 37:1233-51.
Gasparini, P., A. Bonizzato, M. Dognini, and P. F. Pignatti. 1992. Restriction site generating-polymerase chain reaction (RG-PCR) for the probeless detection of hidden genetic variation: application to the study of some common cystic fibrosis mutations. Mol Cell Probes. 6:1-7.
Gautier, C., F. Morvan, B. Rayner, T. Huynh-Dinh, et al. 1987. α-DNA. IV: A-anomeric and β-anomeric tetrathymidylates covalently linked to intercalating oxazolopyridocarbazole. Synthesis, physicochemical properties and poly (rA) binding. Nucleic Acids Res. 15:6625-41.
Gennaro, A. R. 2000. Remington: The science and practice of pharmacy. Lippincott, Williams & Wilkins, Philadelphia, Pa.
Gerber, H. P., A. McMurtrey, J. Kowalski, M. Yan, et al. 1998. Vascular endothelial growth factor regulates endothelial cell survival through the phosphatidylinositol 3′-kinase/Akt signal transduction pathway. Requirement for Flk-1/KDR activation. J Biol Chem. 273:30336-43.
Gibbs, R. A., P. N. Nguyen, and C. T. Caskey. 1989. Detection of single DNA base differences by competitive oligonucleotide priming. Nucleic Acids Res. 17:2437-48.
Gietz, R. D., R. A. Woods, P. Manivasakam, and R. H. Schiestl. 1998. Growth and transformation of Saccharomyces cerevisiae. In Cells: A laboratory manual. Vol. I. D. Spector, R. Goldman, and L. Leinwand, editors. Cold Spring Harbor Press, Cold Spring Harbor, N.Y.
Gille, J., M. Khalik, V. Konig, and R. Kaufmann. 1998. Hepatocyte growth factor/scatter factor (HGF/SF) induces vascular permeability factor (VPF/VEGF) expression by cultured keratinocytes. J Invest Dermatol. 111:1160-5.
Goding, J. W. 1996. Monoclonal antibodies: Principles and Practice. Academic Press, San Diego. 492 pp.
Gorman, C. M., L. F. Moffat, and B. H. Howard. 1982. Recombinant genomes which express chloramphenicol acetyltransferase in mammalian cells. Mol. Cell. Biol. 2:1044-1051.
Graham, F. L., and A. J. van der Eb. 1973. A new technique for the assay of infectivity of human adenovirus 5 DNA. Virology. 52:456-.
Griffin, H. G., and A. M. Griffin. 1993. DNA sequencing. Recent innovations and future trends. Appl Biochem Biotechnol. 38:147-59.
Grompe, M., D. M. Muzny, and C. T. Caskey. 1989. Scanning detection of mutations in human ornithine transcarbamoylase by chemical mismatch cleavage. Proc Natl Acad Sci USA. 86:5888-92.
Gruber, M., B. A. Schodin, E. R. Wilson, and D. M. Kranz. 1994. Efficient tumor cell lysis mediated by a bispecific single chain antibody expressed in Escherichia coli. J Immunol. 152:5368-74.
Guatelli, J. C., K. M. Whitfield, D. Y. Kwoh, K. J. Barringer, et al. 1990. Isothermal, in vitro amplification of nucleic acids by a multienzyme reaction modeled after retroviral replication. Proc Natl Acad Sci USA. 87:1874-8.
Hanahan, D. 1983. Studies on transformation of Escherichia coli with plasmids. J. Mol. Biol. 166:557-580.
Hansen, G., and M.-D. Chilton. 1999. Lessons in gene transfer to plants by a gifted microbe. Curr. Top. Microbiol. Immunol. 240:21-57.
Hansen, G., and M. S. Wright. 1999. Recent advances in the transformation of plants. Trends Plant Sci. 4:226-231.
Harlow, E., and D. Lane. 1988. Antibodies: A laboratory manual. Cold Spring Harbor Laboratory Press, Cold Spring Harbor. 726 pp.
Harlow, E., and D. Lane. 1999. Using antibodies: A laboratory manual. Cold Spring Harbor Laboratory PRess, Cold Spring Harbor, N.Y.
Haseloff, J., and W. L. Gerlach. 1988. Simple RNA enzymes with new and highly specific endoribonuclease activities. Nature. 334:585-91.
Hata, Y., S. L. Rook, and L. P. Aiello. 1999. Basic fibroblast growth factor induces expression of VEGF receptor KDR through a protein kinase C and p44/p42 mitogen-activated protein kinase-dependent pathway. Diabetes. 48:1145-55.
Hayashi, K. 1992. PCR-SSCP: A method for detection of mutations. Genetic and Analytical Techniques Applications. 9:73-79.
Heid, C. A., J. Stevens, K. J. Livak, and P. M. Williams. 1996. Real time quantitative PCR. Genome Res. 6:986-94.
Helene, C. 1991. The anti-gene strategy: control of gene expression by triplex-forming-oligonucleotides. Anticancer Drug Des. 6:569-84.
Helene, C., N. T. Thuong, and A. Harel-Bellan. 1992. Control of gene expression by triple helix-forming oligonucleotides. The antigene strategy. Ann NY Acad Sci. 660:27-36.
Hinnen, A., J. B. Hicks, and G. R. Fink. 1978. Transformation of yeast. Proc. Natl. Acad. Sci. USA. 75:1929-1933.
Hoffman, F. 1996. Laser microbeams for the manipulation of plant cells and subcellular structures. Plant Sci. 113:1-11.
Hogan, B., Beddington, R., Costantini, F., Lacy, E. 1994. Manipulating the Mouse Embryo: A Laboratory Manual. Cold Spring Harbor Laboratory Press. 500 pp.
Holliger, P., T. Prospero, and G. Winter. 1993. “Diabodies”: small bivalent and bispecific antibody fragments. Proc Natl Acad Sci USA. 90:6444-8.
Hoogenboom, H. R., A. D. Griffiths, K. S. Johnson, D. J. Chiswell, et al. 1991. Multi-subunit proteins on the surface of filamentous phage: methodologies for displaying antibody (Fab) heavy and light chains. Nucleic Acids Res. 19:4133-7.
Houghten, R. A., J. R. Appel, S. E. Blondelle, J. H. Cuervo, et al. 1992. The use of synthetic peptide combinatorial libraries for the identification of bioactive peptides. Biotechniques. 13:412-21.
Hsu, I. C., Q. Yang, M. W. Kahng, and J. F. Xu. 1994. Detection of DNA point mutations with DNA mismatch repair enzymes. Carcinogenesis. 15:1657-62.
Hwang, K. J., K. F. Luk, and P. L. Beaumier. 1980. Hepatic uptake and degradation of unilamellar sphingomyelin/cholesterol liposomes: a kinetic study. Proc Natl Acad Sci USA. 77:4030-4.
Hyder, S. M., and G. M. Stancel. 1999. Regulation of angiogenic growth factors in the female reproductive tract by estrogens and progestins. Mol Endocrinol. 13:806-11.
Hyrup, B., and P. E. Nielsen. 1996. Peptide nucleic acids (PNA): synthesis, properties and potential applications. Bioorg Med Chem. 4:5-23.
Inaba, N., H. Ishige, M. Ijichi, N. Satoh, et al. 1982. Immunohistochemical detection of pregnancy-specific protein (SP1) and placenta-specific tissue proteins (PP5, PP10, PP11 and PP12) in ovarian adenocarcinomas. Oncodev Biol Med. 3:379-89.
Inoue, H., Y. Hayase, A. Imura, S. Iwai, et al. 1987a. Synthesis and hybridization studies on two complementary nona(2′-O-methyl)ribonucleotides. Nucleic Acids Res. 15:6131-48.
Inoue, H., Y. Hayase, S. Iwai, and E. Ohtsuka. 1987b. Sequence-dependent hydrolysis of RNA using modified oligonucleotide splints and RNase H. FEBS Lett. 215:327-30.
Ishiura, M., S. Hirose, T. Uchida, Y. Hamada, et al. 1982. Phage particle-mediated gene transfer to cultured mammalian cells. Molecular and Cellular Biology. 2:607-616.
Ito, H., Y. Fukuda, K. Murata, and A. Kimura. 1983. Transformation of intact yeast cells treated with alkali cations. J. Bacteriol. 153:163-168.
Iwabuchi, K., B. Li, P. Bartel, and S. Fields. 1993. Use of the two-hybrid system to identify the domain of p53 involved in oligomerization. Oncogene. 8:1693-6.
Jahroudi, N., J. Kitney, J. S. Greenberger, and R. Bowser. 1998. Endothelial cell dysfunction in response to intracellular overexpression of amyloid precursor protein. J Neurosci Res. 54:828-39.
Jayasena, S. D. 1999. Aptamers: an emerging class of molecules that rival antibodies in diagnostics. Clin Chem. 45:1628-50.
Jones, P. T., P. H. Dear, J. Foote, M. S. Neuberger, et al. 1986. Replacing the complementarity-determining regions in a human antibody with those from a mouse. Nature. 321:522-5.
Joshi, B., L. Li, B. G. Taffe, Z. Zhu, et al. 1999. Apoptosis induction by a novel anti-prostate cancer compound, BMD 188 (a fatty acid-containing hydroxamic acid), requires the mitochondrial respiratory chain. Cancer Res. 59:4343-55.
Kahn, J., F. Mehraban, G. Ingle, X. Xin, et al. 2000. Gene expression profiling in an in vitro model of angiogenesis. Am J Pathol. 156:1887-900.
Kaufman, R. J. 1990. Vectors used for expression in mammalian cells. Methods Enzymol. 185:487-511.
Kaufman, R. J., P. Murtha, D. E. Ingolia, C.-Y. Yeung, et al. 1986. Selection and amplification of heterologous genes encoding adenosine deaminase in mammalian cells. Proc. Natl. Acad. Sci. USA. 83:3136-3140.
Kawai, S., and M. Nishizawa. 1984. New procedure for DNA transfection with polycation and dimethyl sulfoxide. Mol. Cell. Biol. 4:1172.
Kayanoki, Y., S. Higashiyama, K. Suzuki, M. Asahi et al. 1999. The requirement of both intracellular reactive oxygen species and intracellular calcium elevation for the induction of heparin-binding EGF-like growth factor in vascular endothelial cells and smooth muscle cells. Biochem Biophys Res Commun. 259:50-5.
Keen, J., D. Lester, C. Inglehearn, A. Curtis, et al. 1991. Rapid detection of single base mismatches as heteroduplexes on Hydrolink gels. Trends Genet. 7:5.
Kelly, J. M., and M. J. Hynes. 1985. Transformation of Aspergillus niger by the amdS gene of Aspergillus nidulans. Embo J. 4:475-9.
Kostelny, S. A., M. S. Cole, and J. Y. Tso. 1992. Formation of a bispecific antibody by the use of leucine zippers. J Immunol. 148:1547-53.
WO94/16101. DNA SEQUENCING BY MASS SPECTROMETRY. 1994.
Kozal, M. J., N. Shah, N. Shen, R. Yang, et al. 1996. Extensive polymorphisms observed in HIV-1 clade B protease gene using high-density oligonucleotide arrays. Nat Med. 2:753-9.
Kozbor, D., P. Tripputi, J. C. Roder, and C. M. Croce. 1984. A human hybrid myeloma for production of human monoclonal antibodies. J Immunol. 133:3001-5.
Kriegler, M. 1990. Gene transfer and expression: A laboratory manual. Stockton Press, New York. 242 pp.
WO 91/01140. HOMOLOGOUS RECOMBINATION FOR UNIVERSAL DONOR CELLS AND CHIMERIC MAMMALIAN HOSTS. 1991.
Kwoh, D. Y., G. R. Davis, K. M. Whitfield, H. L. Chappelle, et al. 1989. Transcription-based amplification system and detection of amplified human immunodeficiency virus type 1 with a bead-based sandwich hybridization format. Proc Natl Acad Sci USA. 86:1173-7.
U.S. Pat. No. 5,223,409. Directed evolution of novel binding proteins. 1993.
Lakso, M., B. Sauer, B. Mosinger, E. J. Lee, et al. 1992. Targeted oncogene activation by site-specific recombination in transgenic mice. Proc Natl Acad Sci USA. 89:6232-6.
Lam, K. S. 1997. Application of combinatorial library methods in cancer research and drug discovery. Anticancer Drug Design. 12:145-167.
Lam, K. S., S. E. Salmon, E. M. Hersh, V. J. Hruby, et al. 1991. General method for rapid synthesis of multicomponent peptide mixtures. Nature. 354:82-84.
Landegren, U., R. Kaiser, J. Sanders, and L. Hood. 1988. A ligase-mediated gene detection technique. Science. 241:1077-80.
WO 90/11354. Process for the specific replacement of a copy of a gene present in the receiver genome via the integration of a gene. 1990.
Leahy, K. P., K. J. Davies, M. Dull, J. J. Kort, et al. 1999. adapt78, a stress-inducible mRNA, is related to the glucose-regulated protein family of genes. Arch Biochem Biophys. 368:67-74.
U.S. Pat. No. 4,736,866. Transgenic non-human animals. 1988.
Leduc, N., and e. al. 1996. Isolated maize zygotes mimic in vivo embryogenic development and express microinjected genes when cultured in vitro. Dev. Biol. 10:190-203.
Lee, J. S., D. A. Johnson, and A. R. Morgan. 1979. Complexes formed by (pyrimidine)n (purine)n DNAs on lowering the pH are three-stranded. Nucleic Acids Res. 6:3073-91.
Lee, V. H. L. 1990. Peptide and protein drug delivery. Marcel Dekker, New York N.Y.
Lemaitre, M., B. Bayard, and B. Lebleu. 1987. Specific antiviral activity of a poly(L-lysine)-conjugated oligodeoxyribonucleotide sequence complementary to vesicular stomatitis virus N protein mRNA initiation site. Proc Natl Acad Sci USA. 84:648-52.
Lemischka, I. R., D. H. Raulet, and R. C. Mulligan. 1986. Developmental potential and dynamic behavior of hematopoietic stem cells. Cell. 45:917-927.
Letsinger, R. L., G. R. Zhang, D. K. Sun, T. Ikeuchi et al. 1989. Cholesteryl-conjugated oligonucleotides: synthesis, properties, and activity as inhibitors of replication of human immunodeficiency virus in cell culture. Proc Natl Acad Sci USA. 86:6553-6.
Li, E., T. H. Bestor, and R. Jaenisch. 1992. Targeted mutation of the DNA methyltransferase gene results in embryonic lethality. Cell. 69:915-26.
Li, J. M., J. S. Han, Y. Huang, P. K. Tain, et al. 1999. A novel gene delivery system targeting cells expressing VEGF receptors. Cell Res. 9:11-25.
Linder, M. W., R. A. Prough, and R. Valdes. 1997. Pharmacogenetics: a laboratory tool for optimizing therapeutic efficiency. Clin Chem. 43:254-66.
Lipshutz, R. J., S. P. Fodor, T. R. Gingeras, and D. J. Lockhart. 1999. High density synthetic oligonucleotide arrays. Nat Genet. 21:20-4.
Littlefield, J. W. 1964. Selection of hybrids from matings of fibroblasts in vitro and their presumed recombinants. Science. 145:709-710.
Lizardi, P. M., C. E. Guerra, H. Lomeli, I. Tussie-Luna, et al. 1988. Exponential amplification of recombinant-RNA hybridization probes. Biotechnology. 6:1197-1202.
Lohi, J., I. Leivo, J. Oivula, V. P. Lehto, et al. 1998. Extracellular matrix in renal cell carcinomas. Histol Histopathol. 13:785-96.
Lonberg, N., and D. Huszar. 1995. Human antibodies from transgenic mice. Int Rev Immunol. 13:65-93.
Lonberg, N., L. D. Taylor, F. A. Harding, M. Trounstine, et al. 1994. Antigen-specific human antibodies from mice comprising four distinct genetic modifications [see comments]. Nature. 368:856-9.
Lopata, M. A., D. W. Cleveland, and B. Sollner-Webb. 1984. High-level expression of a chloramphenicol acetyltransferase gene by DEAEdextran-mediated DNA transfection couled with a dimethylsulfoxide or glycerol shock treatment. Nucleic Acids Research. 12:5707.
Luckow, V. A. 1991. Cloning and expression of heterologous genes in insect cells with baculovirus vectors. In Recombinant DNA technology and applications. A. Prokop, R. K. Bajpai, and C. Ho, editors. McGraw-Hill, New York. 97-152.
Madura, K., R. J. Dohmen, and A. Varshavsky. 1993. N-recognin/Ubc2 interactions in the N-end rule pathway. J Biol Chem. 268:12046-54.
Maher, L. J. 1992. DNA triple-helix formation: an approach to artificial gene repressors? Bioessays. 14:807-15.
Majewski, M., M. Nieborowska-Skorska, P. Salomoni, A. Slupianek, et al. 1999. Activation of mitochondrial Raf-1 is involved in the antiapoptotic effects of Akt. Cancer Res. 59:2815-9.
Mak, I. T., P. Boehme, and W. B. Weglicki. 1995. Protective effects of calcium channel blockers against free radical-impaired endothelial cell proliferation. Biochem Pharmacol. 50:1531-4.
Mandel, M., and A. Higa. 1970. Calcium-dependent bacteriophage DNA infection. J. Mol. biol. 53:159-162.
Marasco, W. A., W. A. Haseltine, and S. Y. Chen. 1993. Design, intracellular expression, and activity of a human anti-human immunodeficiency virus type 1 gp120 single-chain antibody. Proc Natl Acad Sci USA. 90:7889-93.
Marks, J. D., A. D. Griffiths, M. Malmqvist, T. P. Clackson, et al. 1992. By-passing immunization: building high affinity human antibodies by chain shuffling. Biotechnology (NY). 10:779-83.
Marks, J. D., H. R. Hoogenboom, T. P. Bonnert, J. McCafferty, et al. 1991. By-passing immunization. Human antibodies from V-gene libraries displayed on phage. J Mol Biol. 222:581-97.
Martin, F. J., and D. Papahadjopoulos, 1982. Irreversible coupling of immunoglobulin fragments to preformed vesicles. An improved method for liposome targeting. J Biol Chem. 257:286-8.
Marzo, I., C. Brenner, and G. Kroemer. 1998. The central role of the mitochondrial megachannel in apoptosis: evidence obtained with intact cells, isolated mitochondria, and purified protein complexes. Biomed Pharmacother. 52:248-51.
Masood, R., J. Cai, T. Zheng, D. L. Smith, et al. 1997. Vascular endothelial growth factor/vascular permeability factor is an autocrine growth factor for AIDS-Kaposi sarcoma. Proc Natl Acad Sci USA. 94:979-84.
Maxam, A. M., and W. Gilbert. 1977. A new method for sequencing DNA. Proc Natl Acad Sci USA. 74:560-4.
Migdal, M., B. Huppertz, S. Tessler, A. Comforti, et al. 1998. Neuropilin-1 is a placenta growth factor-2 receptor. J Biol Chem. 273:22272-8.
Miller, A. D., and C. Buttimore. 1986. Redesign of retrovirus packaging cell lines to avoid recombination leading to helper virus production. Mol. Cell biol. 6:2895-2902.
Miller, L. K. 1988. Baculoviruses as gene expression vectors. Annu. Rev. Microbiol. 42:177-199.
Milstein, C., and A. C. Cuello. 1983. Hybrid hybridomas and their use in immunohistochemistry. Nature. 305:537-40.
U.S. Pat. No. 5,459,039. Methods for mapping genetic mutations. 1995.
EP 233838. NEURITE-PROMOTING FACTOR AND PROCESS FOR THE MANUFACTURE THEREOF. 1990.
Morita, T., M. Yoshizumi, H. Kurihara, K. Maemura, et al. 1993. Shear stress increases heparin-binding epidermal growth factor-like growth factor mRNA levels in human vascular endothelial cells. Biochem Biophys Res Commun. 197:256-62.
Morrison, S. L., L. Wims, S. Wallick, L. Tan, et al. 1987. Genetically engineered antibody molecules and their application. Ann NY Acad Sci. 507:187-98.
U.S. Pat. No. 4,683,202. Process for amplifying nucleic acid sequences. 1987.
U.S. Pat. No. 4,683,195. Process for amplifying, detecting, and/or cloning nucleic acid sequences. 1987.
Munson, P. J., and D. Rodbard. 1980. Ligand: a versatile computerized approach for characterization of ligand-binding systems. Anal Biochem. 107:220-39.
Myers, R. M., Z. Larin, and T. Maniatis. 1985. Detection of single base substitutions by ribonuclease cleavage at mismatches in RNA:DNA duplexes. Science. 230:1242-6.
U.S. Pat. No. 5,328,470. Treatment of diseases by site-specific instillation of cells or site-specific transformation of cells and kits therefor. 1994.
Naeve, C. W., G. A. Buck, R. L. Niece, R. T. Pon, et al. 1995. Accuracy of automated DNA sequencing: a multi-laboratory comparison of sequencing results. Biotechniques. 19:448-53.
Nakai, K., and P. Horton. 1999. PSORT: a program for detecting sorting signals in proteins and predicting their subcellular localization. Trends Biochem Sci. 24:34-6.
Nakazawa, H., D. English, P. L. Randell, K. Nakazawa, et al. 1994. UV and skin cancer: specific p53 gene mutation in normal skin as a biologically relevant exposure measurement. Proc Natl Acad Sci USA. 91:360-4.
Nauert, J. B., T. M. Klauck, L. K. Langeberg, and J. D. Scott. 1997. Gravin, an autoantigen recognized by serum from myasthenia gravis patients, is a kinase scaffold protein. Curr Biol. 7:52-62.
Neumann, E., M. Schaefer-Ridder, Y. Wang, and P. H. Hofschneider. 1982. Gene transfer into mouse lyoma cells by electroporation in high electric fields. EMBO J. 1:841-845.
Nicosia, R. F., E. Bonanno, M. Smith, and P. Yurchenco. 1994. Modulation of angiogenesis in vitro by laminin-entactin complex. Dev Biol. 164:197-206.
O'Gorman, S., D. T. Fox, and G. M. Wahl. 1991. Recombinase-mediated gene activation and site-specific integration in mammalian cells. Science. 251:1351-5.
Oivula, J., J. Lohi, T. Tani, L. Kangas, et al. 1999. Renal cell carcinomas and pancreatic adenocarcinomas produce nidogen in vitro and in vivo. J Pathol. 187:455-61.
Okano, H., J. Aruga, T. Nakagawa, C. Shiota, et al. 1991. Myelin basic protein gene and the function of antisense RNA in its repression in myelin-deficient mutant mouse. J Neurochem. 56:560-7.
O'Reilly, D. R., L. K. Miller, and V. A. Luckow. 1992. Baculovirus expression vectors. W.H. Freeman and Company, New York.
Orita, M., H. Iwahana, H. Kanazawa, K. Hayashi, et al. 1989. Detection of polymorphisms of human DNA by gel electrophoresis as single-strand conformation polymorphisms. Proc Natl Acad Sci USA. 86:2766-70.
Ou-Lee, T. M., R. Turgeon, and R. Wu. 1986. Uptake and expression of a foreign gene linked to either a plant virus or Drosophila promoter in protoplasts of rice, wheat and sorghum. Proc. Natl. Acad. Sci. USA. 83:6815-6819.
Palmer, T. D., R. A. Hock, W. R. A. osborne, and A. D. Miller. 1987. Efficient retrovirus-mediated transfer and expression of a human adenosine deaminase gene in diploid skin fibroblasts from an adenosie-deficient human. Proc. Natl. Acad. Sci. USA. 84:1055-1059.
Pear, W., G. Nolan, M. Scott, and D. Baltimore. 1993. Production of high-titer helper-free retroviruses by transient transfection. Proc. Natl. Acad. Sci. USA. 90:8392-8396.
Peng, Y., E. J. Schwarz, M. A. Lazar, A. Genin, et al. 1997. Cloning, human chromosomal assignment, and adipose and hepatic expression of the CL-6/INSIG1 gene. Genomics. 43:278-84.
Perry-O'Keefe, H., X. W. Yao, J. M. Coull, M. Fuchs, et al. 1996. Peptide nucleic acid pre-gel hybridization: an alternative to southern hybridization. Proc Natl Acad Sci USA. 93:14670-5.
Petersen, K. H., D. K. Jensen, M. Egholm, O. Buchardt, et al. 1976. A PNA-DNA linker synthesis of N-((4,4′-dimethoxytrityloxy)ethyl)-N-(thymin-1-ylacetyl)glycine. Biorganic and Medicianl Chemistry Letters. 5:1119-1124.
Potter, H. 1988. Electroporation in biology: Methods, applications, and instrumentation. Analytical Biochemistry. 174:361-373.
Potter, H., L. Weir, and P. Leder. 1984. Enhancer-dependent expression of human kappa immunoglobulin genes introduced into mouse pre-B lymphocytes by electroporation. Proc. Natl. Acad. Sci. USA. 81:7161-7165.
Presta, L. G. 1992. Antibody engineering. Curr Opin Biotechnol. 3:394-8.
Prosser, J. 1993. Detecting single-base mutations. Trends Biotechnol. 11:238-46.
Radisky, D. C., W. B. Snyder, S. D. Emr, and J. Kaplan. 1997. Characterization of VPS41, a gene required for vacuolar trafficking and high-affinity iron transport in yeast. Proc Natl Acad Sci USA. 94:5662-6.
Rassoulzadegan, M., B. Binetruy, and F. Cuzin. 1982. High frequency of gene transfer after fusion between bacteria and eukaryotic cells. Nature. 295:257.
Reisfeld, R. A., and S. Sell. 1985. Monoclonal antibodies and cancer therapy: Proceedings of the Roche-UCLA symposium held in Park City, Utah, Jan. 26-Feb. 2, 1985. Alan R. Liss, New York. 609 pp.
Rhodes, C. A., D. A. Pierce, I. J. Mettler, D. Mascarenhas, et al. 1988. Genetically transformed maize plants from protoplasts. Science. 240:204-207.
Riechmann, L., M. Clark, H. Waldmann, and G. Winter. 1988. Reshaping human antibodies for therapy. Nature. 332:323-7.
Risau, W. 1995. Differentiation of endothelium. Faseb J. 9:926-33.
Risau, W., and I. Flamme. 1995. Vasculogenesis. Annu Rev Cell Dev Biol. 11:73-91.
Rose, J. K., L. Buonocore, and M. Whitt. 1991. A new cationic liposome reagent mediating nearly quantitative transfection of animal cells. BioTechniques. 10:520-525.
Rossi, J. J. 1994. Practical ribozymes. Making ribozymes work in cells. Curr Biol. 4:469-71.
Rossiter, B. J., and C. T. Caskey. 1990. Molecular scanning methods of mutation detection. J Biol Chem. 265:12753-6.
U.S. Pat. No. 5,871,697. Method and apparatus for identifying, classifying, or quantifying DNA sequences in a sample without sequencing. 1999.
U.S. Pat. No. 5,283,317. Intermediates for conjugation of polypeptides with high molecular weight polyalkylene glycols. 1994.
Saiki, R. K., T. L. Bugawan, G. T. Horn, K. B. Mullis, et al. 1986. Analysis of enzymatically amplified β-globin and HLA-DQ α DNA with allele-specific oligonucleotide probes. Nature. 324:163-6.
Saiki, R. K., P. S. Walsh, C. H. Levenson, and H. A. Erlich. 1989. Genetic analysis of amplified DNA with immobilized sequence-specific oligonucleotide probes. Proc Natl Acad Sci USA. 86:6230-4.
Saleeba, J. A., and R. G. Cotton. 1993. Chemical cleavage of mismatch to detect mutations. Methods Enzymol. 217:286-95.
Sambrook, J. 1989. Molecular cloning: a laboratory manual. Cold Spring Harbor Laboratory, Cold Spring Harbor.
Sandri-Goldin, R. M., A. L. Goldin, J. C. Glorioso, and M. Levine. 1981. High-frequency transfer of cloned herpes simjplex virus type I sequences to mammalian cells by protoplast fusion. Mol. Cell. Biol. 1:7453-752.
Sanger, F., S. Nicklen, and A. R. Coulson. 1977. DNA sequencing with chain-terminating inhibitors. Proc Natl Acad Sci USA. 74:5463-7.
Sato, N., K. Kokame, K. Shimokado, H. Kato, et al. 1998. Changes of gene expression by lysophosphatidylcholine in vascular endothelial cells: 12 up-regulated distinct genes including 5 cell growth-related, 3 thrombosis-related, and 4 others. J Biochem (Tokyo). 123:1119-26.
Saunders, J. A., B. F. Matthews, and P. D. Miller. 1989. Plant gene transfer using electrofusion and electroporation. In Electroporation and electrofusion in cell biology. E. Neumann, A. E. Sowers, and C. A. Jordan, editors. Plenum Press, New York. 343-354.
Schade, R., C. Staak, C. Hendriksen, M. Erhard, et al. 1996. The production of avian (egg yold) antibodies: IgY. The report and recommendations of ECVAM workshop. Alternatives to Laboratory Animals (ATLA). 24:925-934.
Schaffner, W. 1980. Direct transfer of cloned genes from bacteria to mammalian cells. Proc. Natl. Acad. Sci. USA. 77:2163.
Schook, L. B. 1987. Monoclonal antibody production techniques and applications. Marcel Dekker, Inc., New York. 336 pp.
Scimeca, J. C., M. J. Servant, J. O. Dyer, and S. Meloche. 1997. Essential role of calcium in the regulation of MAP kinase phosphatase-1 expression. Oncogene. 15:717-25.
Scott, J. K., and G. P. Smith. 1990. Searching for peptide ligands with an epitope library. Science. 249:386-90.
Selden, R. F., K. Burke-Howie, M. E. Rowe, H. M. Goodman, et al. 1986. Human growth hormone as a reporter gene in regulation studies employing transient gene expression. Molecular and Cellular Biology. 6:3173-3179.
Shalaby, F., J. Rossant, T. P. Yamaguchi, M. Gertsenstein, et al. 1995. Failure of blood-island formation and vasculogenesis in Flk-1-deficient mice. Nature. 376:62-6.
Shalaby, M. R., H. M. Shepard, L. Presta, M. L. Rodrigues, et al. 1992. Development of humanized bispecific antibodies reactive with cytotoxic lymphocytes and tumor cells overexpressing the HER2 protooncogene. J Exp Med. 175:217-25.
Shigekawa, K., and W. J. Dower. 1988. Electroporation of eukaryotes and prokaryotes: A general approach to the introduction of macomolecules into cells. BioTechniques. 6:742-751.
Shih, M., F. Lin, J. D. Scott, H. Y. Wang, et al. 1999. Dynamic complexes of β2-adrenergic receptors with protein kinases and phosphatases and the role of gravin. J Biol Chem. 274:1588-95.
Shillito, R. 1999. Methods of genetic transformations: Electroporation and polyethylene glycol treatment. In Molecular improvement of cereal crop. I. Vasil, editor. Kluwer, Dordrecht, The Netherlands. 9-20.
Shilo, B. Z., and R. A. Weinberg. 1981. DNA sequences homologous to vertebrate oncogenes are conserved in Drosophila melanogaster. Proc Natl Acad Sci USA. 78:6789-92.
Shimkets, R. A., D. G. Lowe, J. T. Tai, P. Sehl, et al. 1999. Gene expression analysis by transcript profiling coupled to a gene database query. Nat Biotechnol. 17:798-803.
Shopes, B. 1992. A genetically engineered human IgG mutant with enhanced cytolytic activity. J Immunol. 148:2918-22.
Shridhar, R., V. Shridhar, S. Rivard, J. M. Siegfried, et al. 1996a. Mutations in the arginine-rich protein gene, in lung, breast, and prostate cancers, and in squamous cell carcinoma of the head and neck. Cancer Res. 56:5576-8.
Shridhar, V., S. Rivard, R. Shridhar, C. Mullins, et al. 1996b. A gene from human chromosomal band 3p21.1 encodes a highly conserved arginine-rich protein and is mutated in renal cell carcinomas. Oncogene. 12:1931-9.
Shridhar, V., S. Rivard, X. Wang, R. Shridhar, et al. 1997. Mutations in the arginine-rich protein gene (ARP) in pancreatic cancer. Oncogene. 14:2213-6.
Simonsen, C. C., and A. D. Levinson. 1983. Isolation and expression of an altered mouse dihydrofolate reductase cDNA. Proc. Natl. Acad Sci. USA. 80:2495-2499.
Singer-Kruger, B., and S. Ferro-Novick. 1997. Use of a synthetic lethal screen to identify yeast mutants impaired in endocytosis, vacuolar protein sorting and the organization of the cytoskeleton. Eur J Cell Biol. 74:365-75.
Smith-Swintosky, V. L., S. Zimmer, J. W. Fenton, and M. P. Mattson. 1995. Protease nexin-1 and thrombin modulate neuronal Ca2+ homeostasis and sensitivity to glucose deprivation-induced injury. J Neurosci. 15:5840-50.
U.S. Pat. No. 5,272,057. Method of detecting a predisposition to cancer by the use of restriction fragment length polymorphism of the gene for human poly (ADP-ribose) polymerase. 1993.
Southern, P. J., and P. Berg. 1982. Transformation of mammalian cells to antibiotic resistanced with a bacterial gene under control of the SV40 early region promoter. J. Mol. Appl. Gen. 1:327-341.
Speirs, V., and S. L. Atkin. 1999. Production of VEGF and expression of the VEGF receptors Flt-1 and KDR in primary cultures of epithelial and stromal cells derived from breast tumours. Br J Cancer. 80:898-903.
Sreekrishna, K., R. H. Potenz, J. A. Cruze, W. R. McCombie, et al. 1988. High level expression of heterologous proteins in methylotrophic yeast Pichia pastoris. J Basic Microbiol. 28:265-78.
Stein, C. A., and J. S. Cohen. 1988. Oligodeoxynucleotides as inhibitors of gene expression: a review. Cancer Res. 48:2659-68.
Stevenson, G. T., A. Pindar, and C. J. Slade. 1989. A chimeric antibody with dual Fc regions (bisFabFc) prepared by manipulations at the IgG hinge. Anticancer Drug Des. 3:219-30.
Suresh, M. R., A. C. Cuello, and C. Milstein. 1986. Bispecific monoclonal antibodies from hybrid hybridomas. Methods Enzymol. 121:210-28.
Takemura, T., S. Hino, Y. Murata, H. Yanagida, et al. 1999. Coexpression of CD9 augments the ability of membrane-bound heparin-binding epidermal growth factor-like growth factor (proHB-EGF) to preserve renal epithelial cell viability. Kidney Int. 55:71-81.
Takemura, T., S. Kondo, T. Homma, M. Sakai et al. 1997. The membrane-bound form of heparin-binding epidermal growth factor-like growth factor promotes survival of cultured renal epithelial cells. J Biol Chem. 272:31036-42.
Thomas, K. R., and M. R. Capecchi. 1987. Site-directed mutagenesis by gene targeting in mouse embryo-derived stem cells. Cell. 51:503-12.
Thompson, J. A., and e. al. 1995. Maize transformation utilizing silicon carbide whiskers: A review. Euphytica. 85:75-80.
Tilburn, J., C. Scazzocchio, G. G. Taylor, J. H. Zabicky-Zissman, et al. 1983. Transformation by integration in Aspergillus nidulans. Gene. 26:205-21.
Touraev, A., and e. al. 1997. Plant male germ line transformation. Plant J. 12:949-956.
Traunecker, A., F. Oliveri, and K. Karjalainen. 1991. Myeloma based expression system for production of large mammalian proteins. Trends Biotechnol. 9:109-13.
Trick, H. N., and e. al. 1997. Recent advances in soybean transformation. Plant Tissue Cult. Biotechnol. 3:9-26.
Tuerk, C., and L. Gold. 1990. Systematic evolution of ligands by exponential enrichment: RNA ligands to bacteriophage T4 DNA polymerase. Science. 249:505-10.
Turner, D. L., E. Y. Snyder, and C. L. Cepko. 1990. Lineage-independent determination of cell type in the embryonic mouse retina. Neuron. 4:833-845.
Tutt, A., G. T. Stevenson, and M. J. Glennie. 1991. Trispecific F(ab′)3 derivatives that use cooperative signaling via the TCR/CD3 complex and CD2 to activate and redirect resting cytotoxic T cells. J Immunol. 147:60-9.
Van Belle, E., L. Maillard, F. O. Tio, and J. M. Isner. 1997. Accelerated endothelialization by local delivery of recombinant human vascular endothelial growth factor reduces in-stent intimal formation. Biochem Biophys Res Commun. 235:311-6.
van der Krol, A. R., J. N. Mol, and A. R. Stuitje. 1988a. Modulation of eukaryotic gene expression by complementary RNA or DNA sequences. Biotechniques. 6:958-76.
van der Krol, A. R., J. N. Mol, and A. R. Stuitje. 1988b. Modulation of eukaryotic gene expression by complementary RNA or DNA sequences. Biotechniques. 6:958-76.
Verhoeyen, M., C. Milstein, and G. Winter. 1988. Reshaping human antibodies: grafting an antilysozyme activity. Science. 239:1534-6.
Vinante, F., A. Rigo, E. Papini, M. A. Cassatella, et al. 1999. Heparin-binding epidermal growth factor-like growth factor/diphtheria toxin receptor expression by acute myeloid leukemia cells. Blood. 93:1715-23.
Vitetta, E. S., R. J. Fulton, R. D. May, M. Till, et al. 1987. Redesigning nature's poisons to create anti-tumor reagents. Science. 238:1098-104.
U.S. Pat. No. 4,873,191. Genetic transformation of zygotes. 1989.
Warren, R. S., H. Yuan, M. R. Matli, N. A. Gillett, et al. 1995. Regulation by vascular endothelial growth factor of human colon cancer tumorigenesis in a mouse model of experimental liver metastasis. J Clin Invest. 95:1789-97.
Wells, J. A, M. Vasser, and D. B. Powers. 1985. Cassette mutagenesis: an efficient method for generation of multiple mutations at defined sites. Gene. 34:315-23.
Whitt, M. A., L. Buonocore, J. K. Rose, V. Ciccarone, et al. 1990. TransfectACE reagent promotes transient transfection frequencies greater than 90%. Focus. 13:8-12.
Wigler, M., A. Pellicer, S. Silversttein, and R. Axel. 1978. Biochemical transfer of single-copy eucaryotic genes using total cellular DNA as donor. Cell. 14:725.
Wilkinson, M. G., and J. B. Millar. 2000. Control of the eukaryotic cell cycle by MAP kinase signaling pathways. Faseb J. 14:2147-57.
Williams, D. A., I. R. Lemischka, D. G. Nathan, and R. C. Mulligan. 1984. Introduction of a new genetic material into pluripotent haematopoietic stem cells of the mouse. Nature. 310:476-480.
Wilmut, I., A. E. Schnieke, J. McWhir, A. J. Kind, et al. 1997. Viable offspring derived from fetal and adult mammalian cells. Nature. 385:810-3.
Winter, C., J. Schenkel, M. Zimmermann, and T. Herdegen. 1998. MAP kinase phosphatase 1 is expressed and enhanced by FK506 in surviving mamillary, but not degenerating nigral neurons following axotomy. Brain Res. 801:198-205.
Wolff, E. A., G. J. Schreiber, W. L. Cosand, and H. V. Raff. 1993. Monoclonal antibody homodimers: enhanced antitumor activity in nude mice. Cancer Res. 53:2560-5.
Wong, T. K., and E. Neumann. 1982. Electric field mediated gene transfer. Biochemical and Biophysical Research Communications. 107:584-587.
Wyborski, D. L., L. C. DuCoeur, and J. M. Short. 1996. Parameters affecting the use of the lac repressor system in eukaryotic cells and transgenic animals. Environ Mol Mutagen. 28:447-58.
Wyborski, D. L., and J. M. Short. 1991. Analysis of inducers of the E. coli lac repressor system in mammalian cells and whole animals. Nucleic Acids Res. 19:4647-53.
Xu, X., D. Yang, T. Wyss-Coray, J. Yan, et al. 1999. Wild-type but not Alzheimer-mutant amyloid precursor protein confers resistance against p53-mediated apoptosis. Proc Natl Acad Sci USA. 96:7547-52.
Yang, S., J. Graham, J. W. Kahn, E. A. Schwartz, et al. 1999. Functional roles for PECAM-1 (CD31) and VE-cadherin (CD144) in tube assembly and lumen formation in three-dimensional collagen gels. Am J Pathol. 155:887-95.
Yang, Y., L. Martin, F. Cuzin, M. G. Mattei et al. 1996. Genomic structure and chromosomal localization of the mouse CDEI-binding protein CDEBP (APLP2) gene and promoter sequences. Genomics. 35:24-9.
Yelton, M. M., J. E. Hamer, and W. E. Timberlake. 1984. Transformation of Aspergillus nidulans by using a trpC plasmid. Proc Natl Acad Sci USA. 81:1470-4.
Yuan, F., Y. Chen, M. Dellian, N. Safabakhsh, et al. 1996. Time-dependent vascular regression and permeability changes in established human tumor xenografts induced by an anti-vascular endothelial growth factor/vascular permeability factor antibody. Proc Natl Acad Sci USA. 93:14765-70.
Zervos, A. S., J. Gyuris, and R. Brent. 1993. Mxi1, a protein that specifically interacts with Max to bind Myc-Max recognition sites. Cell. 72:223-32.
Zhou, G., and e. al. 1983. Introduction of exogenous DNA into cotton embryos. Methods Enzymol. 101:433-481.
Zoller, M. J., and M. Smith. 1987. Oligonucleotide-directed mutagenesis: a simple method using two oligonucleotide primers and a single-stranded DNA template. Methods Enzymol. 154:329-50.
Zon, G. 1988. Oligonucleotide analogues as potential chemotherapeutic agents. Pharm Res. 5:539-49.
Zuckermann, R. N., E. J. Martin, D. C. Spellmeyer, G. B. Stauber, et al. 1994. Discovery of nanomolar ligands for 7-transmembrane G-protein-coupled receptors from a diverse N-(substituted)glycine peptoid library. J Med Chem. 37:2678-85.

1-31. (canceled)

32. A method of screening a tissue sample for tumorigenic potential, comprising:
measuring expression of at least one VEGF-modulated gene in said tissue sample.

33. The method of claim 32, wherein said measuring is measuring an amount of a polypeptide encoded by said at least one VEGF-modulated gene.

34. The method of claim 32, wherein said measuring expression is measuring an amount of mRNA corresponding to said at least one VEGF-modulated gene.

35-55. (canceled)

56. A method of determining the clinical stage of tumor comprising comparing expression of at least one VEGF-modulated gene in a sample with expression of said at least one gene in control samples.

57. The method of claim 56, wherein said at least one VEGF-modulated gene comprises at least one member selected from the group consisting of DSCR1 and ARP.

58. The method of claim 56, wherein said sample is a sample from an ovarian tumor.

59-61. (canceled)

62. A method for detecting a disease or disorder associated with a change in expression of at least one VEGF-modulated gene in a subject, wherein said at least one gene encodes a polypeptide is selected from the group consisting of nexin, placental protein 5 (PP5), amyloid precursor-like protein 2 (APLP2), regulator of G-protein signaling-3 (RGS3), gravin, arginine-rich protein (ARP), Down's syndrome critical region protein-1 (DSCR1), insulin induced gene-1 (INSIG1), decidual protein induced by progesterone (DEPP), NADH-ubiquinone oxidoreductase chain 1 (ND1), MKP-1 like protein tyrosine phosphatase, osteonidogen, amyloid precursor protein (APP), human gene similar to yeast VPS41 (hVPS41p), cytochrome oxidase subunit I (MTCO1), and NADH-ubiquinone oxidoreductase chain 4 (ND4), comprising:
a) contacting a biological sample from the subject with an agent capable of detecting the presence of said at least one VEGF-modulated gene or polypeptide; and
b) determining the amount of said at least one VEGF-modulated gene or polypeptide in the biological sample, wherein an increase or decrease in the amount of the gene or polypeptide is indicative of a disease or disorder associated with a change in expression of said at least one VEGF-modulated gene or polypeptide.

63. The method of claim 62, wherein the agent is a labeled probe that can hybridize to said at least one VEGF-modulated gene or mRNA encoding said at least one VEGF-modulated gene.

64. The method of claim 62, wherein the agent is an antibody or antibody fragment that is capable of binding to said at least one VEGF-modulated gene polypeptide.

65. The method of claim 62, wherein the disease or disorder is cancer or tumor.

66. The method of claim 65, wherein the cancer or tumor is selected from the group consisting of pulmonary adenocarcinoma, ductal mammary carcinoma, renal cell carcinoma, hepatocellular carcinoma, squamous cell carcinoma, osteosarcoma, chrondosarcoma, and ovarian carcinoma.

67. The method of claim 65, wherein the cancer is ovarian cancer.

68. The method of claim 67, wherein said at least one VEGF-modulated gene or polypeptide comprises DSCR-1, ARP, or a combination thereof.

69. A method for identifying an agent that is a modulator of at least one polypeptide encoded by a VEGF modulated gene, comprising:
(a) contacting isolated cells or tissue expressing a polypeptide selected from the group consisting of nexin, PP5, APLP2, RGS3, gravin, ARP, DSCR1, INSIG1, DEPP, ND1, HB-EGF, MKP-1 like protein tyrosine phosphatase, osteonidogen and CTGF with the agent; and
(b) detecting a change in angiogenic activity of the contacted cells or tissue, wherein an agent that decreases said angiogenic activity is identified as a modulator.

70. The method of claim 69, wherein the agent is selected from the group consisting of a small molecule, a small peptide, an antibody or derivative thereof, a polypeptide closely related to a polypeptide encoded by a VEGF modulated gene, antisense DNA or RNA, ribozymes, triple DNA helices, and nucleic acid aptamers.

71. A method for identifying an agent that is an agonist of at least one polypeptide encoded by a VEGF modulated gene, comprising:
(a) contacting isolated cells or tissue expressing a polypeptide selected from the group consisting of nexin, PP5, APLP2, RGS3, gravin, ARP, DSCR1, INSIG1, DEPP, ND1, HB-EGF, MKP-1 like protein tyrosine phosphatase, osteonidogen and CTGF with the agent; and
(b) detecting a change in angiogenic activity of the contacted cells or tissue, wherein an agent that increases said angiogenic activity is identified as an agonist.

72. A method for identifying an agent that is a modulator of at least one polypeptide encoded by a VEGF modulated gene, comprising:
(a) contacting isolated cells or tissue expressing a polypeptide selected from the group consisting of APP, hVPS41p, MTCO1 and ND4 with the agent; and
(b) detecting a change in angiogenic activity of the contacted cells or tissue, wherein an agent that increases said angiogenic activity is identified as a modulator.

73. The method of claim 72, wherein the agent is selected from the group consisting of a small molecule, a small peptide, an antibody or derivative thereof, a polypeptide closely related to a polypeptide encoded by a VEGF modulated gene, antisense DNA or RNA, ribozymes, triple DNA helices, and nucleic acid aptamers.

74. A method for identifying an agent that is an agonist of at least one polypeptide encoded by a VEGF modulated gene, comprising:
(a) contacting isolated cells or tissue expressing a polypeptide selected from the group consisting of APP, hVPS41p, MTCO1 and ND4 with the agent; and
(b) detecting a change in angiogenic activity of the contacted cells or tissue, wherein an agent that decreases said angiogenic activity is identified as an agonist.

75. A method of determining the effectiveness of a treatment for angiogenesis, comprising detecting expression or activity of a polypeptide selected from the group consisting of nexin, PP5, APLP2, RGS3, gravin, ARP, DSCR1, INSIG1, DEPP, ND1, HB-EGF, MKP-1 like protein tyrosine phosphatase, osteonidogen and CTGF in a cell or tissue sample from a subject undergoing said treatment, wherein a decrease in expression or activity of the polypeptide indicates a decrease in angiogenesis.

76. A method of determining the effectiveness of a treatment for angiogenesis, comprising detecting expression or activity of a polypeptide selected from the group consisting of APP, hVPS41p, MTCO1 and ND4 in a cell or tissue sample from a subject undergoing said treatment, wherein an increase in expression or activity of the polypeptide indicates a decrease in angiogenesis.
